SEQUENCE LISTING (343 sequences)

SEQ ID NO: 1 - protein, 133 aa, Mus musculus
MAQMMTLSLLSLVLALCIPWTQGSDGGGQDCCLKYSQKKIPYSIVRGYRKQEPSLGCPIPAILFLPRKHSKPELCANPEEGWVQNLMRRLDQPPAPGKQSPGCRKNRGTSKSGKKGKGSKGCKRTEQTQPSRG

SEQ ID NO: 2 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 3 - protein, 303 aa, Mus musculus
MWPLAAALLLGSCCCGSAQLLFSNVNSIEFTSCNETVVIPCIVRNVEAQSTEEMFVKWKLNKSYIFIYDGNKNSTTTDQNFTSAKISVSDLINGIASLKMDKRDAMVGNYTCEVTELSREGKTVIELKNRTVSWFSPNEKILIVIFPILAILLFWGKFGILTLKYKSSHTNKRIILLLVAGLVLTVIVVVGAILLIPGEKPVKNASGLGLIVISTGILILLQYNVFMTAFGMTSFTIAILITQVLGYVLALVGLCLCIMACEPVHGPLLISGLGIIALAELLGLVYMKFVASNQRTIQPPRNR

SEQ ID NO: 4 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 5 - protein, 278 aa, Mus musculus
MGSLVFRRPFCHLSTYSLIWGMAAVALSTAQVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYVQPIVHLHYNYFEDHLNITCSATARPAPAISWKGTGTGIENSTESHFHSNGTTSVTSILRVKDPKTQVGKEVICQVLYLGNVIDYKQSLDKGFWFSVPLLLSIVSLVILLVLISILLYWKRHRNQERGESSQGMQRMK

SEQ ID NO: 6 - protein, 278 aa, Homo sapiens
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDRGELSQGVQKMT

SEQ ID NO: 7 - protein, 374 aa, Mus musculus
MNTLSEGNGTFAIHLLKMLCQSNPSKNVCYSPASISSALAMVLLGAKGQTAVQISQALGLNKEEGIHQGFQLLLRKLNKPDRKYSLRVANRLFADKTCEVLQTFKESSLHFYDSEMEQLSFAEEAEVSRQHINTWVSKQTEGKIPELLSGGSVDSETRLVLINALYFKGKWHQPFNKEYTMDMPFKINKDEKRPVQMMCREDTYNLAYVKEVQAQVLVMPYEGMELSLVVLLPDEGVDLSKVENNLTFEKLTAWMEADFMKSTDVEVFLPKFKLQEDYDMESLFQRLGVVDVFQEDKADLSGMSPERNLCVSKFVHQSVVEINEEGTEAAAASAIIEFCCASSVPTFCADHPFLFFIRHNKANSILFCGRFSSP

SEQ ID NO: 8 - protein, 376 aa, Homo sapiens
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 9 - protein, 279 aa, Mus musculus
MQQPMNYPCPQIFWVDSSATSSWTPPGSVFPCPSSGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKELRSVAHLTGNPHSRSIPLEWEDTYGTALISGVKYKKGSLVINEAGLYFVYSKVYFRGQSCNNQPLNHKVYMRNSKYPGDLVLMEEKRLNYCTTGQIWAHSSYLGAVFNLTSADHLYVNISQLSLINFEESKTFFGLYKL

SEQ ID NO: 10 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 11 - protein, 290 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 12 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 13 - protein, 463 aa, Mus musculus
MQVSRVLAALCGMLLCASGLFAASGDFCDSSLCLNGGTCLTGQDNDIYCLCPEGFTGLVCNETERGPCSPNPCYNDAKCLVTLDTQRGDIFTEYICQCPVGYSGIHCETETNYYNLDGEYMFTTAVPNTAVPTPAPTPDLSNNLASRCSTQLGMEGGAIADSQISASSVYMGFMGLQRWGPELARLYRTGIVNAWTASNYDSKPWIQVNLLRKMRVSGVMTQGASRAGRAEYLKTFKVAYSLDGRKFEFIQDESGGDKEFLGNLDNNSLKVNMFNPTLEAQYIKLYPVSCHRGCTLRFELLGCELHGCSEPLGLKNNTIPDSQMSASSSYKTWNLRAFGWYPHLGRLDNQGKINAWTAQSNSAKEWLQVDLGTQRQVTGIITQGARDFGHIQYVASYKVAHSDDGVQWTVYEEQGSSKVFQGNLDNNSHKKNIFEKPFMARYVRVLPVSWHNRITLRLELLGC

SEQ ID NO: 14 - protein, 387 aa, Homo sapiens
MPRPRLLAALCGALLCAPSLLVALDICSKNPCHNGGLCEEISQEVRGDVFPSYTCTCLKGYAGNHCETKCVEPLGMENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 15 - protein, 249 aa, Mus musculus
SIEEIPRMEPRAPWMEKERPEYWKELKLKVKNIAQSARANLRTLLRYYNQSEGGSHILQWMVSCEVGPDMRLLGAHYQAAYDGSDYITLNEDLSSWTAVDMVSQITKSRLESAGTAEYFRAYVEGECLELLHRFLRNGKEILQRADPPKAHVAHHPRPKGDVTLRCWALGFYPADITLTWQKDEEDLTQDMELVETRPSGDGTFQKWAAVVVPSGEEQRYTCYVHHEGLTEPLALKWGRSSQSSVVIMV

SEQ ID NO: 16 - protein, 338 aa, Homo sapiens
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 17 - protein, 439 aa, synthetic construct
  REGION 1..439: note = Synthetic Construct
MGPQAAAGRMILLVVLMLSAKVGSGALTSTEDPEPPSVPVPTNVLIKSYNLNPVVCWEYQNMSQTPIFTVQVKVYSGSWTDSCTNISDHCCNIYGQIMYPDVSAWARVKAKVGQKESDYARSKEFLMCLKGKVGPPGLEIRRKKEEQLSVLVFHPEVVVNGESQGTMFGDGSTCYTFDYTVYVEHNRSGEILHTKHTVEKEECNETLCELNISVSTLDSRYCISVDGISSFWQVRTEKSKDVCIPPFHDDRKDSIWILVVAPLTVFTVVILVFAYWYTKKNSFKRKSIMLPKSLLSVVKSATLETKPESKYSLVTPHQPAVLESETVICEEPLSTVTAPDSPEAAEQEELSKETKALEAGGSTSAMTPDSPPTPTQRRSFSLLSSNQSGPCSLTAYHSRNGSDSGLVGSGSSISDLESLPNNNSETKMAEHDPPPVRKA

SEQ ID NO: 18 - protein, 9 aa, synthetic construct
  REGION 1..9: note = Synthetic Construct
FLGYLILGV

SEQ ID NO: 19 - protein, 15 aa, synthetic construct
  REGION 1..15: note = Synthetic Construct
SVSESDTIRSISIAS

SEQ ID NO: 20 - protein, 15 aa, synthetic construct
  REGION 1..15: note = Synthetic Construct
LLANGRMPTVLQCVN

SEQ ID NO: 21 - protein, 15 aa, synthetic construct
  REGION 1..15: note = Synthetic Construct
RMPTVLQCVNVSVVS

SEQ ID NO: 22 - protein, 12 aa, synthetic construct
  REGION 1..12: note = Synthetic Construct
CTACRWKKACQR

SEQ ID NO: 23 - protein, 9 aa, synthetic construct
  REGION 1..9: note = Synthetic Construct
VLDGLDVLL

SEQ ID NO: 24 - protein, 10 aa, synthetic construct
  REGION 1..10: note = Synthetic Construct
SLYSFPEPEA

SEQ ID NO: 25 - protein, 10 aa, synthetic construct
  REGION 1..10: note = Synthetic Construct
ALYVDSLFFL

SEQ ID NO: 26 - protein, 9 aa, synthetic construct
  REGION 1..9: note = Synthetic Construct
SLLQHLIGL

SEQ ID NO: 27 - protein, 9 aa, synthetic construct
  REGION 1..9: note = Synthetic Construct
LYVDSLFFL

SEQ ID NO: 28 - protein, 11 aa, synthetic construct
  REGION 1..11: note = Synthetic Construct
TSEKRPFMCAY

SEQ ID NO: 29 - protein, 9 aa, synthetic construct
  REGION 1..9: note = Synthetic Construct
CMTWNQMNL

SEQ ID NO: 30 - protein, 11 aa, synthetic construct
  REGION 1..11: note = Synthetic Construct
LSHLQMHSRKH

SEQ ID NO: 31 - protein, 16 aa, synthetic construct
  REGION 1..16: note = Synthetic Construct
KRYFKLSHLQMHSRKH

SEQ ID NO: 32 - protein, 8 aa, synthetic construct
  REGION 1..8: note = Synthetic Construct
CYMEAVAL

SEQ ID NO: 33 - protein, 9 aa, synthetic construct
  REGION 1..9: note = Synthetic Construct
SLLFLLFSL

SEQ ID NO: 34 - protein, 9 aa, synthetic construct
  REGION 1..9: note = Synthetic Construct
VLPLTVAEV

SEQ ID NO: 35 - protein, 9 aa, synthetic construct
  REGION 1..9: note = Synthetic Construct
ALQGGGPPY

SEQ ID NO: 36 - protein, 9 aa, synthetic construct
  REGION 1..9: note = Synthetic Construct
LYPKARLAF

SEQ ID NO: 37 - protein, 9 aa, synthetic construct
  REGION 1..9: note = Synthetic Construct
AFLPWHRLF

SEQ ID NO: 38 - protein, 9 aa, synthetic construct
  REGION 1..9: note = Synthetic Construct
SLLMWITQC

SEQ ID NO: 39 - protein, 9 aa, synthetic construct
  REGION 1..9: note = Synthetic Construct
LAMPFATPM

SEQ ID NO: 40 - protein, 9 aa, synthetic construct
  REGION 1..9: note = Synthetic Construct
ARGPESRLL

SEQ ID NO: 41 - protein, 11 aa, synthetic construct
  REGION 1..11: note = Synthetic Construct
MLMAQEALAFL

SEQ ID NO: 42 - protein, 11 aa, synthetic construct
  REGION 1..11: note = Synthetic Construct
YLAMPFATPME

SEQ ID NO: 43 - protein, 10 aa, synthetic construct
  REGION 1..10: note = Synthetic Construct
ASGPGGGAPR

SEQ ID NO: 44 - protein, 10 aa, synthetic construct
  REGION 1..10: note = Synthetic Construct
LAAQERRVPR

SEQ ID NO: 45 - protein, 10 aa, synthetic construct
  REGION 1..10: note = Synthetic Construct
TVSGNILTIR

SEQ ID NO: 46 - protein, 13 aa, synthetic construct
  REGION 1..13: note = Synthetic Construct
APRGPHGGAASGL

SEQ ID NO: 47 - protein, 11 aa, synthetic construct
  REGION 1..11: note = Synthetic Construct
MPFATPMEAEL

SEQ ID NO: 48 - protein, 12 aa, synthetic construct
  REGION 1..12: note = Synthetic Construct
KEFTVSGNILTI

SEQ ID NO: 49 - protein, 9 aa, synthetic construct
  REGION 1..9: note = Synthetic Construct
MPFATPMEA

SEQ ID NO: 50 - protein, 9 aa, synthetic construct
  REGION 1..9: note = Synthetic Construct
FATPMEAEL

SEQ ID NO: 51 - protein, 11 aa, synthetic construct
  REGION 1..11: note = Synthetic Construct
FATPMEAELAR

SEQ ID NO: 52 - protein, 9 aa, synthetic construct
  REGION 1..9: note = Synthetic Construct
LAMPFATPM

SEQ ID NO: 53 - protein, 14 aa, synthetic construct
  REGION 1..14: note = Synthetic Construct
SLLMWITQCFLPVF

SEQ ID NO: 54 - protein, 25 aa, synthetic construct
  REGION 1..25: note = Synthetic Construct
LLEFYLAMPFATPMEAELARRSLAQ

SEQ ID NO: 55 - protein, 12 aa, synthetic construct
  REGION 1..12: note = Synthetic Construct
EFYLAMPFATPM

SEQ ID NO: 56 - protein, 25 aa, synthetic construct
  REGION 1..25: note = Synthetic Construct
PGVLLKEFTVSGNILTIRLTAADHR

SEQ ID NO: 57 - protein, 12 aa, synthetic construct
  REGION 1..12: note = Synthetic Construct
RLLEFYLAMPFA

SEQ ID NO: 58 - protein, 20 aa, synthetic construct
  REGION 1..20: note = Synthetic Construct
QGAMLAAQERRVPRAAEVPR

SEQ ID NO: 59 - protein, 13 aa, synthetic construct
  REGION 1..13: note = Synthetic Construct
PFATPMEAELARR

SEQ ID NO: 60 - protein, 20 aa, synthetic construct
  REGION 1..20: note = Synthetic Construct
PGVLLKEFTVSGNILTIRLT

SEQ ID NO: 61 - protein, 10 aa, synthetic construct
  REGION 1..10: note = Synthetic Construct
VLLKEFTVSG

SEQ ID NO: 62 - protein, 18 aa, synthetic construct
  REGION 1..18: note = Synthetic Construct
AADHRQLQLSISSCLQQL

SEQ ID NO: 63 - protein, 15 aa, synthetic construct
  REGION 1..15: note = Synthetic Construct
LKEFTVSGNILTIRL

SEQ ID NO: 64 - protein, 14 aa, synthetic construct
  REGION 1..14: note = Synthetic Construct
LLEFYLAMPFATPM

SEQ ID NO: 65 - protein, 14 aa, synthetic construct
  REGION 1..14: note = Synthetic Construct
AGATGGRGPRGAGA

SEQ ID NO: 66 - protein, 13 aa, synthetic construct
  REGION 1..13: note = Synthetic Construct
TYYRPGVNLSLSC

SEQ ID NO: 67 - protein, 13 aa, synthetic construct
  REGION 1..13: note = Synthetic Construct
EIIYPNASLLIQN

SEQ ID NO: 68 - protein, 15 aa, synthetic construct
  REGION 1..15: note = Synthetic Construct
YACFVSNLATGRNNS

SEQ ID NO: 69 - protein, 13 aa, synthetic construct
  REGION 1..13: note = Synthetic Construct
LWWVNNQSLPVSP

SEQ ID NO: 70 - protein, 13 aa, synthetic construct
  REGION 1..13: note = Synthetic Construct
NSIVKSITVSASG

SEQ ID NO: 71 - protein, 9 aa, synthetic construct
  REGION 1..9: note = Synthetic Construct
KTWGQYWQV

SEQ ID NO: 72 - protein, 10 aa, synthetic construct
  REGION 1..10: note = Synthetic Construct
AMLGTHTMEV

SEQ ID NO: 73 - protein, 9 aa, synthetic construct
  REGION 1..9: note = Synthetic Construct
ITDQVPFSV

SEQ ID NO: 74 - protein, 9 aa, synthetic construct
  REGION 1..9: note = Synthetic Construct
YLEPGPVTA

SEQ ID NO: 75 - protein, 10 aa, synthetic construct
  REGION 1..10: note = Synthetic Construct
LLDGTATLRL

SEQ ID NO: 76 - protein, 10 aa, synthetic construct
  REGION 1..10: note = Synthetic Construct
VLYRYGSFSV

SEQ ID NO: 77 - protein, 10 aa, synthetic construct
  REGION 1..10: note = Synthetic Construct
SLADTNSLAV

SEQ ID NO: 78 - protein, 9 aa, synthetic construct
  REGION 1..9: note = Synthetic Construct
RLMKQDFSV

SEQ ID NO: 79 - protein, 9 aa, synthetic construct
  REGION 1..9: note = Synthetic Construct
RLPRIFCSC

SEQ ID NO: 80 - protein, 9 aa, synthetic construct
  REGION 1..9: note = Synthetic Construct
LIYRRRLMK

SEQ ID NO: 81 - protein, 9 aa, synthetic construct
  REGION 1..9: note = Synthetic Construct
ALLAVGATK

SEQ ID NO: 82 - protein, 10 aa, synthetic construct
  REGION 1..10: note = Synthetic Construct
IALNFPGSQK

SEQ ID NO: 83 - protein, 9 aa, synthetic construct
  REGION 1..9: note = Synthetic Construct
RSYVPLAHR

SEQ ID NO: 84 - protein, 9 aa, synthetic construct
  REGION 1..9: note = Synthetic Construct
VVPCEPPEV

SEQ ID NO: 85 - protein, 9 aa, synthetic construct
  REGION 1..9: note = Synthetic Construct
HLYQGCQVV

SEQ ID NO: 86 - protein, 10 aa, synthetic construct
  REGION 1..10: note = Synthetic Construct
YLVPQQGFFC

SEQ ID NO: 87 - protein, 9 aa, synthetic construct
  REGION 1..9: note = Synthetic Construct
PLQPEQLQV

SEQ ID NO: 88 - protein, 9 aa, synthetic construct
  REGION 1..9: note = Synthetic Construct
TLEEITGYL

SEQ ID NO: 89 - protein, 9 aa, synthetic construct
  REGION 1..9: note = Synthetic Construct
ALIHHNTHL

SEQ ID NO: 90 - protein, 9 aa, synthetic construct
  REGION 1..9: note = Synthetic Construct
PLTSIISAV

SEQ ID NO: 91 - protein, 9 aa, synthetic construct
  REGION 1..9: note = Synthetic Construct
VLRENTSPK

SEQ ID NO: 92 - protein, 9 aa, synthetic construct
  REGION 1..9: note = Synthetic Construct
TYLPTNASL

SEQ ID NO: 93 - protein, 9 aa, synthetic construct
  REGION 1..9: note = Synthetic Construct
RYQLDPKFI

SEQ ID NO: 94 - protein, 9 aa, synthetic construct
  REGION 1..9: note = Synthetic Construct
ILFTINFTI

SEQ ID NO: 95 - protein, 9 aa, synthetic construct
  REGION 1..9: note = Synthetic Construct
VLFTINFTI

SEQ ID NO: 96 - protein, 9 aa, synthetic construct
  REGION 1..9: note = Synthetic Construct
TLNFTITNL

SEQ ID NO: 97 - protein, 9 aa, synthetic construct
  REGION 1..9: note = Synthetic Construct
VLQGLLKPL

SEQ ID NO: 98 - protein, 9 aa, synthetic construct
  REGION 1..9: note = Synthetic Construct
VLQGLLRPV

SEQ ID NO: 99 - protein, 9 aa, synthetic construct
  REGION 1..9: note = Synthetic Construct
RLDPKSPGV

SEQ ID NO: 100 - protein, 9 aa, synthetic construct
  REGION 1..9: note = Synthetic Construct
QLYWELSKL

SEQ ID NO: 101 - protein, 9 aa, synthetic construct
  REGION 1..9: note = Synthetic Construct
KLTRGIVEL

SEQ ID NO: 102 - protein, 9 aa, synthetic construct
  REGION 1..9: note = Synthetic Construct
QLTNGITEL

SEQ ID NO: 103 - protein, 9 aa, synthetic construct
  REGION 1..9: note = Synthetic Construct
QLTHNITEL

SEQ ID NO: 104 - protein, 9 aa, synthetic construct
  REGION 1..9: note = Synthetic Construct
TLDRNSLYV

SEQ ID NO: 105 - protein, 9 aa, synthetic construct
  REGION 1..9: note = Synthetic Construct
FLLSLALML

SEQ ID NO: 106 - protein, 9 aa, synthetic construct
  REGION 1..9: note = Synthetic Construct
NLGPWIQQV

SEQ ID NO: 107 - protein, 9 aa, synthetic construct
  REGION 1..9: note = Synthetic Construct
ILDSSEEDK

SEQ ID NO: 108 - protein, 10 aa, synthetic construct
  REGION 1..10: note = Synthetic Construct
YLPKSWTIQV

SEQ ID NO: 109 - protein, 12 aa, synthetic construct
  REGION 1..12: note = Synthetic Construct
WIHEQMERDLKT

SEQ ID NO: 110 - protein, 9 aa, synthetic construct
  REGION 1..9: note = Synthetic Construct
LLANAYIYV

SEQ ID NO: 111 - protein, 9 aa, synthetic construct
  REGION 1..9: note = Synthetic Construct
YLLCKAFGA

SEQ ID NO: 112 - protein, 9 aa, synthetic construct
  REGION 1..9: note = Synthetic Construct
KLSPYVHYT

SEQ ID NO: 113 - protein, 20 aa, synthetic construct
  REGION 1..20: note = Synthetic Construct
PDTRPAPGSTAPPAHGVTSA

SEQ ID NO: 114 - protein, 9 aa, synthetic construct
  REGION 1..9: note = Synthetic Construct
STAPPVHNV

SEQ ID NO: 115 - protein, 9 aa, synthetic construct
  REGION 1..9: note = Synthetic Construct
LLLLTVLTV

SEQ ID NO: 116 - protein, 12 aa, synthetic construct
  REGION 1..12: note = Synthetic Construct
PGSTAPPAHGVT

SEQ ID NO: 117 - protein, 9 aa, synthetic construct
  REGION 1..9: note = Synthetic Construct
MLAVISCAV

SEQ ID NO: 118 - protein, 9 aa, synthetic construct
  REGION 1..9: note = Synthetic Construct
RQKRILVNL

SEQ ID NO: 119 - protein, 9 aa, synthetic construct
  REGION 1..9: note = Synthetic Construct
NYNNFYRFL

SEQ ID NO: 120 - protein, 10 aa, synthetic construct
  REGION 1..10: note = Synthetic Construct
EYSKECLKEF

SEQ ID NO: 121 - protein, 9 aa, synthetic construct
  REGION 1..9: note = Synthetic Construct
EYLSLSDKI

SEQ ID NO: 122 - protein, 9 aa, synthetic construct
  REGION 1..9: note = Synthetic Construct
ELVRRILSR

SEQ ID NO: 123 - protein, 9 aa, synthetic construct
  REGION 1..9: note = Synthetic Construct
APRGVRMAV

SEQ ID NO: 124 - protein, 20 aa, synthetic construct
  REGION 1..20: note = Synthetic Construct
QGAMLAAQERRVPRAAEVPR

SEQ ID NO: 125 - protein, 22 aa, synthetic construct
  REGION 1..22: note = Synthetic Construct
CLSRRPWKRSWSAGSCPGMPHL

SEQ ID NO: 126 - protein, 13 aa, synthetic construct
  REGION 1..13: note = Synthetic Construct
ILSRDAAPLPRPG

SEQ ID NO: 127 - protein, 9 aa, synthetic construct
  REGION 1..9: note = Synthetic Construct
EVDPASNTY

SEQ ID NO: 128 - protein, 10 aa, synthetic construct
  REGION 1..10: note = Synthetic Construct
GVYDGREHTV

SEQ ID NO: 129 - protein, 9 aa, synthetic construct
  REGION 1..9: note = Synthetic Construct
NYKRCFPVI

SEQ ID NO: 130 - protein, 8 aa, synthetic construct
  REGION 1..8: note = Synthetic Construct
SESLKMIF

SEQ ID NO: 131 - protein, 20 aa, synthetic construct
  REGION 1..20: note = Synthetic Construct
INKTSGPKRGKHAWTHRLRE

SEQ ID NO: 132 - protein, 20 aa, synthetic construct
  REGION 1..20: note = Synthetic Construct
YFSKKEWEKMKSSEKIVYVY

SEQ ID NO: 133 - protein, 20 aa, synthetic construct
  REGION 1..20: note = Synthetic Construct
MKLNYEVMTKLGFKVTLPPF

SEQ ID NO: 134 - protein, 20 aa, synthetic construct
  REGION 1..20: note = Synthetic Construct
KHAWTHRLRERKQLVVYEEI

SEQ ID NO: 135 - protein, 20 aa, synthetic construct
  REGION 1..20: note = Synthetic Construct
LGFKVTLPPFMRSKRAADFH

SEQ ID NO: 136 - protein, 20 aa, synthetic construct
  REGION 1..20: note = Synthetic Construct
KSSEKIVYVYMKLNYEVMTK

SEQ ID NO: 137 - protein, 9 aa, synthetic construct
  REGION 1..9: note = Synthetic Construct
SLGWLFLLL

SEQ ID NO: 138 - protein, 9 aa, synthetic construct
  REGION 1..9: note = Synthetic Construct
LSRLSNRLL

SEQ ID NO: 139 - protein, 9 aa, synthetic construct
  REGION 1..9: note = Synthetic Construct
TMNGSKSPV

SEQ ID NO: 140 - protein, 9 aa, synthetic construct
  REGION 1..9: note = Synthetic Construct
PLLENVISK

SEQ ID NO: 141 - protein, 9 aa, synthetic construct
  REGION 1..9: note = Synthetic Construct
SLSKILDTV

SEQ ID NO: 142 - protein, 9 aa, synthetic construct
  REGION 1..9: note = Synthetic Construct
AARAVFLAL

SEQ ID NO: 143 - protein, 9 aa, synthetic construct
  REGION 1..9: note = Synthetic Construct
EADPTGHSY

SEQ ID NO: 144 - protein, 9 aa, synthetic construct
  REGION 1..9: note = Synthetic Construct
KVLEYVIKV

SEQ ID NO: 145 - protein, 9 aa, synthetic construct
  REGION 1..9: note = Synthetic Construct
SLFRAVITK

SEQ ID NO: 146 - protein, 10 aa, synthetic construct
  REGION 1..10: note = Synthetic Construct
EVYDGREHSA

SEQ ID NO: 147 - protein, 9 aa, synthetic construct
  REGION 1..9: note = Synthetic Construct
RVRFFFPSL

SEQ ID NO: 148 - protein, 10 aa, synthetic construct
  REGION 1..10: note = Synthetic Construct
REPVTKAEML

SEQ ID NO: 149 - protein, 10 aa, synthetic construct
  REGION 1..10: note = Synthetic Construct
KEADPTGHSY

SEQ ID NO: 150 - protein, 9 aa, synthetic construct
  REGION 1..9: note = Synthetic Construct
DPARYEFLW

SEQ ID NO: 151 - protein, 11 aa, synthetic construct
  REGION 1..11: note = Synthetic Construct
ITKKVADLVGF

SEQ ID NO: 152 - protein, 9 aa, synthetic construct
  REGION 1..9: note = Synthetic Construct
SAFPTTINF

SEQ ID NO: 153 - protein, 9 aa, synthetic construct
  REGION 1..9: note = Synthetic Construct
SAYGEPRKL

SEQ ID NO: 154 - protein, 15 aa, synthetic construct
  REGION 1..15: note = Synthetic Construct
TSCILESLFRAVITK

SEQ ID NO: 155 - protein, 15 aa, synthetic construct
  REGION 1..15: note = Synthetic Construct
PRALAETSYVKVLEY

SEQ ID NO: 156 - protein, 16 aa, synthetic construct
  REGION 1..16: note = Synthetic Construct
FLLLKYRAREPVTKAE

SEQ ID NO: 157 - protein, 12 aa, synthetic construct
  REGION 1..12: note = Synthetic Construct
EYVIKVSARVRF

SEQ ID NO: 158 - protein, 10 aa, synthetic construct
  REGION 1..10: note = Synthetic Construct
YLQLVFGIEV

SEQ ID NO: 159 - protein, 9 aa, synthetic construct
  REGION 1..9: note = Synthetic Construct
EYLQLVFGI

SEQ ID NO: 160 - protein, 9 aa, synthetic construct
  REGION 1..9: note = Synthetic Construct
EGDCAPEEK

SEQ ID NO: 161 - protein, 14 aa, synthetic construct
  REGION 1..14: note = Synthetic Construct
LLKYRAREPVTKAE

SEQ ID NO: 162 - protein, 9 aa, synthetic construct
  REGION 1..9: note = Synthetic Construct
KASEKIFYV

SEQ ID NO: 163 - protein, 16 aa, synthetic construct
  REGION 1..16: note = Synthetic Construct
EKIQKAFDDIAKYFSK

SEQ ID NO: 164 - protein, 11 aa, synthetic construct
  REGION 1..11: note = Synthetic Construct
FGRLQGISPKI

SEQ ID NO: 165 - protein, 18 aa, synthetic construct
  REGION 1..18: note = Synthetic Construct
WEKMKASEKIFYVYMKRK

SEQ ID NO: 166 - protein, 15 aa, synthetic construct
  REGION 1..15: note = Synthetic Construct
KIFYVYMKRKYEAMT

SEQ ID NO: 167 - protein, 14 aa, synthetic construct
  REGION 1..14: note = Synthetic Construct
KIFYVYMKRKYEAM

SEQ ID NO: 168 - protein, 15 aa, synthetic construct
  REGION 1..15: note = Synthetic Construct
CEFHACWPAFTVLGE

SEQ ID NO: 169 - protein, 9 aa, synthetic construct
  REGION 1..9: note = Synthetic Construct
KFLDALISL

SEQ ID NO: 170 - protein, 10 aa, synthetic construct
  REGION 1..10: note = Synthetic Construct
ALGGHPLLGV

SEQ ID NO: 171 - protein, 9 aa, synthetic construct
  REGION 1..9: note = Synthetic Construct
LPRWPPPQL

SEQ ID NO: 172 - protein, 9 aa, synthetic construct
  REGION 1..9: note = Synthetic Construct
ILAKFLHWL

SEQ ID NO: 173 - protein, 9 aa, synthetic construct
  REGION 1..9: note = Synthetic Construct
RLVDDFLLV

SEQ ID NO: 174 - protein, 15 aa, synthetic construct
  REGION 1..15: note = Synthetic Construct
RPGLLGASVLGLDDI

SEQ ID NO: 175 - protein, 15 aa, synthetic construct
  REGION 1..15: note = Synthetic Construct
LTDLQPYMRQFVAHL

SEQ ID NO: 176 - protein, 9 aa, synthetic construct
  REGION 1..9: note = Synthetic Construct
VVVGAVGVG

SEQ ID NO: 177 - protein, 9 aa, synthetic construct
  REGION 1..9: note = Synthetic Construct
HLSTAFARV

SEQ ID NO: 178 - protein, 9 aa, synthetic construct
  REGION 1..9: note = Synthetic Construct
KIFGSLAFL

SEQ ID NO: 179 - protein, 9 aa, synthetic construct
  REGION 1..9: note = Synthetic Construct
IISAVVGIL

SEQ ID NO: 180 - protein, 9 aa, synthetic construct
  REGION 1..9: note = Synthetic Construct
ALCRWGLLL

SEQ ID NO: 181 - protein, 9 aa, synthetic construct
  REGION 1..9: note = Synthetic Construct
ILHNGAYSL

SEQ ID NO: 182 - protein, 9 aa, synthetic construct
  REGION 1..9: note = Synthetic Construct
RLLQETELV

SEQ ID NO: 183 - protein, 9 aa, synthetic construct
  REGION 1..9: note = Synthetic Construct
VVKGVVFGI

SEQ ID NO: 184 - protein, 10 aa, synthetic construct
  REGION 1..10: note = Synthetic Construct
YMIMVKCWMI

SEQ ID NO: 185 - protein, 9 aa, synthetic construct
  REGION 1..9: note = Synthetic Construct
SLLSGDWVL

SEQ ID NO: 186 - protein, 9 aa, synthetic construct
  REGION 1..9: note = Synthetic Construct
GLQLGVQAV

SEQ ID NO: 187 - protein, 9 aa, synthetic construct
  REGION 1..9: note = Synthetic Construct
PLTEYIQPV

SEQ ID NO: 188 - protein, 9 aa, synthetic construct
  REGION 1..9: note = Synthetic Construct
SPRWWPTCL

SEQ ID NO: 189 - protein, 9 aa, synthetic construct
  REGION 1..9: note = Synthetic Construct
GVALQTMKQ

SEQ ID NO: 190 - protein, 9 aa, synthetic construct
  REGION 1..9: note = Synthetic Construct
FMNKFIYEI

SEQ ID NO: 191 - protein, 10 aa, synthetic construct
  REGION 1..10: note = Synthetic Construct
QLAVSVILRV

SEQ ID NO: 192 - protein, 14 aa, synthetic construct
  REGION 1..14: note = Synthetic Construct
LPAVVGLSPGEQEY

SEQ ID NO: 193 - protein, 9 aa, synthetic construct
  REGION 1..9: note = Synthetic Construct
NYARTEDFF

SEQ ID NO: 194 - protein, 9 aa, synthetic construct
  REGION 1..9: note = Synthetic Construct
FLIIWQNTM

SEQ ID NO: 195 - protein, 14 aa, synthetic construct
  REGION 1..14: note = Synthetic Construct
TLYQDDTLTLQAAG

SEQ ID NO: 196 - protein, 10 aa, synthetic construct
  REGION 1..10: note = Synthetic Construct
SLYKFSPFPL

SEQ ID NO: 197 - protein, 9 aa, synthetic construct
  REGION 1..9: note = Synthetic Construct
KELEGILLL

SEQ ID NO: 198 - protein, 9 aa, synthetic construct
  REGION 1..9: note = Synthetic Construct
RLSSCVPVA

SEQ ID NO: 199 - protein, 10 aa, synthetic construct
  REGION 1..10: note = Synthetic Construct
VLLQAGSLHA

SEQ ID NO: 200 - protein, 9 aa, synthetic construct
  REGION 1..9: note = Synthetic Construct
LLNAFTVTV

SEQ ID NO: 201 - protein, 8 aa, synthetic construct
  REGION 1..8: note = Synthetic Construct
VLFYLGQY

SEQ ID NO: 202 - protein, 10 aa, synthetic construct
  REGION 1..10: note = Synthetic Construct
FIASNGVKLV

SEQ ID NO: 203 - protein, 9 aa, synthetic construct
  REGION 1..9: note = Synthetic Construct
ETVSEQSNV

SEQ ID NO: 204 - protein, 9 aa, synthetic construct
  REGION 1..9: note = Synthetic Construct
FLDEFMEGV

SEQ ID NO: 205 - protein, 8 aa, synthetic construct
  REGION 1..8: note = Synthetic Construct
QQITKTEV

SEQ ID NO: 206 - protein, 8 aa, synthetic construct
  REGION 1..8: note = Synthetic Construct
YRPRPRRY

SEQ ID NO: 207 - protein, 9 aa, synthetic construct
  REGION 1..9: note = Synthetic Construct
MVKISGGPR

SEQ ID NO: 208 - protein, 9 aa, synthetic construct
  REGION 1..9: note = Synthetic Construct
EVDPIGHVY

SEQ ID NO: 209 - protein, 9 aa, synthetic construct
  REGION 1..9: note = Synthetic Construct
ISGGPRISY

SEQ ID NO: 210 - protein, 9 aa, synthetic construct
  REGION 1..9: note = Synthetic Construct
RQKKIRIQL

SEQ ID NO: 211 - protein, 16 aa, synthetic construct
  REGION 1..16: note = Synthetic Construct
HLGSRQKKIRIQLRSQ

SEQ ID NO: 212 - protein, 17 aa, synthetic construct
  REGION 1..17: note = Synthetic Construct
CATWKVICKSCISQTPG

SEQ ID NO: 213 - protein, 9 aa, synthetic construct
  REGION 1..9: note = Synthetic Construct
MIAVFLPIV

SEQ ID NO: 214 - protein, 15 aa, synthetic construct
  REGION 1..15: note = Synthetic Construct
HQQYFYKIPILVINK

SEQ ID NO: 215 - protein, 9 aa, synthetic construct
  REGION 1..9: note = Synthetic Construct
FLFLLFFWL

SEQ ID NO: 216 - protein, 9 aa, synthetic construct
  REGION 1..9: note = Synthetic Construct
TLMSAMTNL

SEQ ID NO: 217 - protein, 9 aa, synthetic construct
  REGION 1..9: note = Synthetic Construct
ALDVYNGLL

SEQ ID NO: 218 - protein, 10 aa, synthetic construct
  REGION 1..10: note = Synthetic Construct
FLTPKKLQCV

SEQ ID NO: 219 - protein, 10 aa, synthetic construct
  REGION 1..10: note = Synthetic Construct
VISNDVCAQV

SEQ ID NO: 220 - protein, 11 aa, synthetic construct
  REGION 1..11: note = Synthetic Construct
KEFTVSGNILT

SEQ ID NO: 221 - protein, 9 aa, synthetic construct
  REGION 1..9: note = Synthetic Construct
YYWPRPRRY

SEQ ID NO: 222 - protein, 9 aa, synthetic construct
  REGION 1..9: note = Synthetic Construct
NTYASPRFK

SEQ ID NO: 223 - protein, 10 aa, synthetic construct
  REGION 1..10: note = Synthetic Construct
SLFEGIDIYT

SEQ ID NO: 224 - protein, 9 aa, synthetic construct
  REGION 1..9: note = Synthetic Construct
ALSVMGVYV

SEQ ID NO: 225 - protein, 15 aa, synthetic construct
  REGION 1..15: note = Synthetic Construct
YSVYFNLPADTIYTN

SEQ ID NO: 226 - protein, 29 aa, synthetic construct
  REGION 1..29: note = Synthetic Construct
EDLTVKIGDFGLATEKSRWSGSHQFEQLS

SEQ ID NO: 227 - protein, 9 aa, synthetic construct
  REGION 1..9: note = Synthetic Construct
SYLDSGIHF

SEQ ID NO: 228 - protein, 12 aa, synthetic construct
  REGION 1..12: note = Synthetic Construct
FSWAMDLDPKGA

SEQ ID NO: 229 - protein, 10 aa, synthetic construct
  REGION 1..10: note = Synthetic Construct
ACDPHSGHFV

SEQ ID NO: 230 - protein, 9 aa, synthetic construct
  REGION 1..9: note = Synthetic Construct
CILGKLFTK

SEQ ID NO: 231 - protein, 9 aa, synthetic construct
  REGION 1..9: note = Synthetic Construct
AVCPWTWLR

SEQ ID NO: 232 - protein, 9 aa, synthetic construct
  REGION 1..9: note = Synthetic Construct
ILDKVLVHL

SEQ ID NO: 233 - protein, 9 aa, synthetic construct
  REGION 1..9: note = Synthetic Construct
GLFGDIYLA

SEQ ID NO: 234 - protein, 14 aa, synthetic construct
  REGION 1..14: note = Synthetic Construct
MIFEKHGFRRTTPP

SEQ ID NO: 235 - protein, 10 aa, synthetic construct
  REGION 1..10: note = Synthetic Construct
SLADEAEVYL

SEQ ID NO: 236 - protein, 10 aa, synthetic construct
  REGION 1..10: note = Synthetic Construct
TLDWLLQTPK

SEQ ID NO: 237 - protein, 9 aa, synthetic construct
  REGION 1..9: note = Synthetic Construct
ILNAMIAKI

SEQ ID NO: 238 - protein, 9 aa, synthetic construct
  REGION 1..9: note = Synthetic Construct
WRRAPAPGA

SEQ ID NO: 239 - protein, 9 aa, synthetic construct
  REGION 1..9: note = Synthetic Construct
PVTWRRAPA

SEQ ID NO: 240 - protein, 11 aa, synthetic construct
  REGION 1..11: note = Synthetic Construct
FLEGNEVGKTY

SEQ ID NO: 241 - protein, 9 aa, synthetic construct
  REGION 1..9: note = Synthetic Construct
KTLTSVFQK

SEQ ID NO: 242 - protein, 9 aa, synthetic construct
  REGION 1..9: note = Synthetic Construct
EEKLIVVLF

SEQ ID NO: 243 - protein, 11 aa, synthetic construct
  REGION 1..11: note = Synthetic Construct
SELFRSGLDSY

SEQ ID NO: 244 - protein, 9 aa, synthetic construct
  REGION 1..9: note = Synthetic Construct
FRSGLDSYV

SEQ ID NO: 245 - protein, 9 aa, synthetic construct
  REGION 1..9: note = Synthetic Construct
EAFIQPITR

SEQ ID NO: 246 - protein, 11 aa, synthetic construct
  REGION 1..11: note = Synthetic Construct
RVIKNSIRLTL

SEQ ID NO: 247 - protein, 9 aa, synthetic construct
  REGION 1..9: note = Synthetic Construct
KINKNPKYK

SEQ ID NO: 248 - protein, 9 aa, synthetic construct
  REGION 1..9: note = Synthetic Construct
YTDFHCQYV

SEQ ID NO: 249 - protein, 10 aa, synthetic construct
  REGION 1..10: note = Synthetic Construct
LLLDDLLVSI

SEQ ID NO: 250 - protein, 16 aa, synthetic construct
  REGION 1..16: note = Synthetic Construct
PYYFAAELPPRNLPEP

SEQ ID NO: 251 - protein, 10 aa, synthetic construct
  REGION 1..10: note = Synthetic Construct
ILDTAGREEY

SEQ ID NO: 252 - protein, 9 aa, synthetic construct
  REGION 1..9: note = Synthetic Construct
RPHVPESAF

SEQ ID NO: 253 - protein, 9 aa, synthetic construct
  REGION 1..9: note = Synthetic Construct
KIFSEVTLK

SEQ ID NO: 254 - protein, 9 aa, synthetic construct
  REGION 1..9: note = Synthetic Construct
SHETVIIEL

SEQ ID NO: 255 - protein, 15 aa, synthetic construct
  REGION 1..15: note = Synthetic Construct
GELIGILNAAKVPAD

SEQ ID NO: 256 - protein, 9 aa, synthetic construct
  REGION 1..9: note = Synthetic Construct
LYSACFWWL

SEQ ID NO: 257 - protein, 9 aa, synthetic construct
  REGION 1..9: note = Synthetic Construct
VLHWDPETV

SEQ ID NO: 258 - protein, 9 aa, synthetic construct
  REGION 1..9: note = Synthetic Construct
MSLQRQFLR

SEQ ID NO: 259 - protein, 20 aa, synthetic construct
  REGION 1..20: note = Synthetic Construct
ISPNSVFSQWRVVCDSLEDY

SEQ ID NO: 260 - protein, 10 aa, synthetic construct
  REGION 1..10: note = Synthetic Construct
SLPYWNFATG

SEQ ID NO: 261 - protein, 15 aa, synthetic construct
  REGION 1..15: note = Synthetic Construct
SQWRVVCDSLEDYDT

SEQ ID NO: 262 - protein, 9 aa, synthetic construct
  REGION 1..9: note = Synthetic Construct
SVYDFFVWL

SEQ ID NO: 263 - protein, 9 aa, synthetic construct
  REGION 1..9: note = Synthetic Construct
TLDSQVMSL

SEQ ID NO: 264 - protein, 9 aa, synthetic construct
  REGION 1..9: note = Synthetic Construct
LLGPGRPYR

SEQ ID NO: 265 - protein, 9 aa, synthetic construct
  REGION 1..9: note = Synthetic Construct
ANDPIFVVL

SEQ ID NO: 266 - protein, 15 aa, synthetic construct
  REGION 1..15: note = Synthetic Construct
QCTEVRADTRPWSGP

SEQ ID NO: 267 - protein, 10 aa, synthetic construct
  REGION 1..10: note = Synthetic Construct
ALPYWNFATG

SEQ ID NO: 268 - protein, 9 aa, synthetic construct
  REGION 1..9: note = Synthetic Construct
KCDICTDEY

SEQ ID NO: 269 - protein, 11 aa, synthetic construct
  REGION 1..11: note = Synthetic Construct
SSDYVIPIGTY

SEQ ID NO: 270 - protein, 9 aa, synthetic construct
  REGION 1..9: note = Synthetic Construct
MLLAVLYCL

SEQ ID NO: 271 - protein, 10 aa, synthetic construct
  REGION 1..10: note = Synthetic Construct
CLLWSFQTSA

SEQ ID NO: 272 - protein, 9 aa, synthetic construct
  REGION 1..9: note = Synthetic Construct
YMDGTMSQV

SEQ ID NO: 273 - protein, 11 aa, synthetic construct
  REGION 1..11: note = Synthetic Construct
IYMDGTADFSF

SEQ ID NO: 274 - protein, 9 aa, synthetic construct
  REGION 1..9: note = Synthetic Construct
QCSGNFMGF

SEQ ID NO: 275 - protein, 12 aa, synthetic construct
  REGION 1..12: note = Synthetic Construct
TPRLPSSADVEF

SEQ ID NO: 276 - protein, 9 aa, synthetic construct
  REGION 1..9: note = Synthetic Construct
LPSSADVEF

SEQ ID NO: 277 - protein, 10 aa, synthetic construct
  REGION 1..10: note = Synthetic Construct
LHHAFVDSIF

SEQ ID NO: 278 - protein, 15 aa, synthetic construct
  REGION 1..15: note = Synthetic Construct
QNILLSNAPLGPQFP

SEQ ID NO: 279 - protein, 13 aa, synthetic construct
  REGION 1..13: note = Synthetic Construct
SYLQDSDPDSFQD

SEQ ID NO: 280 - protein, 21 aa, synthetic construct [sequence omitted]

SEQ ID NO: 281 - protein, 30 aa, synthetic construct
  REGION 1..30: note = Synthetic Construct
YTTAEEAAGIGILTVILGVLLLIGCWYCRR

SEQ ID NO: 282 - protein, 9 aa, synthetic construct
  REGION 1..9: note = Synthetic Construct
ALNFPGSQK

SEQ ID NO: 283 - protein, 9 aa, synthetic construct
  REGION 1..9: note = Synthetic Construct
VYFFLPDHL

SEQ ID NO: 284 - protein, 9 aa, synthetic construct
  REGION 1..9: note = Synthetic Construct
RTKQLYPEW

SEQ ID NO: 285 - protein, 10 aa, synthetic construct
  REGION 1..10: note = Synthetic Construct
HTMEVTVYHR

SEQ ID NO: 286 - protein, 9 aa, synthetic construct
  REGION 1..9: note = Synthetic Construct
SSPGCQPPA

SEQ ID NO: 287 - protein, 10 aa, synthetic construct
  REGION 1..10: note = Synthetic Construct
VPLDCVLYRY

SEQ ID NO: 288 - protein, 9 aa, synthetic construct
  REGION 1..9: note = Synthetic Construct
LPHSSSHWL

SEQ ID NO: 289 - protein, 8 aa, synthetic construct
  REGION 1..8: note = Synthetic Construct
SNDGPTLI

SEQ ID NO: 290 - protein, 15 aa, synthetic construct
  REGION 1..15: note = Synthetic Construct
GRAMLGTHTMEVTVY

SEQ ID NO: 291 - protein, 16 aa, synthetic construct
  REGION 1..16: note = Synthetic Construct
WNRQLYPEWTEAQRLD

SEQ ID NO: 292 - protein, 18 aa, synthetic construct
  REGION 1..18: note = Synthetic Construct
TTEWVETTARELPIPEPE

SEQ ID NO: 293 - protein, 17 aa, synthetic construct
  REGION 1..17: note = Synthetic Construct
TGRAMLGTHTMEVTVYH

SEQ ID NO: 294 - protein, 10 aa, synthetic construct
  REGION 1..10: note = Synthetic Construct
VLPDVFIRCV

SEQ ID NO: 295 - protein, 10 aa, synthetic construct
  REGION 1..10: note = Synthetic Construct
RYCNLEGPPI

SEQ ID NO: 296 - protein, 24 aa, synthetic construct
  REGION 1..24: note = Synthetic Construct
KWTEPYCVIAAVKIFPRFFMVAKQ

SEQ ID NO: 297 - protein, 20 aa, synthetic construct
  REGION 1..20: note = Synthetic Construct
KCCKIRYCNLEGPPINSSVF

SEQ ID NO: 298 - protein, 9 aa, synthetic construct
  REGION 1..9: note = Synthetic Construct
FLWGPRALV

SEQ ID NO: 299 - protein, 9 aa, synthetic construct
  REGION 1..9: note = Synthetic Construct
VRIGHLYIL

SEQ ID NO: 300 - protein, 15 aa, synthetic construct
  REGION 1..15: note = Synthetic Construct
REPFTKAEMLGSVIR

SEQ ID NO: 301 - protein, 14 aa, synthetic construct
  REGION 1..14: note = Synthetic Construct
AELVHFLLLKYRAR

SEQ ID NO: 302 - protein, 10 aa, synthetic construct
  REGION 1..10: note = Synthetic Construct
LLFGLALIEV

SEQ ID NO: 303 - protein, 9 aa, synthetic construct
  REGION 1..9: note = Synthetic Construct
ALKDVEERV

SEQ ID NO: 304 - protein, 9 aa, synthetic construct
  REGION 1..9: note = Synthetic Construct
SESIKKKVL

SEQ ID NO: 305 - protein, 9 aa, synthetic construct
  REGION 1..9: note = Synthetic Construct
ASSTLYLVF

SEQ ID NO: 306 - protein, 15 aa, synthetic construct
  REGION 1..15: note = Synthetic Construct
SSTLYLVFSPSSFST

SEQ ID NO: 307 - protein, 9 aa, synthetic construct
  REGION 1..9: note = Synthetic Construct
QGQHFLQKV

SEQ ID NO: 308 - protein, 10 aa, synthetic construct
  REGION 1..10: note = Synthetic Construct
EVISCKLIKR

SEQ ID NO: 309 - protein, 9 aa, synthetic construct
  REGION 1..9: note = Synthetic Construct
FPSDSWCYF

SEQ ID NO: 310 - protein, 9 aa, synthetic construct
  REGION 1..9: note = Synthetic Construct
LYATVIHDI

SEQ ID NO: 311 - protein, 9 aa, synthetic construct
  REGION 1..9: note = Synthetic Construct
SSKALQRPV

SEQ ID NO: 312 - protein, 9 aa, synthetic construct
  REGION 1..9: note = Synthetic Construct
GFKQSSKAL

SEQ ID NO: 313 - protein, 17 aa, synthetic construct
  REGION 1..17: note = Synthetic Construct
ATGFKQSSKALQRPVAS

SEQ ID NO: 314 - protein, 16 aa, synthetic construct
  REGION 1..16: note = Synthetic Construct
TMKQICKKEIRRLHQY

SEQ ID NO: 315 - protein, 10 aa, synthetic construct
  REGION 1..10: note = Synthetic Construct
KILDAVVAQK

SEQ ID NO: 316 - protein, 9 aa, synthetic construct
  REGION 1..9: note = Synthetic Construct
RIAECILGM

SEQ ID NO: 317 - protein, 15 aa, synthetic construct
  REGION 1..15: note = Synthetic Construct
IGRIAECILGMNPSR

SEQ ID NO: 318 - protein, 10 aa, synthetic construct
  REGION 1..10: note = Synthetic Construct
YVDFREYEYY

SEQ ID NO: 319 - protein, 9 aa, synthetic construct
  REGION 1..9: note = Synthetic Construct
FLDRFLSCM

SEQ ID NO: 320 - protein, 11 aa, synthetic construct
  REGION 1..11: note = Synthetic Construct
SLIAAAAFCLA

SEQ ID NO: 321 - protein, 9 aa, synthetic construct
  REGION 1..9: note = Synthetic Construct
VVMSWAPPV

SEQ ID NO: 322 - protein, 25 aa, synthetic construct
  REGION 1..25: note = Synthetic Construct
NSNHVASGAGEAAIETQSSSSEEIV

SEQ ID NO: 323 - protein, 10 aa, synthetic construct
  REGION 1..10: note = Synthetic Construct
ILFGISLREV

SEQ ID NO: 324 - protein, 9 aa, synthetic construct
  REGION 1..9: note = Synthetic Construct
KVVEFLAML

SEQ ID NO: 325 - protein, 13 aa, synthetic construct
  REGION 1..13: note = Synthetic Construct
SSALLSIFQSSPE

SEQ ID NO: 326 - protein, 9 aa, synthetic construct
  REGION 1..9: note = Synthetic Construct
SFSYTLLSL

SEQ ID NO: 327 - protein, 9 aa, synthetic construct
  REGION 1..9: note = Synthetic Construct
VSSFFSYTL

SEQ ID NO: 328 - protein, 15 aa, synthetic construct
  REGION 1..15: note = Synthetic Construct
KPLFRRMSSLELVIA

SEQ ID NO: 329 - protein, 9 aa, synthetic construct
  REGION 1..9: note = Synthetic Construct
EVDPIGHLY

SEQ ID NO: 330 - protein, 9 aa, synthetic construct
  REGION 1..9: note = Synthetic Construct
KVAELVHFL

SEQ ID NO: 331 - protein, 9 aa, synthetic construct
  REGION 1..9: note = Synthetic Construct
TFPDLESEF

SEQ ID NO: 332 - protein, 9 aa, synthetic construct
  REGION 1..9: note = Synthetic Construct
VAELVHFLL

SEQ ID NO: 333 - protein, 10 aa, synthetic construct
  REGION 1..10: note = Synthetic Construct
MEVDPIGHLY

SEQ ID NO: 334 - protein, 9 aa, synthetic construct
  REGION 1..9: note = Synthetic Construct
WQYFFPVIF

SEQ ID NO: 335 - protein, 16 aa, synthetic construct
  REGION 1..16: note = Synthetic Construct
KKLLTQHFVQENYLEY

SEQ ID NO: 336 - protein, 15 aa, synthetic construct
  REGION 1..15: note = Synthetic Construct
RKVAELVHFLLLKYR

SEQ ID NO: 337 - protein, 16 aa, synthetic construct
  REGION 1..16: note = Synthetic Construct
ACYEFLWGPRALVETS

SEQ ID NO: 338 - protein, 12 aa, synthetic construct
  REGION 1..12: note = Synthetic Construct
VIFSKASSSLQL

SEQ ID NO: 339 - protein, 15 aa, synthetic construct
  REGION 1..15: note = Synthetic Construct
VFGIELMEVDPIGHL

SEQ ID NO: 340 - protein, 15 aa, synthetic construct
  REGION 1..15: note = Synthetic Construct
GDNQIMPKAGLLIIV

SEQ ID NO: 341 - protein, 15 aa, synthetic construct
  REGION 1..15: note = Synthetic Construct
TSYVKVLHHMVKISG

SEQ ID NO: 342 - protein, 9 aa, synthetic construct
  REGION 1..9: note = Synthetic Construct
AELVHFLLL

SEQ ID NO: 343 - protein, 8 aa, synthetic construct
  REGION 1..8: note = Synthetic Construct
GLQLGVQA

(19) United States
(12) Patent Application Publication
(10) Pub. No.: US 2026/0216252 A1
(43) Pub. Date: Jul. 30, 2026

(54) IMMUNOMODULATORY CELLS AND USES THEREOF

(71) Applicant: Sinai Health System, Toronto (CA)

(72) Inventors: Andras Nagy, Toronto (CA); Jeffrey Harding, Toronto (CA); Kristina Nagy, Toronto (CA)

(73) Assignee: Sinai Health System, Toronto (CA)

(21) Appl. No.: 19/573,455

(22) Filed: Mar. 20, 2026

Related U.S. Application Data
(63) Continuation of application No. 17/413,314, filed on Dec. 13, 2019, now Pat. No. 12,622,932.
(60) Provisional application No. 62/779,449, filed on Dec. 13, 2018.

(51) Int. Cl. A61K 35/545; A61K 40/10; A61K 40/22; A61K 40/41; A61K 40/42; A61K 40/50; A61K 45/06; A61P 37/06; C07K 14/47; C07K 14/52; C07K 14/705; C07K 14/81

(52) U.S. Cl. CPC A61K 35/545; A61K 40/10; A61K 40/22; A61K 40/416; A61K 40/418; A61K 40/428; A61K 45/06; A61P 37/06; C07K 14/47; C07K 14/521; C07K 14/70503; C07K 14/70532; C07K 14/70575; C07K 14/811; A61K 40/50

(57) ABSTRACT

Featured are cells and methods of use thereof for modulating an antigen-specific immune response in a subject. The cells comprise a set of transgenes that shield the cells from immune surveillance. The cells can be used to induce immune tolerance to an antigen (e.g., a donor alloantigen or a self-antigen), or to induce an immune response to (e.g., induce the production of antibodies directed against) a non-self antigen.

### CROSS REFERENCE TO RELATED APPLICATIONS

This application is a continuation patent application based on U.S. National Phase Patent application Ser. No. 17/413,314 filed Jun. 11, 2021, which is based on International Patent Application No. PCT/CA2019/051808, filed on Dec. 13, 2019, which claims the benefit of U.S. Provisional Patent Application No. 62/779,449, file don Dec. 13, 2018, the entire contents each of which are incorporated herein by reference in their entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 3LG5045.XML. The file is 427,114 bytes, was created Mar. 17, 2026, and is being submitted electronically via Patent Center.

### BACKGROUND OF THE DISCLOSURE

The immune system is essential for the maintenance of health and protection against disease. The immune system is also capable of causing harm or impeding therapeutic treatment. For example, although the immune system is designed to detect and attack foreign antigens from bacteria, viruses, and other pathogens, an immune response can be mistakenly mounted against endogenous “self” antigens, which may lead to autoimmune disease. In addition, immune system activation against foreign antigens can make treatments, such as organ and tissue transplants, challenging and uncertain due a possibility that the immune system of a recipient will reject the organ or tissue transplant as foreign. Both transplant recipients and subjects with an autoimmune disease may be treated with immunosuppressive medication to dampen the response of the immune system, but this approach is accompanied by undesirable side effects and an increased risk of infection.

The adaptive immune response to foreign antigens involves the generation of antibodies, which leads to immunity against such antigens given that the antibodies can be rapidly generated by the immune system upon detection of the same antigen in the future. This feature of the immune response can be harnessed by vaccine technology, and a number of vaccines have been created using antigens from common viruses to produce widespread immunity. There is interest in using vaccines to treat a variety of different diseases and conditions, but the development of vaccines is a long and complex process that often lasts 10-15 years. For this reason, some have turned to other approaches to induce antibody-based immunity in a subject. One approach involves cell-based immunization, in which a cell is used to present an antigen to the immune system. This approach can be unreliable and cumbersome and may lead to the production of multiple non-specific antibodies against various proteins contained in the cell rather than production of a specific therapeutic antibody against the target antigen.

There is a need for targeted immunomodulatory therapies that can be used to generate a desired immune response to an antigen without off target effects or the need for widespread suppression of the immune system.

### SUMMARY OF THE DISCLOSURE

Featured are cells and methods of use thereof for modulating an antigen-specific immune response in a subject. The cells contain a set of transgenes that shield the cells from immune surveillance. The cells may further contain a polypeptide containing a donor alloantigen or a polypeptide containing a self-antigen. The cells can be used to induce immune tolerance to an antigen (e.g., a donor alloantigen or a self-antigen). The cells can also be used to induce immune tolerance in a subject in a method of inducing an immune response to (e.g., induce the production of antibodies directed against) a non-self antigen.

In a first aspect, the invention features a cell genetically modified to contain at least one mechanism for inducing immune tolerance to an antigen when administered to a subject, the genetically modified cell containing a transgene selected from one or more (e.g., one, two, three, four, five, six, seven, or all eight, e.g., a set of transgenes containing two, three, four, five, six, seven, or all eight) of the following genes: PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and a transgene encoding a polypeptide containing an antigen.

In some embodiments, each transgene of the set of transgenes encodes a gene product that is cytoplasmic, membrane bound, or local acting, and that has one or more of the following functions: a) to mitigate antigen presenting cell activation and function; b) to mitigate graft attacking leukocyte activity or cytolytic function; c) to mitigate macrophage cytolytic function and phagocytosis of allograft cells; d) to induce apoptosis in graft attacking leukocytes; e) to mitigate local inflammatory proteins; and f) to protect against leukocyte-mediated apoptosis.

In some embodiments, the cell contains a transgene selected from two or more (e.g., a set of transgenes containing two, three, four, five, six, seven, or all eight) of the following genes: PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6.

In some embodiments, the antigen is a donor alloantigen.

In some embodiments, the antigen is an antigen associated with an autoimmune disease or condition.

In some embodiments, the antigen is a self-antigen to which the subject's immune system mounts an aberrant immune response.

In some embodiments, the antigen is selected from an antigen listed in Table 2.

In some embodiments, the antigen is a food antigen or an allergen antigen.

In some embodiments, the antigen is not endogenous to the cell.

In some embodiments, the transgene encoding the polypeptide containing the antigen is incorporated into the genome of the cell at a non-endogenous locus.

In some embodiments, the polypeptide containing the antigen is expressed at a level that corresponds to the expression level of the polypeptide in a target cell. In some embodiments, the antigen is a donor alloantigen and the target cell is a donor cell. In some embodiments, the antigen is a self-antigen to which the subject's immune system mounts an aberrant immune response and the target cell is a cell from the subject that endogenously expresses the polypeptide.

In some embodiments, the polypeptide containing the antigen is expressed at a level that corresponds to the expression level of a polypeptide encoded by a housekeeping gene. In some embodiments, the polypeptide encoded by the housekeeping gene is β-actin, GAPDH, or Rosa26.

In another aspect, the invention features a method of inducing immune tolerance in a subject to a donor alloantigen, including the steps of: (i) providing a donor cell expressing a polypeptide containing the donor alloantigen; (ii) expressing in the cell a set of transgenes containing two or more (e.g., two, three, four, five, six, seven, or all eight) of the following genes: PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and (iii) administering the cell to the subject to induce immune tolerance to the donor alloantigen.

In another aspect, the invention features a method of inducing immune tolerance in a subject to a donor alloantigen, including the steps of: (i) providing a cell; (ii) expressing in the cell a set of transgenes containing two or more (e.g., two, three, four, five, six, seven, or all eight) of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; (iii) modifying the cell to express a transgene encoding a polypeptide containing the donor alloantigen; and (iv) administering the cell to the subject to induce immune tolerance to the donor alloantigen.

In some embodiments of any of the foregoing aspects, the donor alloantigen is a blood group antigen or a histocompatibility antigen.

In some embodiments, the polypeptide containing the donor alloantigen is expressed at a level corresponding to the expression level of the polypeptide containing the donor alloantigen in a donor cell.

In some embodiments, the cell is administered to or near a body site that will receive an organ or tissue transplant from the donor, or the cell is administered to a subcutaneous site.

In some embodiments, the method further includes measuring a response of an immune cell from the subject (e.g., immune cell activation, differentiation, polarization, proliferation, migration, pro-inflammatory cytokine production, degranulation, phagocytosis, or cytotoxicity) to the donor alloantigen after administration of the cell.

In some embodiments, the method prepares the subject to receive an organ or tissue transplant from the donor.

In some embodiments, the method further includes transplanting an organ or tissue from the donor into the subject after inducing immune tolerance to the donor alloantigen.

In some embodiments, the subject does not require treatment with an immunosuppressive medication.

In some embodiments, the method further includes administering to the subject an immunosuppressive medication. In some embodiments, the immunosuppressive medication is administered at a dose that is reduced relative to a standard dose of the immunosuppressive medication administered to a recipient of an organ or tissue transplant.

In another aspect, the invention features a cell of any of the foregoing aspects or embodiments for use in a method of inducing immune tolerance in a subject to an antigen.

In another aspect, the invention features a cell of any of the foregoing aspects or embodiments for use in a method of treating a subject having or at risk of developing an autoimmune disease or condition.

In another aspect, the invention features the use of the cell of any of the foregoing aspects or embodiments in the manufacture of a medicament for inducing immune tolerance in a subject to an antigen.

In another aspect, the invention features the use of the cell of any of the foregoing aspects or embodiments in the manufacture of a medicament for treating a subject having or at risk of developing an autoimmune disease or condition.

In another aspect, the invention features the use of the cell of any of the foregoing aspects or embodiments in the manufacture of a medicament for preparing a subject to receive an organ or tissue transplant.

In another aspect, the invention features a method of inducing immune tolerance in a subject to an antigen, including the steps of: (i) providing a cell; and (ii) expressing in the cell a set of transgenes containing two or more (e.g., two, three, four, five, six, seven, or all eight) of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; (iii) expressing in the cell a transgene encoding a polypeptide containing the antigen; and (iv) administering the cell to the subject to induce immune tolerance to the antigen.

In some embodiments, the antigen is an antigen associated with an autoimmune disease or condition.

In another aspect, the invention features a method of treating a subject having or at risk of developing an autoimmune disease or condition by administering to the subject a cell modified to express a transgene encoding a polypeptide containing an antigen associated with the autoimmune disease or condition and a set of transgenes including two or more (e.g., two, three, four, five, six, seven, or all eight) of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6.

In some embodiments of any of the foregoing aspects, the antigen is a self-antigen to which the subject's immune system mounts an aberrant immune response. In some embodiments, the polypeptide containing the self-antigen is expressed at a level that corresponds to the endogenous expression level of the polypeptide in a cell of the subject.

In some embodiments of any of the foregoing aspects, the antigen is selected from an antigen listed in Table 2.

In some embodiments of any of the foregoing aspects, the antigen is a food antigen or an allergen antigen.

In some embodiments, the cell is administered to or near a body site associated with an autoimmune disease or condition, or the cell is administered to a subcutaneous site.

In some embodiments, the method or use further includes measuring a response of an immune cell from the subject (e.g., immune cell activation, differentiation, polarization, proliferation, migration, pro-inflammatory cytokine production, degranulation, phagocytosis, or cytotoxicity) to the antigen after administration of the cell.

In some embodiments, the autoimmune disease or condition is multiple sclerosis (MS) and the polypeptide containing the antigen is myelin oligodendrocyte glycoprotein (MOG).

In some embodiments, the autoimmune disease or condition is type 1 diabetes and the polypeptide containing the antigen is insulin or glutamic acid decarboxylase 65 (GAD65).

In another aspect, the invention features a method of inducing an immune response in a subject to an antigen, including the steps of: (i) administering to the subject a first cell expressing a set of transgenes including two or more (e.g., two, three, four, five, six, seven, or all eight) of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and (ii) administering to the subject a second cell of the same type as the first cell that is modified to express a transgene encoding a polypeptide comprising the antigen and that does not express the set of transgenes.

In another aspect, the invention features the use of a combination of a first cell and a second cell for inducing an immune response in a subject to an antigen, the use including the steps of (i) administering to the subject a first cell expressing a set of transgenes containing two or more of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and (ii) administering to the subject a second cell of the same type as the first cell that is modified to express a transgene encoding a polypeptide containing the antigen and that does not express the set of transgenes.

In another aspect, the invention features the use of a first cell in the manufacture of a medicament for inducing an immune response in a subject to an antigen, characterized in that the medicament is for administration in a treatment regimen with a second cell, in which the first cell expresses a set of transgenes containing two or more of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and in which the second cell is a cell of the same type as the first cell that does not express the set of transgenes and that is modified to express a transgene encoding a polypeptide containing the antigen.

In some embodiments, the method or use further includes measuring a response of an immune cell from the subject (e.g., immune cell activation, differentiation, polarization, proliferation, migration, pro-inflammatory cytokine production, degranulation, phagocytosis, or cytotoxicity) to the first cell before administration of the second cell.

In some embodiments, the second cell is administered after inducing immune tolerance in the subject to the first cell.

In some embodiments, the antigen is a non-self antigen. In some embodiments, the non-self antigen is a cancer antigen, a viral antigen, a bacterial antigen, a fungal antigen, or a parasitic antigen.

In some embodiments, the cancer antigen is derived from a leukemia, lymphoma, sarcoma, carcinoma, blastoma, myeloma, melanoma, neuroma, glioma, liver cancer, bone cancer, lung cancer, brain cancer, bladder cancer, gastrointestinal cancer, urogenital cancer, breast cancer cardiac cancer, gynecological cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, skin cancer, thyroid cancer, bone and soft tissue sarcoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, Hodgkin lymphoma, hypopharyngeal cancer, islet cell tumors, pancreatic neuroendocrine tumors, Wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, stomach cancer, renal cancer, pelvic cancer, bronchus cancer, oropharyngeal cancer, larynx cancer, biliary tract cancer, a cancer of the central nervous system, a cancer of the respiratory system, and a cancer of the urinary system, cancer of the peritoneum, hepatocellular cancer, hepatoma, endometrial or uterine carcinoma, salivary gland carcinoma, hepatic carcinoma, anal carcinoma, penile carcinoma, post-transplant lymphoproliferative disorder (PTLD), bladder carcinoma, a pharynx cancer, a tumor of the tongue, a synovial cell sarcoma, a neuroblastoma, pheochromocytoma, pituitary tumor, pleuropulmonary blastoma, primary peritoneal cancer, rectal cancer, retinoblastoma, rhabdomyosarcoma, salivary gland cancer, Kaposi sarcoma, rhabdomyosarcoma, Sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, Waldenström macroglobulinemia, meningioma, malignant mesothelioma, or a virally induced cancer.

In some embodiments, the viral antigen is derived from a virus of a viral family selected from the group consisting of Retroviridae, Flaviviridae, Arenaviridae, Bunyaviridae, Filoviridae, Togaviridae, Poxviridae, Herpesviridae, Orthomyxoviridae, Coronaviridae, Rhabdoviridae, Paramyxoviridae, Picornaviridae, Hepadnaviridae, Papillomaviridae, Parvoviridae, Astroviridae, Polyomaviridae, Caliciviridae, and Reoviridae. In some embodiments, the viral antigen is derived from human immunodeficiency virus (HIV), human papillomavirus (HPV), hepatitis A virus (Hep A), hepatitis B virus (HBV), hepatitis C virus (HCV), Variola major, Variola minor, monkeypox virus, measles virus, rubella virus, mumps virus, varicella zoster virus (VZV), poliovirus, rabies virus, Japanese encephalitis virus, herpes simplex virus (HSV), cytomegalovirus (CMV), rotavirus, influenza, Ebola virus, yellow fever virus, Zika virus, or Marburg virus. In some embodiments, the polypeptide containing the viral antigen is Gag, Pol, Nef, Tat, Rev, Vif, Vpr, Vpu, 9D antigen, or Env.

In some embodiments, the bacterial antigen is derived from Acinetobacter spp., Bacteroides distasonis, Bacteroides fragilis, Bacteroides ovatus, Bacteroides thetaiotaomicron, Bacteroides uniformis, Bacteroides vulgatus, B. cepacia, Citrobacter freundii, Citrobacter koseri, Clostridium clostridioforme, Clostridium perfringens, C. sordellii, Enterobacter aerogenes, Enterobacter cloacae, Enterococcus faecalis, Enterococcus spp. Escherichia coli, Eubacterium lentum, Fusobacterium spp., Haemophilus influenza, Haemophilus parainfluenzae, Klebsiella pneumoniae, Klebsiella oxytoca, Legionella pneumophilia, Moraxella catarrhalis, Morganella morganii, Mycoplasma spp., Peptostreptococcus spp., Porphyromonas saccharolytica, Prevotella bivia, Proteus mirabilis, Proteus vulgaris, Providencia rettgeri, Providencia stuartii, Pseudomonas aeruginosa, Serratia marcescens, Streptococcus anginosus, Staphylococcus aureus, Staphylococcus epidermidis, Stenotrophomonas maltophilia, Streptococcus agalactiae, Streptococcus constellatus, Streptococcus pneumoniae, Streptococcus pyogenes, Streptococcus pyogenes, Mycobacterium tuberculosis, Mycobacterium bovis, Mycobacterium africanum, Mycobacterium microti, Mycobacterium leprae, Salmonella typhimurium, Francisella tularensis, Brucella, Burkholderia mallei, Yersinia pestis, Corynebacterium diphtheria, Neisseria meningitidis, Bordetella pertussis, Clostridium tetani, or Bacillus anthracis. In some embodiments, the polypeptide containing the bacterial antigen is 10.4, 85A, 85B, 86C, CFP-10, Rv3871, ESAT-6, O antigen, H antigen, K antigen, or protective antigen (PA).

In some embodiments, the parasitic antigen is derived from Toxoplasma gondii, Plasmodium falciparum, Plasmodium vivax, Plasmodium ovale, Plasmodium malariae, Trypanosoma spp., or Legionella spp. In some embodiments, the polypeptide containing the parasitic antigen is circumsporozoite (CS) protein, gamete surface protein Pfs230 or Pfs48/45, or Liver Specific Antigen 1 or 3 (LSA-1 or LSA-3). In some embodiments, the fungal antigen is derived from a mold pathogen from phylum Ascomycota, phylum Basidomycota, phylum Chytridiomycota, phylum, or phylum Oomycota in the Stramenopila kingdom. In some embodiments, the fungal antigen is derived from Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus. In some embodiments, the polypeptide containing the fungal antigen is a cell wall mannoprotein or a surface-expressed glycoprotein.

In some embodiments of any of the foregoing aspects, the polypeptide containing the antigen is expressed at a level that corresponds to the expression level of a polypeptide encoded by a housekeeping gene. In some embodiments, the polypeptide encoded by the housekeeping gene is β-actin, GAPDH, or Rosa26.

In some embodiments, the first cell and/or the second cell is administered to a subcutaneous site.

In some embodiments, the method or use further includes assessing a blood sample from the subject for antibodies directed against the antigen.

In some embodiments, the method or use immunizes the subject to the antigen.

In some embodiments, the method or use induces the production of antibodies directed against the antigen by the immune system of the subject.

In some embodiments of any of the foregoing aspects, the set of transgenes includes three, four, five, six, seven, or all eight of the following genes: PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6.

In some embodiments of any of the foregoing aspects, the set of transgenes includes PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, and Serpin B9 or Spi6.

In some embodiments of any of the foregoing aspects, the cell further contains one or more (e.g., one, two, three, four, five, six, seven, eight, nine, ten, or all eleven) of the following transgenes: TGF-β, Cd73, Cd39, Lag3, Il1r2, Ackr2, Tnfrsf22, Tnfrs23, Tnfrsf10, Dad1, and IFNγR1 d39 or a gene encoding a biologic that acts as an agonist of TGF-β, Cd73, Cd39, Lag3, Il1r2, Ackr2, Tnfrsf22, Tnfrs23, Tnfrsf10, Dad1, or IFNγR1 d39. In some embodiments, the TGF-β or biologic is local acting in the graft environment.

In some embodiments of any of the foregoing aspects, the cell is a stem cell, an autologous cell, a somatic cell, a germ cell, a syngeneic cell, or a cell amenable for genome editing.

In some embodiments of any of the foregoing aspects, the cell is an embryonic stem cell, a pluripotent stem cell, an induced pluripotent stem cell (iPSC), a hematopoietic stem cell, a mesenchymal stem cell, an endothelial stem cell, an epithelial stem cell, an adipose stem or progenitor cell, a germline stem cell, a lung stem or progenitor cells, a mammary stem cell, an olfactory adult stem cell, a hair follicle stem cell, an intestinal stem or progenitor cell, a multipotent stem cell, an amniotic stem cell, a cord blood stem cell, a neural stem or progenitor cell, an adult stem cell, a somatic stem cell, a tissue-specific stem cell, a totipotent stem cell, a fibroblast, a monocytic precursor, a B cell, an exocrine cell, a pancreatic progenitor, an endocrine progenitor, a hepatoblast, a myoblast, a preadipocyte, a hepatocyte, a chondrocyte, a smooth muscle cell, a K562 human erythroid leukemia cell line, a bone cell, a synovial cell, a tendon cell, a ligament cell, a meniscus cell, an adipose cell, a dendritic cell, a neutrophil, a basophil, a mast cell, a monocyte, an innate lymphoid cell, a natural killer cell, a skeletal muscle cell, a cardiac muscle cell, an erythroid-megakaryocytic cell, an eosinophil, a macrophage, a T cell, an islet beta-cell, a neuron, a cardiomyocyte, a blood cell, an exocrine progenitor, a ductal cell, an acinar cell, an alpha cell, a beta cell, a delta cell, a PP cell, a cholangiocyte, a white or brown adipocyte, a hormone-secreting cell, an epidermal keratinocyte, an epithelial cell, a kidney cell, a skeletal joint synovium cell, a periosteum cell, a perichondrium cell, a cartilage cell, an endothelial cell, a pericardium cell, a meningeal cell, a keratinocyte precursor cell, a keratinocyte stem cell, a pericyte, a glial cell, an ependymal cell, a cell isolated from an amniotic or placental membrane, a serosal cell, or a cell derived from skin, heart, brain or spinal cord, liver, lung, kidney, pancreas, bladder, bone marrow, spleen, intestine, or stomach.

In some embodiments of any of the foregoing aspects, the cell is an autologous cell.

In some embodiments of any of the foregoing aspects, the cell further includes at least one mechanism for controlling cell proliferation (e.g., reducing or inhibiting proliferation of a cell or ablating (e.g., killing) a cell), including: a genetic modification of one or more (e.g., one, two, three, or more) cell division locus/loci (CDL), the CDL being one or more endogenous or exogenous genetic loci encoding a transcription product(s) (e.g., a product resulting from transcription of a gene, the loss of expression of which would result in cell death or the inability of a cell to proliferate) that is expressed by dividing cells (e.g., cells actively undergoing cell division), cells capable of dividing but not currently undergoing cell division, or non-dividing cells (e.g., post-mitotic cells, such as a neuron). The genetic modification includes the incorporation at the CDL of one or more of: i) an ablation link (ALINK) system that includes a DNA sequence encoding a negative selectable marker that is transcriptionally linked to a DNA sequence of the CDL (e.g., the DNA sequence encoding the negative selectable marker is inserted into the CDL such that expression of the negative selectable marker is linked to expression of the transcription product of the CDL, e.g., such that the negative selectable marker and the transcription product of the CDL are co-expressed); and ii) an exogenous activator of regulation of a CDL (EARC) system that includes an inducible activator-based gene expression system that is operably linked to the CDL. In some embodiments, the genetic modification of the CDL includes performing targeted replacement of the CDL with one or more of: a) a DNA vector containing the ALINK system; b) a DNA vector containing the EARC system; and c) a DNA vector containing the ALINK system and the EARC system; wherein the ALINK and/or EARC systems are each operably linked to the CDL. In some embodiments, the genetic modification of the CDL includes performing targeted insertion into a coding or non-coding region of the CDL with one or more of a) a DNA sequence encoding the negative selectable marker; b) a DNA sequence encoding the inducible activator-based gene expression system; or c) a DNA sequence encoding the negative selectable marker and a DNA sequence encoding the inducible activator-based gene expression system. In some embodiments, the genetic modification of the CDL including the ALINK system is homozygous, heterozygous, hemizygous or compound heterozygous and/or wherein the genetic modification of the CDL including the EARC system results in activation of the CDL solely by an inducer of the inducible activator-based gene expression system. In some embodiments, the CDL encodes a gene product that functions in one or more of: cell cycle, DNA replication, RNA transcription, protein translation, and metabolism. In some embodiments, the CDL is a gene that is required for cell viability (e.g., cell survival, e.g., a gene without which the cell dies). In some embodiments, the CDL is a gene that is required for cell proliferation (e.g., a gene without which the cell cannot proliferate). In some embodiments, the CDL is one or more of Cdk1/CDK1, Top2A/TOP2A, Cenpa/CENPA, Birc5/BIRC5, and Eef2/EEF2, preferably wherein the CDL is Cdk1 or CDK1. In some embodiments, the CDL is two or more of Cdk1/CDK1, Top2A/TOP2A, Cenpa/CENPA, Birc5/BIRC5, and Eef2/EEF2, preferably wherein the CDL is Cdk1/CDK1 and Top2A/TOP2A or Cdk1/CDK1 and Eef2/EEF2. In some embodiments, the ALINK system includes a herpes simplex virus-thymidine kinase/ganciclovir system, a cytosine deaminase/5-fluorocytosine system, a carboxyl esterase/irinotecan system or an iCasp9/AP1903 system, preferably wherein the ALINK system is a herpes simplex virus-thymidine kinase/ganciclovir system. In some embodiments, the EARC system is a dox-bridge system, a cumate switch inducible system, an ecdysone inducible system, a radio wave inducible system, or a ligand-reversible dimerization system, preferably wherein the EARC system is a dox-bridge system.

In some embodiments of any of the foregoing aspects, the method or use further includes: i) permitting proliferation of the cell containing the ALINK system by maintaining the cell containing the ALINK system in the absence of an inducer of the negative selectable marker or ablating (e.g., killing) and/or inhibiting proliferation of the cell containing the ALINK system by exposing the cell containing the ALINK system to the inducer of the negative selectable marker; and/or ii) permitting proliferation of the cell containing the EARC system by exposing the cell containing the EARC system to an inducer of the inducible activator-based gene expression system or preventing or inhibiting proliferation of the cell containing the EARC system by maintaining the cell containing the EARC system in the absence of the inducer of the inducible activator-based gene expression system.

In some embodiments of any of the foregoing aspects, the method or use further includes removing the cell expressing the polypeptide containing the antigen after the immune system of the subject has responded to the antigen. In some embodiments, the cell is removed surgically. In some embodiments, the cell is removed by the use of the ALINK and/or EARC systems. In some embodiments, one or more (e.g., one, two, three, four, or more) ALINK and/or EARC systems are used to eliminate all of the cloaked cells and/or all of the cells expressing the polypeptide containing the antigen.

In some embodiments of any of the foregoing aspects, one or more (e.g., one, two, three, four, five, six, seven, or all eight) of PD-L1, HLA-G or H2-M3, Cd47, Cd200, FASLG or FasL, Ccl21 or Ccl21b, Mfge8, and Serpin B9 or Spi6 is expressed at a level that is equal to or greater than the expression level of the corresponding endogenous gene in an activated leukocyte (e.g., a T cell, e.g., the expression level of the cloaking transgene is equal to the level of expression of the endogenous gene in activated leukocytes, or is 1.5, 2, 3, 4, 5, 6, 7, 8, 9, 10-fold or more higher than the level of expression of the endogenous gene in activated leukocytes). In some embodiments, all eight of PD-L1, HLA-G or H2-M3, Cd47, Cd200, FASLG or FasL, Ccl21 or Ccl21b, Mfge8, and Serpin B9 or Spi6 are expressed at a level that is equal to or greater than the expression level of the corresponding endogenous gene in an activated leukocyte.

In some embodiments of any of the foregoing aspects, one or more (e.g., one, two, three, four, five, six, seven, or all eight) of PD-L1, HLA-G or H2-M3, Cd47, Cd200, FASLG or FasL, Ccl21 or Ccl21b, Mfge8, and Serpin B9 or Spi6 is expressed at a level that is in the top 5% of gene expression for all genes in the genome of the cell. In some embodiments, all eight of PD-L1, HLA-G or H2-M3, Cd47, Cd200, FASLG or FasL, Ccl21 or Ccl21b, Mfge8, and Serpin B9 or Spi6 are expressed at a level that is in the top 5% of gene expression for all genes in the genome of the cell.

In some embodiments of any of the foregoing aspects, one or more (e.g., one, two, three, four, five, six, seven, or all eight) of PD-L1, HLA-G (H2-M3), Cd47, Cd200, FASLG (FasL), Ccl21 (Ccl21b), Mfge8, and Serpin B9 (Spi6) is expressed at a level that is greater than the expression level of the corresponding endogenous gene in a wild-type stem cell (e.g., a wild-type ES cell from the same species, e.g., the expression level of the cloaking transgene is at least 2, 3, 4, 5, 6, 7, 8, 9, 10, 20, 30, 40, 50, 100, 500, 1,000-fold or more higher in cloaked cells compared to expression of the endogenous gene in unmodified wild-type ES cells from the same species). In some embodiments, all 8 of PD-L1, HLA-G (H2-M3), Cd47, Cd200, FASLG (FasL), Ccl21 (Ccl21b), Mfge8, and Serpin B9 (Spi6) are expressed at a level that is greater (e.g., 2, 3, 4, 5, 6, 7, 8, 9, 10, 20, 30, 40, 50, or 100-fold higher or more) than the expression level of the endogenous gene in a wild-type stem cell (e.g., an embryonic stem cell from the same species as the cloaked cell). In some embodiments, one or more (e.g., one, two, three, four, five, six, seven, or all eight) of PD-L1, HLA-G (H2-M3), Cd47, Cd200, FASLG (FasL), Ccl21 (Ccl21b), Mfge8, and Serpin B9 (Spi6) is expressed at a level that is in the top 5% of gene expression for all genes in the ES cell genome. In some embodiments, one or more (e.g., one, two, three, four, five, six, seven, or all eight) of PD-L1, HLA-G (H2-M3), Cd47, Cd200, FASLG (FasL), Ccl21 (Ccl21b), Mfge8, and Serpin B9 (Spi6) is expressed at a level that is in the top 1% of gene expression for all genes in the ES cell genome. In some embodiments, all of PD-L1, HLA-G (H2-M3), Cd47, Cd200, FASLG (FasL), Ccl21 (Ccl21b), Mfge8, and Serpin B9 (Spi6) are expressed at a level that is in the top 5% of gene expression for all genes in the ES cell genome.

In some embodiments of any of the foregoing aspects, the PD-L1 transgene encodes a protein having at least 85% identity (e.g., 85%, 90%, 95%, 96%, 97%, 98%, 99% sequence identity or more) to the amino acid sequence of SEQ ID NO: 11 or SEQ ID NO: 12.

In some embodiments of any of the foregoing aspects, the HLA-G or H2-M3 transgene encodes a protein having at least 85% identity (e.g., 85%, 90%, 95%, 96%, 97%, 98%, 99% sequence identity or more) to the amino acid sequence of SEQ ID NO: 16 or SEQ ID NO: 15.

In some embodiments of any of the foregoing aspects, the Cd47 transgene encodes a protein having at least 85% identity (e.g., 85%, 90%, 95%, 96%, 97%, 98%, 99% sequence identity or more) to the amino acid sequence of SEQ ID NO: 3 or SEQ ID NO: 4.

In some embodiments of any of the foregoing aspects, the CD200 transgene encodes a protein having at least 85% identity (e.g., 85%, 90%, 95%, 96%, 97%, 98%, 99% sequence identity or more) to the amino acid sequence of SEQ ID NO: 5 or SEQ ID NO: 6.

In some embodiments of any of the foregoing aspects, the FASLG or FasL transgene encodes a protein having at least 85% identity (e.g., 85%, 90%, 95%, 96%, 97%, 98%, 99% sequence identity or more) to the amino acid sequence of SEQ ID NO: 10 or SEQ ID NO: 9.

In some embodiments of any of the foregoing aspects, the Ccl21 or Ccl21b transgene encodes a protein having at least 85% identity (e.g., 85%, 90%, 95%, 96%, 97%, 98%, 99% sequence identity or more) to the amino acid sequence of SEQ ID NO: 2 or SEQ ID NO: 1.

In some embodiments of any of the foregoing aspects, the Mfge8 transgene encodes a protein having at least 85% identity (e.g., 85%, 90%, 95%, 96%, 97%, 98%, 99% sequence identity or more) to the amino acid sequence of SEQ ID NO: 13 or SEQ ID NO: 14.

In some embodiments of any of the foregoing aspects, the Serpin B9 or Spi6 transgene encodes a protein having at least 85% identity (e.g., 85%, 90%, 95%, 96%, 97%, 98%, 99% sequence identity or more) to the amino acid sequence of SEQ ID NO: 8 or SEQ ID NO: 7.

In some embodiments of any of the foregoing aspects, the IFNγR1 d39 transgene encodes a protein having at least 85% identity (e.g., 85%, 90%, 95%, 96%, 97%, 98%, 99% sequence identity or more) to the amino acid sequence of SEQ ID NO: 17.

In some embodiments of any of the foregoing aspects, the two or more transgenes (e.g., of the set of PD-L1, HLA-G or H2-M3, Cd47, Cd200, FASLG or FasL, Ccl21 or Ccl21b, Mfge8, and Serpin B9 or Spi6) are operably linked to a constitutive promoter.

In some embodiments of any of the foregoing aspects, the transgene encoding the polypeptide containing the antigen is operably linked to a constitutive promoter.

In some embodiments of any of the foregoing aspects, the constitutive promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

In some embodiments of any of the foregoing aspects, the polypeptide containing the antigen is expressed using an inducible expression system selected from the group consisting of a tetracycline response element, a light inducible system, a radiogenetic system, a cumate switch inducible system, an ecdysone inducible system, a destabilization domain system, or a ligand-reversible dimerization system. In some embodiments of any of the foregoing aspects, the cell includes an EARC system, and wherein expression of the EARC system and the polypeptide containing the antigen is controlled by two different inducible expression systems.

In some embodiments of any of the foregoing aspects, the cells are administered intravenously, subcutaneously, intramuscularly, percutaneously, intradermally, parenterally, intraarterially, intravascularly, or by perfusion.

In some embodiments, the cells are administered as a tissue. In some embodiments, the tissue is administered with a gel, biocompatible matrix, or cellular scaffold.

In some embodiments of any of the foregoing aspects, the cells are administered in an amount of 25,000 to 5,000,000,000 cells (e.g., 2.5×10⁴, 5×10⁴, 7.5×10⁴, 1×10⁵, 2×10⁵, 3×10⁵, 4×10⁵, 6×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, or 5×10⁹ cells).

In some embodiments of any of the foregoing aspects, the cells are administered in an amount of 800,000,000 to 100,000,000,000 cells (e.g., 8×10⁸, 9×10⁸, 1×10⁹, 2×10⁹, or 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ cells).

In some embodiments of any of the foregoing aspects, the method further includes administering an additional therapeutic agent. In some embodiments, the additional therapeutic agent is administered after administration of the cells. In some embodiments, the additional therapeutic agent is administered concurrently with administration of the cells. In some embodiments, the additional therapeutic agent is an immunosuppressive agent, a disease-modifying anti-rheumatic drug (DMARD), a biologic response modifier (a type of DMARD), a corticosteroid, or a nonsteroidal anti-inflammatory medication (NSAID), prednisone, prednisolone, methylprednisolone, methotrexate, hydroxychloroquine, sulfasalazine, leflunomide, cyclophosphamide, azathioprine, tofacitinib, adalimumab, abatacept, anakinra, kineret, certolizumab, etanercept, golimumab, infliximab, rituximab or tocilizumab, 6-mercaptopurine, 6-thioguanine, abatacept, adalimumab, alemtuzumab, an aminosalicylate, an antibiotic, an anti-histamine, Anti-TNFα, azathioprine, belimumab, beta interferon, a calcineurin inhibitor, certolizumab, a corticosteroid, cromolyn, cyclosporin A, cyclosporine, dimethyl fumarate, etanercept, fingolimod, fumaric acid esters, glatiramer acetate, golimumab, hydroxyurea, IFNγ, IL-11, leflunomide, leukotriene receptor antagonist, long-acting beta2 agonist, mitoxantrone, mycophenolate mofetil, natalizumab, ocrelizumab, pimecrolimus, a probiotic, a retinoid, salicylic acid, short-acting beta2 agonist, sulfasalazine, tacrolimus, teriflunomide, theophylline, tocilizumab, ustekinumab, vedolizumab, interferon beta-1b, gliatrimer acetate, daclizumab, teriflunomide, fingolimod, dimethyl fumarate, alemtuzumab, mitoxantrone, ocrelizumab, natalizumab, an antiviral compound, a nucleoside-analog reverse transcriptase inhibitor (NRTI), a non-nucleoside reverse transcriptase inhibitor (NNRTI), a protease inhibitor, an antibacterial compound, an antifungal compound, an antiparasitic compound, insulin, a sulfonylurea, a biguanide, a meglitinide, a thiazolidinedione, a DPP-4 inhibitor, an SGLT2 inhibitor, an alpha-glucosidase inhibitor, a bile acid sequestrant, aspirin, a dietary regimen, an immunosuppressive agent, a checkpoint inhibitor, a chemotherapeutic drug, a biologic drug, radiation therapy, cryotherapy, hyperthermia, surgical excision or tumor tissue, or an anti-cancer vaccine. In some embodiments of any of the foregoing aspects, the subject is a mammal. In some embodiments, the mammal is a mouse. In some embodiments, the mammal is a human.

In some embodiments of any of the foregoing aspects, the cell includes two of the set of transgenes PD-L1, HLA-G (H2-M3), Cd47, Cd200, FASLG (FasL), Ccl21 (Ccl21b), Mfge8, and Serpin B9 (Spi6) (e.g., PD-L1 and HLA-G (H2-M3); PD-L1 and Cd47; PD-L1 and Cd200; PD-L1 and FASLG (FasL); PD-L1 and Ccl21 (Ccl21b); PD-L1 and Mfge8; PD-L1 and Serpin B9 (Spi6); HLA-G (H2-M3) and Cd47; HLA-G (H2-M3) and Cd200; HLA-G (H2-M3) and FASLG (FasL); HLA-G (H2-M3) and Ccl21 (Ccl21b); HLA-G (H2-M3) and Mfge8; HLA-G (H2-M3) and Serpin B9 (Spi6); Cd47 and Cd200; Cd47 and FASLG (FasL); Cd47 and Ccl21 (Ccl21b); Cd47 and Mfge8; Cd47 and Serpin B9 (Spi6); Cd200 and FASLG (FasL); Cd200 and Ccl21 (Ccl21b); Cd200 and Mfge8; Cd200 and Serpin B9 (Spi6); FASLG (FasL) and Ccl21 (Ccl21b); FASLG (FasL) and Mfge8; FASLG (FasL) and Serpin B9 (Spi6); Ccl21 (Ccl21b) and Mfge8; Ccl21 (Ccl21b) and Serpin B9 (Spi6); or Mfge8 and Serpin B9 (Spi6)).

In some embodiments of any of the foregoing aspects, the cell includes three of the set of transgenes PD-L1, HLA-G (H2-M3), Cd47, Cd200, FASLG (FasL), Ccl21 (Ccl21b), Mfge8, and Serpin B9 (Spi6) (e.g., PD-L1, HLA-G (H2-M3), and Cd47; PD-L1, HLA-G (H2-M3), and Cd200; PD-L1, HLA-G (H2-M3), and FASLG (FasL); PD-L1, HLA-G (H2-M3), and Ccl21 (Ccl21b); PD-L1, HLA-G (H2-M3), and Mfge8; PD-L1, HLA-G (H2-M3), and Serpin B9 (Spi6); PD-L1, Cd47, and Cd200; PD-L1, Cd47, and FASLG (FasL); PD-L1, Cd47, and Ccl21 (Ccl21b); PD-L1, Cd47, and Mfge8; PD-L1, Cd47, and Serpin B9; PD-L1, Cd200, and FASLG (FasL); PD-L1, Cd200, and Ccl21 (Ccl21b); PD-L1, Cd200, and Mfge8; PD-L1, Cd200, and Serpin B9 (Spi6); PD-L1, FASLG (FasL), and Ccl21 (Ccl21b); PD-L1, FASLG (FasL), and Mfge8; PD-L1, FASLG (FasL), and Serpin B9 (Spi6); PD-L1, Ccl21 (Ccl21b), and Mfge8; PD-L1, Ccl21 (Ccl21b), and Serpin B9 (Spi6); PD-L1, Mfge8, and Serpin B9 (Spi6); HLA-G (H2-M3), Cd47, and Cd200; HLA-G (H2-M3), Cd47, and FASLG (FasL); HLA-G (H2-M3), Cd47, and Ccl21 (Ccl21b); HLA-G (H2-M3), Cd47, and Mfge8; HLA-G (H2-M3), Cd47, and Serpin B9; HLA-G (H2-M3), Cd200, and FASLG (FasL); HLA-G (H2-M3), Cd200, and Ccl21 (Ccl21b); HLA-G (H2-M3), Cd200, and Mfge8; HLA-G (H2-M3), Cd200, and Serpin B9; HLA-G (H2-M3), FASLG (FasL), and Ccl21 (Ccl21b); HLA-G (H2-M3), FASLG (FasL), and Mfge8; HLA-G (H2-M3), FASLG (FasL), and Serpin B9 (Spi6); HLA-G (H2-M3), Ccl21 (Ccl21b), and Mfge8; HLA-G (H2-M3), Ccl21 (Ccl21b), and Serpin B9 (Spi6); HLA-G (H2-M3), Mfge8, and Serpin B9 (Spi6); Cd47, Cd200, and FASLG (FasL); Cd47, Cd200, and Ccl21 (Ccl21b); Cd47, Cd200, and Mfge8; Cd47, Cd200, and Serpin B9 (Spi6); Cd47, FASLG (FasL), and Ccl21 (Ccl21b); Cd47, FASLG (FasL), and Mfge8; Cd47, FASLG (FasL), and Serpin B9 (Spi6); Cd47, Ccl21 (Ccl21b), and Mfge8; Cd47, Ccl21 (Ccl21b), and Serpin B9 (Spi6); Cd47, Mfge8, and Serpin B9 (Spi6); Cd200, FASLG (FasL), and Ccl21 (Ccl21b); Cd200, FASLG (FasL), and Mfge8; Cd200, FASLG (FasL), and Serpin B9 (Spi6); Cd200, Ccl21 (Ccl21b), and Mfge8; Cd200, Ccl21 (Ccl21b), and Serpin B9 (Spi6); Cd200, Mfge8, and Serpin B9 (Spi6); FASLG (FasL), Ccl21 (Ccl21b), and Mfge8; FASLG (FasL), Ccl21 (Ccl21b), and Serpin B9 (Spi6); Ccl21 (Ccl21b), Mfge8, and Serpin B9 (Spi6)).

In some embodiments of any of the foregoing aspects, the cell includes four of the set of transgenes PD-L1, HLA-G (H2-M3), Cd47, Cd200, FASLG (FasL), Ccl21 (Ccl21b), Mfge8, and Serpin B9 (Spi6) (e.g., PD-L1, HLA-G (H2-M3), Cd47, and Cd200; PD-L1, HLA-G (H2-M3), Cd47, and FASLG (FasL); PD-L1, HLA-G (H2-M3), Cd47, and Ccl21 (Ccl21b); PD-L1, HLA-G (H2-M3), Cd47, and Mfge8; PD-L1, HLA-G (H2-M3), Cd47, and Serpin B9 (Spi6); PD-L1, HLA-G (H2-M3), Cd200, and FASLG (FasL); PD-L1, HLA-G (H2-M3), Cd200, and Ccl21 (Ccl21b); PD-L1, HLA-G (H2-M3), Cd200, and Mfge8; PD-L1, HLA-G (H2-M3), Cd200, and Serpin B9 (Spi6); PD-L1, HLA-G (H2-M3), FASLG (FasL), and Ccl21 (Ccl21b); PD-L1, HLA-G (H2-M3), FASLG (FasL), and Mfge8; PD-L1, HLA-G (H2-M3), FASLG (FasL), and Serpin B9 (Spi6); PD-L1, HLA-G (H2-M3), Ccl21 (Ccl21b), and Mfge8; PD-L1, HLA-G (H2-M3), Ccl21 (Ccl21b), and Serpin B9 (Spi6); PD-L1, HLA-G (H2-M3), Mfge8, and Serpin B9 (Spi6); PD-L1, Cd47, Cd200, and FASLG (FasL); PD-L1, Cd47, Cd200, and Ccl21 (Ccl21b); PD-L1, Cd47, Cd200, and Mfge8; PD-L1, Cd47, Cd200, and Serpin B9 (Spi6); PD-L1, Cd47, FASLG (FasL), and Ccl21 (Ccl21b); PD-L1, Cd47, FASLG (FasL), and Mfge8; PD-L1, Cd47, FASLG (FasL), and Serpin B9 (Spi6); PD-L1, Cd47, Ccl21 (Ccl21b), and Mfge8; PD-L1, Cd47, Ccl21 (Ccl21b), and Serpin B9 (Spi6); PD-L1, Cd47, Mfge8, and Serpin B9 (Spi6); PD-L1, Cd200, FASLG (FasL), and Ccl21 (Ccl21b); PD-L1, Cd200, FASLG (FasL), and Mfge8; PD-L1, Cd200, FASLG (FasL), and Serpin B9 (Spi6); PD-L1, Cd200, Ccl21 (Ccl21b), and Mfge8; PD-L1, Cd200, Ccl21 (Ccl21b), and Serpin B9 (Spi6); PD-L1, Cd200, Mfge8, and Serpin B9 (Spi6); PD-L1, FASLG (FasL), Ccl21 (Ccl21b), and Mfge8; PD-L1, FASLG (FasL), Ccl21 (Ccl21b), and Serpin B9 (Spi6); PD-L1, FASLG (FasL), Mfge8, and Serpin B9 (Spi6); PD-L1, Ccl21 (Ccl21b), Mfge8, and Serpin B9 (Spi6); HLA-G (H2-M3), Cd47, Cd200, and FASLG (FasL); HLA-G (H2-M3), Cd47, Cd200, and Ccl21 (Ccl21b); HLA-G (H2-M3), Cd47, Cd200, and Mfge8; HLA-G (H2-M3), Cd47, Cd200, and Serpin B9 (Spi6); HLA-G (H2-M3), Cd47, FASLG (FasL), and Ccl21 (Ccl21b); HLA-G (H2-M3), Cd47, FASLG (FasL), and Mfge8; HLA-G (H2-M3), Cd47, FASLG (FasL), and Serpin B9 (Spi6); HLA-G (H2-M3), Cd47, Ccl21 (Ccl21b), and Mfge8; HLA-G (H2-M3), Cd47, Ccl21 (Ccl21b), and Serpin B9 (Spi6); HLA-G (H2-M3), Cd47, Mfge8, and Serpin B9 (Spi6); HLA-G (H2-M3), Cd200, FASLG (FasL), and Ccl21 (Ccl21b); HLA-G (H2-M3), Cd200, FASLG (FasL), and Mfge8; HLA-G (H2-M3), Cd200, FASLG (FasL), and Serpin B9 (Spi6); HLA-G (H2-M3), Cd200, Ccl21 (Ccl21b), and Mfge8; HLA-G (H2-M3), Cd200, Ccl21 (Ccl21b), and Serpin B9 (Spi6); HLA-G (H2-M3), Cd200, Mfge8, and Serpin B9 (Spi6); HLA-G (H2-M3), FASLG (FasL), Ccl21 (Ccl21b), and Mfge8; HLA-G (H2-M3), FASLG (FasL), Ccl21 (Ccl21b), and Serpin B9 (Spi6); HLA-G (H2-M3), FASLG (FasL), Mfge8, and Serpin B9 (Spi6); HLA-G (H2-M3), Ccl21 (Ccl21b), Mfge8, and Serpin B9 (Spi6); Cd47, Cd200, FASLG (FasL), and Ccl21 (Ccl21b); Cd47, Cd200, FASLG (FasL), and Mfge8; Cd47, Cd200, FASLG (FasL), and Serpin B9 (Spi6); Cd47, Cd200, Ccl21 (Ccl21b), and Mfge8; Cd47, Cd200, Ccl21 (Ccl21b), and Serpin B9 (Spi6); Cd47, Cd200, Mfge8, and Serpin B9 (Spi6); Cd47, FASLG (FasL), Ccl21 (Ccl21b), and Mfge8; Cd47, FASLG (FasL), Ccl21 (Ccl21b), and Serpin B9 (Spi6); Cd47, Ccl21 (Ccl21b), Mfge8, and Serpin B9 (Spi6); Cd200, FASLG (FasL), Ccl21 (Ccl21b), and Mfge8; Cd200, FASLG (FasL), Ccl21 (Ccl21b), and Serpin B9 (Spi6); Cd200, Ccl21 (Ccl21b), Mfge8, and Serpin B9 (Spi6); or FASLG (FasL), Ccl21 (Ccl21b), Mfge8, and Serpin B9 (Spi6)).

In some embodiments of any of the foregoing aspects, the cell includes five of the set of transgenes PD-L1, HLA-G (H2-M3), Cd47, Cd200, FASLG (FasL), Ccl21 (Ccl21b), Mfge8, and Serpin B9 (Spi6) (e.g., PD-L1, HLA-G (H2-M3), Cd47, Cd200, and FASLG (FasL); PD-L1, HLA-G (H2-M3), Cd47, Cd200, and Ccl21 (Ccl21b); PD-L1, HLA-G (H2-M3), Cd47, Cd200, and Mfge8; PD-L1, HLA-G (H2-M3), Cd47, Cd200, and Serpin B9 (Spi6); PD-L1, HLA-G (H2-M3), Cd47, FASLG (FasL), and Ccl21 (Ccl21b); PD-L1, HLA-G (H2-M3), Cd47, FASLG (FasL), and Mfge8; PD-L1, HLA-G (H2-M3), Cd47, FASLG (FasL), and Serpin B9 (Spi6); PD-L1, HLA-G (H2-M3), Cd47, Ccl21 (Ccl21b), and Mfge8; PD-L1, HLA-G (H2-M3), Cd47, Ccl21 (Ccl21b), and Serpin B9 (Spi6); PD-L1, HLA-G (H2-M3), Cd47, Mfge8, and Serpin B9 (Spi6); PD-L1, HLA-G (H2-M3), Cd200, FASLG (FasL), and Ccl21 (Ccl21b); PD-L1, HLA-G (H2-M3), Cd200, FASLG (FasL), and Mfge8, PD-L1, HLA-G (H2-M3), Cd200, FASLG (FasL), and Serpin B9 (Spi6); PD-L1, HLA-G (H2-M3), Cd200, Ccl21 (Ccl21b), and Mfge8; PD-L1, HLA-G (H2-M3), Cd200, Ccl21 (Ccl21b), and Serpin B9 (Spi6); PD-L1, HLA-G (H2-M3), Cd200, Mfge8, and Serpin B9 (Spi6); PD-L1, HLA-G (H2-M3), FASLG (FasL), Ccl21 (Ccl21b), and Mfge8; PD-L1, HLA-G (H2-M3), FASLG (FasL), Ccl21 (Ccl21b), and Serpin B9 (Spi6); PD-L1, HLA-G (H2-M3), FASLG (FasL), Mfge8, and Serpin B9 (Spi6); PD-L1, HLA-G (H2-M3), Ccl21 (Ccl21b), Mfge8, and Serpin B9 (Spi6); PD-L1, Cd47, Cd200, FASLG (FasL), and Ccl21 (Ccl21b); PD-L1, Cd47, Cd200, FASLG (FasL), and Mfge8, PD-L1, Cd47, Cd200, FASLG (FasL), and Serpin B9 (Spi6); PD-L1, Cd47, Cd200, Ccl21 (Ccl21b), and Mfge8; PD-L1, Cd47, Cd200, Ccl21 (Ccl21b), and Serpin B9 (Spi6); PD-L1, Cd47, Cd200, Mfge8, and Serpin B9 (Spi6); PD-L1, Cd47, FASLG (FasL), Ccl21 (Ccl21b), and Mfge8; PD-L1, Cd47, FASLG (FasL), Ccl21 (Ccl21b), and Serpin B9 (Spi6); PD-L1, Cd47, FASLG (FasL), Mfge8, and Serpin B9 (Spi6); PD-L1, Cd47, Ccl21 (Ccl21b), Mfge8, and Serpin B9 (Spi6); PD-L1, Cd200, FASLG (FasL), Ccl21 (Ccl21b), and Mfge8; PD-L1, Cd200, FASLG (FasL), Ccl21 (Ccl21b), and Serpin B9 (Spi6); PD-L1, Cd200, FASLG (FasL), Mfge8, and Serpin B9 (Spi6); PD-L1, Cd200, Ccl21 (Ccl21b), Mfge8, and Serpin B9 (Spi6); PD-L1, FASLG (FasL), Ccl21 (Ccl21b), Mfge8, and Serpin B9 (Spi6); HLA-G (H2-M3), Cd47, Cd200, FASLG (FasL), and Ccl21 (Ccl21b); HLA-G (H2-M3), Cd47, Cd200, FASLG (FasL), and Mfge8; HLA-G (H2-M3), Cd47, Cd200, FASLG (FasL), and Serpin B9 (Spi6); HLA-G (H2-M3), Cd47, Cd200, Ccl21 (Ccl21b), and Mfge8; HLA-G (H2-M3), Cd47, Cd200, Ccl21 (Ccl21b), and Serpin B9 (Spi6); HLA-G (H2-M3), Cd47, Cd200, Mfge8, and Serpin B9 (Spi6); HLA-G (H2-M3), Cd47, FASLG (FasL), Ccl21 (Ccl21b), and Mfge8; HLA-G (H2-M3), Cd47, FASLG (FasL), Ccl21 (Ccl21b), and Serpin B9 (Spi6); HLA-G (H2-M3), Cd47, FASLG (FasL), Mfge8, and Serpin B9 (Spi6); HLA-G (H2-M3), Cd47, Ccl21 (Ccl21b), Mfge8, and Serpin B9 (Spi6); HLA-G (H2-M3), Cd200, FASLG (FasL), Ccl21 (Ccl21b), and Mfge8; HLA-G (H2-M3), Cd200, FASLG (FasL), Ccl21 (Ccl21b), and Serpin B9 (Spi6); HLA-G (H2-M3), Cd200, FASLG (FasL), Mfge8, and Serpin B9 (Spi6); HLA-G (H2-M3), Cd200, Ccl21 (Ccl21b), Mfge8, and Serpin B9 (Spi6); HLA-G (H2-M3), FASLG (FasL), Ccl21 (Ccl21b), Mfge8, and Serpin B9 (Spi6); Cd47, Cd200, FASLG (FasL), Ccl21 (Ccl21b), and Mfge8; Cd47, Cd200, FASLG (FasL), Ccl21 (Ccl21b), and Serpin B9 (Spi6); Cd47, Cd200, FASLG (FasL), Mfge8, and Serpin B9 (Spi6); Cd47, Cd200, Ccl21 (Ccl21b), Mfge8, and Serpin B9 (Spi6); Cd47, FASLG (FasL), Ccl21 (Ccl21b), Mfge8, and Serpin B9 (Spi6); or Cd200, FASLG (FasL), Ccl21 (Ccl21b), Mfge8, and Serpin B9 (Spi6)).

In some embodiments of any of the foregoing aspects, the cell includes six of the set of transgenes PD-L1, HLA-G (H2-M3), Cd47, Cd200, FASLG (FasL), Ccl21 (Ccl21b), Mfge8, and Serpin B9 (Spi6) (e.g., PD-L1, HLA-G (H2-M3), Cd47, Cd200, FASLG (FasL), and Ccl21 (Ccl21b); PD-L1, HLA-G (H2-M3), Cd47, Cd200, FASLG (FasL), and Mfge8; PD-L1, HLA-G (H2-M3), Cd47, Cd200, FASLG (FasL), and Serpin B9 (Spi6); PD-L1, HLA-G (H2-M3), Cd47, Cd200, Ccl21 (Ccl21b), and Mfge8; PD-L1, HLA-G (H2-M3), Cd47, Cd200, Ccl21 (Ccl21b), and Serpin B9 (Spi6); PD-L1, HLA-G (H2-M3), Cd47, Cd200, Mfge8, and Serpin B9 (Spi6); PD-L1, HLA-G (H2-M3), Cd47, FASLG (FasL), Ccl21 (Ccl21b), and Mfge8; PD-L1, HLA-G (H2-M3), Cd47, FASLG (FasL), Ccl21 (Ccl21b), and Serpin B9 (Spi6); PD-L1, HLA-G (H2-M3), Cd47, FASLG (FasL), Mfge8, and Serpin B9 (Spi6); PD-L1, HLA-G (H2-M3), Cd200, FASLG (FasL), Ccl21 (Ccl21b), and Mfge8; PD-L1, HLA-G (H2-M3), Cd200, FASLG (FasL), Ccl21 (Ccl21b), and Serpin B9 (Spi6); PD-L1, HLA-G (H2-M3), Cd200, FASLG (FasL), Mfge8, and Serpin B9 (Spi6); PD-L1, HLA-G (H2-M3), Cd200, Ccl21 (Ccl21b), Mfge8, and Serpin B9 (Spi6); PD-L1, Cd47, Cd200, FASLG (FasL), Ccl21 (Ccl21b), and Mfge8; PD-L1, Cd47, Cd200, FASLG (FasL), Ccl21 (Ccl21b), and Serpin B9 (Spi6); PD-L1, Cd47, Cd200, FASLG (FasL), Mfge8, and Serpin B9 (Spi6); PD-L1, Cd47, Cd200, Ccl21 (Ccl21b), Mfge8, and Serpin B9 (Spi6); PD-L1, Cd47, FASLG (FasL), Ccl21 (Ccl21b), Mfge8, and Serpin B9 (Spi6); PD-L1, Cd200, FASLG (FasL), Ccl21 (Ccl21b), Mfge8, and Serpin B9 (Spi6); HLA-G (H2-M3), Cd47, Cd200, FASLG (FasL), Ccl21 (Ccl21b), and Mfge8; HLA-G (H2-M3), Cd47, Cd200, FASLG (FasL), Ccl21 (Ccl21b), and Serpin B9 (Spi6); HLA-G (H2-M3), Cd47, Cd200, FASLG (FasL), Mfge8, and Serpin B9 (Spi6); HLA-G (H2-M3), Cd47, Cd200, Ccl21 (Ccl21b), Mfge8, and Serpin B9 (Spi6); HLA-G (H2-M3), Cd47, FASLG (FasL), Ccl21 (Ccl21b), Mfge8, and Serpin B9 (Spi6); HLA-G (H2-M3), Cd200, FASLG (FasL), Ccl21 (Ccl21b), Mfge8, and Serpin B9 (Spi6); or Cd47, Cd200, FASLG (FasL), Ccl21 (Ccl21b), Mfge8, and Serpin B9 (Spi6)).

In some embodiments of any of the foregoing aspects, the cell includes seven of the set of transgenes PD-L1, HLA-G (H2-M3), Cd47, Cd200, FASLG (FasL), Ccl21 (Ccl21b), Mfge8, and Serpin B9 (Spi6) (e.g., PD-L1, HLA-G (H2-M3), Cd47, Cd200, FASLG (FasL), Ccl21 (Ccl21b), and Mfge8; PD-L1, HLA-G (H2-M3), Cd47, Cd200, FASLG (FasL), Ccl21 (Ccl21b), and Serpin B9 (Spi6); PD-L1, HLA-G (H2-M3), Cd47, Cd200, FASLG (FasL), Mfge8, and Serpin B9 (Spi6); PD-L1, HLA-G (H2-M3), Cd47, Cd200, Ccl21 (Ccl21b), Mfge8, and Serpin B9 (Spi6); PD-L1, HLA-G (H2-M3), Cd47, FASLG (FasL), Ccl21 (Ccl21b), Mfge8, and Serpin B9 (Spi6); PD-L1, HLA-G (H2-M3), Cd200, FASLG (FasL), Ccl21 (Ccl21b), Mfge8, and Serpin B9 (Spi6); PD-L1, Cd47, Cd200, FASLG (FasL), Ccl21 (Ccl21b), Mfge8, and Serpin B9 (Spi6); or HLA-G (H2-M3), Cd47, Cd200, FASLG (FasL), Ccl21 (Ccl21b), Mfge8, and Serpin B9 (Spi6)).

In some embodiments of any of the foregoing aspects, the cell includes all eight of the set of transgenes PD-L1, HLA-G (H2-M3), Cd47, Cd200, FASLG (FasL), Ccl21 (Ccl21b), Mfge8, and Serpin B9 (Spi6).

In some embodiments of any of the foregoing aspects, the cell includes one or more (e.g., one, two, three, four, five, six, or all seven) of the set of transgenes HLA-G (H2-M3), Cd47, Cd200, FASLG (FasL), Ccl21 (Ccl21b), Mfge8, and Serpin B9 (Spi6).

In some embodiments of any of the foregoing aspects, the cell includes one or more (e.g., one, two, three, four, five, six, or all seven) of the set of transgenes PD-L1, HLA-G (H2-M3), Cd47, Cd200, Ccl21 (Ccl21b), Mfge8, and Serpin B9 (Spi6).

In some embodiments of any of the foregoing aspects, the cell includes one or more (e.g., one, two, three, four, five, or all six) of the set of transgenes HLA-G (H2-M3), Cd47, Cd200, Ccl21 (Ccl21b), Mfge8, and Serpin B9 (Spi6).

In some embodiments of any of the foregoing aspects, the cell is not modified to express PD-L1. In some embodiments of any of the foregoing aspects, the cell is not modified to express FasL. In some embodiments of any of the foregoing aspects, the cell is not modified to express TGF-β. In some embodiments of any of the foregoing aspects, the cell is not modified to express CTLA4 or CLTA4-Ig. In some embodiments of any of the foregoing aspects, the cell is not modified to express IDO. In some embodiments of any of the foregoing aspects, the cell is not modified to express IL-35. In some embodiments of any of the foregoing aspects, the cell is not modified to express IL-10. In some embodiments of any of the foregoing aspects, the cell is not modified to express VEGF. In some embodiments of any of the foregoing aspects, the cell is not modified to express an NFKb decoy receptor. In some embodiments of any of the foregoing aspects, the cell is not modified to express soluble TNFR. In some embodiments of any of the foregoing aspects, the cell is not modified to express CCR7. In some embodiments of any of the foregoing aspects, the cell is not modified to express SOCS1. In some embodiments of any of the foregoing aspects, the cell is not modified to express HLA-E. In some embodiments of any of the foregoing aspects, the cell is not modified to express siRNA directed to IL-12.

In another aspect, the invention features a population of cells described herein.

In another aspect, the invention features composition including a cell described herein.

In another aspect, the invention features a composition including a population of cells described herein.

In some embodiments of any of the foregoing aspects, the composition further includes a pharmaceutically acceptable excipient.

In another aspect, the invention features a kit containing a cell described herein or a composition described herein.

### Definitions

Unless defined otherwise, all technical and scientific terms used herein have the same meaning as commonly understood by one of ordinary skill in the art to which this disclosure belongs. Terms such as “a”, “an,” and “the” are not intended to refer to only a singular entity, but include plural referents unless context clearly indicates otherwise.

As used herein, the term “about” refers to a value that is no more than 10% above or below the value being described. For example, the term “about 5 nM” indicates a range of from 4.5 nM to 5.5 nM.

As used herein, the term “activated leukocyte” refers to the state of a leukocyte (e.g., a granulocyte, such as a neutrophil, eosinophil, or basophil; a monocyte, or a lymphocyte, such as a B or T cell) caused by response to a perceived insult. When leukocytes become activated, they can proliferate, secrete cytokines, differentiate, present antigens, become more polarized, become more phagocytic, and/or become more cytotoxic. Factors that stimulate immune cell activation include pro-inflammatory cytokines, pathogens, and non-self antigen presentation. Activated leukocytes can be isolated from lymphoid organs. Leukocytes, such as T cells, can also be activated in vitro using anti-CD3/CD28 beads or other methods employed by those of skill in the art (see, e.g., Frauwith and Thompson, J.Clin Invest 109:295-299 (2002); and Trickett and Kwan, J Immunol Methods 275:251-255 (2003)).

As used herein, “allogeneic” means cells, tissue, DNA, or factors taken or derived from a different subject of the same species.

As used herein, the term “alloantigen” refers to a non-self antigen from a member of the same species. Two major types of alloantigens are blood group antigens (e.g., Antigen A, Antigen B, or an Rh antigen) and histocompatibility antigens (e.g., human leukocyte antigens (HLA), such as class I antigens (e.g., HLA-A, HLA-B, and HLA-C) and class II antigens (e.g., HLA-DR, HLA-DQ, and HLA-DP)).

As used herein, the term “antigen” refers to a substance (e.g., a protein, peptide, or polysaccharide) that can bind to (e.g., can be bound by) Ag-specific receptors, antibodies, and/or T lymphocyte receptors. The term “antigen” is used herein to refer to a polypeptide or a portion of a polypeptide (e.g., a full-length protein) that can stimulate an immune response. The antigen can be produced in vivo by proteolytic processing of the polypeptide (e.g., fragments of 10-20 amino acids in length may be produced that may be recognized by the immune system). Antigens can be classified as self or non-self antigens based on their relationship to a subject.

As used herein, the term “autologous” refers to cells, tissue, DNA, or factors taken or derived from a subject that can be modified and used to treat the same subject.

As used herein, the term “immune tolerance” refers to the failure to mount an immune response (e.g., unresponsiveness) to an antigen that has the capacity to elicit an immune response in a subject.

As used herein, the term “stem cell” refers to a cell that can differentiate into one or more specialized cells and has the capacity for self-renewal. Stem cells include pluripotent stem cells (PSCs), such as embryonic stem cells (ESCs) and induced pluripotent stem cells (iPSCs), and multipotent stem cells, such as cord blood stem cells, mesenchymal stromal cells and adult stem cells, which are found in various tissues. The term “stem cell” also includes cells amenable for genome editing, cells that can serve as a source of a therapeutic cell type (e.g., cells that can be directed to differentiate into a lineage restricted or terminally differentiated cell that is used for cell therapy, or cells of a desired target tissue), and cells with “artificial” cell acquired stem cell properties (e.g., pluripotency or multipotency or self-renewal).

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability propagate indefinitely under suitable in vitro culture conditions. See, for example, Thomson et al., Science 282:1145 (1998).

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Soxl5), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. See, for example, Takahashi and Yamanaka, Cell 126:663 (2006).

As used herein, the term “mitigate antigen presenting cell activation and function” refers to a transgene that encodes a gene product whose function is to inhibit antigen presenting cell activation or the ability of an antigen presenting cell to promote the activation of graft attacking leukocytes (Fiorentino et al., J Immunol. 146:3444-51 (1991); Salio et al., Eur J Immunol. 29:3245-53 (1999)). In an embodiment, mitigation of antigen presenting cell activation and function refers to a decrease in APC activation and function of at least about 10%, 20%, 30%, 40%, 50%, 60%, 70%, 80%, 90%, 95% or 100%, relative to a control (e.g., as determined using an assay for antigen presenting cell activation, such as reduced proliferation, reduced secretion of pro-inflammatory cytokines (e.g., interleukin-1 (IL-1, e.g., IL-1B), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF), which can be measured using an ELISA or Western Blot analysis of culture media or a patient sample, such as a blood sample), or reduced levels of cell surface markers (e.g., CD11c, CD11b, HLA molecules (e.g., MHC-II), CD40, B7, IL-2, CD80 or CD86, which can be assessed using flow cytometry, immunohistochemistry, in situ hybridization, and other assays that allow for measurement of cell surface markers)). Antigen presenting cells include dendritic cells, B cells, and macrophages. Mast cells and neutrophils can also be induced to present antigens. Methods for determining mitigation of antigen presenting cell activation and function are known in the art. Examples of gene products that mitigate antigen presenting cell activation and function include, but are not limited to: Ccl21 (Ccl21b) and PD-L1. Such transgenes may be referred herein to “cloaking” or “cloaked” genes.

As used herein, the term “mitigate graft attacking leukocyte activity or cytolytic function” refers to a transgene that encodes a gene product whose function is to inhibit or prevent graft attacking leukocyte activity or cytolytic function near allograft cells (MacDonald et al., J Immunol. 126:1671-5 (1981); Bongrand et al., Eur J Immunol. 13:424-9 (1983); MacDonald et al., Eur J Immunol. 9:466-70 (1979)). In an embodiment, mitigation of graft attacking leukocyte activity or cytolytic function refers to a decrease in leukocyte activity or cytolytic function of at least about 10%, 20%, 30%, 40%, 50%, 60%, 70%, 80%, 90%, 95% or 100%, relative to a control (e.g., as determined using an assay for leukocyte activation, such as reduced proliferation, reduced secretion of pro-inflammatory cytokines (e.g., interleukin-1 (IL-1, e.g., IL-1B), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF), which can be measured using an ELISA or Western Blot analysis of culture media or a patient sample, such as a blood sample), or reduced polarization (e.g., a reduction in the level of IL-12, TNF, IL-1B, IL-6, IL-23, MARCO, MHC-II, CD86, INOS, CXCL9, and CXCL10 in a macrophage or monocyte, or a reduction in the level of a Th1-specific marker (e.g., T-bet, IL-12R, STAT4), a chemokine receptor (e.g., CCR5, CXCR6, or CXCR3); or a Th2-specific marker: (e.g., CCR3, CXCR4, STAT6, GATA3, or IL-4Ra) in a T cell, which can be assessed using flow cytometry, immunohistochemistry, situ hybridization, qPCR, or western blot analysis for cell surface markers or intracellular proteins, and ELISA or western blot analysis for secreted proteins); or as determined using an assay for cytolytic function (e.g., by incubating leukocytes with a target cell line that has been pre-coated with antibodies to a surface antigen expressed by the target cell line and measuring the number of surviving target cells with a fluorescent viability stain, or by measuring the secretion of cytolytic granules (e.g., perforin, granzymes, or other cytolytic proteins released from immune cells) from the leukocytes). Methods for determining mitigation of graft attacking leukocyte activity or cytolytic function are known in the art. Examples of gene products that mitigate graft attacking leukocyte activity or cytolytic function include, but are not limited to: PD-L1, HLA-G (H2-M3), Cd39, Cd73, and Lag3. Such transgenes may be referred herein to “cloaking” or “cloaked” genes.

As used herein, the term “mitigate macrophage cytolytic function and phagocytosis of allograft cells” refers to a transgene that encodes a gene product whose function is to inhibit or prevent macrophage cytolytic function and/or phagocytosis of allograft cells (Fish et al., Toxicology. 19:127-38. (1981); Sung et al., J Biol Chem. 260:546-54 (1985); Amash et al., J Immunol. 196:3331-40 (2016)). In an embodiment, mitigation of macrophage cytolytic function and phagocytosis of allograft cells refers to a decrease in macrophage cytolytic function and/or phagocytosis of allograft cells of at least about 10%, 20%, 30%, 40%, 50%, 60%, 70%, 80%, 90%, 95% or 100%, relative to a control (e.g., as determined using an assay for macrophage cytolytic function (e.g., by incubating macrophages with a target cell line that has been pre-coated with antibodies to a surface antigen expressed by the target cell line and measuring the number of surviving target cells with a fluorescent viability stain, or by measuring the secretion of cytolytic granules (e.g., perforin, granzymes, or other cytolytic proteins released from immune cells) released from the macrophages; or as determined using an assay for macrophage phagocytosis (e.g., culturing macrophages with fluorescent beads or a target cell line that has been pre-coated with antibodies to a surface antigen expressed by the target cell line and measuring fluorescence inside the immune cell or quantifying the number of beads or cells engulfed)). Methods for determining mitigation of macrophage cytolytic function and phagocytosis of allograft cells are known in the art. Examples of gene products that mitigate macrophage cytolytic function include, but are not limited to: Cd47, Cd200, Mfge8, and ll1r2. Such transgenes may be referred herein to “cloaking” or “cloaked” genes.

As used herein, the term “induce apoptosis in graft attacking leukocytes” refers to a transgene that encodes a gene product whose function is to kill graft attacking leukocytes near allograft cells (Huang et al., Proc Natl Acad Sci USA. 96:14871-6 (1999); Suzuki et al., Proc Natl Acad Sci USA. 97:1707-12 (2000); Simon et al., Proc Natl Acad Sci USA. 98:5158-63 (2001)). In an embodiment, induction of apoptosis in graft attacking leukocytes refers to an increase in apoptosis in graft attacking leukocytes of at least about 10%, 20%, 30%, 40%, 50%, 60%, 70%, 80%, 90%, 95% or 100%, relative to a control (e.g., as determined using an assay for apoptosis, such as TUNEL staining, caspase staining, or Annexin-V staining, or use of fluorescent viability stains). Methods for determining induction of apoptosis in graft attacking leukocytes are known in the art. Examples of gene products that can induce apoptosis in graft attacking leukocytes include, but are not limited to: FASLG (FasL) and Tnfsf10. Such transgenes may be referred herein to “cloaking” or “cloaked” genes.

As used herein, the term “mitigate local inflammatory proteins” refers to a transgene that encodes a gene product whose function is to inhibit the activity of local proteins, where the function of said proteins is to promote graft attacking leukocyte accumulation, and/or their cytolytic function (Felix et al., Nat Rev Immunol. 17:112-29 (2017)). In an embodiment, mitigation of local inflammatory proteins refers to a reduction in local inflammatory proteins of at least about 10%, 20%, 30%, 40%, 50%, 60%, 70%, 80%, 90%, 95% or 100%, relative to a control (e.g., as determined using an assay for inflammatory proteins that promote leukocyte activation or migration to a site of inflammation (e.g., a chemokine, such as CCL2, CCL3, CCL5, CXCL1, CXCL2, and CXCL8, or a pro-inflammatory cytokine, such as IL-1B, IL-5, IL-6, IL-8, IL-10, IL-12, IL-13, IL-18, TNFα, IFNγ, or GMCSF, which can be measured using an ELISA, Western blot analysis, or other techniques known in the art for measuring secreted proteins)). Methods for determining mitigation of local inflammatory proteins are known in the art. Examples of gene products that mitigate local inflammatory proteins include, but are not limited to: PD-L1, Il1r2, and Ackr2. Such transgenes may be referred herein to “cloaking” or “cloaked” genes.

As used herein, the term “protect against leukocyte-mediated apoptosis” refers to a transgene that encodes a gene product whose function is to inhibit any cell component that may induce apoptosis or cytolysis of an allograft cell (Abdullah et al., J Immunol. 178:3390-9 (2007)). In an embodiment, protection against leukocyte-mediated apoptosis refers to a decrease in leukocyte-mediated apoptosis of at least about 10%, 20%, 30%, 40%, 50%, 60%, 70%, 80%, 90%, 95% or 100%, relative to a control (e.g., as determined using an assay for leukocyte-mediated apoptosis (e.g., by incubating leukocytes with a target cell line that has been pre-coated with antibodies to a surface antigen expressed by the target cell line and measuring the number of surviving target cells with a fluorescent viability stain, or by measuring the secretion of cytolytic granules (e.g., perforin, granzymes, or other cytolytic proteins released from immune cells) released from the leukocyte). Methods for determining protection against leukocyte-mediated apoptosis are known in the art.

Examples of gene products that protect against leukocyte-mediated apoptosis include, but are not limited to: Serpin B9 (Spi6) and Dad1. Such transgenes may be referred herein to “cloaking” or “cloaked” genes.

As used herein, the term “activation” refers to the response of an immune cell to a perceived insult. When immune cells become activated, they proliferate, secrete pro-inflammatory cytokines, differentiate, present antigens, become more polarized, and become more phagocytic and cytotoxic. Factors that stimulate immune cell activation include pro-inflammatory cytokines, pathogens, and non-self antigen presentation (e.g., antigens from pathogens presented by dendritic cells, macrophages, or B cells).

As used herein, the term “antigen presentation” refers to a process in which fragments of antigens are displayed on the cell surface of immune cells. Antigens are presented to T cells and B cells to stimulate an immune response. Antigen presenting cells include dendritic cells, B cells, and macrophages. Mast cells and neutrophils can also be induced to present antigens.

As used herein, the term “cytokine” refers to a small protein involved in cell signaling. Cytokines can be produced and secreted by immune cells, such as T cells, B cells, macrophages, and mast cells, and include chemokines, interferons, interleukins, lymphokines, and tumor necrosis factors.

As used herein, the term “cytokine production” refers to the expression, synthesis, and secretion (e.g., release) of cytokines by an immune cell.

As used herein, the term “cytotoxicity” refers to the ability of immune cells to kill other cells. Immune cells with cytotoxic functions release toxic proteins (e.g., perforin and granzymes) capable of killing nearby cells. Natural killer cells and cytotoxic T cells (e.g., CD8⁺ T cells) are the primary cytotoxic effector cells of the immune system, although dendritic cells, neutrophils, eosinophils, mast cells, basophils, macrophages, and monocytes have been shown to have cytotoxic activity.

As used herein, the term “degranulation” refers to a cellular process in which molecules, including antimicrobial and cytotoxic molecules, are released from intracellular secretory vesicles called granules. Degranulation is part of the immune response to pathogens and invading microorganisms by immune cells such as granulocytes (e.g., neutrophils, basophils, and eosinophils), mast cells, and lymphocytes (e.g., natural killer cells and cytotoxic T cells). The molecules released during degranulation vary by cell type and can include molecules designed to kill the invading pathogens and microorganisms or to promote an immune response, such as inflammation.

As used herein, the term “phagocytosis” refers to the process in which a cell engulfs or ingests material, such as other cells or parts of cells (e.g., bacteria), particles, or dead or dying cells. A cell that is capable of performing this function is called a phagocyte. Immune phagocytes include neutrophils, monocytes, macrophages, mast cells, B cells, eosinophils, and dendritic cells.

As used herein, the term “polarization” refers to the ability of an immune cell to shift between different functional states. A cell that is moving toward one of two functional extremes is said to be in the process of becoming more polarized. The term polarization is often used to refer to macrophages, which can shift between states known as M1 and M2. M1, or classically activated, macrophages secrete pro-inflammatory cytokines (e.g., IL-12, TNF, IL-6, IL-8, IL-1B, MCP-1, and CCL2), are highly phagocytic, and respond to pathogens and other environmental insults. M1 macrophages can also be detected by expression of Nos2. M2, or alternatively activated, macrophages secrete a different set of cytokines (e.g., IL-10) and are less phagocytic. M2 macrophages can detected by expression of Arg1, IDO, PF4, CCL24, IL10, and IL4Ra. Cells become polarized in response to external cues such as cytokines, pathogens, injury, and other signals in the tissue microenvironment.

As used herein, the term “pro-inflammatory cytokine” refers to a cytokine secreted from immune cells that promotes inflammation. Immune cells that produce and secrete pro-inflammatory cytokines include T cells (e.g., Th cells) macrophages, B cells, and mast cells. Pro-inflammatory cytokines include interleukin-1 (IL-1, e.g., IL-1B), IL-5, IL-6, IL-8, IL-10, IL-12, IL-13, IL-18, tumor necrosis factor (TNF, e.g., TNFα), interferon gamma (IFNγ), and granulocyte macrophage colony stimulating factor (GMCSF).

As used herein, the term “biologic” refers to a designed polypeptide and corresponding encoding DNA, which can be expressed as a transgene. The polypeptide may agonize or inhibit the function of an endogenous gene or inhibit or activate a biological process. Methods for determining whether a polypeptide has agonist or antagonist activity or function are generally known in the art. In an embodiment, the agonist function is at least about 10%, 20%, 30%, 40%, 50%, 60%, 70%, 80%, 90, 95% or 100% of the function, relative to the function of a control. In an embodiment, the antagonist function is at least about 10%, 20%, 30%, 40%, 50%, 60%, 70%, 80%, 90, 95% or 100% of the function, relative to the function of a control.

As used herein, the term “operably linked” refers to a first molecule joined to a second molecule, wherein the molecules are so arranged that the first molecule affects the function or expression of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “promoter” refers to a recognition site on DNA that is bound by an RNA polymerase. The polymerase drives transcription of the transgene.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutical composition” refers to a mixture containing a therapeutic agent, optionally in combination with one or more pharmaceutically acceptable excipients, diluents, and/or carriers, to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and/or other problem complications commensurate with a reasonable benefit/risk ratio.

As used herein, the term “wild-type” refers to a genotype with the highest frequency for a particular gene in a given organism.

The terms “cell division locus”, “cell division loci”, and “CDL” as used herein, refer to a genomic locus (or loci) whose transcription product(s) is required to maintain cell viability (which means that loss of such a gene leads to cell death) or cell proliferative ability (which means that loss of such a gene impairs or prevents cell proliferation). The gene can be expressed by dividing cells (e.g., cells actively undergoing cell division), cells capable of cell division (e.g., cells that are not currently undergoing cell division but that are capable of proliferating), and/or by non-diving cells (e.g., post-mitotic cells such as neurons). When a CDL comprises a single locus, absence of CDL expression in a cell (or its derivatives) means that tumor initiation and/or formation is prohibited either because the cell(s) will be ablated (e.g., killed) in the absence of CDL expression (e.g., the CDL is required for cell viability, which means that cells die in the absence of CDL expression) or because proliferation of the cell(s) will be blocked or compromised in the absence of CDL expression (e.g., the CDL is required for cell proliferation, which means that cells fail to proliferate in the absence of CDL expression). When a CDL comprises multiple loci, absence of expression by all or subsets of the loci in a cell (or its derivatives) means that tumor initiation and/or formation is prohibited either because the cell(s) will be ablated in the absence of CDL expression or because proliferation of the cell(s) will be blocked or compromised in the absence of CDL expression. A CDL may or may not be expressed in non-dividing and/or non-proliferating cells. A CDL may be endogenous to a host cell or it may be a transgene. If a CDL is a transgene, it may be from the same or different species as a host cell or it may be of synthetic origin. In an embodiment, a CDL is a single locus that is transcribed during cell division. For example, in an embodiment, a single locus CDL is CDK1. In an embodiment, a CDL comprises two or more loci that are transcribed during cell division. For example, in an embodiment, a multi-locus CDL comprises two MYC genes (β-Myc and N-myc) (Scognamiglio et al., 2016). In an embodiment, a multi-locus CDL comprises AURORA B and C kinases, which may have overlapping functions (Fernandez-Miranda et al., 2011). Cell division and cell proliferation are terms that may be used interchangeably herein.

The terms “normal rate of cell division”, “normal cell division rate”, “normal rate of cell proliferation”, and “normal cell proliferation rate” as used herein, refer to a rate of cell division and/or proliferation that is typical of a non-cancerous healthy cell. A normal rate of cell division and/or proliferation may be specific to cell type. For example, it is widely accepted that the number of cells in the epidermis, intestine, lung, blood, bone marrow, thymus, testis, uterus and mammary gland is maintained by a high rate of cell division and a high rate of cell death. In contrast, the number of cells in the pancreas, kidney, cornea, prostate, bone, heart and brain is maintained by a low rate of cell division and a low rate of cell death (Pellettieri and Sánchez Alvarado, 2007).

The terms “inducible negative effector of proliferation” and “iNEP” as used herein, refer to a genetic modification that facilitates use of CDL expression to control cell division and/or proliferation by: i) inducibly stopping or blocking CDL expression, thereby prohibiting cell division and proliferation; ii) inducibly ablating at least a portion of CDL-expressing cells (i.e., killing at least a portion of proliferating cells); or iii) inducibly slowing the rate of cell division relative to a cell's normal cell division rate, such that the rate of cell division would not be fast enough to contribute to tumor formation.

The terms “ablation link” and “ALINK” as used herein, refer to an example of an iNEP, which comprises a transcriptional link between a CDL and a sequence encoding a negative selectable marker (e.g., a modification of a coding or non-coding region of the CDL such that the negative selectable marker is co-expressed with the gene product of the CDL). The ALINK modification allows a user to inducibly kill proliferating host cells containing the ALINK or inhibit the host cell's proliferation by killing at least a portion of proliferating cells by exposing the ALINK-modified cells to an inducer of the negative selectable marker. For example, a cell modified to comprise an ALINK at a CDL may be treated with an inducer (e.g., a prodrug) of the negative selectable marker in order to ablate proliferating cells or to inhibit cell proliferation by killing at least a portion of proliferating cells.

The terms “exogenous activator of regulation of CDL” and “EARC” as used herein, refer to an example of an iNEP, which comprises a mechanism or system that facilitates exogenous alteration of non-coding or coding DNA transcription or corresponding translation via an activator. An EARC modification allows a user to inducibly stop or inhibit division of cells containing the EARC by removing from the EARC-modified cells an inducer that permits transcription and/or translation of the EARC-modified CDL. For example, an inducible activator-based gene expression system may be operably linked to a CDL and used to exogenously control expression of a CDL or CDL translation, such that the presence of a drug inducible activator and corresponding inducer drug are required for CDL transcription and/or translation. In the absence of the inducer drug, cell division and/or proliferation would be stopped or inhibited (e.g., slowed to a normal cell division rate). For example, the CDL Cdk1/CDK1 may be modified to comprise a dox-bridge, such that expression of Cdk1/CDK1 and cell division and proliferation are only possible in the presence of an inducer (e.g., doxycycline).

The term “proliferation antagonist system” as used herein, refers to a natural or engineered compound(s) whose presence inhibits (completely or partially) proliferation of a cell.

The term “dox-bridge” as used herein, refers to a mechanism for separating activity of a promoter from a target transcribed region by expressing rtTA (Gossen et al., 1995) by the endogenous or exogenous promoter and rendering the transcription of target region under the control of TRE. As used herein, “rtTA” refers to the reverse tetracycline transactivator elements of the tetracycline inducible system (Gossen et al., 1995) and “TRE” refers to a promoter consisting of TetO operator sequences upstream of a minimal promoter. Upon binding of rtTA to the TRE promoter in the presence of doxycycline, transcription of loci downstream of the TRE promoter increases. The rtTA sequence may be inserted in the same transcriptional unit as the CDL or in a different location of the genome, so long as the transcriptional expression's permissive or non-permissive status of the target region is controlled by doxycycline. A dox-bridge is an example of an EARC.

As used herein, the term “fail-safe cell” refers to a cell that contains one or more homozygous, heterozygous, hemizygous or compound heterozygous ALINKs or EARCs in one or more CDLs (e.g., at least two, three, four, or five CDLs). Fail-safe cells may contain either ALINKs or EARCs or both ALINK and EARC modifications (e.g., ALINK and EARC modifications in different CDLs or in a single CDL).

As used herein, the term “fail-safe” refers to a property of a cell that is unlikely to exhibit uncontrolled (e.g., tumorigenic) proliferation. A cell can be considered “fail safe” when cell proliferation is under the control of a negative regulator or inducer, and the possibility of the cell losing the activity of the system that controls proliferation due to genetic mutation is low. The fail-safe volume will depend on the number of ALINKs and the number of ALINK-targeted CDLs (e.g., a cell with homozygous modifications of two different CDLs has a higher fail safe volume (e.g., it is less likely to lose all systems that control proliferation through genetic mutation) than a cell with a heterozygous modification of a single CDL). The fail-safe property is further described in Table 3.

### DESCRIPTION OF THE DRAWINGS

FIGS. 1A-1C are images showing that administration of cloaked allogeneic cells induces immune tolerance to the cells. FIG. 1A shows a C3H mouse that received an initial subcutaneous injection of allogenic C57BL/6 cloaked embryonic stem cells (ESCs) (boxes near the flanks, “B”), and, approximately two months later, received a subcutaneous injection of uncloaked allogeneic cells of the same type as the cloaked cells (boxes near the shoulders, “A”). The uncloaked cells were not rejected by the immune system of the mouse, indicating that the cloaked cells induced immune tolerance (FIG. 1A). FIGS. 1B and 1C show H&E staining of the teratomas formed by both the cloaked (FIG. 1C) and uncloaked (FIG. 1B) allogeneic cells showing that the teratomas are well differentiated tissues with no sign of excessive lymphocyte infiltration or necrosis.

### DETAILED DESCRIPTION OF THE DISCLOSURE

Described herein are compositions and methods for modulating the immune system to produce an antigen-specific immune response in a subject (e.g., a human subject). The invention features cells that express one or more cloaking transgenes in addition to a polypeptide containing a donor alloantigen, and methods of using such cells to induce immune tolerance to the donor alloantigen in a subject prior to transplantation of an organ or tissue from the donor into the subject. The invention also features cells that express one or more cloaking transgenes in addition to a polypeptide containing an endogenous or “self” antigen, which can be used to induce tolerance to the antigen in a subject with an autoimmune disease or condition (e.g., a subject whose immune system aberrantly mounts an immune response against the antigen). Finally, the invention features a method of using cloaked cells (e.g., cells that express one or more cloaking transgenes) to induce the production of antibodies against an antigen (e.g., a disease-related antigen) in a subject (e.g., a human subject). The methods and compositions described herein provide targeted, longer lasting, and lower risk therapeutic approaches compared to the current standard of care.

### Cloaked Cells for Antigen-Specific Modulation of the Immune System

Self versus non-self discrimination is a feature of a properly functioning immune system, as the immune system must detect and mount an immune response against non-self or foreign antigens from viruses, bacteria, and other pathogens, while recognizing and not responding to endogenous “self” antigens. The adaptive immune system responds to foreign antigens by producing antibodies, which promotes long lasting immunity and a rapid response to the same antigen should the immune system encounter it again. The ability to detect and mount an immune response against foreign antigens is a characteristic of the immune system. Yet, this function can pose a challenge for cell-based therapies and transplantation, as the immune system may attack any foreign cell or tissue that contains non-self proteins, including intentionally transplanted cells, tissues, and organs. Therefore, cell-based therapies and transplantation are often accompanied by administration of immunosuppressive medications that limit the immune system from mounting an immune response. Unfortunately, administration of immunosuppressive medications carries a number of risks.

The present invention is based, in part, on the discovery that one can suppress the immune response to a non-self cell by modifying the cell to express one or more cloaking transgenes, which encode proteins that inhibit or negatively regulate an immune response (e.g., a response of macrophages, T cells, NK cells, or other antigen presenting cells), or induce cell death of an immune cell (e.g., a T cell or macrophage). The cloaking transgenes are listed below in Table 1, and cells that express one or more (e.g., one, two, three, four, five, six, seven, or all eight) of PD-L1, H2-M3, Cd47, Cd200, FasL, Ccl21b, Mfge8, and Spi6 are referred to herein as cloaked cells.

In addition to suppressing the immune response of a subject to the cloaked cell, the present inventors have also discovered that administration of a cell modified to express one or more cloaking transgenes (e.g., one, two, three, four, five, six, seven, or all eight of PD-L1, H2-M3, Cd47, Cd200, FasL, Ccl21b, Mfge8, and Spi6) induces immune tolerance to an uncloaked cell of the same type (e.g., the same type of cell from the same source that is not modified to express any of the cloaking transgenes). These findings indicate that the immune system of a subject treated with a cloaked cell learns to treat all of the proteins expressed by the cloaked cell as “self” proteins. In view of this discovery, cells can be modified to express one or more cloaking transgenes (e.g., cloaked) and one or more polypeptides containing one or more antigens, and then administered to a subject to induce immune tolerance to the one or more antigens. In other embodiments, the ability to induce immune tolerance to a cloaked cell can be harnessed to promote an immune response to (e.g., produce antibodies directed against) a specific antigen. The cloaking transgenes used in the compositions and methods described herein are characterized in more detail below and in International Patent Application No. PCT/CA2018/050706, which is incorporated herein by reference in its entirety.

TABLE 1
Genes that can be expressed to promote immune tolerance

| Gene | Function |
|---|---|
| PD-L1 | Induces cell death in PD-L1 expressing T cells and |
| | macrophages |
| HLA-G | Inhibits NK cells from attacking cells lacking MHC molecules |
| (mouse gene: H2-M3) | |
| Cd47 | Negative regulator of macrophages and killer T cells |
| Cd200 | Inhibits macrophage activation |
| FASLG | Induces apoptosis in Fas expressing CD8 + T cells |
| (mouse gene: FasL) | |
| Clc21 | Chemo-attractant for antigen presenting cells (APCs) |
| (mouse gene: Ccl21b) | |
| Mfge8 | Inhibition of macrophage phagocytosis |
| Serpin B9 | Inhibition of granzyme/perforin attack |
| (mouse gene: Spi6) | |
| Dad1 | Negative regulator of programmed cell death |
| Tnfrsf10 | Induces apoptosis in leukocytes expressing the TRAIL receptor |
| Cd39 | Converts ATP to AMP, inhibits T-cells |
| Cd73 | Converts AMP to adenosine, inhibits T-cells, suppresses |
| | dendritic cells |
| Lag3 | Inhibits T-cell activation, proliferation, function |
| II1r2 | Blocks IL-1B activity, blocks inflammation and innate cell |
| | activation |
| Ackr2 | Decoy receptor for chemokines, prevents leukocyte |
| | accumulation |
| Tnfrsf22 | Decoy receptor, blocks TRAIL-induced apoptosis from T-cells |
| Tnfrsf23 | Decoy receptor, blocks TRAIL-induced apoptosis from T-cells |
| IFNγR1 d39 | Dominant negative interferon gamma receptor 1, prevents |
| | IFNγ-mediated upregulation of MHCs in ES cells |

C-C motif chemokine ligand 21 (Ccl21) is expressed by local lymph nodes where it acts to attract activated antigen presenting cells (APCs). This key function offers an opportunity to “reverse” the migration of APCs by overexpressing this gene on grafted cells. Indeed, some melanomas express Ccl21 and recruit CCR7⁺ cells that, in turn, can reorganize portions of their tumor stroma as “self”. This leads to a stromal reconstruction that directs the recruitment and maintenance of Cd4⁺Tregs (Zindl et al., Science. 328:697-8 (2010)). In fact, the expression of Ccl21 on tumors can protect co-implanted Ccl21 deficient tumor cells from rejection in a syngeneic allograft setting (Shields et al., Science. 328:749-52 (2010)). Ccl21b is the mouse ortholog of human Ccl21.

The amino acid sequences of mouse and human Ccl21 are:

Mouse Ccl21
(SEQ ID NO: 1)
MAQMMTLSLLSLVLALCIPWTQGSDGGGQDCCLKYSQKKIPYSIVRGYRK
QEPSLGCPIPAILFLPRKHSKPELCANPEEGWVQNLMRRLDQPPAPGKQS
PGCRKNRGTSKSGKKGKGSKGCKRTEQTQPSRG
Human Ccl21
(SEQ ID NO: 2)
MAQSLALSLLILVLAFGIPRTQGSDGGAQDCCLKYSQRKIPAKVVRSYRK
QEPSLGCSIPAILFLPRKRSQAELCADPKELWVQQLMQHLDKTPSPQKPA
QGCRKDRGASKTGKKGKGSKGCKRTERSQTPKGP

Expression of Cd47 in umbilical cord blood can promote the development of hyporesponsive T-cells (Avice et al., J Immunol. 167:2459-68 (2001)). Erythrocytes also up-regulate Cd47 to avoid dendritic cell activation due to their lack of “self” presentation (van den Berg et al., Immunity. 43:622-4 (2015)). More recently, it was shown that expression of human Cd47 increases engraftment in a mouse model of pig-to-human hematopoietic cell transplantation (Tena et al., Am J Transplant. 14:2713-22 (2014)).

The amino acid sequences of mouse and human Cd47 are:

Mouse Cd47
(SEQ ID NO: 3)
MWPLAAALLLGSCCCGSAQLLFSNVNSIEFTSCNETVVIPCIVRNVEAQS
TEEMFVKWKLNKSYIFIYDGNKNSTTTDQNFTSAKISVSDLINGIASLKM
DKRDAMVGNYTCEVTELSREGKTVIELKNRTVSWFSPNEKILIVIFPILA
ILLFWGKFGILTLKYKSSHTNKRIILLLVAGLVLTVIVVVGAILLIPGEK
PVKNASGLGLIVISTGILILLQYNVFMTAFGMTSFTIAILITQVLGYVLA
LVGLCLCIMACEPVHGPLLISGLGIIALAELLGLVYMKFVASNQRTIQPP
RNR
Human Cd47
(SEQ ID NO: 4)
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVASNQKTIQ
PPRKAVEEPLNAFKESKGMMNDE

Cd200 is also as an important immunoregulatory molecule; increased expression can reduce the severity of allograft rejection, autoimmunity, and allergic disease (Gorczynski et al., J Immunol. 172:7744-9 (2004)). It has been shown that, in vitro, APC expression of Cd200 suppresses production of interferon gamma (IFN-γ) and cytolytic granules by activated Cd8+ T-cells (Misstear et al., J Virol. 86:6246-57 (2012)). Most interestingly, overexpression of Cd200 increases the survival of skin and cardiac allografts in mice by promoting of Foxp3+ Treg cells (Gorczynski et al., Transplantation. 98:1271-8 (2014)).

The amino acid sequences of mouse and human Cd200 are:

Mouse Cd200
(SEQ ID NO: 5)
MGSLVFRRPFCHLSTYSLIWGMAAVALSTAQVEVVTQDERKALHTTASLR
CSLKTSQEPLIVTWQKKKAVSPENMVTYSKTHGVVIQPAYKDRINVTELG
LWNSSITFWNTTLEDEGCYMCLFNTFGSQKVSGTACLTLYVQPIVHLHYN
YFEDHLNITCSATARPAPAISWKGTGTGIENSTESHFHSNGTTSVTSILR
VKDPKTQVGKEVICQVLYLGNVIDYKQSLDKGFWFSVPLLLSIVSLVILL
VLISILLYWKRHRNQERGESSQGMQRMK
Human Cd200
(SEQ ID NO: 6)
MERLVIRMPFSHLSTYSLVWVMAAVVLCTAQVQVVTQDEREQLYTPASLK
CSLQNAQEALIVTWQKKKAVSPENMVTFSENHGVVIQPAYKDKINITQLG
LQNSTITFWNITLEDEGCYMCLFNTFGFGKISGTACLTVYVQPIVSLHYK
FSEDHLNITCSATARPAPMVFWKVPRSGIENSTVTLSHPNGTTSVTSILH
IKDPKNQVGKEVICQVLHLGTVTDFKQTVNKGYWFSVPLLLSIVSLVILL
VLISILLYWKRHRNQDRGELSQGVQKMT

Spi6 is an endogenous inhibitor of the cytotoxic effector molecule granzyme B released by activated Cd8⁺T-cells (Sun et al., J Biol Chem. 272:15434-41 (1997)). Some data shows that Mesenchymal Stem Cells (MSCs) escape immune rejection by upregulating this molecule (El Haddad et al., Blood. 117:1176-83 (2011)). It has also recently been demonstrated that the ability of dendritic cells to present antigen to cytotoxic T cells without themselves being killed through contact mediated cytotoxicity is mediated by Spi6 (Lovo et al., J Immunol. 188:1057-63 (2012)). Spi6 is also known as Serpin B9.

The amino acid sequences of mouse Spi6 and the human counterpart, Serpin B9, are:

Mouse Spi6
(SEQ ID NO: 7)
MNTLSEGNGTFAIHLLKMLCQSNPSKNVCYSPASISSALAMVLLGAKGQT
AVQISQALGLNKEEGIHQGFQLLLRKLNKPDRKYSLRVANRLFADKTCEV
LQTFKESSLHFYDSEMEQLSFAEEAEVSRQHINTWVSKQTEGKIPELLSG
GSVDSETRLVLINALYFKGKWHQPFNKEYTMDMPFKINKDEKRPVQMMCR
EDTYNLAYVKEVQAQVLVMPYEGMELSLVVLLPDEGVDLSKVENNLTFEK
LTAWMEADFMKSTDVEVFLPKFKLQEDYDMESLFQRLGVVDVFQEDKADL
SGMSPERNLCVSKFVHQSVVEINEEGTEAAAASAIIEFCCASSVPTFCAD
HPFLFFIRHNKANSILFCGRFSSP
Human Serpin B9
(SEQ ID NO: 8)
METLSNASGTFAIRLLKILCQDNPSHNVFCSPVSISSALAMVLLGAKGNT
ATQMAQALSLNTEEDIHRAFQSLLTEVNKAGTQYLLRTANRLFGEKTCQF
LSTFKESCLQFYHAELKELSFIRAAEESRKHINTWVSKKTEGKIEELLPG
SSIDAETRLVLVNAIYFKGKWNEPFDETYTREMPFKINQEEQRPVQMMYQ
EATFKLAHVGEVRAQLLELPYARKELSLLVLLPDDGVELSTVEKSLTFEK
LTAWTKPDCMKSTEVEVLLPKFKLQEDYDMESVLRHLGIVDAFQQGKADL
SAMSAERDLCLSKFVHKSFVEVNEEGTEAAAASSCFVVAECCMESGPRFC
ADHPFLFFIRHNRANSILFCGRFSSP

Activated, cytotoxic, Cd8⁺can kill target cells by expression of FasL, which binds to the FAS receptor and activates a caspase-mediated apoptosis in targeted cells. However, many tumors have developed a “counterattack” by upregulating FasL on their surface (Chen et al., J Immunol. 171:1183-91 (2003)). Selective expression of FasL in the vasculature of human and mouse solid tumors has been associated with scarce Cd8+ T-cell infiltration and a predominance of FoxP3⁺ Treg cells (Motz et al. Nat Med. 20:607-15 (2014)). Most recently, it was shown that β-lymphocytes also use the expression of FasL to kill T helper cells at the effector stage of immune responses (Lundy et al., Front Immunol. 6:122 (2015)). FasL is the mouse ortholog of human FASLG.

The amino acid sequences of mouse FasL and the human counterpart, FASLG, are:

Mouse FasL
(SEQ ID NO: 9)
MQQPMNYPCPQIFWVDSSATSSWTPPGSVFPCPSSGPRGPDQRRPPPPPP
PVSPLPPPSQPLPLPPLTPLKKKDHNTNLWLPVVFFMVLVALVGMGLGMY
QLFHLQKELAELREFTNQSLKVSSFEKQIANPSTPSEKKELRSVAHLTGN
PHSRSIPLEWEDTYGTALISGVKYKKGSLVINEAGLYFVYSKVYFRGQSC
NNQPLNHKVYMRNSKYPGDLVLMEEKRLNYCTTGQIWAHSSYLGAVFNLT
SADHLYVNISQLSLINFEESKTFFGLYKL
Human FASLG
(SEQ ID NO: 10)
MQQPFNYPYPQIYWVDSSASSPWAPPGTVLPCPTSVPRRPGQRRPPPPPP
PPPLPPPPPPPPLPPLPLPPLKKRGNHSTGLCLLVMFFMVLVALVGLGLG
MFQLFHLQKELAELRESTSQMHTASSLEKQIGHPSPPPEKKELRKVAHLT
GKSNSRSMPLEWEDTYGIVLLSGVKYKKGGLVINETGLYFVYSKVYFRGQ
SCNNLPLSHKVYMRNSKYPQDLVMMEGKMMSYCTTGQMWARSSYLGAVFN
LTSADHLYVNVSELSLVNFEESQTFFGLYKL

PD-L1 is a critical immune modulatory molecule that binds to Programmed Cell Death 1 (PD-1). PD-1 is expressed on T-cells, and binding to PD-L1 results in T-cell anergy (MacDonald et al., J Immunol. 126:1671-5 (1981)).

The amino acid sequences of mouse and human PD-L1 are:

Mouse PD-L1
(SEQ ID NO: 11)
MRIFAGIIFTACCHLLRAFTITAPKDLYVVEYGSNVTMECRFPVERELDL
LALVVYWEKEDEQVIQFVAGEEDLKPQHSNFRGRASLPKDQLLKGNAALQ
ITDVKLQDAGVYCCIISYGGADYKRITLKVNAPYRKINQRISVDPATSEH
ELICQAEGYPEAEVIWTNSDHQPVSGKRSVTTSRTEGMLLNVTSSLRVNA
TANDVFYCTFWRSQPGQNHTAELIIPELPATHPPQNRTHWVLLGSILLFL
IVVSTVLLFLRKQVRMLDVEKCGVEDTSSKNRNDTQFEET
Human PDL1 (CD274)
(SEQ ID NO: 12)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET

Inflammatory environments, like those induced by allograft transplants, attracts macrophages and inflammatory monocytes, among many other innate immune cells. The milk fat globule epidermal growth factor-8 (Mfge-8) is expressed by many murine tumors (Neutzner et al., Cancer Res. 67:6777-85 (2007)) and has been shown to contribute to local immune suppression by polarizing incoming monocytes to suppressive, M2-like macrophages (Soki et al., J Biol Chem. 289:24560-72 (2014)).

The amino acid sequences of mouse and human MFGE-8 are:

Mouse MFGE8
(SEQ ID NO: 13)
MQVSRVLAALCGMLLCASGLFAASGDFCDSSLCLNGGTCLTGQDNDIYCL
CPEGFTGLVCNETERGPCSPNPCYNDAKCLVTLDTQRGDIFTEYICQCPV
GYSGIHCETETNYYNLDGEYMFTTAVPNTAVPTPAPTPDLSNNLASRCST
QLGMEGGAIADSQISASSVYMGFMGLQRWGPELARLYRTGIVNAWTASNY
DSKPWIQVNLLRKMRVSGVMTQGASRAGRAEYLKTFKVAYSLDGRKFEFI
QDESGGDKEFLGNLDNNSLKVNMFNPTLEAQYIKLYPVSCHRGCTLRFEL
LGCELHGCSEPLGLKNNTIPDSQMSASSSYKTWNLRAFGWYPHLGRLDNQ
GKINAWTAQSNSAKEWLQVDLGTQRQVTGIITQGARDFGHIQYVASYKVA
HSDDGVQWTVYEEQGSSKVFQGNLDNNSHKKNIFEKPFMARYVRVLPVSW
HNRITLRLELLGC
Human MFGE8
(SEQ ID NO: 14)
MPRPRLLAALCGALLCAPSLLVALDICSKNPCHNGGLCEEISQEVRGDVF
PSYTCTCLKGYAGNHCETKCVEPLGMENGNIANSQIAASSVRVTFLGLQH
WVPELARLNRAGMVNAWTPSSNDDNPWIQVNLLRRMWVTGVVTQGASRLA
SHEYLKAFKVAYSLNGHEFDFIHDVNKKHKEFVGNWNKNAVHVNLFETPV
EAQYVRLYPTSCHTACTLRFELLGCELNGCANPLGLKNNSIPDKQITASS
SYKTWGLHLFSWNPSYARLDKQGNFNAWVAGSYGNDQWLQVDLGSSKEVT
GIITQGARNFGSVQFVASYKVAYSNDSANWTEYQDPRTGSSKIFPGNWDN
HSHKKNLFETPILARYVRILPVAWHNRIALRLELLGC

The potent killing potential of NK cells is also absolutely critical in graft rejection. NK cells can kill targets cells that lack MHC class I molecules, as well as other cells within an inflammatory setting. H2-M3, the murine homologue of human HLA-G has recently been shown to have a regulatory effect on NK cells, licensing them to ignore cells that lack “self molecules” (Andrews et al., Nat Immunol. 13:1171-7 (2012)). This is thought to be achieved by binding of HLA-G, immunosuppressive receptors on both NK and T-cells (Carosella et al., Adv Immunol. 127:33-144 (2015)). H2-M3 is the mouse ortholog of human HLA-G.

The amino acid sequences of mouse H2-M3 and the human counterpart, HLA-G, are:

Mouse H2-M3
(SEQ ID NO: 15)
SIEEIPRMEPRAPWMEKERPEYWKELKLKVKNIAQSARANLRTLLRYYNQ
SEGGSHILQWMVSCEVGPDMRLLGAHYQAAYDGSDYITLNEDLSSWTAVD
MVSQITKSRLESAGTAEYFRAYVEGECLELLHRFLRNGKEILQRADPPKA
HVAHHPRPKGDVTLRCWALGFYPADITLTWQKDEEDLTQDMELVETRPSG
DGTFQKWAAVVVPSGEEQRYTCYVHHEGLTEPLALKWGRSSQSSVVIMV
Human HLA-G
(SEQ ID NO: 16)
MVVMAPRTLFLLLSGALTLTETWAGSHSMRYFSAAVSRPGRGEPRFIAMG
YVDDTQFVRFDSDSACPRMEPRAPWVEQEGPEYWEEETRNTKAHAQTDRM
NLQTLRGYYNQSEASSHTLQWMIGCDLGSDGRLLRGYEQYAYDGKDYLAL
NEDLRSWTAADTAAQISKRKCEAANVAEQRRAYLEGTCVEWLHRYLENGK
EMLQRADPPKTHVTHHPVFDYEATLRCWALGFYPAEIILTWQRDGEDQTQ
DVELVETRPAGDGTFQKWAAVVVPSGEEQRYTCHVQHEGLPEPLMLRWKQ
SSLPTIPIMGIVAGLVVLAAVVTGAAVAAVLWRKKSSD

Each of the transgenes in the set of transgenes (e.g., PD-L1, H2-M3, Cd47, Cd200, FasL, Ccl21b, Mfge8, and Spi6) encodes a gene product that is cytoplasmic, membrane bound, or local acting, whose function is one or more of mitigating antigen presenting cell activation and function; mitigating graft attacking leukocyte activity or cytolytic function; mitigating macrophage cytolytic function and phagocytosis of allograft cells; inducing apoptosis in graft attacking leukocytes; mitigating local inflammatory proteins; and protecting against leukocyte-mediated apoptosis. These cellular functions can serve to suppress the immune response to a cell expressing one or more e.g., one, two, three, four, five, six, seven, or all eight) of the transgenes.

A set of transgenes that includes one or more (e.g., one, two, three, four, five, six, seven, or all eight) of PD-L1, H2-M3, Cd47, Cd200, FasL, Ccl21b, Mfge8, and Spi6 can be expressed in cells. The cells may be, for example, stem cells or a cell that is amenable to genome editing, such as a cell that can be used for therapy and/or differentiated into a therapeutic cell type. The stem cells may be, for example, embryonic stem(ES) cells or induced pluripotent stem (iPS) cells. The cells may be allogeneic or autologous cells. The set of transgenes may comprise 1, 2, 3, 4, 5, 6, 7, or all 8 of these genes or may comprise at least 1, at least 2, at least 3, at least 4, at least 5, at least 6, or at least 7 of these genes. The cell may be further genetically modified to express one or more of TGF-β, Cd73, Cd39, Lag3, Il1r2, Ackr2, Tnfrsf22, Tnfrs23, Tnfrsf10, Dad1, and/or IFNγR1 d39. The TGF-β transgene may be modified to express the gene product in a membrane-bound form (i.e., such that the gene product is expressed on the surface of the cell), using methods known to those skilled in the art. For example, a method for localizing TGF-β to the membrane is to co-express TGF-β with an additional transgene encoding the LRRC32 protein or any other polypeptide that results in localization of TGF-β to the cell membrane. This protein anchors TGF-β to the membrane. (Tran DQ et al., Proc Natl Acad Sci U.S.A 106:13445-50 (2009)).

The amino acid sequence of IFNγR1 d39 is:

(SEQ ID NO: 17)
MGPQAAAGRMILLVVLMLSAKVGSGALTSTEDPEPPSVPVPTNVLIKSYN
LNPVVCWEYQNMSQTPIFTVQVKVYSGSWTDSCTNISDHCCNIYGQIMYP
DVSAWARVKAKVGQKESDYARSKEFLMCLKGKVGPPGLEIRRKKEEQLSV
LVFHPEVVVNGESQGTMFGDGSTCYTFDYTVYVEHNRSGEILHTKHTVEK
EECNETLCELNISVSTLDSRYCISVDGISSFWQVRTEKSKDVCIPPFHDD
RKDSIWILVVAPLTVFTVVILVFAYWYTKKNSFKRKSIMLPKSLLSVVKS
ATLETKPESKYSLVTPHQPAVLESETVICEEPLSTVTAPDSPEAAEQEEL
SKETKALEAGGSTSAMTPDSPPTPTQRRSFSLLSSNQSGPCSLTAYHSRN
GSDSGLVGSGSSISDLESLPNNNSETKMAEHDPPPVRKA

The genes may be human genes or murine genes. In an embodiment, the gene is of the same species as the recipient in which the cell is to be transplanted. In an embodiment, the gene is of any species in which the function of the gene is conserved or in which a designed biologic has the agonist function of the endogenous counterpart. Methods for introducing and expressing these transgenes in cells are described herein and are also known to those skilled in the art. Cells expressing these transgenes may be referred to as “cloaked” due to their ability to evade allorejection without systemic immunosuppression and without the need for immunosuppressive medication.

The cloaked cells can be further modified to express one or more polypeptides or portions thereof containing one or more antigens (e.g., a non-self antigen, a self-antigen, a food antigen, or an allergen antigen). In other embodiments, an uncloaked cell of the same type as the cloaked cell (e.g., a cell of the same type derived from the same source as the cloaked cell that is not modified to express any of the cloaking transgenes) can be modified to express a polypeptide or a portion of a polypeptide containing an antigen (e.g., a disease-associated antigen). The cells may also be modified to express an antigen itself if the immunogenic fragment of the polypeptide is known.

Before or after generating the cloaked cells of the disclosure, the cells can be modified to be FAILSAFE™ cells. FAILSAFE™ cells contain one or more cell division loci (CDLs) that control cell proliferation in animal cells. CDLs, as provided herein, may be loci whose transcription product(s) are expressed during cell division. CDLs may be genetically modified, as described herein, to comprise a negative selectable marker and/or an inducible activator-based gene expression system, which allows a user to permit, ablate, and/or inhibit proliferation of the genetically modified cell(s) by adding or removing an appropriate inducer. Methods for making and using FAILSAFE™ cells are described, for example, in WO 2016/141480, the entire teachings of which are incorporated herein by reference. A cell may be made FAILSAFE™ first and then cloaked afterwards. Alternatively, a cell may be cloaked first and then made FAILSAFE™ afterwards. In another embodiment, an uncloaked cell of the same type as the cloaked cell (e.g., a cell of the same type derived from the same source as the cloaked cell that is not modified to express any of the cloaking transgenes) that is modified to express a polypeptide or a portion of a polypeptide containing an antigen (e.g., a disease-associated antigen) can be made FAILSAFE™ (e.g., either before or after it is modified to express the disease-associated antigen).

The cell may be a vertebrate cell, for example, a mammalian cell, such as a human cell or a mouse cell. The cell may also be a vertebrate stem cell, for example, a mammalian stem cell, such as a human stem cell or a mouse stem cell. Preferably, the cell or stem cell is amenable to genetic modification. Preferably, the cell or stem cell is deemed by a user to have therapeutic value, meaning that the cell or stem cell may be used to treat a disease, disorder, defect or injury in a subject in need of treatment for same.

In some embodiments, the cell is a stem cell or progenitor cell (e.g., iPSC, embryonic stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, or neural stem or progenitor cells). In some embodiments, the stem cells are adult stem cells (e.g., somatic stem cells or tissue specific stem cells). In some embodiments, the stem or progenitor cell is capable of being differentiated (e.g., the stem cell is totipotent, pluripotent, or multipotent). In some embodiments, the cell is isolated from embryonic or neonatal tissue. In some embodiments, the cell is a fibroblast, monocytic precursor, B cell, exocrine cell, pancreatic progenitor, endocrine progenitor, hepatoblast, myoblast, preadipocyte, progenitor cell, hepatocyte, chondrocyte, smooth muscle cell, K562 human erythroid leukemia cell line, bone cell, synovial cell, tendon cell, ligament cell, meniscus cell, adipose cell, dendritic cell, neutrophil, basophil, mast cell, monocyte, innate lymphoid cell, or natural killer cell. In some embodiments, the cell is manipulated (e.g., converted or differentiated) into a muscle cell, erythroid-megakaryocytic cell, eosinophil, iPS cell, macrophage, T cell, islet beta-cell, neuron, cardiomyocyte, blood cell (e.g., red blood cell, white blood cell, or platelet), endocrine progenitor, exocrine progenitor, ductal cell, acinar cell, alpha cell, beta cell, delta cell, PP cell, hepatocyte, cholangiocyte, or brown adipocyte. In some embodiments, the cell is a muscle cell (e.g., skeletal, smooth, or cardiac muscle cell), erythroid-megakaryocytic cell, eosinophil, iPS cell, macrophage, T cell, islet beta-cell, neuron, cardiomyocyte, blood cell (e.g., red blood cell, white blood cell, or platelet), endocrine progenitor, exocrine progenitor, ductal cell, acinar cell, alpha cell, beta cell, delta cell, PP cell, hepatocyte, cholangiocyte, or white or brown adipocyte. In some embodiments, the cell is a hormone-secreting cell (e.g., a neuroendocrine cell, e.g., a cell that secretes insulin, oxytocin, endorphin, vasopressin, serotonin, somatostatin, gastrin, secretin, glucagon, thyroid hormone, bombesin, cholecystokinin, testosterone, estrogen, or progesterone, renin, ghrelin, amylin, or pancreatic polypeptide), an epidermal keratinocyte, an epithelial cell (e.g., an exocrine secretory epithelial cell, a thyroid epithelial cell, a keratinizing epithelial cell, a gall bladder epithelial cell, or a surface epithelial cell of the cornea, tongue, oral cavity, esophagus, anal canal, distal urethra, or vagina), a kidney cell, a germ cell, a skeletal joint synovium cell, a periosteum cell, a bone cell (e.g., osteoclast or osteoblast), a perichondrium cell (e.g., a chondroblast or chondrocyte), a cartilage cell (e.g., chondrocyte), a fibroblast, an endothelial cell, a pericardium cell, a meningeal cell, a keratinocyte precursor cell, a keratinocyte stem cell, a pericyte, a glial cell (e.g., oligodendrocyte, Schwann cell, astrocyte, or microglial cell), an ependymal cell, a cell isolated from an amniotic or placental membrane, or a serosal cell (e.g., a serosal cell lining body cavities). In some embodiments, the cell is a somatic cell. In some embodiments, the cells are derived from skin or other organs, e.g., heart, brain or spinal cord, liver, lung, kidney, pancreas, bladder, bone marrow, spleen, intestine, or stomach. The cells can be from humans or other mammals (e.g., rodent, non-human primate, bovine, or porcine cells). The cells can be from a cell line (e.g., a human cell line). The cells can be allogeneic or autologous cells. The cells may be syngeneic cells.

In some embodiments, the cloaked cells described herein survive in a host without stimulating the host immune response for one week or more (e.g., one week, two weeks, one month, two months, three months, 6 months, one year, two years, three years, four years, five years or more, e.g., for the life of the cell and/or its progeny). The cells maintain expression of the cloaking transgenes for as long as they survive in the host (e.g., if cloaking transgenes are no longer expressed, the cloaked cells may be removed by the host's immune system). In some embodiments, the cloaked cells further express a transgene encoding a protein that allows the cloaked cells to be detected in vivo (e.g., a fluorescent protein, such as GFP, or other detectable marker).

It is contemplated herein that the combination of cloaked and fail-safe cells may be of use in cell-based therapies wherein it may be desirable to induce antigen-specific immune tolerance or an antigen-specific immune response, while also being able to eliminate cells exhibiting undesirable growth rates, irrespective of whether such cells are generated before or after grafting the cells into a host. The combination of the cloaking and fail-safe technologies allows for targeted modulation of the immune response while addressing the risk that the recipient will develop a malignancy due to uncontrolled proliferation of the cloaked cells or because the cells are providing local immunosuppression.

### Methods of Producing Cloaked Cells

The compositions and methods described herein can be used modulate the response of the immune system to specific antigens through expression of cloaking transgenes concurrently with or prior to the expression of a polypeptide containing an antigen. A wide array of methods has been established for the delivery of proteins to mammalian cells and for the stable expression of genes encoding proteins in mammalian cells, which can be used to produce the cloaked cells described herein.

### Polynucleotides Encoding Cloaking Proteins or Polypeptides Containing Antigens

One platform that can be used to achieve therapeutically effective expression of cloaking proteins and/or polypeptides containing antigens in mammalian cells is via the stable expression of a gene encoding a cloaking protein or a gene encoding a polypeptide containing an antigen (e.g., by integration into the nuclear or mitochondrial genome of a mammalian cell, or by episomal concatemer formation in the nucleus of a mammalian cell). The gene is a polynucleotide that encodes the primary amino acid sequence of the corresponding protein. In order to introduce exogenous genes into a mammalian cell, genes can be incorporated into a vector. Vectors can be introduced into a cell by a variety of methods, including transformation, transfection, transduction, direct uptake, projectile bombardment, and by encapsulation of the vector in a liposome. Examples of suitable methods of transfecting or transforming cells include calcium phosphate precipitation, electroporation, microinjection, infection, lipofection and direct uptake. Such methods are described in more detail, for example, in Green, et al., Molecular Cloning: A Laboratory Manual, Fourth Edition (Cold Spring Harbor University Press, New York 2014); and Ausubel, et al., Current Protocols in Molecular Biology (John Wiley & Sons, New York 2015), the disclosures of each of which are incorporated herein by reference.

Cloaking proteins or polypeptides containing antigens can also be introduced into a mammalian cell by targeting vectors containing portions of a gene encoding a cloaking protein or a gene encoding a polypeptide containing an antigen to cell membrane phospholipids. For example, vectors can be targeted to the phospholipids on the extracellular surface of the cell membrane by linking the vector molecule to a VSV-G protein, a viral protein with affinity for all cell membrane phospholipids. Such a construct can be produced using methods well known to those of skill in the field.

Recognition and binding of the polynucleotide encoding a cloaking protein or the polynucleotide encoding a polypeptide containing an antigen by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase.

Polynucleotides suitable for use in the compositions and methods described herein also include those that encode a cloaking protein or encode a polypeptide containing an antigen downstream of a mammalian promoter. Promoters that are useful for the expression of a cloaking protein or a polypeptide containing an antigen in mammalian cells include constitutive promoters. Constitutive promoters include the CAG promoter, the cytomegalovirus (CMV) promoter, the EF1a promoter, and the PGK promoter. Alternatively, promoters derived from viral genomes can also be used for the stable expression of these agents in mammalian cells. Examples of functional viral promoters that can be used to promote mammalian expression of these agents include adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

Once a polynucleotide encoding a cloaking protein or encoding a polypeptide containing an antigen described herein below has been incorporated into the nuclear DNA of a mammalian cell, the transcription of this polynucleotide can be induced by methods known in the art. For example expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms include tetracycline and doxycycline. These reagents are commercially available (Life Technologies, Carlsbad, CA) and can be administered to a mammalian cell in order to promote gene expression according to established protocols.

Other DNA sequence elements that may be included in the nucleic acid vectors for use in the compositions and methods described herein include enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode a cloaking protein or encode a polypeptide containing an antigen and additionally include a mammalian enhancer sequence. Many enhancer sequences are now known from mammalian genes, and examples include enhancers from the genes that encode mammalian globin, elastase, albumin, a-fetoprotein, and insulin. Enhancers for use in the compositions and methods described herein also include those that are derived from the genetic material of a virus capable of infecting a eukaryotic cell. Examples include the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv, et al., Nature 297:17 (1982). An enhancer may be spliced into a vector containing a polynucleotide encoding a cloaking protein or a polynucleotide encoding a polypeptide containing an antigen, for example, at a position 5′ or 3′ to this gene. In a preferred orientation, the enhancer is positioned at the 5′ side of the promoter, which in turn is located 5′ relative to the polynucleotide encoding a cloaking protein or a polypeptide containing an antigen.

The nucleic acid vectors described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cell. The addition of the WPRE to a vector can result in a substantial improvement in the level of transgene expression from several different promoters, both in vitro and in vivo.

In some embodiments, the nucleic acid vectors for use in the compositions and methods described herein include a reporter sequence, which can be useful in verifying gene expression, for example, in specific cells and tissues. Reporter sequences that may be provided in a transgene include DNA sequences encoding B-lactamase, B-galactosidase (LacZ), alkaline phosphatase, thymidine kinase, green fluorescent protein (GFP), chloramphenicol acetyltransferase (CAT), luciferase, and others well known in the art. When associated with regulatory elements which drive their expression, the reporter sequences provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA), and immunohistochemistry. For example, where the marker sequence is the LacZ gene, the presence of the vector carrying the signal is detected by assays for B-galactosidase activity. Where the transgene is green fluorescent protein or luciferase, the vector carrying the signal may be measured visually by color or light production in a luminometer.

Techniques for Introducing Transgenes into Cells

### Transfection

Techniques that can be used to introduce a transgene, such as a cloaking transgene or a transgene encoding a polypeptide containing an antigen described herein, into a target cell (e.g., a mammalian cell) are well known in the art. For instance, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, NUCLEOFECTION™, utilizes an applied electric field in order to stimulate the uptake of exogenous polynucleotides into the nucleus of a eukaryotic cell. NUCLEOFECTION™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., Journal of Visualized Experiments 81: e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) polyethylenimine, and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect target cells in a mild and efficient manner, as this methodology utilizes an applied magnetic field in order to direct the uptake of nucleic acids. This technology is described in detail, for instance, in US 2010/0227406, the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:1870 (2010), the disclosure of which is incorporated herein by reference.

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Therapy 9:102 (2002), the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a target cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyzes the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Viral Infection

In addition to achieving high rates of transcription and translation, stable expression of an exogenous gene in a mammalian cell can be achieved by integration of the polynucleotide containing the gene into the nuclear genome of the mammalian cell. A variety of vectors for the delivery and integration of polynucleotides encoding exogenous proteins into the nuclear DNA of a mammalian cell have been developed. Examples of expression vectors are disclosed in, e.g., WO 1994/011026 and are incorporated herein by reference. Expression vectors for use in the compositions and methods described herein contain a cloaking transgene or a transgene encoding a polypeptide containing an antigen, as well as, e.g., additional sequence elements used for the expression of these agents and/or the integration of these polynucleotide sequences into the genome of a mammalian cell. Certain vectors that can be used for the expression of cloaking transgenes or transgenes encoding a polypeptide containing an antigen include plasmids that contain regulatory sequences, such as promoter and enhancer regions, which direct gene transcription. Other useful vectors for expression of cloaking transgenes or transgenes encoding a polypeptide containing an antigen contain polynucleotide sequences that enhance the rate of translation of these genes or improve the stability or nuclear export of the mRNA that results from gene transcription. These sequence elements include, e.g., 5′ and 3′ untranslated regions and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

Viral vectors for nucleic acid delivery Viral genomes provide a rich source of vectors that can be used for the efficient delivery of a gene of interest into the genome of a target cell (e.g., a mammalian cell, such as a human cell). Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, 1996)). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, U.S. Pat. No. 5,801,030, the disclosure of which is incorporated herein by reference as it pertains to viral vectors for use in gene therapy.

### AAV Vectors for Nucleic Acid Delivery

In some embodiments, cloaking transgenes or transgenes encoding a polypeptide containing an antigen described herein are incorporated into rAAV vectors and/or virions in order to facilitate their introduction into a cell. rAAV vectors useful in the compositions and methods described herein are recombinant nucleic acid constructs that include (1) a promoter, (2) a heterologous sequence to be expressed (e.g., a cloaking transgene or a transgene encoding a polypeptide containing an antigen described herein), and (3) viral sequences that facilitate integration and expression of the heterologous genes. The viral sequences may include those sequences of AAV that are required in cis for replication and packaging (e.g., functional ITRs) of the DNA into a virion. Such rAAV vectors may also contain marker or reporter genes. Useful rAAV vectors have one or more of the AAV WT genes deleted in whole or in part, but retain functional flanking ITR sequences. The AAV ITRs may be of any serotype suitable for a particular application. Methods for using rAAV vectors are described, for example, in Tal et al., J. Biomed. Sci. 7:279 (2000), and Monahan and Samulski, Gene Delivery 7:24 (2000), the disclosures of each of which are incorporated herein by reference as they pertain to AAV vectors for gene delivery.

The transgenes and vectors described herein (e.g., a promoter operably linked to a cloaking transgene or a transgene encoding a polypeptide containing an antigen) can be incorporated into a rAAV virion in order to facilitate introduction of the polynucleotide or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for instance, in U.S. Pat. Nos. 5,173,414; 5,139,941; 5,863,541; 5,869,305; 6,057,152; and 6,376,237; as well as in Rabinowitz et al., J. Virol. 76:791 (2002) and Bowles et al., J. Virol. 77:423 (2003), the disclosures of each of which are incorporated herein by reference as they pertain to AAV vectors for gene delivery.

rAAV virions useful in conjunction with the compositions and methods described herein include those derived from a variety of AAV serotypes including AAV 1, 2, 3, 4, 5, 6, 7, 8, 9, 10, rh10, rh39, rh43, and rh74. Construction and use of AAV vectors and AAV proteins of different serotypes are described, for instance, in Chao et al., Mol. Ther. 2:619 (2000); Davidson et al., Proc. Natl. Acad. Sci. USA 97:3428 (2000); Xiao et al., J. Virol. 72:2224 (1998); Halbert et al., J. Virol. 74:1524 (2000); Halbert et al., J. Virol. 75:6615 (2001); and Auricchio et al., Hum. Molec. Genet. 10:3075 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). Techniques involving the construction and use of pseudotyped rAAV virions are known in the art and are described, for instance, in Duan et al., J. Virol. 75:7662 (2001); Halbert et al., J.

Virol. 74:1524 (2000); Zolotukhin et al., Methods, 28:158 (2002); and Auricchio et al., Hum. Molec. Genet. 10:3075 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants is described in Wu et al., J. Virol. 74:8635 (2000). Other rAAV virions that can be used in methods described herein include those capsid hybrids that are generated by molecular breeding of viruses as well as by exon shuffling. See, e.g., Soong et al., Nat. Genet., 25:436 (2000) and Kolman and Stemmer, Nat. Biotechnol. 19:423 (2001).

### Genome Editing

In addition to the above, a variety of tools have been developed that can be used for the incorporation of a gene of interest into a target cell, such as a mammalian cell. One such method that can be used for incorporating polynucleotides encoding target genes (e.g., a cloaking transgene or a transgene encoding a polypeptide containing an antigen described herein) into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA: DNA hybridization. As a result, one can design a CRISPR/Cas system to cleave any target DNA molecule of interest. This technique has been exploited in order to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nature Reviews Genetics 11:636 (2010); and in Joung et al., Nature Reviews Molecular Cell Biology 14:49 (2013), the disclosure of each of which are incorporated herein by reference as they pertain to compositions and methods for genome editing.

Additional genome editing techniques that can be used to incorporate polynucleotides encoding target genes into the genome of a target cell include the use of ARCUS™ meganucleases that can be rationally designed so as to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Expression of Cloaking Transgenes

The cloaking transgenes described herein (e.g., one of, or any combination of, PD-L1, HLA-G (H2-M3), Cd47, Cd200, FASLG (FasL), Ccl21 (Ccl21b), Mfge8, and Serpin B9 (Spi6)) are expressed in an amount sufficient to produce a cloaking effect (e.g., in an amount sufficient to prevent rejection when injected into a subject, e.g., a mammalian subject, such as a mouse, rat, or human). Transgene expression can be considered to produce a cloaking effect if subcutaneous injection of cloaked cells generates a teratoma that is not removed by the subject's immune system. The cloaking transgenes are also expressed at a level that is sufficient to promote production of the proteins encoded by said transgenes. Protein production can be detected using routine methods known to those of skill in the art (e.g., immunohistochemistry, Western Blot analysis, or other methods that allow for visualization or proteins). Preferably, the expression of the cloaking transgenes is such that all 8 proteins encoded by the cloaking transgenes (PD-L1, H2-M3, Cd47, Cd200, FasL, Ccl21b, Mfge8, and Spi6) can be detected in cloaked cells (e.g., detected by immunohistochemistry using antibodies directed against the proteins encoded by the cloaking transgenes).

In some embodiments, cloaking transgenes are expressed at similar levels in cloaked cells to levels of endogenous gene expression in activated leukocytes, such as T cells (e.g., activated leukocytes from the same species, such as an activated leukocyte isolated from a lymph organ, for example expression in a cloaked mouse cell is similar to expression in an activated leukocyte isolated from a murine lymphoid organ). The expression of one or more cloaking transgenes (e.g., 1, 2, 3, 4, 5, 6, 7, or 8 of PD-L1, HLA-G (H2-M3), Cd47, Cd200, FASLG (FasL), Ccl21 (Ccl21b), Mfge8, and Serpin B9 (Spi6)) is greater than or equal to expression of the endogenous gene in activated leukocytes (e.g., T cells) from the same species (e.g., expression level of the cloaking transgene is equal to the level of expression of the endogenous gene in activated leukocytes, or is 1.5, 2, 3, 4, 5, 6, 7, 8, 9, 10-fold or more higher than the level of expression of the endogenous gene in activated leukocytes). In some embodiments, all 8 cloaking transgenes are expressed at a level that is greater than or equal to the expression level of the endogenous gene in an activated leukocyte from the same species. Activated leukocytes can be isolated from lymphoid organs, or leukocytes, such as T cells, can activated in vitro using anti-CD3/CD28 beads or other methods employed by those of skill in the art (see, e.g., Frauwith and Thompson, J.Clin Invest 109:295-299 (2002); and Trickett and Kwan, J Immunol Methods 275:251-255 (2003)). Transgene expression in cloaked cells can also be compared to gene expression levels reported in profiling studies of activated T cells (see, e.g., Palacios et al., PLOSone 2: e1222 (2007)). In some embodiments, cloaking transgene expression is compared to expression of the endogenous gene in a wild-type version of the cell (e.g., a stem cell, e.g., an embryonic stem cell from the same species as the cloaked cell). The expression of one or more cloaking transgenes (e.g., 1, 2, 3, 4, 5, 6, 7, or 8 of PD-L1, HLA-G (H2-M3), Cd47, Cd200, FASLG (FasL), Ccl21 (Ccl21b), Mfge8, and Serpin B9 (Spi6)) is at least 2, 3, 4, 5, 6, 7, 8, 9, 10, 20, 30, 40, 50, 100, 500, 1,000-fold or more higher in cloaked cells compared to expression of the endogenous gene in unmodified wild-type cells of the same cell type as the cloaked cell (e.g., stem cells, such as embryonic stem cells from the same species). In some embodiments, all 8 cloaking transgenes are expressed at a level that is greater (e.g., 2, 3, 4, 5, 6, 7, 8, 9, 10, 20, 30, 40, 50, or 100-fold higher or more) than the expression level of the endogenous gene in a wild-type version of the cell (e.g., a stem cell, e.g., an embryonic stem cell from the same species as the cloaked cell). Gene expression can be evaluated through direct comparison to isolated ES cells, or compared to stem cell expression (e.g., ES cell expression) in the Project Grandiose dataset (www.stemformatics.org/project_grandiose). Gene expression can be measured using techniques known in the art (e.g., quantitative polymerase chain reaction (qPCR)). Exemplary expression of cloaking transgenes is described in International Patent Application No. PCT/CA2018/050706, which is incorporated herein by reference in its entirety.

### Cloaked Cells for the Induction of Immune Tolerance

Due to their ability to suppress the immune response, the cloaked cells of the invention can be modified for use in various cell-based therapies. In one example, the set of transgenes described herein can be used to cloak cells for the induction of immune tolerance (e.g., to prevent or reduce the response of the immune system to antigens expressed by cloaked cells). The ability to induce immune tolerance to specific antigens allows for the development of targeted therapeutics to suppress a harmful or aberrant immune response and may reduce or obviate the need for immunosuppressive medication. Immunosuppressive medication renders the body more vulnerable to infections and malignancy; therefore, cell-based therapies that can be used in place of immunosuppressive medication or reduce the dose of immunosuppressive medication that is administered to a subject may be associated with fewer risks and side effects.

### Cloaked Cells for Inducing Tolerance of an Organ or Tissue Transplant

The set of transgenes described herein can be used to cloak cells to induce immune tolerance to alloantigens from a donor (e.g., a human organ or tissue donor). After organ or tissue transplantation, the immune system of the recipient often rejects the new organ or tissue, detecting it as foreign or non-self due to differences in human leukocyte antigen (HLA) between the donor and the recipient. The immune system may then attack the new tissue, which can result in damage to or death of the donated tissue or organ. Immunosuppressive medication can be given to the recipient to suppress this immune response, but it may result in deleterious side effects. The present invention provides an alternative approach, in which the recipient is administered cloaked cells (e.g., cloaked donor cells or cloaked cells expressing an alloantigen from the donor, e.g., an HLA molecule from the donor) in advance of the transplant to induce tolerance prior to the organ or tissue transplant.

In some embodiments, the cloaked cell is a cell from an organ or tissue donor (e.g., a somatic cell or stem cell (e.g., an adult stem cell, an iPSC, a tissue-specific stem cell, a hematopoietic stem cell, a mesenchymal stem cell, an endothelial stem cell, an epithelial stem cell, or a hair follicle stem cell) from an organ or tissue donor, or a cell from the tissue or organ to be donated). Cells from an organ or tissue donor carry alloantigens from the donor to which the recipient of the organ or tissue transplant can be tolerized to reduce or prevent the risk of transplant rejection. The cells from the donor can be modified to express one or more (e.g., two, three, four, five, six, seven, or all eight) of the cloaking transgenes: PD-L1, HLA-G (H2-M3), Cd47, Cd200, FASLG (FasL), Ccl21 (Ccl21b), Mfge8, and Serpin B9 (Spi6). In some embodiments, the donor cells are modified to express all eight of Pd-L1, H2-M3, Cd47, Cd200, FasL, Ccl21b, Mfge8, and Spi6. In addition to the cloaking transgenes, the cells may be further modified to express one or more of TGF-β, Cd73, Cd39, Lag3, Il1r2, Ackr2, Tnfrsf22, Tnfrs23, Tnfrsf10, Dad1, and IFNγR1 d39. The donor cells can be modified to express the one or more cloaking transgenes using the techniques described herein above (e.g., transfection, viral infection, or gene editing) or using other methods known by those of skill in the art. This approach does not require the identification of the alloantigens expressed by the donor cells prior to administration of cloaked donor cells to the recipient. Moreover, this approach allows for the induction of immune tolerance to all of the alloantigens expressed by the donor cell (e.g., the immune system of the recipient will learn to recognize all of the alloantigens expressed by the donor cell and treat them as “self” antigens, and, therefore will not mount an immune response against these antigens when they are presented again in the uncloaked organ or tissue transplant).

In some embodiments, the cloaked cell is not from the organ or tissue donor. The cell may be a stem cell (e.g., an ES cell, an adult stem cell, an iPSC, a tissue-specific stem cell, a hematopoietic stem cell, a mesenchymal stem cell, an endothelial stem cell, an epithelial stem cell, an adipose stem or progenitor cell, a germline stem cell, a lung stem or progenitor cell, a mammary stem cell, an olfactory adult stem cell, a hair follicle stem cell, a multipotent stem cell, an amniotic stem cell, a cord blood stem cell, or a neural stem or progenitor cell), a somatic cell, a cell derived from a cell line, a cell amenable to genome editing, and/or a source of therapeutic cell type (e.g., a cell that can be differentiated into a lineage restricted cell for cell therapy, or a cell of a desired target tissue). In some embodiments, the cell is a skin, heart, brain or spinal cord, liver, lung, kidney, pancreas, bladder, bone marrow, spleen, intestine, or stomach cell. In some embodiments, the cell is a fibroblast, an epithelial cell, or an endothelial cell. The cell may be a vertebrate cell, for example, a mammalian cell, such as a human or mouse cell. In some embodiments, the cell is an autologous cell (e.g., a cell isolated from the subject in need of tolerization). In some embodiments, the cell that is cloaked is of the same cell type as the tissue or organ to be transplanted (e.g., a liver cell is cloaked if the subject will receive a liver transplant). In this embodiment, the cell is modified to express one or more (e.g., two, three, four, five, six, seven, or all eight) of the cloaking transgenes: PD-L1, HLA-G (H2-M3), Cd47, Cd200, FASLG (FasL), Ccl21 (Ccl21b), Mfge8, and Serpin B9 (Spi6) and one or more alloantigen from the organ or tissue donor (e.g., an HLA molecule from the organ or tissue donor). In some embodiments, the cloaked cell is modified to express all eight of Pd-L1, H2-M3, Cd47, Cd200, FasL, Ccl21b, Mfge8, and Spi6 and one or more alloantigen from the organ or tissue donor (e.g., an HLA molecule from the organ or tissue donor). In addition to the cloaking transgenes, the cells may be further modified to express one or more of TGF-β, Cd73, Cd39, Lag3, Il1r2, Ackr2, Tnfrsf22, Tnfrs23, Tnfrsf10, Dad1, and IFNγR1 d39.

The alloantigen from the organ or tissue donor can be any alloantigen capable of stimulating an immune response in the recipient. In some embodiments, the alloantigen is a histocompatibility protein (e.g., an HLA protein), such as an HLA class I or class II protein. In some embodiments, the alloantigen is a blood group antigen. The alloantigens expressed by the organ or tissue donor may be identified using standard methods, such as molecular typing of HLA alleles (using PCR), serologic typing of HLA antigens (using the standard complement-mediated microlymphocytotoxicity technique), sequencing, or mass spectrometry analysis. The cell can be modified to express the cloaking transgenes and one or more (e.g., 1, 2, 3, 4, 5, or more) polypeptide containing an alloantigen from the organ or tissue donor using the techniques described herein above (e.g., transfection, viral infection, or gene editing) or using other methods known by those of skill in the art. To determine whether an alloantigen stimulates an immune response in a recipient (e.g., prior to the administration of a cloaked cell expressing the alloantigen), an immune cell from the recipient can be exposed to the alloantigen and the response of the immune cell can be measured (e.g., an immune cell, such as a dendritic cell, macrophage, T cell (e.g., a cytotoxic T cell/CD8⁺ T cell, T helper cell/CD4⁺ T cell, or regulatory T cell/Treg), B cell, monocyte, granulocyte (e.g., eosinophil, mast cell, neutrophil, or basophil), natural killer (NK) cell, or innate lymphoid cell isolated from the recipient can be contacted with the alloantigen in vitro, and a response of the immune cell (e.g., immune cell activation, differentiation, polarization, proliferation, migration, pro-inflammatory cytokine production, degranulation, phagocytosis, or cytotoxicity) can be measured). If the immune cell from the subject responds to the donor alloantigen (e.g., if the immune cell becomes more activated, releases pro-inflammatory cytokines, differentiates, proliferates, becomes more polarized toward an inflammatory phenotype, or exhibits degranulation, phagocytosis, or cytotoxicity), a cloaked cell can be modified to express the protein containing the donor alloantigen to induce immune tolerance to the alloantigen in the recipient and reduce or prevent rejection of an organ or tissue transplant from the donor. The cloaked cell may be modified to express the polypeptide containing the donor alloantigen at an expression level comparable to the endogenous expression level of the polypeptide containing the donor alloantigen in a cell or tissue from the donor. Alternatively, the cloaked cell may be modified to express the polypeptide containing the donor alloantigen at a level comparable to the expression level of a housekeeping polypeptide (e.g., . β-actin, GAPDH, or Rosa26). Robust expression of the polypeptide can be achieved using strong promoters, such as CAGG or CMV or through the use of a doxycycline inducible system (e.g., a doxycycline inducible TRE).

The cloaked donor cell or the cloaked cell expressing a protein containing a donor alloantigen may be administered to the subject in advance of the organ or tissue transplant to induce immune tolerance (e.g., administered to induce tolerance to a future organ or tissue transplant in order to reduce or prevent transplant rejection and to reduce or obviate the need for immunosuppressive medication, e.g., to reduce the amount of immunosuppressive medication administered to a subject). The organ or tissue to be transplanted need not be cloaked, as the recipient's immune system should no longer recognize the transplant as foreign. The cloaked donor cell or the cloaked cell expressing a protein containing a donor alloantigen may be administered to the recipient prior to the organ or tissue transplant (e.g., 1 day, 2 days, 3 days, 4 days, 5 days, 6 days, 1 week, 2 weeks, 3 weeks, 1 month, 2 months, 3 months, 4 months, 5 months, 6 months, 9 months, 1 year or more before the organ or tissue transplant). The cloaked donor cell or the cloaked cell expressing a protein containing a donor alloantigen can be injected at or near the site of the future organ or tissue transplant, injected as a circulating cell, or injected subcutaneously to produce a cloaked subcutaneous tissue. In some embodiments, twenty five thousand to one hundred billion cloaked cells expressing a protein containing a donor alloantigen (e.g., 2.5×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 6×10⁵, 6×10⁵, 7×10⁵, 8×10⁵, 9×10⁵, 1×10⁶, 2×10⁶, 3×106, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ cloaked cells expressing a donor alloantigen) are administered to a subject to induce immune tolerance to a donor alloantigen. In some embodiments, an immune cell from the recipient can be tested to determine whether immune tolerance has been induced. For example, after administration of the cloaked donor cell or cloaked cell expressing an alloantigen (e.g., 1 day, 2 days, 3 days, 4 days, 5 days, 6 days, 1 week, 2 weeks, 3 weeks, 1 month, 2 months, 3 months, 4 months, 5 months, 6 months, 9 months, or 1 year after administration of the cloaked donor cell or cloaked cell expressing an alloantigen), an immune cell can be isolated from the recipient and exposed to an uncloaked cell from the donor (e.g., an uncloaked cell from the donor, an uncloaked cell from the organ or tissue to be donated, or an uncloaked cell that carries the donor alloantigen to which the recipient's immune system has been tolerized). The response of the recipient immune cell can be measured as described above. If the immune cell from the recipient does not mount an immune response against the uncloaked donor cell or donor alloantigen (e.g., if the immune cell does not become more activated or exhibit a cytotoxic, pro-inflammatory, or phagocytic response to the donor cell or donor alloantigen), the recipient's immune system has been successfully tolerized to the donor cell or donor alloantigen. The transplant can then be performed with minimal risk of transplant rejection and without the use of, or upon administration of a reduced dosage of, immunosuppressive medication. There is also no need to match the HLA antigens in the donor and recipient, as administration of cloaked cells carrying a donor HLA antigen can be used to induce immune tolerance to any donor HLA antigen (e.g., a 0/6 match, a 1/6 match, a 2/6 match, a 3/6 match, a 4/6 match or a 5/6 match).

In some embodiments, the organ that is transplanted after immune tolerance is induced using cloaked cells is a heart, kidney, brain, liver, lung, stomach, pancreas, intestine, testis, penis, hand, face, arm, foot, middle ear, or thymus. In some embodiments, the tissue that is transplanted after immune tolerance is induced using cloaked cells is a bone or tendon (e.g., a musculoskeletal graft), connective tissue, a cornea, skin, a heart valve, islets of Langerhans, bone marrow, or blood vessel (e.g., a nerve or a vein), blood, or a vascularized composite allograft (e.g., a transplant of several structures that may include skin, bone, muscles, blood vessels, nerves, and connective tissue).

The cloaked cells expressing the polynucleotide containing the donor alloantigen can be removed after transplantation of the donor tissue or organ (e.g., 1 week, 1 month, 2 months, 3 months, 4 months, 5 months, 6 months, 9 months, 1 year, 2 years or more after transplantation). The cloaked cells can be removed surgically (e.g., in embodiments in which the cloaked cells formed a tissue, such as a cloaked subcutaneous tissue, the tissue can be surgically removed). In embodiments in which the cloaked cells were modified to contain one or more systems for regulating cell division (e.g., modified to be FAILSAFE™ cells, e.g., by linking the expression of a CDL to an ALINK or EARC), the cloaked cells can be removed by administering an inducer of a negative selectable marker (e.g., the ALINK) or by ceasing to administer an activator of an inducible activator-based gene expression system (e.g., the EARC).

### Cloaked Cells for Inducing Tolerance to an Antigen

A normal, healthy immune system will detect and mount an immune response against foreign, “non-self” antigens expressed by a pathogenic or infectious organism, such as a virus or bacterium. Another feature of a normal, healthy immune system is the ability to recognize “self” antigens, which are endogenous and naturally produced, and refrain from mounting an immune response against these antigens. The recognition of self antigens appears to be disrupted in many autoimmune diseases, which are often characterized by immune system-induced damage to body's own cells. The cloaked cells described herein can be used to treat or mitigate such diseases, as they can be modified to express a polypeptide containing an antigen that the immune system is incorrectly targeting to induce immune tolerance to the antigen.

The cell can be cloaked (e.g., modified to express one or more (e.g., two, three, four, five, six, seven, or all eight) of PD-L1, HLA-G (H2-M3), Cd47, Cd200, FASLG (FasL), Ccl21 (Ccl21b), Mfge8, and Serpin B9 (Spi6)) and further modified to express a polypeptide (e.g., one or more polypeptides, e.g., 1, 2, 3, 4, 5, or more polypeptides) containing an antigen that stimulates an aberrant immune response in a subject, such as an antigen associated with an autoimmune disease or condition or a portion thereof that stimulates an immune response in a subject. In some embodiments, the cloaked cell expresses all eight of PD-L1, HLA-G (H2-M3), Cd47, Cd200, FASLG (FasL), Ccl21 (Ccl21b), Mfge8, and Serpin B9 (Spi6) and an antigen or portion thereof. The cells may be further modified to express one or more of TGF-β, Cd73, Cd39, Lag3, Il1r2, Ackr2, Tnfrsf22, Tnfrs23, Tnfrsf10, Dad1, and IFNγR1 d39. The cloaked cells can be modified to express the cloaking transgenes and the polypeptide containing the antigen or portion thereof using the methods described herein (e.g., transfection, viral infection, or gene editing) or using other methods known by those of skill in the art. The cell may be a stem cell (e.g., an ES cell, an adult stem cell, an iPSC, a tissue-specific stem cell, a hematopoietic stem cell, a mesenchymal stem cell, an endothelial stem cell, an epithelial stem cell, an adipose stem or progenitor cell, a germline stem cell, a lung stem or progenitor cell, a mammary stem cell, an olfactory adult stem cell, a hair follicle stem cell, a multipotent stem cell, an amniotic stem cell, a cord blood stem cell, or a neural stem or progenitor cell), a somatic cell, a cell derived from a cell line, a cell amenable to genome editing, and/or a source of therapeutic cell type (e.g., a cell that can be differentiated into a lineage restricted cell for cell therapy, or a cell of a desired target tissue). In some embodiments, the cell is a skin, heart, brain or spinal cord, liver, lung, kidney, pancreas, bladder, bone marrow, spleen, intestine, or stomach cell. In some embodiments, the cell is a fibroblast, an epithelial cell, or an endothelial cell. The cell may be a vertebrate cell, for example, a mammalian cell, such as a human or mouse cell. In some embodiments, the cell is an autologous cell (e.g., a cell isolated from the subject in need of tolerization).

The antigen to be provided by the cloaked cell may be selected based on the autoimmune disease or condition with which the subject is afflicted, as many autoimmune diseases and conditions are associated with aberrant immune responses to specific antigens. A list of polypeptides containing antigens and the autoimmune disease or condition associated with each polypeptide (e.g., the autoimmune disease or condition in which autoantibodies directed against the antigen have been found, e.g., the autoimmune disease or condition that could be treated by modifying cloaked cells to express the polypeptide containing the antigen to induce immune tolerance) are provided in Table 2 below. The portion of a polypeptide containing an antigen that induces an immune response in the subject can be determined prior to producing the cloaked cells by exposing an immune cell from the subject (e.g., a dendritic cell, macrophage, T cell (e.g., a cytotoxic T cell/CD8⁺ T cell, T helper cell/CD4⁺ T cell, or regulatory T cell/Treg), B cell, monocyte, granulocyte (e.g., eosinophil, mast cell, neutrophil, or basophil), natural killer (NK) cell, or innate lymphoid cell isolated from the subject) to different portions of the polypeptide (e.g., exposing isolated immune cells from the subject to different portions of the polypeptide in vitro) and determining whether the portion of the polypeptide produces an immune response (e.g., immune cell activation, differentiation, polarization, proliferation, migration, pro-inflammatory cytokine production, degranulation, phagocytosis, or cytotoxicity). If the polypeptide containing an antigen that produces or exacerbates the autoimmune disease or condition is unknown, immune cells from the subject can be exposed to a panel of antigens (e.g., a panel of wild-type human proteins) to determine which of the proteins may be responsible for inducing the aberrant immune response.

The cloaked cell may be modified to express the polypeptide containing the antigen at an expression level comparable to the endogenous expression level of the polypeptide containing the antigen in a cell of the subject to be treated (e.g., the subject having an autoimmune disease or condition). Alternatively, the cloaked cell may be modified to express the polypeptide containing the antigen at a level comparable to the expression level of a housekeeping polypeptide (e.g., B-actin, GAPDH, or Rosa26). Robust expression of the polypeptide can be achieved using strong promoters, such as CAGG or CMV or CMV or through the use of a doxycycline inducible system (e.g., a doxycycline inducible TRE).

The cloaked cells that are modified to provide a polypeptide containing an antigen may be administered to a subject having (e.g., identified to have, or diagnosed with) an autoimmune disease or condition, to a subject in the early stages of developing an autoimmune disease or condition (e.g., a subject with an autoimmune disease or condition that was recently identified or diagnosed, or an autoimmune disease or condition that is associated with mild, but worsening, symptoms), or to a subject at risk of developing an autoimmune disease or condition (e.g., a subject carrying a genetic mutation associated with an autoimmune disease or condition, a subject having a family history of an autoimmune disease or condition, or a subject exposed to pathogens or environmental risk factors that may increase the probability of developing an autoimmune disease or condition). The cloaked cells that are modified to provide a polypeptide containing an antigen for the induction of immune tolerance may treat or alleviate some or all of the symptoms of the autoimmune disease or condition, may reduce the severity of the autoimmune disease or condition, may slow or inhibit the progression of the autoimmune disease or condition, or may prevent or delay the development of the autoimmune disease or condition. In some embodiments, the administration of cloaked cells expressing a polypeptide containing an antigen and the subsequent induction of immune tolerance to the antigen allows other treatments for the autoimmune disease or condition to be more effective. If the antigen is transmembrane or membrane bound (e.g., naturally transmembrane or membrane bound, or modified to include a sequence that localizes the antigen to the membrane, such as a transmembrane domain or linker e.g., a GPI linker), the cloaked cell expressing a polypeptide containing the antigen can be injected at or near the site of the aberrant immune response (e.g., cloaked cells expressing insulin and/or GAD65 can be injected to or near the pancreas to treat type 1 diabetes, and cloaked cells expressing melanin associated antigen (MAA), retinal arrestin, and/or interphotoreceptor retinoid binding protein (IRBP) can be injected to or near the eye to treat uveitis), or injected subcutaneously to produce a cloaked subcutaneous tissue. If the antigen is secreted (e.g., naturally secreted, or modified to remove a sequence that localizes the antigen to the membrane, such as by deletion of a transmembrane domain or linker e.g., a GPI linker), the cloaked cell expressing a polypeptide containing the antigen can be injected as a circulating cell, injected at or near the site of the aberrant immune response, or injected subcutaneously to produce a cloaked subcutaneous tissue that secretes the antigen. Twenty five thousand to one hundred billion cloaked cells expressing an antigen (e.g., 2.5×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 6×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ cloaked cells expressing an antigen) can be administered to a subject to induce immune tolerance to the antigen depending on the route of administration and the site to which the cells are administered.

In some embodiments, an immune cell from the subject can be tested after administration of the cloaked cell expressing a polypeptide containing the antigen to determine whether immune tolerance has been induced. For example, after administration of the cloaked cell expressing an antigen (e.g., 1 day, 2 days, 3 days, 4 days, 5 days, 6 days, 1 week, 2 weeks, 3 weeks, 1 month, 2 months, 3 months, 4 months, 5 months, 6 months, 9 months, or 1 year after administration of the cloaked cell expressing an antigen), an immune cell can be isolated from the subject and exposed to the antigen (e.g., the antigen or an uncloaked cell expressing the polypeptide containing the antigen to which the subject's immune system has been tolerized). The response of the immune cell isolated from the subject can be measured as described above. If the immune cell from the subject does not mount an immune response against the antigen (e.g., if the immune cell does not become more activated or exhibit a cytotoxic, pro-inflammatory, or phagocytic response to the antigen or an uncloaked cell expressing the antigen), the subject's immune system has been successfully tolerized to the antigen.

The cloaked cells expressing a polypeptide containing the antigen can be removed after the subject's immune system has been tolerized to the antigen or after symptoms of the subject's autoimmune disease or condition have improved (e.g., 1 week, 1 month, 2 months, 3 months, 4 months, 5 months, 6 months, 9 months, 1 year, 2 years or more after the subject's immune system has been tolerized to the antigen or after symptoms of the subject's autoimmune disease or condition have improved). The cloaked cells can be removed surgically (e.g., in embodiments in which the cloaked cells formed a tissue, such as a cloaked subcutaneous tissue, the tissue can be surgically removed). In embodiments in which the cloaked cells were modified to contain one or more systems for regulating cell division (e.g., modified to be FAILSAFET cells, e.g., by linking the expression of a CDL to an ALINK or EARC), the cloaked cells can be removed by administering an inducer of a negative selectable marker (e.g., the ALINK) or by ceasing to administer an activator of an inducible activator-based gene expression system (e.g., the EARC).

In some embodiments, the antigen is a “self” antigen (e.g., a wild-type human protein), such as an antigen associated with an autoimmune disease or condition. In some embodiments, the antigen is a food antigen (e.g., a protein found in food, or a protein produced by the normal digestive process). In some embodiments, the antigen is an allergen (e.g., an antigen that causes an allergic reaction, such as food allergy, seasonal allergy, pet allergy, hives, hay fever, allergic conjunctivitis, poison ivy allergy, oak allergy, mold allergy, drug allergy, dust allergy, cosmetic allergy, and chemical allergy).

The self-antigen expressed by a cloaked cell described herein to induce immune tolerance may be an antigen associated with the development of an autoimmune disease, such as such as Acute Disseminated Encephalomyelitis (ADEM); Acute necrotizing hemorrhagic leukoencephalitis; Addison's disease; Adjuvant-induced arthritis; Agammaglobulinemia; Alopecia areata; Amyloidosis; Ankylosing spondylitis; Anti-GBM/Anti-TBM nephritis; Antiphospholipid syndrome (APS); Autoimmune angioedema; Autoimmune aplastic anemia; Autoimmune dysautonomia; Autoimmune gastric atrophy; Autoimmune hemolytic anemia; Autoimmune hepatitis; Autoimmune hyperlipidemia; Autoimmune immunodeficiency; Autoimmune inner ear disease (AIED); Autoimmune myocarditis; Autoimmune oophoritis; Autoimmune pancreatitis; Autoimmune retinopathy; Autoimmune thrombocytopenia purpura (ATP); Autoimmune thyroid disease; Autoimmune urticaria; Axonal & neuronal neuropathies; Balo disease; Behcet's disease; Bullous pemphigoid; Cardiomyopathy; Castleman disease; Celiac disease; Celiac sprue-dermatitis; Chagas disease; Chronic fatigue immune dysfunction syndrome (CFIDS); Chronic inflammatory demyelinating polyneuropathy (CIDP); Chronic recurrent multifocal osteomyelitis (CRMO); Churg-Strauss syndrome; Cicatricial pemphigoid/benign mucosal pemphigoid; Crohn's disease; Cogans syndrome; Collagen-induced arthritis; Cold agglutinin disease; Congenital heart block; Coxsackie myocarditis; Limited scleroderma (CREST syndrome/disease); Essential mixed cryoglobulinemia; Demyelinating neuropathies; Dermatitis herpetiformis; Dermatomyositis; Devic's disease (neuromyelitis optica); Discoid lupus; Dressler's syndrome; Endometriosis; Eosinophilic esophagitis; Eosinophilic fasciitis; Erythema nodosum Experimental allergic encephalomyelitis; Experimental autoimmune encephalomyelitis; Evans syndrome; Fibromyalgia; Fibromyositis; Fibrosing alveolitis; Giant cell arteritis (temporal arteritis); Giant cell myocarditis; Glomerulonephritis; Goodpasture's syndrome; Granulomatosis with Polyangiitis (GPA) (formerly called Wegener's Granulomatosis); Graves' disease; Guillain-Barre syndrome; Hashimoto's encephalitis; Hashimoto's thyroiditis; Hemolytic anemia; Henoch-Schonlein purpura; Herpes gestationis; Hypogammaglobulinemia; Hypothyroidism; Idiopathic thrombocytopenia purpura (ITP); Idiopathic pulmonary fibrosis; IgA nephropathy; IgG4-related sclerosing disease; Immunoregulatory lipoproteins; Inclusion body myositis; Interstitial cystitis; Inflammatory bowel disease; Juvenile arthritis; Juvenile oligoarthritis; Juvenile diabetes (Type 1 diabetes); Juvenile myositis; Kawasaki syndrome; Lambert-Eaton syndrome; Leukocytoclastic vasculitis; Lichen planus; Lichen sclerosus; Ligneous conjunctivitis; Linear IgA disease (LAD); Lupus (SLE); Lyme disease, chronic; Autoimmune lymphoproliferative syndrome (ALPS); Meniere's disease; Microscopic polyangiitis; Mixed connective tissue disease (MCTD); Mooren's ulcer; Mucha-Habermann disease; Multiple sclerosis; Myasthenia gravis; Myositis; Narcolepsy; Neuromyelitis optica (Devic's); Neutropenia; Non-obese diabetes; Ocular cicatricial pemphigoid; Optic neuritis; Palindromic rheumatism; PANDAS (Pediatric Autoimmune Neuropsychiatric Disorders Associated with Streptococcus); Paraneoplastic cerebellar degeneration; Paroxysmal nocturnal hemoglobinuria (PNH); Parry Romberg syndrome; Parsonnage-Turner syndrome; Pars planitis (peripheral uveitis); Pemphigus; Pemphigus vulgaris; Peripheral neuropathy; Perivenous encephalomyelitis; Pernicious anemia; POEMS syndrome; Polyarteritis nodosa; Type I, II, & III autoimmune polyglandular syndromes; Polychondritis; Polymyalgia rheumatica; Polymyositis; Postmyocardial infarction syndrome; Postpericardiotomy syndrome; Progesterone dermatitis; Primary biliary cirrhosis; Primary sclerosing cholangitis; Primary agammaglobulinemia; Psoriasis; Plaque Psoriasis; Psoriatic arthritis; Idiopathic pulmonary fibrosis; Pyoderma gangrenosum; Pure red cell aplasia; Raynauds phenomenon; Reactive Arthritis; Reflex sympathetic dystrophy; Reiter's syndrome; Relapsing polychondritis; Restless legs syndrome; Retroperitoneal fibrosis; Rheumatic fever; Rheumatoid arthritis; Sarcoidosis; Schmidt syndrome; Scleritis; Scleroderma; Sclerosing cholangitis; Sclerosing sialadenitis; Sjogren's syndrome; Sperm & testicular autoimmunity; Stiff person syndrome; Subacute bacterial endocarditis (SBE); Susac's syndrome; Sympathetic ophthalmia; Systemic lupus erythematosus (SLE); Systemic sclerosis; Takayasu's arteritis; Temporal arteritis/Giant cell arteritis; Thrombocytopeniarpura (TTP); Tolosa-Hunt syndrome; Transverse myelitis; Type 1 diabetes; Ulcerative colitis; Undifferentiated connective tissue disease (UCTD); Uveitis; Vasculitis; Vesiculobullous dermatosis; Vitiligo; or Wegener's granulomatosis (now termed Granulomatosis with Polyangiitis (GPA).

Exemplary antigens associated with autoimmune diseases and conditions are provided in Table 2, below.

TABLE 2
Autoimmune diseases and associated polypeptides containing antigens that
can be expressed by cloaked cells to treat or prevent the autoimmune disease

| Autoimmune disease | Antigen |
|---|---|
| Achalasia | PNMA2 (Ma-2/Ta), recoverin, glutamic |
| | acid decarboxylase 65 (GAD65) |
| Addison’s disease | 21-hydroxylase |
| Alopecia areata | Trichohyalin, tyrosinase-related protein |
| | 2, tyrosinase 1, tyrosinase 2, cytokeratin |
| | 16, Pro-opiomelanocortin-1 (POMC-1), |
| | tyrosine hydroxylase isoform B1, |
| | tyrosine hydroxylase isoform B2 |
| Amyloidosis | Amyloid-β, keratin, Ro/SSA, NC1 |
| | domain of Alpha-3 chain of type IV |
| | collagen, mutant transthyretin (V30M), |
| | Scl70 |
| Ankylosing spondylitis | P2RX7, chondromodulin 1, osteoglycin, |
| | melanocortin 4 receptor, osteonectin, |
| | connective tissue growth factor, glypican |
| | 3, glypican 4, Matrix Gla protein, SPARC |
| | Related Modular Calcium Binding 1 |
| | (SMOC1), interleukin 6 (IL-6), collagen I, |
| | II, III, IV, and V, Protein Phosphatase, |
| | Mg2+/Mn2+ Dependent 1A (PPM1A), |
| | 14-3-4η |
| Anti-GBM/Anti-TBM nephritis | alpha-3 chain of type IV collagen, alpha- |
| | 4 chain of type IV collagen, alpha-5 |
| | chain of type IV collagen, |
| | tubulointerstitial nephritis antigen |
| Antiphospholipid syndrome | β₂-glycoprotein-I, cardiolipin |
| | phosphatidylserine |
| Autoimmune angioedema | C1 inhibitor (C1-INH) |
| Acquired hemophilia | Factor VIII |
| Autoimmune dysautonomia | Ganglionic acetylcholine receptor (α3- |
| | nicotinic AChR), M1, M2, and M3 |
| | muscarinic AChRs |
| Autoimmune encephalomyelitis | NMDA receptors, AMPA receptors, |
| | GABA_{A} receptors, GABA_{B} receptors, D2 |
| | receptors, Gly receptors, GAD65, |
| | Leucine Rich Glioma Inactivated 1 |
| | (LGI1), Caspr2, amphiphysin, DPPX, |
| | PCA-1, ANNA-1, Delta/Notch Like EGF |
| | Repeat Containing (DNER), mGluR1, |
| | VGCC |
| Autoimmune hepatitis | Asialoglycoprotein, asialoglycoprotein |
| | receptor, LKM-1, LKM-3, Cytochrome |
| | P450 Family 2 Subfamily D Member |
| | 6 (CYP2D6), Cytochrome P450 Family 1 |
| | Subfamily A Member 2 (CYP1A2), |
| | Cytochrome P450 Family 2 Subfamily E |
| | Member 1 (CYP2E1), Cytochrome P450 |
| | Family 3 Subfamily A Member 4 |
| | (CYP3A4), LC1, UDP- |
| | glucuronosyltransferases, |
| | formiminotransferase cyclodeaminase, |
| | actin, troponin, vimentin, tropomyosin, |
| | proteinase-3, myeloperoxidase, UGA- |
| | suppressor-tRNA-associated protein |
| Autoimmune inner ear disease (AIED) | Sulfoglucuronosyl glycolipids, HSP70, |
| | type II collagen, type IX collagen, Raf-1, |
| | major peripheral myelin protein P0, beta |
| | actin, cochlin, CTL2, cell-density |
| | enhanced protein tyrosine phosphatase- |
| | 1, connexion, proteinase 3, |
| | myeloperoxidase |
| Autoimmune myocarditis | Myosin, β₁-adrendergic receptor, |
| | Muscarinic M2 acetylcholine receptor, |
| | troponin, tropomyosin, laminin. |
| Autoimmune oophoritis | Maternal antigen that embryos require |
| | (Mater/Nalp5), 21β-hydroxylase, 17α- |
| | hydroxylase, P450scc, FSH receptor, LH |
| | receptor |
| Autoimmune orchitis | Hsp60, Hsp70, hnRNP H1, Outer Dense |
| | Fiber of Sperm Tails 2 (ODF-2), |
| | phosphoglycerate kinase 1 |
| Autoimmune pancreatitis | Carbonic anhydrase-II, carbonic |
| | anhydrase-IV, lactoferrin, Hsp10, |
| | amylase-2α, plasminogen-binding |
| | protein, pancreas secretory trypsin |
| | inhibitor, trypsinogen, thyroglobulin, |
| | thyroid peroxidase |
| Autoimmune retinopathy | Carbonic anhydrase II, rhodopsin, |
| | arrestin, phosphodiesterase, enolase |
| Autoimmune urticaria | FcεRIa of FCER1, IgE |
| Axonal & neuronal neuropathy (AMAN) | Gangliosides GM1, GM1b, GD1a |
| Baló’s disease | Aquaporin-4 |
| Behcet’s disease | Carbonic anhydrase I, carbonic |
| | anhydrase II, claudin-1, kinectin, tubulin- |
| | α-1c |
| Benign mucosal pemphigoid | Laminin-5, laminin-6, bullous |
| | pemphigoid antigen 1, bullous |
| | pemphigoid antigen 2, α₆-integrin |
| | subunit, β₄-integrin subunit, collagen VII |
| Bullous pemphigoid | Laminin-5, laminin-6, bullous |
| | pemphigoid antigen 1, bullous |
| | pemphigoid antigen 2, α₆-integrin |
| | subunit, β₄-integrin subunit, collagen VII |
| Castleman disease (CD) | ADAM Metallopeptidase with |
| | Thrombospondin Type 1 Motif 13 |
| | (ADAMTS13), BP180, α3 chain of type |
| | IV collagen |
| Celiac disease | Transglutaminase 2, transglutaminase 3, |
| | transglutaminase 6, actin, GM1, |
| | collagen, calreticulin, zonulin, synapsin I, |
| | ATP synthase β chain, cardiolipin, |
| | enolase α |
| Chagas disease | B1 adrenergic receptor, M2 muscarinic |
| | acetylcholine receptor |
| Chronic inflammatory demyelinating | Contactin-1, Myelin Protein Zero (MPZ), |
| polyneuropathy (CIDP) | Peripheral Myelin Protein 2 (PMP2), |
| | Peripheral Myelin Protein 22 (PMP22), |
| | gliomedin, Neural Cell Adhesion |
| | Molecule 1 (NCAM), NF140, NF186, |
| | Contactin 1 (CNTN1), CASPR1, NF155, |
| | GM1, GD1b |
| Churg-Strauss Syndrome (CSS) or | Myeloperoxidase, proteinase 3 |
| Eosinophilic Granulomatosis (EGPA) | |
| Cicatricial pemphigoid | Laminin 5, BP180, bullous pemphigoid |
| | antigen 2 |
| Cogan’s syndrome | Sulfoglucuronosyl glycolipids, HSP70, |
| | type II collagen, type IX collagen, Raf-1, |
| | major peripheral myelin protein P0, beta |
| | actin, cochlin, CTL2, cell-density |
| | enhanced protein tyrosine phosphatase- |
| | 1, connexion, proteinase 3, |
| | myeloperoxidase |
| Cold agglutinin disease | Cold agglutinins |
| Congenital heart block | Ro52, p200, muscarinic acetylcholine |
| | receptor, p57, α-fodrin, calreticulin, |
| | enolase α |
| Coxsackie myocarditis | Troponin, myosin, adenine nucleotide |
| | translocator |
| CREST syndrome | Scl70, Ro/SSA, La/SSb |
| Crohn’s disease | Nuclear histone 1 of polymorphonuclear |
| | leukocytes, trypsin, Ompc, Cbir1, I2, |
| | major zymogen granule membrane |
| | glycoprotein 2 |
| Dermatitis herpetiformis | Epidermal transglutaminase, HLA |
| Dermatomyositis | Melanoma differentiation antigen 5, |
| | transcriptional intermediary factor 1, |
| | nuclear matrix protein 2, small ubiquitin- |
| | like modifier activating enzyme |
| Devic’s disease (neuromyelitis optica) | Aquaporin-4 |
| Discoid lupus | Ro/SSA, RNP |
| Dressler’s syndrome | Myosin, actin |
| Endometriosis | Alpha(2)-HSG, transferrin, carbonic |
| | anhydrase |
| Eosinophilic esophagitis (EoE) | Desmoglein 3, transglutaminase |
| Eosinophilic fasciitis | Gliadin, serum andolase |
| Erythema nodosum | Thyroglobulin, HLA-B27 |
| Essential mixed cryoglobulinemia | Rheumatoid factor, GOR |
| Evans syndrome | Glycophorin A, Kpb antigen of Kell blood |
| | group system |
| Fibromyalgia | Thyroid peroxidase, thyroid stimulating |
| | hormone, thyroglobulin |
| Fibrosing alveolitis | DNA topoisomerase II, thyroglobulin |
| Giant cell arteritis (temporal arteritis) | Ferritin, lamin C, lamin A, vinculin, |
| | annexin 5, 14-3-3 (γ, ε and ζ), cardiolipin |
| Giant cell myocarditis | Myosin |
| Glomerulonephritis | C3 convertase, C1q, complement |
| | factors B & H |
| Goodpasture’s syndrome | Alpha 3 chain of type IV collagen |
| Granulomatosis with Polyangiitis | Proteinase 3, myeloperoxidase |
| Graves’ disease | Thyroid peroxidase, thyrotropin receptor, |
| | thyroglobulin |
| Guillain-Barre syndrome | Neurofascin-186 (NF186), gliomedin, |
| | contactin, neuronal cell adhesion |
| | molecule (NrCAM), ganglioside GM1, |
| | ganglioside GD1a, ganglioside GQ1b, |
| | ganglioside GT1a |
| Hashimoto’s thyroiditis | Thyroid peroxidase, thyroglobulin |
| Hemolytic anemia | Red blood cell antigens |
| Henoch-Schonlein purpura (HSP) | Cardiolipin, rheumatoid factor |
| Herpes gestationis or pemphigoid gestationis | BP180 |
| (PG) | |
| IgA Nephropathy | Protein Kinase D1 (PRKD1), Matrilin 2 |
| | (MATN2), DEAD-Box Helicase 17 |
| | (DDX17), Ubiquitin Conjugating Enzyme |
| | E2 W (UBE2W), Cyclin Dependent |
| | Kinase Inhibitor 1B (CDKN1B), |
| | Superoxide dismutase 2 (SOD2), ICQK, |
| | Basic Leucine Zipper Nuclear Factor 1 |
| | (BLZF1), Ephrin A3 (EFNA3), Eukaryotic |
| | Translation Initiation Factor 4A2 |
| | (EIF4A2), FLII actin remodeling protein |
| | (FLII), LIM And Calponin Homology |
| | Domains 1 (LIMCH1), MAGE Family |
| | Member A4 (MAGEA4), Myocyte |
| | Enhancer Factor 2D (MEF2D), MLLT6, |
| | PHD Finger Containing (MLLT6), |
| | Cytokine Induced Apoptosis Inhibitor 1 |
| | (CIAPIN1), GDP Dissociation Inhibitor 2 |
| | (GDI2), Heat Shock Protein Family A |
| | (Hsp70) Member 8 (HSPA8), Serpin |
| | family A member 5 (SERPINA5), |
| | Transglutaminase 1 (TGM1) |
| IgG4-related sclerosing disease | Carbonic anhydrase II, carbonic |
| | anhydrase IV, amylase-alpha-2α |
| | lactoferrin, rheumatoid factor, pancreatic |
| | trypsinogen, pancreatic secretory trypsin |
| | inhibitor |
| Immune thrombocytopenic purpura (ITP) | Glycoprotein IIb/IIIa (GPIIb/IIIa) and |
| | GPIb/IX |
| Inclusion body myositis (IBM) | Cytosolic 5′-nucleotidase 1A |
| Interstitial cystitis (IC) | Muscarinic M3 receptor |
| Juvenile arthritis | Rheumatoid factor, Cytosolic 5′- |
| | nucleotidase 1A, transthyretin |
| Juvenile diabetes (Type 1 diabetes) | Insulin, glutamic acid decarboxylase, |
| | protein tyrosine phosphatase, |
| | insulinoma antigen 2 protein, zinc |
| | transporter 8 |
| Juvenile myositis (JM) | Cytosolic 5′-nucleotidase 1A, p155/140, |
| | signal recognition particle, Mi2, |
| | synthetase, MJ |
| Kawasaki disease | Peroxiredoxin, cardiac myosin, 4- |
| | trimethylaminobutyraldehyde |
| | dehydrogenase |
| Lambert-Eaton syndrome | α(1) or β(3) subunits of voltage gated |
| | calcium channels |
| Leukocytoclastic vasculitis | Synthetase, proteinase 3, |
| | myeloperoxidase |
| Lichen planus | Desmogleins 1 and 3, thyroglobulin, |
| | thyroid peroxidase |
| Lichen sclerosus | Extracellular matrix protein 1 |
| Linear IgA disease (LAD) | Bullous pemphigoid antigen 180 |
| | (BP180), bullous pemphigoid antigen |
| | 230 (BP230), LABD97 (97 kDa fragment |
| | of bullous pemphigoid antigen 180), |
| | LAD-1 (120 kDa fragment of bullous |
| | pemphigoid antigen 180) |
| Lupus | U1A, Sm/RNP, Ro/La, thyroglobulin, |
| | Protein Tyrosine Phosphatase Non- |
| | Receptor Type 22 (PTPN22), Bruton’s |
| | tyrosine kinase, Lyn tyrosine kinase, B- |
| | Cell activating factor, Ro/SSA |
| Lyme disease chronic | CK10, LFA-1, enolase, Matrix |
| | metalloproteinase 10 (MMP10), ECGF, |
| | beta-2-glycoprotein-1, cardiolipin |
| Meniere’s disease | Hsp70, 68-kD inner ear protein, myelin |
| | peroxidase zero, thyroid peroxidase, |
| | Immunoglobulin Heavy Constant |
| | Gamma 1 (IGHG1), Regulator Of G |
| | Protein Signaling 10 (RGS10), C2orf34, |
| | SH3 Domain Containing GRB2 Like, |
| | Endophilin B1 (SH3GLB1), |
| | Calcium/Calmodulin Dependent Protein |
| | Kinase IV (CAMK4), GSG1 Like |
| | (GSG1L), NIMA Related Kinase 7 |
| | (NEK7), Neural Cell Adhesion Molecule |
| | 2 (NCAM2), Aminoacylase 1 (ACY1) |
| Microscopic polyangiitis (MPA) | Proteinase 3, myeloperoxidase |
| Mixed connective tissue disease (MCTD) | U1 ribonucleoprotein (U1-RNP) |
| Mooren’s ulcer | Cornea-associated antigen |
| Multifocal Motor Neuropathy (MMN) or | Neurofascin-186, gliomedin, GM1 |
| MMNCB | |
| Multiple sclerosis | Myelin oligodendrocyte glycoprotein |
| | (MOG) |
| Myasthenia gravis | Nicotinic acetylcholine receptor, LDL |
| | Receptor Related Protein 4 (LRP4), |
| | Muscle Associated Receptor Tyrosine |
| | Kinase (MuSK), rapsyn, titin, myofibrillar |
| | protein, Ryanodine receptor (RyR), |
| | Kv1.4 voltage-gated potassium channel |
| Myositis | Cytosolic 5′-nucleotidase 1A, p155/140, |
| | signal recognition particle, Mi2, |
| | synthetase, MJ, TIF1-gamma, NXP-2, |
| | MDA5, SAE, 3-Hydroxy-3- |
| | Methylglutaryl-CoA Reductase |
| | (HMGCR), Pm/Sc, Ku |
| Narcolepsy | Methyltransferase-like 22, 5′- |
| | nucleotidase cytosolic I, Poly(ADP- |
| | Ribose) Polymerase Family Member 3 |
| | (PARP3), AT-Rich Interaction Domain |
| | 4B (ARID4B), Glutaminase 2 (GLS2), |
| | Cyclin Dependent Kinase Like 1 |
| | (CDKL1) |
| Neonatal Lupus | Ro/SSA, La/SSB, U1-RNP |
| Neuromyelitis optica | Aquaporin-4, thyroid peroxidase |
| Neutropenia | FcyRIIIb, gp50-64, gp70-95, Integrin |
| | Subunit Alpha M (CD11b), Integrin |
| | Subunit Alpha L (CD11a) |
| Ocular cicatricial pemphigoid | B4-integrin, laminin 5 α3 subunit |
| Optic neuritis | Aquaporin-4, myelin oligodendrocyte |
| | glycoprotein |
| Palindromic rheumatism (PR) | Enolase, fibrin, vimentin, keratin, |
| | filaggrin, fibrinogen |
| PANDAS | Dopamine D2 receptor, |
| | Ca2+/calmodulin-dependent protein |
| | kinase II |
| | (CamKII) |
| Paraneoplastic cerebellar degeneration (PCD) | mGluR1, DNER, Yo |
| Paroxysmal nocturnal hemoglobinuria (PNH) | P & I antigens of red blood cells |
| Parry Romberg syndrome | Histone proteins |
| Pars planitis (peripheral uveitis) | Hsp70 |
| Parsonage-Turner syndrome | GA1 |
| Pemphigus | Desmocollin 1 (DSC1), Desmocollin 3 |
| | (DSC3), ATPase Secretory Pathway |
| | Ca2 +Transporting 1 (ATP2C1), |
| | Plakophilin 3 (PKP3), Muscarinic |
| | Acetylcholine Receptor M3 (CHRM3), |
| | Collagen Type XXI Alpha 1 Chain |
| | (COL21A1), Annexin A8 Like 1 |
| | (ANXA8L1), cluster of differentiation 88 |
| | (CD88), Cholinergic Receptor Nicotinic |
| | Epsilon Subunit (CHRNE), CD33 |
| | molecule (CD33), GP1BA, Cholinergic |
| | Receptor Nicotinic Delta Subunit |
| | (CHRND), Solute Carrier Family 36 |
| | Member 4 (SLC36A4), CD1b Molecule |
| | (CD1B), Fc Fragment Of IgG Receptor |
| | Ila (CD32), Cadherin 8 (CDH8), |
| | Cadherin 9 (CDH9), PMP22, Major |
| | Histocompatibility Complex, Class I, E |
| | (HLA-E), NADH:Ubiquinone |
| | Oxidoreductase Core Subunit S1 |
| | (NDUFS1), Cytochrome B5 Type B |
| | (CYB5B), SO2, Pyruvate |
| | Dehydrogenase E1 Alpha 1 Subunit |
| | (PDHA1), FH, ATPase Secretory |
| | Pathway Ca2+Transporting 1 (ATP2C1) |
| Peripheral neuropathy | Myelin associated glycoprotein (MAG), |
| | ganglioside GM1, Hu, ganglioside GQ1b |
| Perivenous encephalomyelitis | Myelin oligodendrocyte glycoprotein |
| Pernicious anemia (PA) | IF-R7 |
| POEMS syndrome | C3 convertase |
| Polyarteritis nodosa | Proteinase 3, phospholipid cofactor, |
| | cardiolipin, β2-glycoprotein I |
| | phosphatidylserine-prothrombin complex |
| Polyglandular syndromes type I, II, III | 17 alpha-Hydroxylase, side-chain- |
| | cleavage enzyme, GAD65, |
| | thyroperoxidase, thyroglobulin, H+/K(+)- |
| | ATPase |
| Polymyalgia rheumatica | Ferritin |
| Polymyositis | Aminoacyl-tRNA synthetase, Mi-2, Jo-1, |
| | helicase/histone deacetylase protein |
| | complex, TIF1-γ, MDA5, NXP2, SAE, |
| | HMGCR |
| Postpericardiotomy syndrome | Cardiolipin |
| Primary biliary cirrhosis | E2 subunit of pyruvate dehydrogenase |
| Primary sclerosing cholangitis | M2, M4, M8, M9, SP100, PML, NDP52, |
| | SP140, GP120, P62, laminin, laminin B |
| | receptor, CENP-A, CENP-B, CENP-C, |
| | CENP-O, Scl70, Sm, SSA, SSb, RNP, |
| | Jo-1, U1-RNP |
| Progesterone dermatitis | Progesterone |
| Psoriasis | Rheumatoid factor, calpastatin, fibrillin, |
| | desmocollin, keratin, nebulin-related |
| | anchoring protein |
| Psoriatic arthritis | Rheumatoid factor, calpastatin, fibrillin, |
| | desmocollin, keratin, nebulin-related |
| | anchoring protein |
| Pure red cell aplasia (PRCA) | Erythropoietin |
| Raynaud’s phenomenon | Synthetase, cytokeratin 10 |
| Reactive Arthritis | Human leukocyte antigen B27 (HLA B27) |
| Reflex sympathetic dystrophy | α1-adrenaline receptor |
| Relapsing polychondritis | Collagens II, IX, XI |
| Rheumatic fever | Myosin, glycogen, Vascular cell |
| | adhesion molecule 1 (VCAM-1) |
| Rheumatoid arthritis | Rheumatoid factor, Sa, glucose-6- |
| | phosphate isomerase, collagen I and II, |
| | Hsp65, Bip, a enolase, calpastatin, |
| | RA33/hnRNP A2, filaggrin, fibrin, |
| | fibrinogen, vimentin |
| Sarcoidosis | Ro, Scl-70, cardiolipin, La |
| Schmidt syndrome | Transglutaminase, 21-hydroxylase, |
| | thyroglobulin, thyroperoxidase |
| Scleritis | Laminin, keratin |
| Scleroderma | Sc1-70, U1-RNP, Th/To, RNAP I, II, Ill |
| Sjögren’s syndrome | Ro/SSA |
| Sperm & testicular autoimmunity | Hsp70, Hsp70-2, ER60, caspase-3, |
| | proteasome complex component 2 and |
| | zeta chain, SAGA-1 |
| Stiff person syndrome (SPS) | GAD65, glycine receptor α1, GABA_{A} |
| | receptor associated protein, |
| | amphiphysin |
| Subacute bacterial endocarditis (SBE) | Proteinase 3, rheumatoid factor |
| Sympathetic ophthalmia (SO) | Retinal soluble antigen, rhodopsin, |
| | interphotoreceptor retinoid-binding |
| | protein, recoverin |
| Takayasu’s arteritis | Ferritin, cardiolipin |
| Tolosa-Hunt syndrome (THS) | Ganglioside GQ1b |
| Transverse myelitis | Aquaporin-4, Ro/SSA |
| Type 1 diabetes | Insulin, Glutamic acid decarboxylase |
| | (GAD, e.g., GAD65) |
| Ulcerative colitis (UC) | Tropomyosin (e.g., tropomyosin 5), |
| | histone H1, cathepsin G, elastase, β- |
| | glucuronidase, lactoferrin, bactericidal |
| | permeability increasing (BPI) protein, α- |
| | Enolase, high-mobility group (HMG)-1, |
| | HMG-2 |
| Undifferentiated connective tissue disease | RNP, Ro/SSA |
| (UCTD) | |
| Uveitis | Melanin associated antigen (MAA), |
| | retinal arrestin, interphotoreceptor |
| | retinoid binding protein (IRBP) |
| Vasculitis | β2 glycoprotein 1 (β2-GPI) |
| Vitiligo | Tyrosinase (Tyr), tyrosinase-related |
| | protein 1 (TRP1), tyrosinase-related |
| | protein 2 (TRP2), MART-1/Melan A, |
| | Pmel17/gp100 |
| Vogt-Koyanagi-Harada Disease | Tyrosinase (Tyr), tyrosinase-related |
| | protein 1 (TRP1), tyrosinase-related |
| | protein 2 (TRP2), MART-1/Melan A, |
| | Pmel17/gp100 |

### Treatment of Multiple Sclerosis

In one example, the cloaked cells described herein can be used to treat Multiple Sclerosis (MS). MS is a disease of the brain and spinal cord characterized by immune system-induced damage to the myelin sheath surrounding neurons. T cells in subjects with MS react to MOG, a protein expressed by myelinating oligodendrocytes and Schwann cells. To treat a subject suffering from MS, cloaked cells can be modified to express MOG or a fragment of MOG that induces an inflammatory response (e.g., activation, release of pro-inflammatory cytokines, or cytotoxicity) in T cells isolated from the subject. Cloaked cells can be modified to produce MOG by expression of a transgene encoding MOG operably linked to a constitutive or inducible promoter. Cloaked cells (e.g., stem cells) that express MOG can be directly administered to a subject or differentiated into oligodendrocytes or Schwann cells prior to administration using methods known by those of skill in the art, or isolated oligodendrocytes or Schwann cells (e.g., oligodendrocytes or Schwann cells isolated from a human subject, animal model, or cell line) can be modified to express cloaking transgenes and MOG. Eight hundred million to three billion cloaked cells expressing MOG (e.g., 8×10⁸, 9×10⁸, 1×10⁹, 2×10⁹, or 3×10⁹ cloaked stem cells, oligodendrocytes, or Schwann cells) can be injected subcutaneously in a subject to create a cloaked subcutaneous tissue that produces MOG to induce immune tolerance to MOG for the treatment of MS, or 2.5×10⁴ to 1×10⁵ cloaked cells expressing MOG (e.g., 2.5×10⁴, 3×10⁴, 4×10⁴, 5×10⁴, 6×10⁴, 7×10⁴, 8×10⁴, 9×10⁴, or 1×10⁵ cloaked cells) can be administered (e.g., injected) to the spinal cord or to a specific brain region to induce immune tolerance to MOG for the treatment of MS. This therapy may lead to the deactivation or death of T cells that react to MOG, preventing further damage to myelinating cells in the subject.

### Treatment of Type 1 Diabetes

The cloaked cells described herein can also be used to treat type 1 diabetes. Type 1 diabetes results from immune system-induced damage to the insulin-producing islet beta cells of the pancreas. Subjects with type 1 diabetes often present with auto-antibodies directed against beta cell epitopes, such as GAD65 and insulin. To treat a subject suffering from type 1 diabetes, cloaked cells can be modified to express insulin and/or GAD65, or a fragment thereof that induces an inflammatory response (e.g., activation, release of pro-inflammatory cytokines, or cytotoxicity) in an immune cell isolated from the subject. Cloaked cells can be modified to produce insulin and/or GAD65 by expression of transgenes encoding insulin and/or GAD65 operably linked to a constitutive or inducible promoter. Cloaked cells (e.g., stem cells) that express insulin and/or GAD65 can be differentiated into pancreatic beta cells prior to administration using methods known by those of skill in the art or the cloaked cells can be administered without differentiation. Eight hundred million to three billion cloaked cells (e.g., 8×10⁸, 9×10⁸, 1×10⁹, 2×10⁹, or 3×10⁹ cloaked cells) expressing insulin and/or GAD65 to can be injected subcutaneously in a subject to create a cloaked subcutaneous tissue that produces insulin and/or GAD65 for treating type 1 diabetes. This therapy may reduce the autoimmunity to insulin and/or GAD65 in the subject, preventing further damage to pancreatic beta cells and allowing other therapies that restore pancreatic beta cell function to work more effectively.

### Methods of Inducing an Immune Response to a Non-Self Antigen

The cloaked cells described herein may also be used in a cell-based method of immunization. The method involves two steps: 1) administering to a subject a cloaked cell (e.g., a cell that the immune system would reject or attack as foreign if not cloaked, e.g., an allogeneic cell) to induce immune tolerance to the cloaked cell, and 2) administering an uncloaked cell of the same type that expresses a polypeptide containing a non-self antigen (e.g., one or more polypeptides containing a non-self antigen, e.g., 1, 2, 3, 4, 5, or more of such polypeptides) that is not endogenous to the uncloaked cell to induce an immune response to the non-self antigen. Administration of the cloaked cell without the non-self antigen tolerizes the subject's immune response to the cell and the proteins and antigens the cell expresses or contains. Upon administration of the uncloaked cell of the same type that is modified to express a polypeptide containing a non-self antigen, the subject's immune system will mount a specific immune response against the non-self antigen that is expressed by the uncloaked cell as the immune system has not previously encountered the non-self antigen. The subject's immune system may proceed to produce antibodies directed to the non-self antigen, creating immunity to the non-self antigen should the subject be exposed to the antigen in the future. This targeted approach allows the immune system to respond specifically to the one or more non-self antigen expressed by the uncloaked cell, a notable improvement over cell-based vaccines, such as cellular cancer vaccines, in which both normal cellular antigens and one or more tumor antigens are presented to the immune system with the potential for the large number of normal cellular immunogens to compete with or dilute out the desired response to the one or more tumor antigens. This method may be used to treat a subject in need of immunity to a non-self antigen (e.g., a cancer antigen, a viral antigen, a bacterial antigen, a parasitic antigen, or a fungal antigen), such as a subject at risk of developing cancer (e.g., a subject at risk of developing cancer due to genetic mutation, family history, or environmental factors), or a subject at risk of viral, bacterial, fungal, or parasitic infection (e.g., at risk due to geographic location or other environmental factors).

The cloaked cell administered to the subject in the initial tolerization phase of the method may be any allogeneic cloaked cell described herein. For example, the cloaked cell may be a stem cell (e.g., an ES cell, an adult stem cell, an iPSC, a tissue-specific stem cell, a hematopoietic stem cell, a mesenchymal stem cell, an endothelial stem cell, an epithelial stem cell, an adipose stem or progenitor cell, a germline stem cell, a lung stem or progenitor cell, a mammary stem cell, an olfactory adult stem cell, a hair follicle stem cell, a multipotent stem cell, an amniotic stem cell, a cord blood stem cell, or a neural stem or progenitor cell), a somatic cell, a cell derived from a cell line, a cell amenable to genome editing, and/or a source of therapeutic cell type (e.g., a cell that can be differentiated into a lineage restricted cell for cell therapy, or a cell of a desired target tissue). In some embodiments, the cell is a skin, heart, brain or spinal cord, liver, lung, kidney, pancreas, bladder, bone marrow, spleen, intestine, or stomach cell. In some embodiments, the cell is a fibroblast, an epithelial cell, or an endothelial cell. The cell may be a vertebrate cell, for example, a mammalian cell, such as a human or mouse cell. The cell may be a cell from a cell line. The cell may be a syngeneic cell. The cell may be cloaked by modifying the cell to express one or more (e.g., two, three, four, five, six, seven, or all eight) of PD-L1, HLA-G (H2-M3), Cd47, Cd200, FASLG (FasL), Ccl21 (Ccl21b), Mfge8, and Serpin B9 (Spi6). In some embodiments, the cloaked cell expresses all eight of PD-L1, HLA-G (H2-M3), Cd47, Cd200, FASLG (FasL), Ccl21 (Ccl21b), Mfge8, and Serpin B9 (Spi6). The cell may be further modified to express one or more of TGF-β, Cd73, Cd39, Lag3, Il1r2, Ackr2, Tnfrsf22, Tnfrs23, Tnfrsf10, Dad1, and IFNγR1 d39. The cell can be modified to express the cloaking transgenes using the methods described herein above (e.g., transfection, viral infection, or gene editing) or using other methods known to those of skill in the art.

The uncloaked cell is a cell of the same type derived from the same source as the cloaked cell, and it can be modified to express a polypeptide containing a non-self antigen (e.g., an antigen that is not endogenous to the subject and that is not expressed by the uncloaked cell) using the methods described herein above (e.g., transfection, viral infection, or gene editing) or using other methods known to those of skill in the art. The non-self antigen provided by the uncloaked cell may be a disease-associated antigen (e.g., a bacterial, viral, parasitic, or fungal protein, such as a bacterial, viral, fungal, or parasitic cell surface protein). The non-self antigen may also be a cancer antigen. Any disease-associated antigen that is functionally inert (e.g., not capable of inducing disease when expressed by the uncloaked cell) may be presented to the immune system of a subject by the methods described herein to promote an immune response in the subject (e.g., to induce the subject's immune system to produce antibodies in response to the non-self antigen). The uncloaked cell expressing a polypeptide containing a non-self antigen may be administered with an adjuvant, such as alum, AS04, or AS03, to further stimulate the immune response in a subject.

The uncloaked cell may be modified to express the polypeptide containing the antigen at an expression level comparable to the expression level of a housekeeping polypeptide (e.g., B-actin, GAPDH, or Rosa26). Robust expression of the polypeptide can be achieved using strong promoters, such as CAGG or CMV or through the use of a doxycycline inducible system (e.g., a doxycycline inducible TRE).

The cloaked cell can be injected as a circulating cell or injected subcutaneously to produce a cloaked subcutaneous tissue. In some embodiments, one million to one hundred billion cloaked cells (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ cloaked cells) are administered to a subject to induce immune tolerance to the cloaked cell. The uncloaked cell expressing the polypeptide containing the non-self antigen can be administered after the immune system of the subject has been tolerized to the cloaked cell. To determine whether the immune system of the subject has been tolerized to the cloaked cell, an immune cell isolated from the subject can be exposed to an uncloaked cell and the response of the immune cell can be measured (e.g., an immune cell, such as a dendritic cell, macrophage, T cell (e.g., a cytotoxic T cell/CD8⁺ T cell, T helper cell/CD4⁺ T cell, or regulatory T cell/Treg), B cell, monocyte, granulocyte (e.g., eosinophil, mast cell, neutrophil, or basophil), natural killer (NK) cell, or innate lymphoid cell isolated from the subject can be contacted with the uncloaked cell in vitro, and a response of the immune cell (e.g., immune cell activation, differentiation, polarization, proliferation, migration, pro-inflammatory cytokine production, degranulation, phagocytosis, or cytotoxicity) can be measured). If the immune cell isolated from the subject does not mount an immune response against the uncloaked cell (e.g., if the immune cell does not become more activated or exhibit a cytotoxic, pro-inflammatory, or phagocytic response to the uncloaked cell), the subject's immune system has been successfully tolerized to the cell. The uncloaked cell expressing a polypeptide containing a non-self antigen may be administered to the subject after administration of the cloaked cell (e.g., 1 day, 2 days, 3 days, 4 days, 5 days, 6 days, 1 week, 2 weeks, 3 weeks, 1 month, 2 months, 3 months, 4 months, 5 months, 6 months, 9 months, 1 year or more after the administration of the cloaked cell). The uncloaked cell expressing a polypeptide containing a non-self antigen can be injected as a circulating cell or injected subcutaneously to produce an uncloaked subcutaneous tissue. In some embodiments, one million to one hundred billion uncloaked cells expressing a non-self antigen (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×109, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ uncloaked cells expressing a non-self antigen) are administered to a subject to stimulate an immune response to the non-self antigen. A blood sample can be collected from the subject to determine whether the subject's immune system has produced antibodies directed against the non-self antigen.

The cloaked cells can be removed after the subject's immune system has been tolerized to the cloaked cells (e.g., 1 week, 1 month, 2 months, 3 months, 4 months, 5 months, 6 months, 9 months, 1 year, 2 years or more after the subject's immune system has been tolerized to the cloaked cells). The cloaked cells can be removed before or after the administration of the uncloaked cells expressing a polypeptide containing a non-self antigen. The cloaked cells can be removed surgically (e.g., in embodiments in which the cloaked cells formed a tissue, such as a cloaked subcutaneous tissue, the tissue can be surgically removed). In embodiments in which the cloaked cells were modified to contain one or more systems for regulating cell division (e.g., modified to be FAILSAFE™ cells, e.g., by linking the expression of a CDL to an ALINK or EARC), the cloaked cells can be removed by administering an inducer of a negative selectable marker (e.g., the ALINK) or by ceasing to administer an activator of an inducible activator-based gene expression system (e.g., the EARC). The uncloaked cells expressing the polypeptide containing the non-self antigen can be removed after the subject's immune system has generated antibodies directed against the non-self antigen (e.g., 1 week, 1 month, 2 months, 3 months, 4 months, 5 months, 6 months, 9 months, 1 year, 2 years or more after the subject's immune system has generated antibodies directed against the non-self antigen). The uncloaked cells can be removed surgically (e.g., in embodiments in which the uncloaked cells formed a tissue, such as an uncloaked subcutaneous tissue, the tissue can be surgically removed). In embodiments in which the uncloaked cells were modified to contain one or more systems for regulating cell division (e.g., modified to be FAILSAFE™ cells, e.g., by linking the expression of a CDL to an ALINK or EARC), the uncloaked cells can be removed by administering an inducer of a negative selectable marker (e.g., the ALINK) or by ceasing to administer an activator of an inducible activator-based gene expression system (e.g., the EARC).

In some embodiments, the non-self antigen is an antigen of a bacterial pathogen (e.g., an antigen derived from Acinetobacter spp. (Acinetobacter baumanni), Bacteroides distasonis, Bacteroides fragilis, Bacteroides ovatus, Bacteroides thetaiotaomicron, Bacteroides uniformis, Bacteroides vulgatus, B. cepacia, Citrobacter freundii, Citrobacter koseri, Clostridium clostridioforme, Clostridium perfringens, C. sordellii, Enterobacter aerogenes, Enterobacter cloacae, Enterococcus faecalis, Enterococcus spp. (vancomycin susceptible and resistant isolates), Escherichia coli (including ESBL and KPC producing isolates), Eubacterium lentum, Fusobacterium spp., Haemophilus influenzae (including beta-lactamase positive isolates), Haemophilus parainfluenzae, Klebsiella pneumoniae (including ESBL and KPC producing isolates), Klebsiella oxytoca (including ESBL and KPC producing isolates), Legionella pneumophilia, Moraxella catarrhalis, Morganella morganii, Mycoplasma spp., Peptostreptococcus spp., Porphyromonas saccharolytica, Prevotella bivia, Proteus mirabilis, Proteus vulgaris, Providencia rettgeri, Providencia stuartii, Pseudomonas aeruginosa, Serratia marcescens, Streptococcus anginosus, Staphylococcus aureus (methicillin susceptible and resistant isolates), Staphylococcus epidermidis (methicillin susceptible and resistant isolates), Stenotrophomonas maltophilia, Streptococcus agalactiae, Streptococcus constellatus, Streptococcus pneumoniae (penicillin susceptible and resistant isolates), Streptococcus pyogenes, or Streptococcus pyogenes). The bacterial antigen may also be derived from Mycobacterium tuberculosis, Mycobacterium bovis, Mycobacterium africanum, Mycobacterium microti, Mycobacterium leprae, Salmonella typhimurium, Francisella tularensis, Brucella, Burkholderia mallei, Yersinia pestis, Corynebacterium diphtheria, Neisseria meningitidis, Bordetella pertussis, Clostridium tetani, or Bacillus anthracis. Non-limiting examples of bacterial antigens include 10.4, 85A, 85B, 86C, CFP-10, Rv3871, and ESAT-6 antigens of Mycobacterium; O, H, and K antigens of E. coli; and protective antigen (PA) of Bacillus anthracis.

The non-self can be a viral antigen, which may be derived from a virus of a viral family selected from the group consisting of Retroviridae, Flaviviridae, Arenaviridae, Bunyaviridae, Filoviridae, Togaviridae, Poxviridae, Herpesviridae, Orthomyxoviridae, Coronaviridae, Rhabdoviridae, Paramyxoviridae, Picornaviridae, Hepadnaviridae, Papillomaviridae, Parvoviridae, Astroviridae, Polyomaviridae, Calciviridae, and Reoviridae. The virus may be, e.g., human immunodeficiency virus (HIV), human papillomavirus (HPV), hepatitis A virus (Hep A), hepatitis B virus (HBV), hepatitis C virus (HCV), Variola major, Variola minor, monkeypox virus, measles virus, rubella virus, mumps virus, varicella zoster virus (VZV), poliovirus, rabies virus, Japanese encephalitis virus, herpes simplex virus (HSV), cytomegalovirus (CMV), rotavirus, influenza, Ebola virus, yellow fever virus, Zika virus, or Marburg virus. Non-limiting examples of viral antigens include Gag, Pol, Nef, Tat, Rev, Vif, Vpr, or Vpu of HIV and other retroviruses (see, e.g., U.S. Pub. No. 2012/0076812, incorporated by reference herein); 9D antigen of HSV; Env of all envelope protein-containing viruses. For example, the viral antigen may be an Env protein or a structured protein. In a particular example, the viral antigen may be an HIV or Zika virus Env protein. The viral antigen may also be a Gag, Pol, Env, Nef, Tat, Rev, Vif, Vpr, or Vpu protein.

The non-self antigen can be a parasitic antigen, which may be derived from Toxoplasma gondii, Plasmodium falciparum, Plasmodium vivax, Plasmodium ovale, Plasmodium malariae, Trypanosoma spp., or Legionella spp. Non-limiting examples of parasitic antigens include circumsporozoite (CS) protein, gamete surface proteins Pfs230 and Pfs48/45, and Liver Specific Antigens 1 or 3 (LSA-1 or LSA-3) of Plasmodium falciparum.

In some embodiments, the non-self antigen is an antigen of a fungal pathogen (e.g., an antigen derived from a mold pathogen, such as one from phylum Ascomycota (e.g., Ajellomyces spp., Alternaria spp., Aschersonia spp., Aspergillus spp., Arthroderma spp., Ascochyta spp., Bipolaris spp., Blastomyces spp., Botryotinia spp., Chaetomium spp., Cladosporium spp., Coccidioides spp., Curvularia spp., Emericella spp., Emmonsia spp., Epicoccum spp., Exophiala spp., Fusarium spp., Geomyces spp., Geotrichum spp., Gibberella spp., Histoplasma spp., Magnaporthe spp., Metarhizium spp., Monascus spp., Mycospaerella spp., Nectria spp., Neosartorya spp., Neurospora spp., Paecilomyces spp., Paracoccidioides spp., Penicillium spp., Phaeosphaeria spp., Phialemonium spp., Podospora spp., Pyrenophora spp., Sclerotinia spp., Scopulariopsis spp., Sporothrix spp., Stachybotrys spp., Stemphylium spp., Talaromyces spp., Trichophyton spp., Trichothecium spp., Tricoderma spp., Tuber spp., Uncinocarpus spp., or Verticillium spp.), phylum Basidomycota (e.g., Moniliophthora spp., Sporobolomyces spp., Trichosporon spp., Ustilago spp., Cryptococcus spp, or Rhodotorula spp.), phylum Chytridiomycota, phylum Zygomycota (e.g., Absidia spp., Amylomyces spp,, Pilaira spp., Rhizomucor spp., Rhizopus spp., or Zygomycetes spp.), or phylum Oomycota in the Stramenopila kingdom). The fungal antigen may be from Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus. Non-limiting examples of fungal antigens include any cell wall mannoprotein (e.g., Afmp1 of Aspergillus fumigatus) or surface-expressed glycoprotein (e.g., SOWgp of Coccidioides immitis).

The non-self antigen can be a cancer antigen, which may be derived from a carcinoma, lymphoma, blastoma, sarcoma, and leukemia or lymphoid malignancies. For example, the cancer antigen can be derived from a leukemia, lymphoma, liver cancer, bone cancer, lung cancer (such as small-cell lung cancer, non-small-cell lung cancer, adenocarcinoma of the lung, and squamous carcinoma of the lung), brain cancer, bladder cancer (e.g., urothelial bladder cancer (UBC), muscle invasive bladder cancer (MIBC), or BCG-refractory non-muscle invasive bladder cancer (NMIBC)), gastrointestinal cancer (such as non-metastatic or metastatic colorectal cancer, pancreatic cancer, gastric cancer, esophageal cancer, hepatocellular cancer, cholangiocellular cancer, oral cancer, or lip cancer), urogenital cancer (such as hormone sensitive or hormone refractory prostate cancer, renal cell cancer, bladder cancer, or penile cancer), breast cancer (e.g., triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, or phyllodes tumor), cardiac cancer, gynecological cancer (such as ovarian cancer, cervical cancer, endometrial cancer), uterine cancer, head and neck cancer (e.g., head and neck squamous cell cancer), gallbladder cancer, laryngeal cancer, lip and oral cavity cancer, ocular cancer, melanoma, pancreatic cancer, prostate cancer (such as castration-resistant prostate cancer (CRPC)), colorectal cancer, testicular cancer, CNS cancer including malignant glioma, astrocytoma, retinoblastoma, and brain metastases, throat cancer, skin cancer (such as malignant melanoma, basal and squamous cell skin cancer, Merkel Cell Carcinoma, lymphoma of the skin, Kaposi Sarcoma), thyroid cancer, bone and soft tissue sarcoma, or hematologic neoplasia (such as multiple myeloma, acute myelogenous leukemia, chronic myelogenous leukemia, myelodysplastic syndrome, acute lymphoblastic leukemia, Hodgkin's lymphoma). The cancer antigen can also be derived from an acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, Hodgkin lymphoma, hypopharyngeal cancer, islet cell tumors, pancreatic neuroendocrine tumors, Wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, stomach cancer, renal cancer, pelvic cancer, bronchus cancer, oropharyngeal cancer, larynx cancer, biliary tract cancer, a cancer of the central nervous system, a cancer of the respiratory system, and a cancer of the urinary system, cancer of the peritoneum, hepatocellular cancer, hepatoma, endometrial or uterine carcinoma, salivary gland carcinoma, hepatic carcinoma, anal carcinoma, penile carcinoma, post-transplant lymphoproliferative disorder (PTLD), bladder carcinoma, a pharynx cancer, a tumor of the tongue, a synovial cell sarcoma, a neuroblastoma, pheochromocytoma, pituitary tumor, pleuropulmonary blastoma, primary peritoneal cancer, rectal cancer, retinoblastoma, rhabdomyosarcoma, salivary gland cancer, Kaposi sarcoma, rhabdomyosarcoma, Sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, or Waldenström macroglobulinemia. The cancer antigen can also be derived from a leukemia (e.g., B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), hairy cell leukemia, and erythroleukemia), sarcoma (e.g., angiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leyomyosarcoma, and neurofibrosarcoma), carcinoma (e.g., basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastic carcinoma, adrenocortical carcinoma, cholangiocarcinoma, Merkel cell carcinoma, ductal carcinoma in situ (DCIS), and invasive ductal carcinoma), blastoma (e.g., hepatoblastoma, medulloblastoma, nephroblastoma, neuroblastoma, pancreatoblastoma, pleuropulmonary blastoma, retinoblastoma, and glioblastoma multiforme), lymphoma (e.g., Hodgkin's lymphoma, non-Hodgkin's lymphoma, low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, Waldenstrom's Macroglobulinemia, B cell lymphoma, and Burkitt lymphoma), myeloma (e.g., multiple myeloma, plasmacytoma, localized myeloma, and extramedullary myeloma), melanoma (e.g., superficial spreading melanoma, nodular melanoma, lentigno maligna melanoma, acral lentiginous melanoma, and amelanotic melanoma), neuroma (e.g., ganglioneuroma, Pacinian neuroma, and acoustic neuroma), glioma (e.g., astrocytoma, oligoastrocytoma, ependymoma, brainstem glioma, optic nerve glioma, and oligoastrocytoma), pheochromocytoma, meningioma, malignant mesothelioma, or a virally induced cancer.

In some embodiments, the cancer antigen is an ovarian cancer antigen. In some embodiments, the ovarian cancer antigen is Kallikrein 4 (reported immunogenic epitopes include: FLGYLILGV (SEQ ID NO.: 18); SVSESDTIRSISIAS (SEQ ID NO.: 19); LLANGRMPTVLQCVN (SEQ ID NO.: 20); and RMPTVLQCVNVSVVS (SEQ ID NO.: 21)); PBF (reported immunogenic epitopes include: CTACRWKKACQR (SEQ ID NO.: 22)); PRAME (reported immunogenic epitopes include: VLDGLDVLL (SEQ ID NO.: 23); SLYSFPEPEA (SEQ ID NO.: 24); ALYVDSLFFL (SEQ ID NO.: 25); SLLQHLIGL (SEQ ID NO.: 26); and LYVDSLFFL (SEQ ID NO.: 27)); WT1 (reported immunogenic epitopes include: TSEKRPFMCAY (SEQ ID NO.: 28); CMTWNQMNL (SEQ ID NO.: 29); LSHLQMHSRKH (SEQ ID NO.: 30); KRYFKLSHLQMHSRKH (SEQ ID NO.: 31); and KRYFKLSHLQMHSRKH (SEQ ID NO.: 31)); HSDL1 (reported immunogenic epitopes include: CYMEAVAL (SEQ ID NO.: 32)); Mesothelin (reported immunogenic epitopes include: SLLFLLFSL (SEQ ID NO.: 33); VLPLTVAEV (SEQ ID NO.: 34); ALQGGGPPY (SEQ ID NO.: 35); LYPKARLAF (SEQ ID NO.: 36); and AFLPWHRLF (SEQ ID NO.: 37)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); CEA (reported immunogenic epitopes include: TYYRPGVNLSLSC (SEQ ID NO.: 66); EIIYPNASLLIQN (SEQ ID NO.: 67); YACFVSNLATGRNNS (SEQ ID NO.: 68); LWWVNNQSLPVSP (SEQ ID NO.: 69); LWWVNNQSLPVSP (SEQ ID NO.: 69); LWWVNNQSLPVSP (SEQ ID NO.: 69); EIIYPNASLLIQN (SEQ ID NO.: 67); NSIVKSITVSASG (SEQ ID NO.: 70); KTWGQYWQV (SEQ ID NO.: 71); (A) MLGTHTMEV (SEQ ID NO.: 72); ITDQVPFSV (SEQ ID NO.: 73); YLEPGPVTA (SEQ ID NO.: 74); LLDGTATLRL (SEQ ID NO.: 75); VLYRYGSFSV (SEQ ID NO.: 76); SLADTNSLAV (SEQ ID NO.: 77); RLMKQDFSV (SEQ ID NO.: 78); RLPRIFCSC (SEQ ID NO.: 79); LIYRRRLMK (SEQ ID NO.: 80); ALLAVGATK (SEQ ID NO.: 81); IALNFPGSQK (SEQ ID NO.: 82); and RSYVPLAHR (SEQ ID NO.: 83)); p53 (reported immunogenic epitopes include: VVPCEPPEV (SEQ ID NO.: 84)); Her2/Neu (reported immunogenic epitopes include: HLYQGCQVV (SEQ ID NO.: 85); YLVPQQGFFC (SEQ ID NO.: 86); PLQPEQLQV (SEQ ID NO.: 87); TLEEITGYL (SEQ ID NO.: 88); ALIHHNTHL (SEQ ID NO.: 89); PLTSIISAV (SEQ ID NO.: 90); VLRENTSPK (SEQ ID NO.: 91); and TYLPTNASL (SEQ ID NO.: 92)); EpCAM (reported immunogenic epitopes include: RYQLDPKFI (SEQ ID NO.: 93)); CA125 (reported immunogenic epitopes include: ILFTINFTI (SEQ ID NO.: 94); VLFTINFTI (SEQ ID NO.: 95); TLNFTITNL (SEQ ID NO.: 96); VLQGLLKPL (SEQ ID NO.: 97); VLQGLLRPV (SEQ ID NO.: 98); RLDPKSPGV (SEQ ID NO.: 99); QLYWELSKL (SEQ ID NO.: 100); KLTRGIVEL (SEQ ID NO.: 101); QLTNGITEL (SEQ ID NO.: 102); QLTHNITEL (SEQ ID NO.: 103); and TLDRNSLYV (SEQ ID NO.: 104)); Folate receptor a (reported immunogenic epitopes include: FLLSLALML (SEQ ID NO.: 105); and NLGPWIQQV (SEQ ID NO.: 106)); Sperm protein 17 (reported immunogenic epitopes include: ILDSSEEDK (SEQ ID NO.: 107)); TADG-12 (reported immunogenic epitopes include: YLPKSWTIQV (SEQ ID NO.: 108); and WIHEQMERDLKT (SEQ ID NO.: 109)); MUC-16 (reported immunogenic epitopes include: ILFTINFTI (SEQ ID NO.: 94); VLFTINFTI (SEQ ID NO.: 95); TLNFTITNL (SEQ ID NO.: 96); VLQGLLKPL (SEQ ID NO.: 97); VLQGLLRPV (SEQ ID NO.: 98); RLDPKSPGV (SEQ ID NO.: 99); QLYWELSKL (SEQ ID NO.: 100); KLTRGIVEL (SEQ ID NO.: 101); QLTNGITEL (SEQ ID NO.: 102); QLTHNITEL (SEQ ID NO.: 103); and TLDRNSLYV (SEQ ID NO.: 104); L1CAM (reported immunogenic epitopes include: LLANAYIYV (SEQ ID NO.: 110); YLLCKAFGA (SEQ ID NO.: 111); and KLSPYVHYT (SEQ ID NO.: 112)); Mannan-MUC-1 (reported immunogenic epitopes include: PDTRPAPGSTAPPAHGVTSA (SEQ ID NO.: 113); STAPPVHNV (SEQ ID NO.: 114); LLLLTVLTV (SEQ ID NO.: 115); and PGSTAPPAHGVT (SEQ ID NO.: 116)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); MAGE-A4 (reported immunogenic epitopes include: EVDPASNTY (SEQ ID NO.: 127); GVYDGREHTV (SEQ ID NO.: 128); NYKRCFPVI (SEQ ID NO.: 129); and SESLKMIF (SEQ ID NO.: 130)); SSX-4 (reported immunogenic epitopes include: INKTSGPKRGKHAWTHRLRE (SEQ ID NO.: 131); YFSKKEWEKMKSSEKIVYVY (SEQ ID NO.: 132); MKLNYEVMTKLGFKVTLPPF (SEQ ID NO.: 133); KHAWTHRLRERKQLVVYEEI (SEQ ID NO.: 134); LGFKVTLPPFMRSKRAADFH (SEQ ID NO.: 135); KSSEKIVYVYMKLNYEVMTK (SEQ ID NO.: 136); and KHAWTHRLRERKQLVVYEEI (SEQ ID NO.: 134)); TAG-1 (reported immunogenic epitopes include: SLGWLFLLL (SEQ ID NO.: 137); and LSRLSNRLL (SEQ ID NO.: 138)); or TAG-2 (reported immunogenic epitopes include: LSRLSNRLL (SEQ ID NO.: 138)).

In some embodiments, the cancer antigen is a breast cancer antigen. In some embodiments, the breast cancer antigen is ENAH (hMena) (reported immunogenic epitopes include: TMNGSKSPV (SEQ ID NO.: 139)); mammaglobin-A (reported immunogenic epitopes include: PLLENVISK (SEQ ID NO.: 140)); NY-BR-1 (reported immunogenic epitopes include: SLSKILDTV (SEQ ID NO.: 141)); EpCAM (reported immunogenic epitopes include: RYQLDPKFI (SEQ ID NO.: 93)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); BAGE-1 (reported immunogenic epitopes include: AARAVFLAL (SEQ ID NO.: 142)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); MAGE-A1 (reported immunogenic epitopes include: EADPTGHSY (SEQ ID NO.: 143); KVLEYVIKV (SEQ ID NO.: 144); SLFRAVITK (SEQ ID NO.: 145); EVYDGREHSA (SEQ ID NO.: 146); RVRFFFPSL (SEQ ID NO.: 147); EADPTGHSY (SEQ ID NO.: 143); REPVTKAEML (SEQ ID NO.: 148); KEADPTGHSY (SEQ ID NO.: 149); DPARYEFLW (SEQ ID NO.: 150); ITKKVADLVGF (SEQ ID NO.: 151); SAFPTTINF (SEQ ID NO.: 152); SAYGEPRKL (SEQ ID NO.: 153); RVRFFFPSL (SEQ ID NO.: 147); TSCILESLFRAVITK (SEQ ID NO.: 154); PRALAETSYVKVLEY (SEQ ID NO.: 155); FLLLKYRAREPVTKAE (SEQ ID NO.: 156); and EYVIKVSARVRF (SEQ ID NO.: 157)); MAGE-A2 (reported immunogenic epitopes include: YLQLVFGIEV (SEQ ID NO.: 158); EYLQLVFGI (SEQ ID NO.: 159); REPVTKAEML (SEQ ID NO.: 148); EGDCAPEEK (SEQ ID NO.: 160); and LLKYRAREPVTKAE (SEQ ID NO.: 161)); mucink (reported immunogenic epitopes include: PDTRPAPGSTAPPAHGVTSA (SEQ ID NO.: 113)); Sp17 (reported immunogenic epitopes include: ILDSSEEDK (SEQ ID NO.: 107)); SSX-2 (reported immunogenic epitopes include: KASEKIFYV (SEQ ID NO.: 162); EKIQKAFDDIAKYFSK (SEQ ID NO.: 163); FGRLQGISPKI (SEQ ID NO.: 164); WEKMKASEKIFYVYMKRK (SEQ ID NO.: 165); KIFYVYMKRKYEAMT (SEQ ID NO.: 166); and KIFYVYMKRKYEAM (SEQ ID NO.: 167)); TAG-1 (reported immunogenic epitopes include: SLGWLFLLL (SEQ ID NO.: 137); and LSRLSNRLL (SEQ ID NO.: 138)); TAG-2 (reported immunogenic epitopes include: LSRLSNRLL (SEQ ID NO.: 138)); TRAG-3 (reported immunogenic epitopes include: CEFHACWPAFTVLGE (SEQ ID NO.: 168)); Her2/Neu (reported immunogenic epitopes include: HLYQGCQVV (SEQ ID NO.: 85); YLVPQQGFFC (SEQ ID NO.: 86); PLQPEQLQV (SEQ ID NO.: 87); TLEEITGYL (SEQ ID NO.: 88); ALIHHNTHL (SEQ ID NO.: 89); PLTSIISAV (SEQ ID NO.: 90); VLRENTSPK (SEQ ID NO.: 91); and TYLPTNASL (SEQ ID NO.: 92)); c-myc; cyclin B1; MUC1; p53 (reported immunogenic epitopes include: VVPCEPPEV (SEQ ID NO.: 84)); p62; or Survivin.

In some embodiments, the cancer antigen is a testicular cancer antigen. In some embodiments, the testicular cancer antigen is CD45 (reported immunogenic epitopes include: KFLDALISL (SEQ ID NO.: 169)); DKK1 (reported immunogenic epitopes include: ALGGHPLLGV (SEQ ID NO.: 170)); PRAME (reported immunogenic epitopes include: VLDGLDVLL (SEQ ID NO.: 23); SLYSFPEPEA (SEQ ID NO.: 24); ALYVDSLFFL (SEQ ID NO.: 25); SLLQHLIGL (SEQ ID NO.: 26); and LYVDSLFFL (SEQ ID NO.: 27)); RU2AS (reported immunogenic epitopes include: LPRWPPPQL (SEQ ID NO.: 171)); or Telomerase (reported immunogenic epitopes include: ILAKFLHWL (SEQ ID NO.: 172); RLVDDFLLV (SEQ ID NO.: 173); RPGLLGASVLGLDDI (SEQ ID NO.: 174); and LTDLQPYMRQFVAHL (SEQ ID NO.: 175)).

In some embodiments, the cancer antigen is a pancreatic cancer antigen. In some embodiments, the pancreatic cancer antigen is ENAH (hMena) (reported immunogenic epitopes include: TMNGSKSPV (SEQ ID NO.: 139)); PBF (reported immunogenic epitopes include: CTACRWKKACQR (SEQ ID NO.: 22)); K-ras (reported immunogenic epitopes include: VVVGAVGVG (SEQ ID NO.: 176)); Mesothelin (reported immunogenic epitopes include: SLLFLLFSL (SEQ ID NO.: 33); VLPLTVAEV (SEQ ID NO.: 34); ALQGGGPPY (SEQ ID NO.: 35); LYPKARLAF (SEQ ID NO.: 36); and AFLPWHRLF (SEQ ID NO.: 37)); or mucink (reported immunogenic epitopes include: PDTRPAPGSTAPPAHGVTSA (SEQ ID NO.: 113)).

In some embodiments, the cancer antigen is a liver cancer antigen. In some embodiments, the liver cancer antigen is G250/MN/CAIX (reported immunogenic epitopes include: HLSTAFARV (SEQ ID NO.: 177); KIFGSLAFL (SEQ ID NO.: 178); IISAVVGIL (SEQ ID NO.: 179); ALCRWGLLL (SEQ ID NO.: 180); ILHNGAYSL (SEQ ID NO.: 181); RLLQETELV (SEQ ID NO.: 182); VVKGVVFGI (SEQ ID NO.: 183); and YMIMVKCWMI (SEQ ID NO.: 184)); Hepsin (reported immunogenic epitopes include: SLLSGDWVL (SEQ ID NO.: 185); GLQLGVQAV (SEQ ID NO.: 186); and PLTEYIQPV (SEQ ID NO.: 187)); Intestinal carboxyl esterase (reported immunogenic epitopes include: SPRWWPTCL (SEQ ID NO.: 188)); alpha-foetoprotein (reported immunogenic epitopes include: GVALQTMKQ (SEQ ID NO.: 189); FMNKFIYEI (SEQ ID NO.: 190); and QLAVSVILRV (SEQ ID NO.: 191)); M-CSF (reported immunogenic epitopes include: LPAVVGLSPGEQEY (SEQ ID NO.: 192)); PBF (reported immunogenic epitopes include: CTACRWKKACQR (SEQ ID NO.: 22)); PSMA (reported immunogenic epitopes include: NYARTEDFF (SEQ ID NO.: 193)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); Sp17 (reported immunogenic epitopes include: ILDSSEEDK (SEQ ID NO.: 107)); c-myc; cyclin B1; p53 (reported immunogenic epitopes include: VVPCEPPEV (SEQ ID NO.: 84)); p62; or Survivin.

In some embodiments, the cancer antigen is a colorectal cancer antigen. In some embodiments, the colorectal cancer antigen is ENAH (hMena) (reported immunogenic epitopes include: TMNGSKSPV (SEQ ID NO.: 139)); Intestinal carboxyl esterase (reported immunogenic epitopes include: SPRWWPTCL (SEQ ID NO.: 188)); CASP-5 (reported immunogenic epitopes include: FLIIWQNTM (SEQ ID NO.: 194)); COA-1 (reported immunogenic epitopes include: TLYQDDTLTLQAAG (SEQ ID NO.: 195)); OGT (reported immunogenic epitopes include: SLYKFSPFPL (SEQ ID NO.: 196)); OS-9 (reported immunogenic epitopes include: KELEGILLL (SEQ ID NO.: 197)); TGF-betaRII (reported immunogenic epitopes include: RLSSCVPVA (SEQ ID NO.: 198)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); CEA (reported immunogenic epitopes include: TYYRPGVNLSLSC (SEQ ID NO.: 66); EIIYPNASLLIQN (SEQ ID NO.: 67); YACFVSNLATGRNNS (SEQ ID NO.: 68); LWWVNNQSLPVSP (SEQ ID NO.: 69); LWWVNNQSLPVSP (SEQ ID NO.: 69); LWWVNNQSLPVSP (SEQ ID NO.: 69); EIIYPNASLLIQN (SEQ ID NO.: 67); NSIVKSITVSASG (SEQ ID NO.: 70); KTWGQYWQV (SEQ ID NO.: 71); (A) MLGTHTMEV (SEQ ID NO.: 72); ITDQVPFSV (SEQ ID NO.: 73); YLEPGPVTA (SEQ ID NO.: 74); LLDGTATLRL (SEQ ID NO.: 75); VLYRYGSFSV (SEQ ID NO.: 76); SLADTNSLAV (SEQ ID NO.: 77); RLMKQDFSV (SEQ ID NO.: 78); RLPRIFCSC (SEQ ID NO.: 79); LIYRRRLMK (SEQ ID NO.: 80); ALLAVGATK (SEQ ID NO.: 81); IALNFPGSQK (SEQ ID NO.: 82); and RSYVPLAHR (SEQ ID NO.: 83)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); MAGE-A2 (reported immunogenic epitopes include: YLQLVFGIEV (SEQ ID NO.: 158); EYLQLVFGI (SEQ ID NO.: 159); REPVTKAEML (SEQ ID NO.: 148); EGDCAPEEK (SEQ ID NO.: 160); and LLKYRAREPVTKAE (SEQ ID NO.: 161)); Sp17 (reported immunogenic epitopes include: ILDSSEEDK (SEQ ID NO.: 107)); TAG-1 (reported immunogenic epitopes include: SLGWLFLLL (SEQ ID NO.: 137); and LSRLSNRLL (SEQ ID NO.: 138)); TAG-2 (reported immunogenic epitopes include: LSRLSNRLL (SEQ ID NO.: 138)); c-myc; cyclin B1; MUC1; p53 (reported immunogenic epitopes include: VVPCEPPEV (SEQ ID NO.: 84)); p62; Survivin; or gp70.

In some embodiments, the cancer antigen is a thyroid cancer antigen. In some embodiments, the thyroid cancer antigen is CALCA (reported immunogenic epitopes include: VLLQAGSLHA (SEQ ID NO.: 199)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); and HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46);
MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); or Sp17 (ILDSSEEDK (SEQ ID NO.: 107)).

In some embodiments, the cancer antigen is a lung cancer antigen. In some embodiments, the lung cancer antigen is CD274 (reported immunogenic epitopes include: LLNAFTVTV (SEQ ID NO.: 200)); mdm-2 (reported immunogenic epitopes include: VLFYLGQY (SEQ ID NO.: 201)); alpha-actinin-4 (reported immunogenic epitopes include: FIASNGVKLV (SEQ ID NO.: 202)); Elongation factor 2 (squamous cell carcinoma of the lung) (reported immunogenic epitopes include: ETVSEQSNV (SEQ ID NO.: 203)); ME1 (non-small cell lung carcinoma) (reported immunogenic epitopes include: FLDEFMEGV (SEQ ID NO.: 204)); NFYC (squamous cell carcinoma of the lung) (reported immunogenic epitopes include: QQITKTEV (SEQ ID NO.: 205)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL

(SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); GAGE-1,2,8 (reported immunogenic epitopes include: YRPRPRRY (SEQ ID NO.: 206)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); MAGE-A2 (reported immunogenic epitopes include: YLQLVFGIEV (SEQ ID NO.: 158); EYLQLVFGI (SEQ ID NO.: 159); REPVTKAEML (SEQ ID NO.: 148); EGDCAPEEK (SEQ ID NO.: 160); and LLKYRAREPVTKAE (SEQ ID NO.: 161)); MAGE-A6 (squamous cell lung carcinoma) (reported immunogenic epitopes include: MVKISGGPR (SEQ ID NO.: 207); EVDPIGHVY (SEQ ID NO.: 208); REPVTKAEML (SEQ ID NO.: 148); EGDCAPEEK (SEQ ID NO.: 160); ISGGPRISY (SEQ ID NO.: 209); and LLKYRAREPVTKAE (SEQ ID NO.: 161)); Sp17 (reported immunogenic epitopes include: ILDSSEEDK (SEQ ID NO.: 107)); TAG-1 (reported immunogenic epitopes include: SLGWLFLLL (SEQ ID NO.: 137); and LSRLSNRLL (SEQ ID NO.: 138)); TAG-2 (reported immunogenic epitopes include: LSRLSNRLL (SEQ ID NO.: 138)); TRAG-3 (reported immunogenic epitopes include: CEFHACWPAFTVLGE (SEQ ID NO.: 168)); XAGE-1b/GAGED2a (non-small cell lung cancer) (reported immunogenic epitopes include: RQKKIRIQL (SEQ ID NO.: 210); HLGSRQKKIRIQLRSQ (SEQ ID NO.: 211); and CATWKVICKSCISQTPG (SEQ ID NO.: 212)); c-myc; cyclin B1; Her2/Neu (reported immunogenic epitopes include: HLYQGCQVV (SEQ ID NO.: 85); YLVPQQGFFC (SEQ ID NO.: 86); PLQPEQLQV (SEQ ID NO.: 87); TLEEITGYL (SEQ ID NO.: 88); ALIHHNTHL (SEQ ID NO.: 89); PLTSIISAV (SEQ ID NO.: 90); VLRENTSPK (SEQ ID NO.: 91); and TYLPTNASL (SEQ ID NO.: 92)); MUC1; p53 (reported immunogenic epitopes include: VVPCEPPEV (SEQ ID NO.: 84)); p62; or Survivin. In some embodiments, the cancer antigen is a prostate cancer antigen. In some embodiments, the prostate cancer antigen is DKK1 (reported immunogenic epitopes include: ALGGHPLLGV (SEQ ID NO.: 170)); ENAH (hMena) (reported immunogenic epitopes include: TMNGSKSPV (SEQ ID NO.: 139)); Kallikrein 4 (reported immunogenic epitopes include: FLGYLILGV (SEQ ID NO.: 18); SVSESDTIRSISIAS (SEQ ID NO.: 19); LLANGRMPTVLQCVN (SEQ ID NO.: 20); and RMPTVLQCVNVSVVS (SEQ ID NO.: 21)); PSMA (reported immunogenic epitopes include: NYARTEDFF (SEQ ID NO.: 193)); STEAP1 (reported immunogenic epitopes include: MIAVFLPIV (SEQ ID NO.: 213); and HQQYFYKIPILVINK (SEQ ID NO.: 214)); PAP (reported immunogenic epitopes include: FLFLLFFWL (SEQ ID NO.: 215); TLMSAMTNL (SEQ ID NO.: 216); and ALDVYNGLL (SEQ ID NO.: 217)); PSA (prostate carcinoma) (reported immunogenic epitopes include: FLTPKKLQCV (SEQ ID NO.: 218); and VISNDVCAQV (SEQ ID NO.: 219)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); BAGE-1 (reported immunogenic epitopes include: AARAVFLAL (SEQ ID NO.: 142)); GAGE-1,2,8 (reported immunogenic epitopes include: YRPRPRRY (SEQ ID NO.: 206)); GAGE-3,4,5,6,7 (reported immunogenic epitopes include: YYWPRPRRY (SEQ ID NO.: 221)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); or Sp17 (reported immunogenic epitopes include: ILDSSEEDK (SEQ ID NO.: 107)).

In some embodiments, the cancer antigen is a kidney cancer antigen. In some embodiments, the kidney cancer antigen is FGF5 (reported immunogenic epitopes include: NTYASPRFK (SEQ ID NO.: 222));

Hepsin (reported immunogenic epitopes include: SLLSGDWVL (SEQ ID NO.: 185); GLQLGVQAV (SEQ ID NO.: 186); and PLTEYIQPV (SEQ ID NO.: 187)); Intestinal carboxyl esterase (reported immunogenic epitopes include: SPRWWPTCL (SEQ ID NO.: 188)); M-CSF (reported immunogenic epitopes include: LPAVVGLSPGEQEY (SEQ ID NO.: 192)); RU2AS (reported immunogenic epitopes include: LPRWPPPQL (SEQ ID NO.: 171)); hsp70-2 (renal cell carcinoma) (reported immunogenic epitopes include: SLFEGIDIYT (SEQ ID NO.: 223)); Mannan-MUC-1 (renal cell carcinoma) (reported immunogenic epitopes include: PDTRPAPGSTAPPAHGVTSA (SEQ ID NO.: 113); STAPPVHNV (SEQ ID NO.: 114); LLLLTVLTV (SEQ ID NO.: 115); and PGSTAPPAHGVT (SEQ ID NO.: 116)); or MAGE-A9 (renal cell carcinoma) (reported immunogenic epitopes include: ALSVMGVYV (SEQ ID NO.: 224)).

In some embodiments, the cancer antigen is a melanoma cancer antigen. In some embodiments, the melanoma cancer antigen is Hepsin (reported immunogenic epitopes include: SLLSGDWVL (SEQ ID NO.: 185); GLQLGVQA (SEQ ID NO.: 343); and PLTEYIQPV (SEQ ID NO.: 187)); ARTC1 (reported immunogenic epitopes include: YSVYFNLPADTIYTN (SEQ ID NO.: 225)); B-RAF (reported immunogenic epitopes include: EDLTVKIGDFGLATEKSRWSGSHQFEQLS (SEQ ID NO.: 226)); beta-catenin (reported immunogenic epitopes include: SYLDSGIHF (SEQ ID NO.: 227)); Cdc27 (reported immunogenic epitopes include: FSWAMDLDPKGA (SEQ ID NO.: 228)); CDK4 (reported immunogenic epitopes include: ACDPHSGHFV (SEQ ID NO.: 229)); CDK12 (reported immunogenic epitopes include: CILGKLFTK (SEQ ID NO.: 230)); CDKN2A (reported immunogenic epitopes include: AVCPWTWLR (SEQ ID NO.: 231)); CLPP (reported immunogenic epitopes include: ILDKVLVHL (SEQ ID NO.: 232)); CSNK1A1 (reported immunogenic epitopes include: GLFGDIYLA (SEQ ID NO.: 233)); FN1 (reported immunogenic epitopes include: MIFEKHGFRRTTPP (SEQ ID NO.: 234)); GAS7 (reported immunogenic epitopes include: SLADEAEVYL (SEQ ID NO.: 235)); GPNMB (reported immunogenic epitopes include: TLDWLLQTPK (SEQ ID NO.: 236)); HAUS3 (reported immunogenic epitopes include: ILNAMIAKI (SEQ ID NO.: 237)); LDLR-fucosyltransferase (reported immunogenic epitopes include: WRRAPAPGA (SEQ ID NO.: 238); and PVTWRRAPA (SEQ ID NO.: 239)); MART2 (reported immunogenic epitopes include: FLEGNEVGKTY (SEQ ID NO.: 240)); MATN (reported immunogenic epitopes include: KTLTSVFQK (SEQ ID NO.: 241)); MUM-1 (reported immunogenic epitopes include: EEKLIVVLF (SEQ ID NO.: 242)); MUM-2 (reported immunogenic epitopes include: SELFRSGLDSY (SEQ ID NO.: 243)); and FRSGLDSYV (SEQ ID NO.: 244)); MUM-3 (reported immunogenic epitopes include: EAFIQPITR (SEQ ID NO.: 245)); neo-PAP (reported immunogenic epitopes include: RVIKNSIRLTL (SEQ ID NO.: 246)); Myosin class/(reported immunogenic epitopes include: KINKNPKYK (SEQ ID NO.: 247)); PPP1R3B (reported immunogenic epitopes include: YTDFHCQYV (SEQ ID NO.: 248)); PRDX5 (reported immunogenic epitopes include: LLLDDLLVSI (SEQ ID NO.: 249)); PTPRK (reported immunogenic epitopes include: PYYFAAELPPRNLPEP (SEQ ID NO.: 250)); N-ras (reported immunogenic epitopes include: ILDTAGREEY (SEQ ID NO.: 251)); RBAF600 (reported immunogenic epitopes include: RPHVPESAF (SEQ ID NO.: 252)); SIRT2 (reported immunogenic epitopes include: KIFSEVTLK (SEQ ID NO.: 253)); SNRPD1 (reported immunogenic epitopes include: SHETVIIEL (SEQ ID NO.: 254)); Triosephosphate isomerase (reported immunogenic epitopes include: GELIGILNAAKVPAD (SEQ ID NO.: 255)); OA1 (reported immunogenic epitopes include: LYSACFWWL (SEQ ID NO.: 256)); RAB38/NY-MEL-1 (reported immunogenic epitopes include: VLHWDPETV (SEQ ID NO.: 257)); TRP-1/gp75 (reported immunogenic epitopes include: MSLQRQFLR (SEQ ID NO.: 258); ISPNSVFSQWRVVCDSLEDY (SEQ ID NO.: 259); SLPYWNFATG (SEQ ID NO.: 260); and SQWRVVCDSLEDYDT (SEQ ID NO.: 261)); TRP-2 (reported immunogenic epitopes include: SVYDFFVWL (SEQ ID NO.: 262); TLDSQVMSL (SEQ ID NO.: 263); LLGPGRPYR (SEQ ID NO.: 264); ANDPIFVVL (SEQ ID NO.: 265); QCTEVRADTRPWSGP (SEQ ID NO.: 266); and ALPYWNFATG (SEQ ID NO.: 267)); tyrosinase (reported immunogenic epitopes include: KCDICTDEY (SEQ ID NO.: 268); SSDYVIPIGTY (SEQ ID NO.: 269); MLLAVLYCL (SEQ ID NO.: 270); CLLWSFQTSA (SEQ ID NO.: 271); YMDGTMSQV (SEQ ID NO.: 272); AFLPWHRLF (SEQ ID NO.: 37); IYMDGTADFSF (SEQ ID NO.: 273); QCSGNFMGF (SEQ ID NO.: 274); TPRLPSSADVEF (SEQ ID NO.: 275); LPSSADVEF (SEQ ID NO.: 276); LHHAFVDSIF (SEQ ID NO.: 277); SEIWRDIDF (SEQ ID NO.: 277); QNILLSNAPLGPQFP (SEQ ID NO.: 278); SYLQDSDPDSFQD (SEQ ID NO.: 279); and FLLHHAFVDSIFEQWLQRHRP (SEQ ID NO.: 280)); Melan-A/MART-1 (reported immunogenic epitopes include: YTTAEEAAGIGILTVILGVLLLIGCWYCRR (SEQ ID NO.: 281)); gp100/Pmel17 (reported immunogenic epitopes include: ALNFPGSQK (SEQ ID NO.: 282); ALNFPGSQK (SEQ ID NO.: 282); VYFFLPDHL (SEQ ID NO.: 283); RTKQLYPEW (SEQ ID NO.: 284); HTMEVTVYHR (SEQ ID NO.: 285); SSPGCQPPA (SEQ ID NO.: 286); VPLDCVLYRY (SEQ ID NO.: 287); LPHSSSHWL (SEQ ID NO.: 288); SNDGPTLI (SEQ ID NO.: 289); GRAMLGTHTMEVTVY (SEQ ID NO.: 290); WNRQLYPEWTEAQRLD (SEQ ID NO.: 291); TTEWVETTARELPIPEPE (SEQ ID NO.: 292); TGRAMLGTHTMEVTVYH (SEQ ID NO.: 293); and GRAMLGTHTMEVTVY (SEQ ID NO.: 290)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); BAGE-1 (reported immunogenic epitopes include: AARAVFLAL (SEQ ID NO.: 142)); GAGE-1,2,8 (reported immunogenic epitopes include: YRPRPRRY (SEQ ID NO.: 206)); GAGE-3,4,5,6,7 (cutaneous melanoma) (reported immunogenic epitopes include: YYWPRPRRY (SEQ ID NO.: 221)); GnTVf (reported immunogenic epitopes include: VLPDVFIRC (V) (SEQ ID NO.: 294)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); LY6K (reported immunogenic epitopes include: RYCNLEGPPI (SEQ ID NO.: 295); KWTEPYCVIAAVKIFPRFFMVAKQ (SEQ ID NO.: 296); and KCCKIRYCNLEGPPINSSVF (SEQ ID NO.: 297)); MAGE-A1 (reported immunogenic epitopes include: EADPTGHSY (SEQ ID NO.: 143); KVLEYVIKV (SEQ ID NO.: 144); SLFRAVITK (SEQ ID NO.: 145); EVYDGREHSA (SEQ ID NO.: 146); RVRFFFPSL (SEQ ID NO.: 147); EADPTGHSY (SEQ ID NO.: 143); REPVTKAEML (SEQ ID NO.: 148); KEADPTGHSY (SEQ ID NO.: 149); DPARYEFLW (SEQ ID NO.: 150); ITKKVADLVGF (SEQ ID NO.: 151); SAFPTTINF (SEQ ID NO.: 152); SAYGEPRKL (SEQ ID NO.: 153); RVRFFFPSL (SEQ ID NO.: 147); TSCILESLFRAVITK (SEQ ID NO.: 154); PRALAETSYVKVLEY (SEQ ID NO.: 155); FLLLKYRAREPVTKAE (SEQ ID NO.: 156); and EYVIKVSARVRF (SEQ ID NO.: 157)); MAGE-A6 (reported immunogenic epitopes include: MVKISGGPR (SEQ ID NO.: 207); EVDPIGHVY (SEQ ID NO.: 208); REPVTKAEML (SEQ ID NO.: 148); EGDCAPEEK (SEQ ID NO.: 160); ISGGPRISY (SEQ ID NO.: 209); and LLKYRAREPVTKAE (SEQ ID NO.: 161)); MAGE-A10 (reported immunogenic epitopes include: GLYDGMEHL (SEQ ID NO.: 297)); and DPARYEFLW (SEQ ID NO.: 150)); MAGE-A12 (reported immunogenic epitopes include: FLWGPRALV (SEQ ID NO.: 298); VRIGHLYIL (SEQ ID NO.: 299); EGDCAPEEK (SEQ ID NO.: 160); REPFTKAEMLGSVIR (SEQ ID NO.: 300); and AELVHFLLLKYRAR (SEQ ID NO.: 301)); MAGE-C2 (reported immunogenic epitopes include: LLFGLALIEV (SEQ ID NO.: 302); ALKDVEERV (SEQ ID NO.: 303); SESIKKKVL (SEQ ID NO.: 304); ASSTLYLVF (SEQ ID NO.: 305); and SSTLYLVFSPSSFST (SEQ ID NO.: 306)); NA88-A (reported immunogenic epitopes include: QGQHFLQKV (SEQ ID NO.: 307)); Sp17 (reported immunogenic epitopes include: ILDSSEEDK (SEQ ID NO.: 107)); SSX-2 (reported immunogenic epitopes include: KASEKIFYV (SEQ ID NO.: 162); EKIQKAFDDIAKYFSK (SEQ ID NO.: 163); FGRLQGISPKI (SEQ ID NO.: 164); WEKMKASEKIFYVYMKRK (SEQ ID NO.: 165); KIFYVYMKRKYEAMT (SEQ ID NO.: 166); and KIFYVYMKRKYEAM (SEQ ID NO.: 167)); SSX-4 (reported immunogenic epitopes include: INKTSGPKRGKHAWTHRLRE (SEQ ID NO.: 131); YFSKKEWEKMKSSEKIVYVY (SEQ ID NO.: 132); MKLNYEVMTKLGFKVTLPPF (SEQ ID NO.: 133); KHAWTHRLRERKQLVVYEEI (SEQ ID NO.: 134); LGFKVTLPPFMRSKRAADFH (SEQ ID NO.: 135); KSSEKIVYVYMKLNYEVMTK (SEQ ID NO.: 136); and KHAWTHRLRERKQLVVYEEI (SEQ ID NO.: 134)); TRAG-3 (reported immunogenic epitopes include: CEFHACWPAFTVLGE (SEQ ID NO.: 168)); TRP2-INT2g (reported immunogenic epitopes include: EVISCKLIKR (SEQ ID NO.: 308)); or pgk.

In some embodiments, the cancer antigen is a squamous cell carcinoma antigen. In some embodiments, the squamous cell carcinoma antigen is CASP-8 (reported immunogenic epitopes include: FPSDSWCYF (SEQ ID NO.: 309)); p53 (reported immunogenic epitopes include: VVPCEPPEV (SEQ ID NO.: 84)); or SAGE (reported immunogenic epitopes include: LYATVIHDI (SEQ ID NO.: 310)).

In some embodiments, the cancer antigen is a chronic myeloid leukemia antigen. In some embodiments, the chronic myeloid leukemia antigen is BCR-ABL (reported immunogenic epitopes include: SSKALQRPV (SEQ ID NO.: 311); GFKQSSKAL (SEQ ID NO.: 312); ATGFKQSSKALQRPVAS (SEQ ID NO.: 313); and ATGFKQSSKALQRPVAS (SEQ ID NO.: 313)); dek-can (reported immunogenic epitopes include: TMKQICKKEIRRLHQY (SEQ ID NO.: 314)); EFTUD2 (reported immunogenic epitopes include: KILDAVVAQK (SEQ ID NO.: 315)); or GAGE-3,4,5,6,7 (reported immunogenic epitopes include: YYWPRPRRY (SEQ ID NO.: 221)

In some embodiments, the cancer antigen is an acute lymphoblastic leukemia antigen. In some embodiments, the acute lymphoblastic leukemia antigen is ETV6-AML1 (reported immunogenic epitopes include: RIAECILGM (SEQ ID NO.: 316); and IGRIAECILGMNPSR (SEQ ID NO.: 317)); or GAGE-3,4,5,6,7 (reported immunogenic epitopes include: YYWPRPRRY (SEQ ID NO.: 221)).

In some embodiments, the cancer antigen is an acute myelogenous leukemia antigen. In some embodiments, the acute myelogenous leukemia antigen is FLT3-ITD (reported immunogenic epitopes include: YVDFREYEYY (SEQ ID NO.: 318)); Cyclin-A1 (reported immunogenic epitopes include: FLDRFLSCM (SEQ ID NO.: 319); and SLIAAAAFCLA (SEQ ID NO.: 320)); or GAGE-3,4,5,6,7 (reported immunogenic epitopes include: YYWPRPRRY (SEQ ID NO.: 221)).

In some embodiments, the cancer antigen is a chronic lymphocytic leukemia antigen. In some embodiments, the chronic lymphocytic leukemia antigen is FNDC3B (reported immunogenic epitopes include: VVMSWAPPV (SEQ ID NO.: 321)); or GAGE-3,4,5,6,7 (reported immunogenic epitopes include: YYWPRPRRY (SEQ ID NO.: 221)

In some embodiments, the cancer antigen is a promyelocytic leukemia antigen. In some embodiments, the promyelocytic leukemia antigen is pml-RARalpha (reported immunogenic epitopes include: NSNHVASGAGEAAIETQSSSSEEIV (SEQ ID NO.: 322)); or GAGE-3,4,5,6,7 (reported immunogenic epitopes include: YYWPRPRRY (SEQ ID NO.: 221)).

In some embodiments, the cancer antigen is a multiple myeloma antigen. In some embodiments, the multiple myeloma antigen is MAGE-C1 (reported immunogenic epitopes include: ILFGISLREV (SEQ ID NO.: 323); KVVEFLAML (SEQ ID NO.: 324); SSALLSIFQSSPE (SEQ ID NO.: 325); SFSYTLLSL (SEQ ID NO.: 326); and VSSFFSYTL (SEQ ID NO.: 327)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); or Sp17 (reported immunogenic epitopes include: ILDSSEEDK (SEQ ID NO.: 107)).

In some embodiments, the cancer antigen is a B-cell lymphoma antigen. In some embodiments, the B-cell lymphoma antigen is D393-CD20 (reported immunogenic epitopes include: KPLFRRMSSLELVIA (SEQ ID NO.: 328)).

In some embodiments, the cancer antigen is a bladder carcinoma antigen. In some embodiments, the bladder carcinoma antigen is BAGE-1 (reported immunogenic epitopes include: AARAVFLAL (SEQ ID NO.: 142)); GAGE-1,2,8 (reported immunogenic epitopes include: YRPRPRRY (SEQ ID NO.: 206)); GAGE-3,4,5,6,7 (reported immunogenic epitopes include: YYWPRPRRY (SEQ ID NO.: 221)); MAGE-A4 (transitional cell carcinoma of urinary bladder) (reported immunogenic epitopes include: EVDPASNTY (SEQ ID NO.: 127); GVYDGREHTV (SEQ ID NO.: 128); NYKRCFPVI (SEQ ID NO.: 129); and SESLKMIF (SEQ ID NO.: 130)); MAGE-A6 (reported immunogenic epitopes include: MVKISGGPR (SEQ ID NO.: 207); EVDPIGHVY (SEQ ID NO.: 208); REPVTKAEML (SEQ ID NO.: 148); EGDCAPEEK (SEQ ID NO.: 160); ISGGPRISY (SEQ ID NO.: 209); and LLKYRAREPVTKAE (SEQ ID NO.: 161)); SAGE (reported immunogenic epitopes include: LYATVIHDI (SEQ ID NO.: 310)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); or Sp17 (reported immunogenic epitopes include: ILDSSEEDK (SEQ ID NO.: 107)).

In some embodiments, the cancer antigen is a head and neck cancer antigen. In some embodiments, the head and neck cancer antigen is BAGE-1 (head and neck squamous cell carcinoma) (reported immunogenic epitopes include: AARAVFLAL (SEQ ID NO.: 142)); GAGE-1,2,8 (reported immunogenic epitopes include: YRPRPRRY (SEQ ID NO.: 206)); GAGE-3,4,5,6,7 (reported immunogenic epitopes include: YYWPRPRRY (SEQ ID NO.: 221)); LY6K (reported immunogenic epitopes include: RYCNLEGPPI (SEQ ID NO.: 295; KWTEPYCVIAAVKIFPRFFMVAKQ (SEQ ID NO.: 296); and KCCKIRYCNLEGPPINSSVF (SEQ ID NO.: 297)); MAGE-A3 (head and neck squamous cell carcinoma) (reported immunogenic epitopes include: EVDPIGHLY (SEQ ID NO.: 329); FLWGPRALV (SEQ ID NO.: 298); KVAELVHFL (SEQ ID NO.: 330); TFPDLESEF (SEQ ID NO.: 331); VAELVHFLL (SEQ ID NO.: 332); MEVDPIGHLY (SEQ ID NO.: 333); EVDPIGHLY (SEQ ID NO.: 329); REPVTKAEML (SEQ ID NO.: 148); AELVHFLLL (SEQ ID NO.: 342); MEVDPIGHLY (SEQ ID NO.: 333); WQYFFPVIF (SEQ ID NO.: 334); EGDCAPEEK (SEQ ID NO.: 160); KKLLTQHFVQENYLEY (SEQ ID NO.: 335); RKVAELVHFLLLKYR (SEQ ID NO.: 336); KKLLTQHFVQENYLEY (SEQ ID NO.: 335); ACYEFLWGPRALVETS (SEQ ID NO.: 337); RKVAELVHFLLLKYR (SEQ ID NO.: 336); VIFSKASSSLQL (SEQ ID NO.: 338); VFGIELMEVDPIGHL (SEQ ID NO.: 339); GDNQIMPKAGLLIIV (SEQ ID NO.: 340); TSYVKVLHHMVKISG (SEQ ID NO.: 341); RKVAELVHFLLLKYRA (SEQ ID NO.: 336); and FLLLKYRAREPVTKAE (SEQ ID NO.: 156)); MAGE-A6 (reported immunogenic epitopes include: MVKISGGPR (SEQ ID NO.: 207); EVDPIGHVY (SEQ ID NO.: 208); REPVTKAEML (SEQ ID NO.: 148); EGDCAPEEK (SEQ ID NO.: 160); ISGGPRISY (SEQ ID NO.: 209); and LLKYRAREPVTKAE (SEQ ID NO.: 161)); or SAGE (reported immunogenic epitopes include: LYATVIHDI (SEQ ID NO.: 310)).

In some embodiments, the cancer antigen is an esophageal cancer antigen. In some embodiments, the esophageal cancer antigen is GAGE-3,4,5,6,7 (Esophageal squamous cell carcinoma and esophageal adenocarcinoma) (reported immunogenic epitopes include: YYWPRPRRY (SEQ ID NO.: 221)); MAGE-A2 (reported immunogenic epitopes include: YLQLVFGIEV (SEQ ID NO.: 158); EYLQLVFGI (SEQ ID NO.: 159); REPVTKAEML (SEQ ID NO.: 148); EGDCAPEEK (SEQ ID NO.: 160); and LLKYRAREPVTKAE (SEQ ID NO.: 161)); MAGE-A6 (reported immunogenic epitopes include: MVKISGGPR (SEQ ID NO.: 207); EVDPIGHVY (SEQ ID NO.: 208); REPVTKAEML (SEQ ID NO.: 148); EGDCAPEEK (SEQ ID NO.: 160); ISGGPRISY (SEQ ID NO.: 209); and LLKYRAREPVTKAE (SEQ ID NO.: 161)); NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); or Sp17 (reported immunogenic epitopes include: ILDSSEEDK (SEQ ID NO.: 107)).

In some embodiments, the cancer antigen is a brain cancer antigen. In some embodiments, the brain cancer antigen is TAG-1 (reported immunogenic epitopes include: SLGWLFLLL (SEQ ID NO.: 137); and LSRLSNRLL (SEQ ID NO.: 138)); or TAG-2 (reported immunogenic epitopes include: LSRLSNRLL (SEQ ID NO.: 138)).

In some embodiments, the cancer antigen is a pharynx cancer antigen. In some embodiments, the pharynx cancer antigen is TAG-1 (reported immunogenic epitopes include: SLGWLFLLL (SEQ ID NO.: 137); and LSRLSNRLL (SEQ ID NO.: 138)); or TAG-2 (reported immunogenic epitopes include: LSRLSNRLL (SEQ ID NO.: 138)).

In some embodiments, the cancer antigen is a tumor of the tongue antigen. In some embodiments, the tumor of the tongue antigen is TAG-1 (reported immunogenic epitopes include: SLGWLFLLL (SEQ ID NO.: 137); and LSRLSNRLL (SEQ ID NO.: 138)); or TAG-2 (reported immunogenic epitopes include: LSRLSNRLL (SEQ ID NO.: 138)).

In some embodiments, the cancer antigen is a synovial cell sarcoma antigen. In some embodiments, the synovial cell sarcoma antigen is NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); or Sp17 (reported immunogenic epitopes include: ILDSSEEDK (SEQ ID NO.: 107)).

In some embodiments, the cancer antigen is a neuroblastoma antigen. In some embodiments, the neuroblastoma antigen is NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); or Sp17 (reported immunogenic epitopes include: ILDSSEEDK (SEQ ID NO.: 107)).

In some embodiments, the cancer antigen is a uterine cancer antigen. In some embodiments, the uterine cancer antigen is NY-ESO-1 (reported immunogenic epitopes include: HLA-A2-restricted peptide p157-165 (SLLMWITQC (SEQ ID NO.: 38)); HLA-Cw3-restricted p92-100 (LAMP-FATPM (SEQ ID NO.: 39)); HLA-Cw6-restricted p80-88 (ARGPESRLL (SEQ ID NO.: 40)); SLLMWITQC (SEQ ID NO.: 38); MLMAQEALAFL (SEQ ID NO.: 41); YLAMPFATPME (SEQ ID NO.: 42); ASGPGGGAPR (SEQ ID NO.: 43); LAAQERRVPR (SEQ ID NO.: 44); TVSGNILTIR (SEQ ID NO.: 45); APRGPHGGAASGL (SEQ ID NO.: 46); MPFATPMEAEL (SEQ ID NO.: 47); KEFTVSGNILTI (SEQ ID NO.: 48); MPFATPMEA (SEQ ID NO.: 49); FATPMEAEL (SEQ ID NO.: 50); FATPMEAELAR (SEQ ID NO.: 51); LAMPFATPM (SEQ ID NO.: 52); ARGPESRLL (SEQ ID NO.: 40); SLLMWITQCFLPVF (SEQ ID NO.: 53); LLEFYLAMPFATPMEAELARRSLAQ (SEQ ID NO.: 54); EFYLAMPFATPM (SEQ ID NO.: 55); PGVLLKEFTVSGNILTIRLTAADHR (SEQ ID NO.: 56); RLLEFYLAMPFA (SEQ ID NO.: 57); QGAMLAAQERRVPRAAEVPR (SEQ ID NO.: 58); PFATPMEAELARR (SEQ ID NO.: 59); PGVLLKEFTVSGNILTIRLT (SEQ ID NO.: 60); VLLKEFTVSG (SEQ ID NO.: 61); AADHRQLQLSISSCLQQL (SEQ ID NO.: 62); LKEFTVSGNILTIRL (SEQ ID NO.: 63); PGVLLKEFTVSGNILTIRLTAADHR (SEQ ID NO.: 56); LLEFYLAMPFATPMEAELARRSLAQ (SEQ ID NO.: 54); KEFTVSGNILT (SEQ ID NO.: 220); LLEFYLAMPFATPM (SEQ ID NO.: 64); and AGATGGRGPRGAGA (SEQ ID NO.: 65)); LAGE-1 (reported immunogenic epitopes include: MLMAQEALAFL (SEQ ID NO.: 41); SLLMWITQC (SEQ ID NO.: 38); LAAQERRVPR (SEQ ID NO.: 44); ELVRRILSR (SEQ ID NO.: 122); APRGVRMAV (SEQ ID NO.: 123); SLLMWITQCFLPVF (SEQ ID NO.: 53); QGAMLAAQERRVPRAAEVPR (SEQ ID NO.: 124); AADHRQLQLSISSCLQQL (SEQ ID NO.: 62); CLSRRPWKRSWSAGSCPGMPHL (SEQ ID NO.: 125); ILSRDAAPLPRPG (SEQ ID NO.: 126); and AGATGGRGPRGAGA (SEQ ID NO.: 65)); HERV-K-MEL (reported immunogenic epitopes include: MLAVISCAV (SEQ ID NO.: 117)); KK-LC-1 (reported immunogenic epitopes include: RQKRILVNL (SEQ ID NO.: 118)); KM-HN-1 (reported immunogenic epitopes include: NYNNFYRFL (SEQ ID NO.: 119); EYSKECLKEF (SEQ ID NO.: 120); and EYLSLSDKI (SEQ ID NO.: 121)); or Sp17 (reported immunogenic epitopes include: ILDSSEEDK (SEQ ID NO.: 107)).

### Inducible Systems for Expression of Therapeutic Agents

If continuous administration of an antigen (e.g., a donor alloantigen, a self-antigen, or a non-self antigen) expressed by cloaked cells is needed to modulate an immune response, the antigen can be expressed using a constitutive promoter described herein or known by those of skill in the art (e.g., CAG, CMV, or another constitutive promoter). If the antigen is needed intermittently (e.g., needed during a period of relapse or flare up that occurs during a disease or condition, but not needed when a subject is asymptomatic), it can be expressed by an inducible promoter, which provides the capability of expressing the antigen only when it is needed. Antigens that may have potentially adverse effects if administered continuously can also be expressed intermittently using inducible promoters as described herein. Exemplary inducible expression systems are described below.

### Tetracycline Response Element

One widely used inducible expression system is based on tetracycline-controlled transcriptional activation. In this system, the antibiotic tetracycline, or one of its derivatives (e.g., doxycycline), is used to reversibly activate or inhibit gene expression. To use this system, a tetracycline response element (TRE) is placed upstream of a gene of interest (e.g., an antigen to be expressed by cloaked cells), typically along with a minimal promoter that has very low basal expression. A protein called rtTA, which also needs to be expressed by the cloaked cells, binds to the TRE and activates transcription in the presence of tetracycline or doxycycline. When tetracycline or doxycycline is removed, rtTA no longer binds to the TRE and the gene of interest is no longer expressed. Advanced versions of this system, Tet-On Advanced transactivator (rtTA2s-M2) and Tet-On 3G, may be particularly useful for human therapy as they are human codon optimized and respond to low concentrations of doxycycline,

### Light Inducible Systems

Another method for inducible activation of gene expression involves the use of optogenetics, which uses light sensitive proteins to manipulate gene expression. A recent development in optogenetics that can be used to inducibly express antigens in cloaked cells involves a class of proteins that undergo a conformational change and dimerize in response to blue light. These proteins have been fused to DNA-binding and transcriptional components that have been shown to bind to specific promoter sequences and activate transcription when brought together by exposure to blue light (Wang et al., Nat Methods, 9:266-269, 2012). This method of inducibly activating gene expression could be used to control the production of antigens in cloaked cells that are administered subcutaneously, as blue light can be shone onto the skin near the cloaked subcutaneous tissue to induce production of an antigen by the cloaked cells.

### Radiogenetics

A third method of inducibly activating gene expression (e.g., expression of an antigen by cloaked cells) involves the use of radio waves. In one version of a radio wave-inducible expression system, the TRPV1 receptor is fused to a GFP binding domain and co-expressed with a form of ferritin that is linked to GFP (Stanley et al., Nat Med 21:92-98, 2015). The GFP-ferritin binds to the GFP binding domain of the TRPV1 receptor. When a radio wave of a specific frequency is applied to the cell, ferritin interacts with TRPV1 and allows for an influx of calcium, which activates the transcription factor NFAT. Antigens can be inducibly expressed using this system if they are operably linked to an NFAT-sensitive promoter element, such as SRE-CRE-NFATRE, and co-expressed with TRPV1-GFP and GFP-ferritin. Radio wave-induced expression provides the advantage of being able to induce expression in cells that are further from the outside of the body, as radio waves can pass through tissue. For example, radiogenetics could be used to regulate gene expression in the retina. This method could, therefore, be used to inducibly express antigens in cloaked cells with non-invasive and non-harmful radio waves.

### Destabilization Domain System

Gene expression can also be regulated using destabilization domain systems. A transgene encoding a protein of interest (e.g., an antigen described herein) can also include destabilizing domains, such that the resulting protein product includes the protein of interest fused to a destabilizing domain. Exemplary destabilizing domains include mutants of the human FK506-and rapamycin-binding protein (FKBP12), which confer instability to the proteins to which they are fused. FKBP12 mutants include N-terminal mutants F15S, V24A, H25R, E60G, and L106P, and C-terminal mutants M66T, R71G, D100G, D100N, E102G, and K1051, as characterized in Banaszynski et al., Cell 126:995 (2006), the disclosure of which is incorporated herein by reference as it pertains to FKBP12 destabilizing domains. Destabilizing domains promote protein degradation. A small molecule synthetic ligand can be used to stabilize the destabilizing domain-containing proteins when expression of the protein of interest (e.g., an antigen) is desired. The small molecule ligand Shield-1 (Shld1) can be used to stabilize FKBP12 mutant-containing proteins by protecting them from degradation. Other destabilizing domains that can be used to regulate expression proteins of interest include mutants of the E. coli dihydrofolate reductase (ecDHFR) and mutants of the human estrogen receptor ligand binding domain (ERLBD), which confer instability resulting in degradation when fused to a protein of interest and can be stabilized by small molecule ligand trimethoprim (TMP), or by CMP8 or 4-hydroxytamoxifen (4OHT), respectively, as described in Iwamoto et al., Chem Biol. 17:981 (2010) and Miyazaki et al., J Am Chem Soc., 134:3942 (2012), the disclosures of each of which are incorporated herein by reference as they pertain to destabilization domain systems.

### Cumate Switch Inducible System

Another method for inducible activation of gene expression involves the use of the cumate gene-switch system. In the repressor configuration of this system, regulation is mediated by the binding of the repressor (CymR) to the operator site (CuO), placed downstream of a strong constitutive promoter. Addition of cumate, a small molecule, relieves the repression, allowing for expression of the transgene. Alternatively, a reverse-cumate-Trans-Activator (rcTA) may be inserted upstream of a minimal CMV promoter that is operably linked to a transgene encoding an antigen. A 6-times repeat of a Cumate Operator (6×CuO) may be inserted just before the translational start (ATG) of the transgene encoding the antigen. In the absence of cumate, rcTA cannot bind to the 6×CuO, so the transgene encoding the antigen will not be transcribed because the 6×CuO is not active. When cumate is added, it will form a complex with rcTA, which allows for binding to 6×CuO and transcription of the transgene encoding the antigen (Mullick et al., 2006).

### Ecdysone Inducible System

Another example of an inducible gene expression system is the ecdysone inducible system, in which a retinoid X receptor (RXR) and an N-terminal truncation of ecdysone receptor (EcR) fused to the activation domain of Vp16 (VpEcR) are inserted in the 5′ untranslated region of a gene expressed by the cloaked cell such that they are co-expressed by an endogenous promoter. An ecdysone responsive element (EcRE), with a downstream minimal promoter, can be inserted just upstream of the start codon of the transgene encoding the antigen. Co-expressed RXR and VpEcR can heterodimerize with each other. In the absence of ecdysone or synthetic drug analog muristerone A, dimerized RXR/VpEcR cannot bind to EcRE, so the transgene encoding the antigen is not transcribed. In the presence of ecdysone or muristerone A, dimerized RXR/VpEcR can bind to EcRE, such that the transgene encoding the antigen is transcribed (No et al., 1996). As ecdysone administration has no apparent effect on mammals, its use for regulating genes should be excellent for transient inducible expression of any gene.

### Ligand-Reversible Dimerization System

In another example, the transgene encoding an antigen can be modified so that it is functionally divided in to parts/domains, such as a 5′ portion and a 3′ portion, and an FKBP peptide sequence can be inserted into each domain. An IRES (internal ribosomal entry site) sequence may be placed between the two domains, which allows for simultaneous transcription of the two different domains to generate two separate proteins. In the absence of a dimerization agent, the two separate domains of the antigen will be functionally inactive. Upon introduction of a dimerization agent, such as rapamycin or AP20187, the FKBP peptides will dimerize, bringing together the 5′ and 3′ domains of the antigen and reconstituting an active protein (Rollins et al., 2000).

### Methods of Controlling Division of a Cloaked Cell

In an aspect, a method of controlling proliferation of a cloaked or uncloaked cell expressing a polypeptide containing an antigen is provided (e.g., to reduce the tumorigenic potential of a cloaked cell, to reduce proliferation of a cloaked cell that has become tumorigenic, or to ablate the cloaked or uncloaked cells).

The method comprises: providing a cell (e.g., a cell genetically modified to comprise at least one mechanism for providing antigen-specific immunomodulation when administered to a subject or an uncloaked cell) or a population of such cells; genetically modifying in the cell a cell division locus/loci (CDL), the CDL being one or more loci whose transcription product(s) is expressed by dividing cells (e.g., cells actively undergoing cell division), cells capable of undergoing cell division (e.g., cells that are not currently dividing but that have the capacity to do so), and post-mitotic cells (e.g., neurons), the genetic modification of the CDL containing one or more of: a) an ablation link (ALINK) system, the ALINK system comprising a DNA sequence encoding a negative selectable marker that is transcriptionally linked to a DNA sequence encoding the CDL (e.g., the DNA sequence encoding the negative selectable marker is inserted into the CDL such that expression of the negative selectable marker is linked to expression of the transcription product of the CDL, e.g., such that the negative selectable marker and CDL transcription product are co-expressed); and b) an inducible exogenous activator of regulation of a CDL (EARC) system, the EARC system comprising an inducible activator-based gene expression system that is operably linked to the CDL; permitting proliferation of the genetically modified cell comprising the ALINK system by maintaining the genetically modified cell comprising the ALINK system in the absence of an inducer of the negative selectable marker or ablating and/or inhibiting proliferation of the genetically modified cell comprising the ALINK system by exposing the cell comprising the ALINK system to the inducer of the negative selectable marker; and/or permitting proliferation of the genetically modified cell comprising the EARC system by exposing the genetically modified cell comprising the EARC system to an inducer of the inducible activator-based gene expression system or preventing or inhibiting proliferation of the genetically modified cell comprising the EARC system by maintaining the cell comprising the EARC system in the absence of the inducer of the inducible activator-based gene expression system; and administering the cell or a population of the cells to a subject. This approach can also be used to control cell division after cells containing an ALINK and/or EARC system have been administered to a subject by either administering the inducer (to ablate and/or inhibit proliferation of cells comprising an ALINK system or permit proliferation of cells comprising an EARC system) or withholding the inducer (to permit proliferation of cells comprising an ALINK system or prevent or inhibit proliferation of cells comprising the EARC system). Cells that have been modified to control cell division using one or more ALINK and/or EARC systems in one or more CDLs (e.g., 1, 2, 3, 4, or more CDLs) may be referred to as FAILSAFE™ cells. The number of cells that can be grown from a single FAILSAFE™ cell (clone volume) before the cell loses activity of all of the systems (e.g., ALINKs or EARCs) that control cell division through genetic mutation (e.g., the number of cell divisions it would take for a cell to “escape” from control and exhibit uncontrollable cell proliferation based on mathematical modeling) determines the FAILSAFE™ cell volume.

The FAILSAFE™ cell volume will depend on the number of ALINKs and the number of ALINK-targeted CDLs. The fail-safe property is further described in Table 3.

TABLE 3
FAILSAFE ™ cell volumes and their relationship to a human body were calculated
using mathematical modeling. The model did not take into account an event in which CDL
expression was co-lost with the loss of negative selectable marker activity, compromising
cell proliferation. Therefore the values are underestimates and were calculated assuming
10⁶ forward mutation rate for the negative selectable marker. The estimated number of cells
in a human body as 3.72 × 10¹³ was taken from (Bianconi et al., 2013).

| | | | FAILSAFE ™ | | |
|---|---|---|---|---|---|
| | | Genotype | cell volume | Relative (x) to a human | Estimated weight |
| CDL # | ALINK # | in CDLs | (#cells) | body = 3.72 × 10¹³ cells | of clones |
| 1 | 1 | het | 512 | 0.0000000000137 | 1 μg |
| 1 | 2 | hom | 16777216 | 0.000000451 | 31 mg |
| 2 | 3 | het, hom | 1.374E+11 | 0.004 | 0.26 kg |
| 2 | 4 | hom, hom | 1.13E+15 | 30 | 2100 kg |

In some embodiments, the CDL is a gene that is required for cell viability (e.g., the gene is needed for cell survival), such that the absence or loss of CDL expression in a normal, unmodified cell leads to cell death. In other embodiments, the CDL is a gene that is required for cell proliferation, such that the absence or loss of CDL expression in a normal, unmodified cell inhibits or impairs cell proliferation. Linking the expression of a negative selectable marker (ALINK) or exogenous inducible transcriptional activator (EARC) to the transcription of such a gene is desirable because it prevents the cell from “escaping” from control (e.g., the cell cannot divide uncontrollably in the absence of the gene) when linked to expression of the negative selectable marker (ALINK) or exogenous inducible transcriptional activator (EARC)). If a cell modified to co-express a negative selectable marker (ALINK) or exogenous inducible transcriptional activator (EARC) with a CDL required for cell viability or cell proliferation were to lose expression of the CDL (e.g., due to a mutation), the cell would die or stop proliferating, thereby obviating the need for proliferation of the cell to be controlled using the ALINK or EARC system. A cell may be modified to link the transcription of a negative selectable marker (ALINK) or an exogenous inducible transcriptional activator (EARC) to the transcription of a single gene (e.g., a single CDL, such as CDK1), e.g., by insertion of a DNA sequence encoding the negative selectable marker or exogenous inducible transcriptional activator into the gene locus such that the negative selectable marker or exogenous inducible transcriptional activator are co-expressed with the gene product. Alternatively, a cell may be modified to link the transcription of a negative selectable marker (ALINK) or an exogenous inducible transcriptional activator (EARC) to the transcription of more than one gene (e.g., 1, 2, 3, 4, 5, or more CDLs), e.g., by insertion of a DNA sequence encoding the negative selectable marker or exogenous inducible transcriptional activator into each of multiple CDLs (e.g., CDK1 and TOP2A or CDK1 and EEF2). A cell may be modified to include an ALINK system, an EARC system, or both.

In various embodiments, a CDL is a locus identified as an “essential gene” as set forth in Wang et al., 2015, which is incorporated herein by reference as if set forth in its entirety. Essential genes in Wang et al., 2015, were identified by computing a score (i.e., a CRISPR score) for each gene that reflects the fitness cost imposed by inactivation of the gene. In an embodiment, a CDL has a CRISPR score (CS) of less than about −1.0.

In various embodiments, a CDL is a locus/loci that encodes a gene product that is relevant to cell division and/or replication. For example, in various embodiments, a CDL is a locus/loci that encodes a gene product that is relevant to one or more of: i) cell cycle; ii) DNA replication; iii) RNA transcription and/or protein translation; and iv) metabolism.

In an embodiment, a CDL is one or more cyclin-dependent kinases that are involved with regulating progression of the cell cycle (e.g., control of G1/S G2/M and metaphase-to-anaphase transition), such as CDK1, CDK2, CDK3, CDK4, CDK5, CDK6, CDK7, CDK8, CDK9 and/or CDK11 (Morgan, 2007). In an embodiment, a CDL is one or more cyclins that are involved with controlling progression of the cell cycle by activating one or more CDK, such as, for example, cyclinB, cyclinE, cyclinA, cyclinC, cyclinD, cyclinH, cyclinC, cyclinT, cyclinL and/or cyclinF (FUNG and POON, 2005). In an embodiment, a CDL is one or more loci involved in the anaphase-promoting complex that controls the progression of metaphase to anaphase transition in the M phase of the cell cycle (Peters, 2002). In an embodiment, a CDL is one or more loci involved with kinetochore components that control the progression of metaphase to anaphase transition in the M phase of the cell cycle (Fukagawa, 2007). In an embodiment, a CDL is one or more loci involved with microtubule components that control microtubule dynamics required for the cell cycle (Cassimeris, 1999).

In various embodiments, a CDL is a locus/loci involved with housekeeping. As used herein, the term “housekeeping gene” or “housekeeping locus” refers to one or more genes that are required for the maintenance of basic cellular function. Housekeeping genes are expressed in all cells of an organism under normal and patho-physiological conditions.

In various embodiments, a CDL is a locus/loci that encodes a gene product that is relevant to cell division and/or proliferation and has a CRISPR score of less than about-1.0. For example, in an embodiment, a CDL is a locus/loci that encodes a gene product that is relevant to one or more of: i) cell cycle; ii) DNA replication; iii) RNA transcription and/or protein translation; and iv) metabolism, and has a CRISPR score of less than about-1.0. In an embodiment, the CDL may also be a housekeeping gene.

In some embodiments, the CDL is Cdk1/CDK1, Top2A/TOP2A, Cenpa/CEPNA, Birc5/BIRC5, or Eef2/EEF2. In some embodiments, the CDL is Cdk1/CDK1. In some embodiments, the CDL is Top2A/TOP2A. In some embodiments, the CDL is Eef2/EEF2. In some embodiments, the CDLs are Cdk1/CDK1 and Top2A/TOP2A or Cdk1/CDK1 and Eef2/EEF2.

In some embodiments, the CDL is one or more (e.g., 1, 2, 3, 4, or more) loci (e.g., genes) selected from the group including (presented in the format: mouse gene name/HUMAN GENE NAME (mouse EntrezGene ID/Human EntrezGene ID)) Actr8/ACTR8 (56249/93973); Alg11/ALG11 (207958/440138); Anapc11/ANAPC11 (66156/51529); Anapc2/ANAPC2 (99152/29882); Anapc4/ANAPC4 (52206/29945); Anapc5/ANAPC5 (59008/51433); Aurka/AURKA (20878/6790); Banf1/BANF1 (23825/8815); Birc5/BIRC5 (11799/332); Bub3/BUB3 (12237/9184); Casc5/CASC5 (76464/57082); Ccna2/CCNA2 (12428/890); Conh/CCNH (66671/902); Cdc123/CDCl23 (98828/8872); Cdc16/CDCl6 (69957/8881); Cdc20/CDC20 (107995/991); Cdc23/CDC23 (52563/8697); Cdk1/CDK1 (12534/983); Cenpa/CENPA (12615/1058); Cenpm/CENPM (66570/79019); Chek1/CHEK1 (12649/1111); Chmp2a/CHMP2A (68953/27243); Ckap5/CKAP5 (75786/9793); Cltc/CLTC (67300/1213); Cops5/COPS5 (26754/10987); Dctn2/DCTN2 (69654/10540); Dctn3/DCTN3 (53598/11258); Dhfr/DHFR (13361/1719); Dtl/DTL (76843/51514); Dync1 h1/DYNC1H1 (13424/1778); Ecd/ECD (70601/11319); Ect2/ECT2 (13605/1894); Ep300/EP300 (328572/2033); Ercc3/ERCC3 (13872/2071); Espl1/ESPL1 (105988/9700); Fntb/FNTB (110606/2342); Gadd45gip1/GADD45GIP1 (102060/90480); Gins1/GINS1 (69270/9837); Gnb211/GNB2L1 (14694/10399); Gspt1/GSPT1 (14852/2935); Haus1/HAUS1 (225745/115106); Haus3/HAUS3 (231123/79441); Haus5/HAUS5 (71909/23354); Haus8/HAUS8 (76478/93323); Hdac3/HDAC3 (15183/8841); Kif11/KIF11 (16551/3832); Kif23/KIF23 (71819/9493); Kpnb1/KPNB1 (16211/3837); Mastl/MASTL (67121/84930); Mau2/MAU2 (74549/23383); Mcm3/MCM3 (17215/4172); Mcm4/MCM4 (17217/4173); Mcm7/MCM7 (17220/4176); Mnat1/MNAT1 (17420/4331); Mybbp1a/MYBBP1A (18432/10514); Ncapd2/NCAPD2 (68298/9918); Ncaph/NCAPH (215387/23397); Ndc80/NDC80 (67052/10403); Nle1/NLE1 (217011/54475); Nsl1/NSL1 (381318/25936); Nudc/NUDC (18221/10726); Nuf2/NUF2 (66977/83540); Nup133/NUP133 (234865/55746); Nup160/NUP160 (59015/23279); Nup188/NUP188 (227699/23511); Nup214/NUP214 (227720/8021); n/a/NUP62 (n/a/23636); Nup85/NUP85 (445007/79902); ORC3/Orc3 (50793/23595); Pafah1b1/PAFAH1B1 (18472/5048); Pcid2/PCID2 (234069/55795); Pfas/PFAS (237823/5198); Phb2/PHB2 (12034/11331); Pkmyt1/PKMYT1 (268930/9088); Plk1/PLK1 (18817/5347); Pmf1/PMF1 (67037/11243); Pole2/POLE2 (18974/5427); Ppat/PPAT (231327/5471); Psma6/PSMA6 (26443/5687); Psma7/PSMA7 (26444/5688); Psmb1/PSMB1 (19170/5689) Psmb4/PSMB4 (19172/5692); Psmd12/PSMD12 (66997/5718); Psmd13/PSMD13 (23997/5719); Psmd14/PSMD14 (59029/10213); Psmd7/PSMD7 (17463/5713); Racgap1/RACGAP1 (26934/29127); Rad21/RAD21 (19357/5885); Rae1/RAE1 (66679/8480); Rcc1/RCC1 (100088/1104); Rfc3/RFC3 (69263/5983); Rps27a/RPS27A (78294/6233); Rrm2/RRM2 (20135/6241); Sae1/SAE1 (56459/10055); Sec13/SEC13 (110379/6396); Smarcb1/SMARCB1 (20587/6598); Smc2/SMC2 (14211/10592); Smc4/SMC4 (70099/10051); Son/SON (20658/6651); Spc24/SPC24 (67629/147841); Spc25/SPC25 (66442/57405); Terf2/TERF2 (21750/7014); Tpx2/TPX2 (72119/22974); Tubg1/TUBG1 (103733/7283); Tubgcp2/TUBGCP2 (74237/10844); Tubgcp5/TUBGCP5 (233276/114791); Tubgcp6/TUBGCP6 (328580/85378); Txnl4a/TXNL4A (27366/10907); Usp39/USP39 (28035/10713); Wdr43/WDR43 (72515/23160); Zfp830/ZNF830 (66983/91603); Aatf/AATF (56321/26574); Alyref/ALYREF (21681/10189); Brf2/BRF2 (66653/55290); Cdc45/CDC45 (12544/8318); Cdc6/CDC6 (23834/990); Cdt1/CDT1 (67177/81620); Cinp/CINP (67236/51550); Cirh1a/CIRH1A (21771/84916); Ddb1/DDB1 (13194/1642); (Ercc2/ERCC2 (13871/2068); Gabpb1/GABPB1 (14391/2553); Gtf2b/GTF2B (229906/2959); Gtf2 h4/GTF2H4 (14885/2968); Gtf3a/GTF3A (66596/2971); Gtf3c1/GTF3C1 (233863/2975);Gtf3c2/GTF3C2 (71752/2976); Hinfp/HINFP (102423/25988); n/a/HIST2H2AA3 (n/a/8337); Ints3/INTS3 (229543/65123); Kin/KIN (16588/22944); Mcm2/MCM2 (17216/4171); Mcm6/MCM6 (17219/4175); Mcrs1/MCRS1 (51812/10445); Med11/MED11 (66172/400569); Mtpap/MTPAP (67440/55149); Myc/MYC (17869/4609); Ndnl2/NDNL2 (66647/56160); Nol11/NOL11 (68979/25926); Nol8/NOL8 (70930/55035); Pcna/PCNA (18538/5111); Pola1/POLA1 (18968/5422); Pold2/POLD2 (18972/5425); Pole/POLE (18973/5426); Polr1a/POLR1A (20019/25885); n/a/POLR2J2 (n/a/246721); Polr3a/POLR3A (218832/11128); Polr3c/POLR3C (74414/10623); Polr3 h/POLR3H (78929/171568); Prmt1/PRMT1 (15469/3276); Prmt5/PRMT5 (27374/10419); Puf60/PUF60 (67959/22827); Rad51/RAD51 (19361/5888); Rad51c/RAD51C (114714/5889); Rbx1/RBX1 (56438/9978); Rfc2/RFC2 (19718/5982); Rfc4/RFC4 (106344/5984); Rfc5/RFC5 (72151/5985); Rpa1/RPA1 (68275/6117); Rps3/RPS3 (27050/6188); Rrm1/RRM1 (20133/6240); Ruvbl1/RUVBL1 (56505/8607); Ruvbl2/RUVBL2 (20174/10856); Sap30 bp/SAP30BP (57230/29115); Smc1a/SMC1A (24061/8243); Smc3/SMC3 (13006/9126); Snapc4/SNAPC4 (227644/6621); Snapc5/SNAPC5 (330959/10302); Snip1/SNIP1 (76793/79753); Srrt/SRRT (83701/51593); Ssrp1/SSRP1 (20833/6749); Taf10/TAF10 (24075/6881); Taf1c/TAF1C (21341/9013); Taf6/TAF6 (21343/6878); Taf6l/TAF6L (67706/10629); Ticrr/TICRR (77011/90381); Top1/TOP1 (21969/7150); Top2a/TOP2A (21973/7153); Trrap/TRRAP (100683/8295); Zbtb11/ZBTB11 (271377/27107); Actl6a/ACTL6A (56456/86); Atr/ATR (245000/545); Chd4/CHD4 (107932/1108); Ciao1/CIAO1 (26371/9391); Ddx21/DDX21 (56200/9188); Dnaja3/DNAJA3 (83945/9093); Dnmt1/DNMT1 (13433/1786); Gins2/GINS2 (272551/51659); Gtf2 h3/GTF2H3 (209357/2967); n/a/HIST2H2BF (n/a/440689); Mms221/MMS22L (212377/253714); Mtor/MTOR (56717/2475); Narfl/NARFL (67563/64428); Ndufa13/NDUFA13 (67184/51079); Nol12/NOL12 (97961/79159); Nup107/NUP107 (103468/57122); Oraov1/ORAOV1 (72284/220064); Pam16/PAM16 (66449/51025); Pola2/POLA2 (18969/23649); Ppie/PPIE (56031/10450); Prpf19/PRPF19 (28000/27339); Psmc5/PSMC5 (19184/5705); Rbbp5/RBBP5 (213464/5929); Rbbp6/RBBP6 (19647/5930); Rptor/RPTOR (74370/57521); Rrn3/RRN3 (106298/54700); Smg1/SMG1 (233789/23049); Supt6/SUPT6H (20926/6830); Tada2b/TADA2B (231151/93624); Tfip11/TFIP11 (54723/24144); Tonsl/TONSL (66914/4796); Tpt1/TPT1 (22070/7178); Uba1/UBA1 (22201/7317); Vps25/VPS25 (28084/84313); Wbscr22/WBSCR22 (66138/114049); Wdr5/WDR5 (140858/11091); Xab2/XAB2 (67439/56949); Zmat2/ZMAT2 (66492/153527); Zfp335/ZNF335 (329559/63925); Acly/ACLY (104112/47); Adsl/ADSL (11564/158); Ahcy/AHCY (269378/191); Arl2/ARL2 (56327/402); Chka/CHKA (12660/1119); Coasy/COASY (71743/80347); Cox4i1/COX411 (12857/1327); n/a/COX7C (n/a/1350); n/a/CTPS1 (n/a/1503); Ddx10/DDX10 (77591/1662); Ddx20/DDX20 (53975/11218); Dhdds/DHDDS (67422/79947); Dhx30/DHX30 (72831/22907); Dhx8/DHX8 (217207/1659); Dhx9/DHX9 (13211/1660); DIst/DLST (78920/1743); Dpagt1/DPAGT1 (13478/1798); Gfpt1/GFPT1 (14583/2673); Gmps/GMPS (229363/8833); Gpn1/GPN1 (74254/11321); Gpn3/GPN3 (68080/51184); Guk1/GUK1 (14923/2987); Hsd17b10/HSD17B10 (15108/3028); Lrr1/LRR1 (69706/122769); Mtg2/MTG2 (52856/26164); Myh9/MYH9 (17886/4627); Nampt/NAMPT (59027/10135); Ncbp1/NCBP1 (433702/4686); Nfs1/NFS1 (18041/9054); Ppcdc/PPCDC (66812/60490); Qrsl1/QRSL1 (76563/55278); Rpp14/RPP14 (67053/11102); Smarca4/SMARCA4 (20586/6597); Snrnp200/SNRNP200 (320632/23020); Srbd1/SRBD1 (78586/55133); Srcap/SRCAP (100043597/10847); Ube2i/UBE2I (22196/7329); Ube2m/UBE2M (22192/9040); Vcp/VCP (269523/7415); Aamp/AAMP (227290/14); Acin1/ACIN1 (56215/22985); Aco2/ACO2 (11429/50); Adss/ADSS (11566/159); Alg2/ALG2 (56737/85365); Ap2s1/AP2S1 (232910/1175); Arcn1/ARCN1 (213827/372); Armc7/ARMC7 (276905/79637); Atp2a2/ATP2A2 (11938/488); Atp5a1/ATP5A1 (11946/498); Atp5d/ATP5D (66043/513); Atp50/ATP50 (28080/539); Atp6v0b/ATP6V0B (114143/533); Atp6v0c/ATP6VOC (11984/527); Atp6v1a/ATP6V1A (11964/523); Atp6v1b2/ATP6V1B2 (11966/526); Atp6v1d/ATP6V1D (73834/51382); Aurkaip1/AURKAIP1 (66077/54998); n/a/C1orf109 (n/a/54955); n/a/C21orf59 (n/a/56683); Ccdc84/CCDC84 (382073/338657); Cct2/CCT2 (12461/10576); Cct3/CCT3 (12462/7203); Cct4/CCT4 (12464/10575); Cct5/CCT5 (12465/22948); Cct7/CCT7 (12468/10574); Cct8/CCT8 (12469/10694); Cdipt/CDIPT (52858/10423); Cenpi/CENPI (102920/2491); Chordc1/CHORDC1 (66917/26973); Coa5/COA5 (76178/493753); Cog4/COG4 (102339/25839); Copa/COPA (12847/1314); Copb1/COPB1 (70349/1315); Copb2/COPB2 (50797/9276); Cope/COPE (59042/11316); Copz1/COPZ1 (56447/22818); Coq4/COQ4 (227683/51117); Cox15/COX15 (226139/1355); Cox17/COX17 (12856/10063); Cse1l/CSE1L (110750/1434); Csnk2b/CSNK2B (13001/1460); Cycs/CYCS (13063/54205); Dad1/DAD1 (13135/1603); Dap3/DAP3 (65111/7818); Dctn5/DCTN5 (59288/84516); Ddost/DDOST (13200/1650); Dgcr8/DGCR8 (94223/54487); Dhodh/DHODH (56749/1723); Dnlz/DNLZ (52838/728489); Dnm1l/DNM1L (74006/10059); Dnm2/DNM2 (13430/1785); Dohh/DOHH (102115/83475); Dolk/DOLK (227697/22845); Donson/DONSON (60364/29980); Dph3/DPH3 (105638/285381); Dtymk/DTYMK (21915/1841); Eif2b2/EIF2B2 (217715/8892); Eif2s2/EIF2S2 (67204/8894); Emc1/EMC1 (230866/23065); Emc7/EMC7 (73024/56851); Eno1/ENO1 (13806/2023); Fam50a/FAM50A (108160/9130); Fam96b/FAM96B (68523/51647); Fdps/FDPS (110196/2224); Gapdh/GAPDH (14433/2597); Gart/GART (14450/2618); Gemin4/GEMIN4 (276919/50628); Gemin5/GEMIN5 (216766/25929); Ggps1/GGPS1 (14593/9453); Gmppb/GMPPB (331026/29925); Gnb1l/GNB1L (13972/54584); n/a/GOLGA6L1 (n/a/283767); Gosr2/GOSR2 (56494/9570); Gpkow/GPKOW (209416/27238); Gpn2/GPN2 (100210/54707); Gps1/GPS1 (209318/2873); Grpel1/GRPEL1 (17713/80273); Grwd1/GRWD1 (101612/83743); Hmgcr/HMGCR (15357/3156); Hmgcs1/HMGCS1 (208715/3157); Hspa5/HSPA5 (14828/3309); Hspa9/HSPA9 (15526/3313); Hspd1/HSPD1 (15510/3329); Hspe1/HSPE1 (15528/3336); Hyou1/HYOU1 (12282/10525); Ipo13/IPO13 (230673/9670); Iscu/ISCU (66383/23479); Itpk1/ITPK1 (217837/3705); Kansl2/KANSL2 (69612/54934); Kansl3/KANSL3 (226976/55683); Kri1/KRI1 (215194/65095); Lamtor2/LAMTOR2 (83409/28956); Leng8/LENG8 (232798/114823); Ltv1/LTV1 (353258/84946); Mak16/MAK16 (67920/84549); Mat2a/MAT2A (232087/4144); Mcm3ap/MCM3AP (54387/8888); Mdn1/MDN1 (100019/23195); n/a/MFAP1 (n/a/4236); Mmgt1/MMGT1 (236792/93380); Mrpl16/MRPL16 (94063/54948); Mrpl17/MRPL17 (27397/63875); Mrpl33/MRPL33 (66845/95530); Mrpl38/MRPL38 (60441/64978); Mrpl39/MRPL39 (27393/54148); Mrpl45/MRPL45 (67036/84311); Mrpl46/MRPL46 (67308/26589); Mrpl53/MRPL53 (68499/116540); Mrps22/MRPS22 (64655/56945); Mrps25/MRPS25 (64658/64432); Mrps35/MRPS35 (232536/60488); Mrps5/MRPS5 (77721/64969); Mvd/MVD (192156/4597); Mvk/MVK (17855/4598); Naa25/NAA25 (231713/80018); Napa/NAPA (108124/8775); Nat10/NAT10 (98956/55226); Ndor1/NDOR1 (78797/27158); Ndufab1/NDUFAB1 (70316/4706); Nol10/NOL10 (217431/79954); Nop9/NOP9 (67842/161424); Nrde2/NRDE2 (217827/55051); Nsf/NSF (18195/4905); Nubp1/NUBP1 (26425/4682); Nudcd3/NUDCD3 (209586/23386); Nup155/NUP155 (170762/9631); Nup93/NUP93 (71805/9688); Nus1/NUS1 (52014/116150); Nvl/NVL (67459/4931); Ogdh/OGDH (18293/4967); Osbp/OSBP (76303/5007); Pak1ip1/PAK1IP1 (68083/55003); Pfdn2/PFDN2 (18637/5202); Pgam1/PGAM1 (18648/5223); Pkm/PKM (18746/5315); Pmpcb/PMPCB (73078/9512); Ppil2/PPIL2 (66053/23759); Ppp4c/PPP4C (56420/5531); Prelid1/PRELID1 (66494/27166); Prpf31/PRPF31 (68988/26121); Prpf6/PRPF6 (68879/24148); Psma1/PSMA1 (26440/5682); Psma2/PSMA2 (19166/5683); Psma3/PSMA3 (19167/5684); Psmb2/PSMB2 (26445/5690); Psmb3/PSMB3 (26446/5691); Psmb5/PSMB5 (19173/5693); Psmb6/PSMB6 (19175/5694); Psmb7/PSMB7 (19177/5695); Psmc2/PSMC2 (19181/5701); Psmc3/PSMC3 (19182/5702); Psmc4/PSMC4 (23996/5704); Psmd1/PSMD1 (70247/5707); Psmd2/PSMD2 (21762/5708); Psmd3/PSMD3 (22123/5709); Psmd4/PSMD4 (19185/5710); Psmd6/PSMD6 (66413/9861); Psmg3/PSMG3 (66506/84262); Ptpmt1/PTPMT1 (66461/114971); Ptpn23/PTPN23 (104831/25930); Rabggta/RABGGTA (56187/5875); Rabggtb/RABGGTB (19352/5876); Rbm19/RBM19 (74111/9904); Rfk/RFK (54391/55312); Rheb/RHEB (19744/6009); Riok1/RIOK1 (71340/83732); Rpn1/RPN1 (103963/6184); Rtfdc1/RTFDC1 (66404/51507); Sacm1I/SACM1L (83493/22908); Samm50/SAMM50 (68653/25813); Sco2/SCO2 (100126824/9997); Sdha/SDHA (66945/6389); Sdhb/SDHB (67680/6390); Sec61a1/SEC61A1 (53421/29927); Slc20a1/SLC20A1 (20515/6574); Slc7a6os/SLC7A6OS (66432/84138); Smn1/SMN1 (20595/6606); Smu1/SMU1 (74255/55234); Snrpd1/SNRPD1 (20641/6632); Snrpd3/SNRPD3 (67332/6634); Snrpe/SNRPE (20643/6635); Spata5/SPATA5 (57815/166378); Spata511/SPATA5L1 (214616/79029); Tango6/TANGO6 (272538/79613); n/a/TBC1D3B (n/a/414059); n/a/TBC1D3C (n/a/414060); Tbcb/TBCB (66411/1155); Tbcc/TBCC (72726/6903); Tbcd/TBCD (108903/6904); Tcp1/TCP1 (21454/6950); Telo2/TELO2 (71718/9894); Tex10/TEX10 (269536/54881); Tfrc/TFRC (22042/7037); Timm10/TIMM10 (30059/26519); Timm13/TIMM13 (30055/26517); Timm23/TIMM23 (53600/100287932); Timm44/TIMM44 (21856/10469); Tmx2/TMX2 (66958/51075); Tnpo3/TNPO3 (320938/23534); Trmt112/TRMT112 (67674/51504); Trau1ap/TRNAU1AP (71787/54952); Ttc1/TTC1 (66827/7265); Ttc27/TTC27 (74196/55622); Tti1/TTI1 (75425/9675); Tti2/TTI2 (234138/80185); n/a/TUBB (n/a/203068); Txn2/TXN2 (56551/25828); Uqcrc1/UQCRC1 (22273/7384); Uqcrh/UQCRH (66576/7388); Urb2/URB2 (382038/9816); Vmp1/VMP1 (75909/81671); n/a/VPS28 (n/a/51160); Vps29/VPS29 (56433/51699); Vps52/VPS52 (224705/6293); Wars2/WARS2 (70560/10352); Wdr7/WDR7 (104082/23335); Wdr70/WDR70 (545085/55100); Wdr74/WDR74 (107071/54663); Wdr77/WDR77 (70465/79084); Yae1d1/YAE1D1 (67008/57002); Yrdc/YRDC (230734/79693); Znhit2/ZNHIT2 (29805/741); Aars/AARS (234734/16); Bms1/BMS1 (213895/9790); Bud31/BUD31 (231889/8896); Bysl/BYSL (53414/705); Cars/CARS (27267/833); Cdc5l/CDC5L (71702/988); Cdc73/CDC73 (214498/79577); Cebpz/CEBPZ (12607/10153); Clasrp/CLASRP (53609/11129); Clp1/CLP1 (98985/10978); Cox5b/COX5B (12859/1329); Cpsf1/CPSF1 (94230/29894); Cpsf2/CPSF2 (51786/53981); Cpsf3l/CPSF3L (71957/54973); Dars/DARS (226414/1615); Dbr1/DBR1 (83703/51163); Ddx18/DDX18 (66942/8886); Ddx23/DDX23 (74351/9416); Ddx24/DDX24 (27225/57062); Ddx41/DDX41 (72935/51428); Ddx46/DDX46 (212880/9879); Ddx47/DDX47 (67755/51202); Ddx49/DDX49 (234374/54555); Ddx54/DDX54 (71990/79039); Ddx56/DDX56 (52513/54606); Dgcr14/DGCR14 (27886/8220); Dhx15/DHX15 (13204/1665); Dhx16/DHX16 (69192/8449); Dhx38/DHX38 (64340/9785); Diexf/DIEXF (215193/27042); Dimt1/DIMT1 (66254/27292); Dis3/DIS3 (72662/22894); Dkc1/DKC1 (245474/1736); Dnajc17/DNAJC17 (69408/55192); Ears2/EARS2 (67417/124454); Ebna1bp2/EBNA1BP2 (69072/10969); Eef1a1/EEF1A1 (13627/1915); Eef1g/EEF1G (67160/1937); Eef2/EEF2 (13629/1938); Eftud2/EFTUD2 (20624/9343); Eif1ad/EIF1AD (69860/84285); Eif2b1/EIF2B1 (209354/1967); Eif2b3/EIF2B3 (108067/8891); Eif2s1/EIF2S1 (13665/1965); Eif3c/EIF3C (56347/8663); n/a/EIF3CL (n/a/728689); Eif3d/EIF3D (55944/8664); Eif3f/EIF3F (66085/8665); Eif3g/EIF3G (53356/8666); Eif3i/EIF3| (54709/8668); Eif3l/EIF3L (223691/51386); Eif4a1/EIF4A1 (13681/1973); Eif4a3/EIF4A3 (192170/9775); Eif4g1/EIF4G1 (208643/1981); Eif5b/EIF5B (226982/9669); Eif6/EIF6 (16418/3692); Elac2/ELAC2 (68626/60528); EII/ELL (13716/8178); Etf1/ETF1 (225363/2107); Exosc2/EXOSC2 (227715/23404); Exosc4/EXOSC4 (109075/54512); Exosc5/EXOSC5 (27998/56915); n/a/EXOSC6 (n/a/118460); Exosc7/EXOSC7 (66446/23016); Exosc8/EXOSC8 (69639/11340); Fars2/FARS2 (69955/10667); Farsa/FARSA (66590/2193); Farsb/FARSB (23874/10056); Fau/FAU (14109/2197); Fip111/FIP1L1 (66899/81608); Ftsj3/FTSJ3 (56095/117246); Gle1/GLE1 (74412/2733); Gnl3l/GNL3L (237107/54552); Gtf2e1/GTF2E1 (74197/2960); Gtpbp4/GTPBP4 (69237/23560); Hars/HARS (15115/3035); Hars2/HARS2 (70791/23438); Heatr1/HEATR1 (217995/55127); Hnrnpc/HNRNPC (15381/3183); Hnrnpk/HNRNPK (15387/3190); Hnrnpl/HNRNPL (15388/3191); Hnrnpu/HNRNPU (51810/3192); lars/IARS (105148/3376); lars2/IARS2 (381314/55699); Imp3/IMP3 (102462/55272); Imp4/IMP4 (27993/92856); Ints1/INTS1 (68510/26173); Ints4/INTS4 (101861/92105); Ints5/INTS5 (109077/80789); Ints8/INTS8 (72656/55656); Ints9/INTS9 (210925/55756); Isg2012/ISG20L2 (229504/81875); Kars/KARS (85305/3735); n/a/KIAA0391 (n/a/9692); Lars/LARS (107045/51520); Lars2/LARS2 (102436/23395); Las1l/LAS1L (76130/81887); Lrpprc/LRPPRC (72416/10128); Lsm2/LSM2 (27756/57819); Lsm3/LSM3 (67678/27258); Lsm7/LSM7 (66094/51690); Magoh/MAGOH (17149/4116); Mars/MARS (216443/4141); Mars2/MARS2 (212679/92935); Med17/MED17 (234959/9440); Med20/MED20 (56771/9477); Med22/MED22 (20933/6837); Med27/MED27 (68975/9442); Med30/MED30 (69790/90390); Med8/MED8 (80509/112950); Mepce/MEPCE (231803/56257); Mettl16/METTL16 (67493/79066); Mphosph10/MPHOSPH10 (67973/10199); Mrpl10/MRPL10 (107732/124995); Mrpl12/MRPL12 (56282/6182); Mrpl21/MRPL21 (353242/219927); Mrpl28/MRPL28 (68611/10573); Mrpl3/MRPL3 (94062/11222); Mrpl34/MRPL34 (94065/64981); Mrpl4/MRPL4 (66163/51073); Mrpl41/MRPL41 (107733/64975); Mrpl51/MRPL51 (66493/51258); Mrps14/MRPS14 (64659/63931); Mrps15/MRPS15 (66407/64960); Mrps16/MRPS16 (66242/51021); Mrps18a/MRPS18A (68565/55168); Mrps2/MRPS2 (118451/51116); Mrps21/MRPS21 (66292/Mrps24/MRPS24 (64660/64951); Mrps6/MRPS6 (121022/64968); Nars/NARS (70223/4677); Nars2/NARS2 (244141/79731); Ncbp2/NCBP2 (68092/22916); Nedd8/NEDD8 (18002/4738); Ngdn/NGDN (68966/25983); Nhp2/NHP2 (52530/55651); Nip7/NIP7 (66164/51388); Noc2I/NOC2L (57741/26155); Noc4I/NOC4L (100608/79050); Nol6/NOL6 (230082/65083); 54460); Nol9/NOL9 (74035/79707); Nop16/NOP16 (28126/51491); Nop2/NOP2 (110109/4839); Nop58/NOP58 (55989/51602); Nsa2/NSA2 (59050/10412);Nudt21/NUDT21 (68219/11051); Osgep/OSGEP (66246/55644); Pabpn1/PABPN1 (54196/8106); Pdcd11/PDCD11 (18572/22984); Pes1/PES1 (64934/23481); Phb/PHB (18673/5245); Phf5a/PHF5A (68479/84844); Pnn/PNN (18949/5411); Polr1b/POLR1B (20017/84172); Polr1c/POLR1C (20016/9533); Polr2a/POLR2A (20020/5430); Polr2b/POLR2B (231329/5431); Polr2c/POLR2C (20021/5432); Polr2d/POLR2D (69241/5433); Polr2f/POLR2F (69833/5435); Polr2g/POLR2G (67710/5436); Polr2 h/POLR2H (245841/5437); Polr2i/POLR2I (69920/5438); Polr2j/POLR2J (20022/5439); Polr2I/POLR2L (66491/5441); Polr3e/POLR3E (26939/55718); Pop1/POP1 (67724/10940); Pop4/POP4 (66161/10775); Ppa1/PPA1 (67895/5464); Ppan/PPAN (235036/56342); Ppp2ca/PPP2CA (19052/5515); Prim1/PRIM1 (19075/5557); Prpf38b/PRPF38B (66921/55119); Prpf4/PRPF4 (70052/9128); Prpf8/PRPF8 (192159/10594); Ptcd1/PTCD1 (71799/26024); Pwp2/PWP2 (110816/5822); Qars/QARS (97541/5859); Ran/RAN (19384/5901); Rars/RARS (104458/5917); Rars2/RARS2 (109093/57038); Rbm25/RBM25 (67039/58517); Rbm8a/RBM8A (60365/9939); Rbmx/RBMX (19655/27316); Rcl1/RCL1 (59028/10171); Rngtt/RNGTT (24018/8732); Rnmt/RNMT (67897/8731); Rnpc3/RNPC3 (67225/55599); Rpap1/RPAP1 (68925/26015); Rpl10/RPL10 (110954/6134); Rpl10a/RPL10A (19896/4736); Rpl11/RPL11 (67025/6135); Rpl12/RPL12 (269261/6136); Rpl13/RPL13 (270106/6137); Rpl14/RPL14 (67115/9045); Rpl15/RPL15 (66480/6138); Rpl18/RPL18 (19899/6141); Rpl18a/RPL18A (76808/6142); Rpl23/RPL23 (65019/9349); n/a/RPL23A (n/a/6147); Rpl24/RPL24 (68193/6152); Rpl26/RPL26 (19941/6154); Rpl27/RPL27 (19942/6155); Rpl27a/RPL27A (26451/6157); Rpl3/RPL3 (27367/6122); Rpl30/RPL30 (19946/6156); Rpl31/RPL31 (114641/6160); Rpl32/RPL32 (19951/6161); n/a/RPL34 (n/a/6164); Rpl35/RPL35 (66489/11224); Rpl35a/RPL35A (57808/6165); Rpl36/RPL36 (54217/25873); Rpl37/RPL37 (67281/6167); Rpl37a/RPL37A (19981/6168); Rpl38/RPL38 (67671/6169); Rpl4/RPL4 (67891/6124); Rpl5/RPL5 (100503670/6125); Rpl6/RPL6 (19988/6128); Rpl7/RPL7 (19989/6129); Rpl7a/RPL7A (27176/6130); Rpl711/RPL7L1 (66229/285855); Rpl8/RPL8 (26961/6132); Rpl9/RPL9 (20005/6133); Rplp0/RPLP0 (11837/6175); Rpp21/RPP21 (67676/79897); Rpp30/RPP30 (54364/10556); Rps10/RPS10 (67097/6204); Rps11/RPS11 (27207/6205); Rps12/RPS12 (20042/6206); Rps13/RPS13 (68052/6207); n/a/RPS14 (n/a/6208); Rps15/RPS15 (20054/6209); Rps15a/RPS15A (267019/6210); Rps16/RPS16 (20055/6217); Rps17/RPS17 (20068/6218); Rps19/RPS19 (20085/6223); Rps2/RPS2 (16898/6187); Rps21/RPS21 (66481/6227); Rps23/RPS23 (66475/6228); Rps25/RPS25 (75617/6230); n/a/RPS3A (n/a/6189); Rps4x/RPS4X (20102/6191); Rps5/RPS5 (20103/6193); Rps6/RPS6 (20104/6194); Rps7/RPS7 (20115/6201); Rps8/RPS8 (20116/6202); Rps9/RPS9 (76846/6203); Rpsa/RPSA (16785/3921); Rsl24d1/RSL24D1 (225215/51187); Sars/SARS (20226/6301); Sars2/SARS2 (71984/54938); Sart1/SART1 (20227/9092); Sart3/SART3 (53890/9733); Sdad1/SDAD1 (231452/55153); Sf1/SF1 (22668/7536); Sf3a1/SF3A1 (67465/10291); Sf3a2/SF3A2 (20222/8175); Sf3a3/SF3A3 (75062/10946); Sf3b2/SF3B2 (319322/10992); Sf3b3/SF3B3 (101943/23450); Sf3b4/SF3B4 (107701/10262); Sfpq/SFPQ (71514/6421); Sin3a/SIN3A (20466/25942); Smg5/SMG5 (229512/23381); Smg6/SMG6 (103677/23293); Snrnp25/SNRNP25 (78372/79622); Snrnp27/SNRNP27 (66618/11017); Snrpd2/SNRPD2 (107686/6633); Snrpf/SNRPF (69878/6636); Srrm1/SRRM1 (51796/10250); Srsf1/SRSF1 (110809/6426); Srsf2/SRSF2 (20382/6427); Srsf3/SRSF3 (20383/6428); Srsf7/SRSF7 (225027/6432); Ssu72/SSU72 (68991/29101); Sugp1/SUGP1 (70616/57794); Tars/TARS (110960/6897); Tars2/TARS2 (71807/80222); Tbl3/TBL3 (213773/10607); Thoc2/THOC2 (331401/57187); Thoc5/THOC5 (107829/8563); Thoc7/THOC7 (66231/80145); Timeless/TIMELESS (21853/8914); Tsen2/TSEN2 (381802/80746); Tsr1/TSR1 (104662/55720); Tsr2/TSR2 (69499/90121); Tufm/TUFM (233870/7284); Tut1/TUT1 (70044/64852); Twistnb/TWISTNB (28071/221830); U2af1/U2AF1 (108121/7307); U2af2/U2AF2 (22185/11338); Uba52/UBA52 (22186/7311); Ubl5/UBL5 (66177/59286); Upf1/UPF1 (19704/5976); Upf2/UPF2 (326622/26019); Utp15/UTP15 (105372/84135); Utp20/UTP20 (70683/27340); Utp23/UTP23 (78581/84294); Utp3/UTP3 (65961/57050); Utp6/UTP6 (216987/55813); Vars/VARS (22321/7407); Wars/WARS (22375/7453); Wdr12/WDR12 (57750/55759); Wdr3/WDR3 (269470/10885); Wdr33/WDR33 (74320/55339); Wdr36/WDR36 (225348/134430); Wdr46/WDR46 (57315/9277); Wdr61/WDR61 (66317/80349); Wdr75/WDR75 (73674/84128); Xpo1/XPO1 (103573/7514); Yars/YARS (107271/8565); Yars2/YARS2 (70120/51067); Ythdc1/YTHDC1 (231386/91746); Zbtb8os/ZBTB8OS (67106/339487); and Zc3 h3/ZC3H3 (223642/23144). The CDLs are described in more detail in WO 2016141480, which is incorporated herein by reference in its entirety.

A cell can be modified to be a FAILSAFE™ cell by linking the expression of a CDL with that of a DNA sequence encoding a negative selectable marker (e.g., by inserting the DNA sequence encoding the negative selectable marker into a coding or non-coding region of the CDL), thereby allowing drug-induced ablation (e.g., killing) of mitotically active cells expressing both the CDL and the negative selectable marker. Ablation of proliferating cells may be desirable, for example, when cell proliferation is uncontrolled and/or accelerated relative to a cell's normal division rate (e.g., uncontrolled cell division exhibited by cancerous cells), or when therapeutic need for the cells has passed. Ablation of proliferating cells may be achieved via a genetic modification to the cell, referred to herein as an “ablation link” (ALINK), which links the expression of a DNA sequence encoding a negative selectable marker to that of a CDL, thereby allowing elimination or sufficient inhibition of ALINK-modified proliferating cells consequently expressing the CDL locus (sufficient inhibition being inhibition of cell expansion rate to a rate that is too low to contribute to tumor formation). In the presence of a pro-drug or other inducer of the negatively selectable system, cells expressing the negative selectable marker will stop proliferating or die, depending on the mechanism of action of the selectable marker and the identity of the CDL. Cells may be modified to comprise homozygous, heterozygous, hemizygous or compound heterozygous ALINKs. In one embodiment, to improve fidelity of ablation, a negative selectable marker may be introduced into all functional alleles of a CDL. In one preferred embodiment, a negative selectable marker may be introduced into all functional alleles of a CDL. The FAILSAFE™ system can be used to eliminate all of the cloaked cells, if desired.

An ALINK may be inserted in any position of the CDL, which allows co-expression of the CDL and the negative selectable marker.

In some embodiments, the ALINK system comprises a herpes simplex virus-thymidine kinase/ganciclovir system, a cytosine deaminase/5-fluorocytosine system, a carboxyl esterase/irinotecan system or an iCasp9/AP1903 system.

DNA encoding a negatively selectable marker (e.g., HSV-TK), may be inserted into a CDL (e.g., CDK1) in a host cell, such that expression of the negative selectable marker causes host cells expressing the negative selectable marker and, necessarily, the CDL, to be killed in the presence of an inducer (e.g., prodrug) of the negative selectable marker (e.g., ganciclovir (GCV)). In this example, host cells modified with the ALINK will produce thymidine kinase (TK) and the TK protein will convert GCV into GCV monophosphate, which is then converted into GCV triphosphate by cellular kinases. GCV triphosphate incorporates into the replicating DNA during S phase, which leads to the termination of DNA elongation and cell apoptosis (Halloran and Fenton, 1998).

A modified HSV-TK gene (Preuβ et al., 2010) is disclosed herein as one example of DNA encoding a negative selectable marker that may be used in an ALINK genetic modification to selectively ablate cells comprising undesirable cell division rate.

It is contemplated herein that alternative and/or additional negative selectable systems could be used in the tools and/or methods provided herein. Various negative selectable marker systems are known in the art (e.g., dCK.DM (Neschadim et al., 2012)).

For example, various negative selectable system having clinical relevance have been under active development in the field of “gene-direct enzyme/prodrug therapy” (GEPT), which aims to improve therapeutic efficacy of conventional cancer therapy with no or minimal side-effects (Hedley et al., 2007; Nawa et al., 2008). Frequently, GEPT involves the use of viral vectors to deliver a gene into cancer cells or into the vicinity of cancer cells in an area of the cancer cells that is not found in mammalian cells and that produces enzymes, which can convert a relatively non-toxic prodrug into a toxic agent.

HSV-TK/GCV, cytosine deaminase/5-fluorocytosine (CD/5-FC), and carboxyl esterase/irinotecan (CE/CPT-11) are examples of negative selectable marker systems being evaluated in GEPT pre- and clinical trials (Danks et al., 2007; Shah, 2012).

To overcome the potential immunogenicity of a Herpes Simplex Virus type 1 thymidine kinase/ganciclovir (TK/GCV) system, a “humanized” suicide system has been developed by engineering the human deoxycytidine kinase enzyme to become thymidine-active and to work as a negative selectable (suicide) system with non-toxic prodrugs: bromovinyl-deoxyuridine (BVdU), L-deoxythymidine (LdT) or L-deoxyuridine (LdU) (Neschadim et al., 2012).

The CD/5-FC negative selectable marker system is a widely used “suicide gene” system. Cytosine deaminase (CD) is a non-mammalian enzyme that may be obtained from bacteria or yeast (e.g., from Escherichia coli or Saccharomyces cerevisiae, respectively) (Ramnaraine et al., 2003). CD catalyzes conversion of cytosine into uracil and is an important member of the pyrimidine salvage pathway in prokaryotes and fungi, but it does not exist in mammalian cells. 5-fluorocytosine (5-FC) is an antifungal prodrug that causes a low level of cytotoxicity in humans (Denny, 2003). CD catalyzes conversion of 5-FC into the genotoxic agent 5-FU, which has a high level of toxicity in humans (Ireton et al., 2002).

The CE/CPT-11 system is based on the carboxyl esterase enzyme, which is a serine esterase found in a different tissues of mammalian species (Humerickhouse et al., 2000). The anti-cancer agent CPT-11 is a prodrug that is activated by CE to generate an active referred to as 7-ethyl-10-hydroxycamptothecin (SN-38), which is a strong mammalian topoisomerase I inhibitor (Wierdl et al., 2001). SN-38 induces accumulation of double-strand DNA breaks in dividing cells (Kojima et al., 1998).

Another example of a negative selectable marker system is the iCasp9/AP1903 suicide system, which is based on a modified human caspase 9 fused to a human FK506 binding protein (FKBP) to allow chemical dimerization using a small molecule AP1903, which has tested safely in humans. Administration of the dimerizing drug induces apoptosis of cells expressing the engineered caspase 9 components. This system has several advantages, such as, for example, including low potential immunogenicity, since it consists of human gene products, the dimerizer drug only effects the cells expressing the engineered caspase 9 components (Straathof et al., 2005). The iCasp/AP1903 suicide system is being tested in clinical settings (Di Stasi et al., 2011).

It is contemplated herein that the negative selectable marker system of the ALINK system could be replaced with a proliferation antagonist system. The term “proliferation antagonist” as used herein, refers to a natural or engineered compound(s) whose presence inhibits (completely or partially) division of a cell. For example, Omomyc^{ER} is the fusion protein of MYC dominant negative Omomyc with mutant murine estrogen receptor (ER) domain. When induced with tamoxifen (TAM), the fusion protein Omomyc^{ER} localizes to the nucleus, where the dominant negative Omomyc dimerizes with C-Myc, L-Myc and N-Myc, sequestering them in complexes that are unable to bind the Myc DNA binding consensus sequences (Soucek et al., 2002). As a consequence of the lack of Myc activity, cells are unable to divide (Oricchio et al., 2014). Another example of a proliferation antagonist is A-Fos, a dominant negative to activation protein-1 (AP1) (a heterodimer of the oncogenes Fos and Jun) that inhibits DNA binding in an equimolar competition (Olive et al., 1997). A-Fos can also be fused to ER domain, rendering its nuclear localization to be induced by TAM. Omomyc^{ER}/tamoxifen or A-Fos^{ER}/tamoxifen could be a replacement for TK/GCV to be an ALINK.

A cell can also be modified to be FAILSAFE™ by operably linking the CDL with an EARC, such as an inducible activator-based gene expression system. Under these conditions, the CDL will only be expressed (and the cell can only divide) in the presence of the inducer of the inducible activator-based gene expression system. Under these conditions, EARC-modified cells stop dividing, significantly slow down, or die in the absence of the inducer, depending on the mechanism of action of the inducible activator-based gene expression system and CDL function. Cells may be modified to comprise homozygous or compound heterozygous EARCs or may be altered such that only EARC-modified alleles can produce functional CDLs. In an embodiment, an EARC modification may be introduced into all alleles of a CDL, for example, to provide a mechanism for cell division control.

An EARC may be inserted in any position of CDL that permits co-expression of the CDL and the activator component of the inducible system in the presence of the inducer.

In an embodiment, an “activator” based gene expression system is preferable to a “repressor” based gene expression system. For example, if a repressor is used to suppress a CDL a loss of function mutation of the repressor could release CDL expression, thereby allowing cell proliferation. In a case of an activation-based suppression of cell division, the loss of activator function (mutation) would shut down CDL expression, thereby disallowing cell proliferation.

In some embodiments, the EARC system is a dox-bridge system, a cumate switch inducible system, an ecdysone inducible system, a radio wave inducible system, or a ligand-reversible dimerization system.

A dox-bridge may be inserted into a CDL (e.g., CDK1) in a host cell, such that in the presence of an inducer (e.g., doxycycline or “DOX”) the dox-bridge permits CDL expression, thereby allowing cell division and proliferation. Host cells modified with a dox-bridge EARC may comprise a reverse tetracycline Trans-Activator (rtTA) gene (Urlinger et al., 2000) under the transcriptional control of a promoter, which is active in dividing cells (e.g., in the CDL). This targeted insertion makes the CDL promoter no longer available for CDL transcription. To regain CDL transcription, a tetracycline responder element promoter (for example TRE (Agha-Mohammadi et al., 2004)) is inserted in front of the CDL transcript, which will express the CDL gene only in a situation when rtTA is expressed and doxycycline is present. When the only source of CDL expression is dox-bridged alleles, there is no CDL gene expression in the absence of doxycycline. The lack of CDL expression causes the EARC-modified cells to be compromised in their proliferation, either by death, stopping cell division, or by rendering the cell mitotic rate so slow that the EARC-modified cell could not contribute to tumor formation.

The term “dox-bridge” as used herein, refers to a mechanism for separating activity of a promoter from a target transcribed region by expressing rtTA (Gossen et al., 1995) by the endogenous or exogenous promoter and rendering the transcription of target region under the control of TRE. As used herein, “rtTA” refers to the reverse tetracycline transactivator elements of the tetracycline inducible system (Gossen et al., 1995) and “TRE” refers to a promoter consisting of TetO operator sequences upstream of a minimal promoter. Upon binding of rtTA to the TRE promoter in the presence of doxycycline, transcription of loci downstream of the TRE promoter increases. The rtTA sequence may be inserted in the same transcriptional unit as the CDL or in a different location of the genome, so long as the transcriptional expression's permissive or non-permissive status of the target region is controlled by doxycycline. A dox-bridge is an example of an EARC.

Introduction of an EARC system into the 5′ regulatory region of a CDL is also contemplated herein. It is contemplated herein that alternative and/or additional inducible activator-based gene expression systems could be used in the tools and or methods provided herein to produce EARC modifications. Various inducible activator-based gene expression systems are known in the art.

For example, destabilizing protein domains (Banaszynski et al., 2006) fused with an acting protein product of a coding CDL could be used in conjunction with a small molecule synthetic ligand to stabilize a CDL fusion protein when cell division and/or proliferation is desirable. In the absence of a stabilizer, destabilized-CDL-protein will be degraded by the cell, which in turn would stop proliferation. When the stabilizer compound is added, it would bind to the destabilized-CDL-protein, which would not be degraded, thereby allowing the cell to proliferate.

For example, transcription activator-like effector (TALE) technology (Maeder et al., 2013) could be combined with dimerizer-regulated expression induction (Pollock and Clackson, 2002). The TALE technology could be used to generate a DNA binding domain designed to be specific to a sequence, placed together with a minimal promoter replacing the promoter of a CDL. The TALE DNA binding domain also extended with a drug dimerizing domain. The latter can bind to another engineered protein having corresponding dimerizing domain and a transcriptional activation domain.

For example, a reverse-cumate-Trans-Activator (rcTA) may be inserted in the 5′ untranslated region of the CDL, such that it will be expressed by the endogenous CDL promoter. A 6-times repeat of a Cumate Operator (6×CuO) may be inserted just before the translational start (ATG) of CDL. In the absence of cumate in the system, rcTA cannot bind to the 6×CuO, so the CDL will not be transcribed because the 6×CuO is not active. When cumate is added, it will form a complex with rcTA, enabling binding to 6×CuO and enabling CDL transcription (Mullick et al., 2006).

For example, a retinoid X receptor (RXR) and an N-terminal truncation of ecdysone receptor (EcR) fused to the activation domain of Vp16 (VpEcR) may be inserted in the 5′ untranslated region of a CDL such that they are co-expressed by an endogenous CDL promoter. Ecdysone responsive element (EcRE), with a downstream minimal promoter, may also be inserted in the CDL, just upstream of the starting codon. Co-expressed RXR and VpEcR can heterodimerize with each other. In the absence of ecdysone or a synthetic drug analog muristerone A, dimerized RXR/VpEcR cannot bind to EcRE, so the CDL is not transcribed. In the presence of ecdysone or muristerone A, dimerized RXR/VpEcR can bind to EcRE, such that the CDL is transcribed (No et al., 1996).

For example, a transient receptor potential vanilloid-1 (TRPV1), together with ferritin, may be inserted in the 5′ untranslated region of a CDL and co-expressed by an endogenous CDL promoter. A promoter inducible by NFAT (NFATre) may also be inserted in the CDL, just upstream of the starting codon. In a normal environment, the NFAT promoter is not active. However, upon exposure to low-frequency radio waves, TRPV1 and ferritin create a wave of Ca⁺⁺entering the cell, which in turn converts cytoplasmatic-NFAT (NFATc) to nuclear-NFAT (NFATn), that ultimately will activate the NFATre and transcribe the CDL (Stanley et al., 2015).

For example, a CDL may be functionally divided in to parts/domains: 5′-CDL and 3′CDL, and a FKBP peptide sequence may be inserted into each domain. An IRES (internal ribosomal entry site) sequence may be placed between the two domains, which will be transcribed simultaneously by a CDL promoter but will generate two separate proteins. Without the presence of an inducer, the two separate CDL domains will be functionally inactive. Upon introduction of a dimerization agent, such as rapamycin or AP20187, the FKBP peptides will dimerize, bringing together the 5′ and 3′ CDL parts and reconstituting an active protein (Rollins et al., 2000).

### Pharmaceutical Compositions

The cloaked and uncloaked cells described herein (e.g., cloaked cells expressing a polypeptide comprising a donor alloantigen, cloaked cells expressing a polypeptide comprising a self-antigen, or uncloaked cells expressing a polypeptide comprising a non-self, disease-associated antigen) may be incorporated into a vehicle for administration into a patient, such as a human patient in need of a tissue or organ transplant or having an autoimmune disease or condition described herein. Pharmaceutical compositions containing cloaked or uncloaked cells can be prepared using methods known in the art. For example, such compositions can be prepared using, e.g., physiologically acceptable carriers, excipients or stabilizers (Remington: The Science and Practice of Pharmacology 22nd edition, Allen, L. Ed. (2013); incorporated herein by reference), and in a desired form, e.g., in the form of aqueous solutions.

The cloaked or uncloaked cells described herein can be administered in any physiologically compatible carrier, such as a buffered saline solution. Pharmaceutically acceptable carriers and diluents include saline, aqueous buffer solutions, solvents and/or dispersion media. The use of such carriers and diluents is well known in the art. Other examples include liquid media, for example, Dulbecco's modified eagle's medium (DMEM), sterile saline, sterile phosphate buffered saline, Leibovitz's medium (L15, Invitrogen, Carlsbad, Calif.), dextrose in sterile water, and any other physiologically acceptable liquid. Dispersions may also be prepared in glycerol, liquid polyethylene glycols, and mixtures thereof and in oils. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. In many cases, it will be preferable to include isotonic agents, for example, sugars or sodium chloride. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosol, and the like. Solutions of the invention can be prepared by using a pharmaceutically acceptable carrier or diluent and, as required, other ingredients enumerated above, followed by filtered sterilization, and then incorporating the cloaked or uncloaked cells as described herein.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. The person responsible for administration will, in any event, determine the appropriate dose for the individual subject. Moreover, for human administration, preparations may meet sterility, pyrogenicity, general safety, and purity standards as required by FDA Office of Biologics standards.

Pharmaceutical compositions comprising cloaked or uncloaked cells in a semi-solid or solid carrier are typically formulated for surgical implantation at the site of future transplantation or at the affected site of a disease or condition in the subject. It will be appreciated that liquid compositions also may be administered by surgical procedures. In particular embodiments, semi-solid or solid pharmaceutical compositions may comprise semi-permeable gels, matrices, cellular scaffolds and the like, which may be non-biodegradable or biodegradable. For example, in certain embodiments, it may be desirable or appropriate to sequester the cloaked or uncloaked cells from their surroundings, yet enable the cells to secrete and deliver biological molecules (e.g., an antigen listed in Table 2) to surrounding cells.

In other embodiments, different varieties of degradable gels and networks are utilized for the pharmaceutical compositions of the invention. For example, degradable materials include biocompatible polymers, such as poly(lactic acid), poly(lactic acid-co-glycolic acid), methylcellulose, hyaluronic acid, collagen, and the like.

In another embodiment, one or more hydrogels are used for the pharmaceutical compositions. The one or more hydrogels may include collagen, atelocollagen, fibrin constructs, hydrophilic vinyl and acrylic polymers, polysaccharides such as calcium alginate, and poly(ethylene oxide). Further, the hydrogel may be formed of poly(2-hydroxyethyl methacrylate), poly(acrylic acid), self-assembling peptides (e.g., RAD16), poly(methacrylic acid), poly(N-vinyl-2-pyrrolidinone), poly(vinyl alcohol) and their copolymers with each other and with hydrophobic monomers such as methyl methacrylate, vinyl acetate, and the like. Also preferred are hydrophilic polyurethanes containing large poly(ethylene oxide) blocks. Other preferred materials include hydrogels comprising interpenetrating networks of polymers, which may be formed by addition or by condensation polymerization, the components of which may comprise hydrophilic and hydrophobic monomers such as those just enumerated. In situ-forming degradable networks are also suitable for use in the invention (see, e.g., Anseth, K S et al. J. Controlled Release, 2002; 78:199-209; Wang, D. et al., Biomaterials, 2003; 24:3969-3980; U.S. Patent Publication 2002/0022676). These in situ forming materials are formulated as fluids suitable for injection; then may be induced to form a hydrogel by a variety of means such as change in temperature, pH, and exposure to light in situ or in vivo. In one embodiment, the construct contains fibrin glue containing gels. In another embodiment, the construct contains atelocollagen containing gels.

A polymer used to form a matrix may be in the form of a hydrogel. In general, hydrogels are cross-linked polymeric materials that can absorb more than 20% of their weight in water while maintaining a distinct three-dimensional structure. This definition includes dry cross-linked polymers that will swell in aqueous environments, as well as water-swollen materials. A host of hydrophilic polymers can be cross-linked to produce hydrogels, whether the polymer is of biological origin, semi-synthetic or wholly synthetic. The hydrogel may be produced from a synthetic polymeric material. Such synthetic polymers can be tailored to a range of properties and predictable lot-to-lot uniformity, and represent a reliable source of material that generally is free from concerns of immunogenicity. The matrices may include hydrogels formed from self assembling peptides, such as those discussed in U.S. Pat. Nos. 5,670,483 and 5,955,343, U.S. Patent Application No. 2002/0160471, and PCT Application No. WO 02/062969.

Properties that make hydrogels valuable in drug delivery applications include the equilibrium swelling degree, sorption kinetics, solute permeability, and their in vivo performance characteristics. Permeability to compounds depends, in part, upon the swelling degree or water content and the rate of biodegradation. Since the mechanical strength of a gel may decline in proportion to the swelling degree, it is also well within the contemplation of the present invention that the hydrogel can be attached to a substrate so that the composite system enhances mechanical strength. In some embodiments, the hydrogel can be impregnated within a porous substrate, so as to gain the mechanical strength of the substrate, along with the useful delivery properties of the hydrogel.

In other embodiments, the pharmaceutical composition comprises a biocompatible matrix made of natural, modified natural or synthetic biodegradable polymers, including homopolymers, copolymers and block polymers, as well as combinations thereof.

Examples of suitable biodegradable polymers or polymer classes include any biodegradable polymers discussed within this disclosure, including but not limited to, fibrin, collagen types I, II, III, IV and V, elastin, gelatin, vitronectin, fibronectin, laminin, thrombin, poly(aminoacid), oxidized cellulose, tropoelastin, silk, ribonucleic acids, deoxyribonucleic acids; proteins, polynucleotides, gum arabic, reconstituted basement membrane matrices, starches, dextrans, alginates, hyaluron, chitin, chitosan, agarose, polysaccharides, hyaluronic acid, poly(lactic acid), poly(glycolic acid), polyethylene glycol, decellularized tissue, self-assembling peptides, polypeptides, glycosaminoglycans, their derivatives and mixtures thereof. Suitable polymers also include poly(lactide) (PLA) which can be formed of L(+) and D(−) polymers, polyhydroxybutyrate, polyurethanes, polyphoshazenes, poly(ethylene glycol)-poly(lactide-co-glycolide) co-polymer, degradable polycyanoacrylates and degradable polyurethanes. For both glycolic acid and lactic acid, an intermediate cyclic dimer is may be prepared and purified prior to polymerization. These intermediate dimers are called glycolide and lactide, respectively.

Other useful biodegradable polymers or polymer classes include, without limitation, aliphatic polyesters, poly(alkylene oxalates), tyrosine derived polycarbonates, polyiminocarbonates, polyorthoesters, polyoxaesters, polyamidoesters, polyoxaesters containing amine groups, poly(propylene fumarate), polyfumarates, polydioxanones, polycarbonates, polyoxalates, poly(alpha-hydroxyacids), poly(esters), polyurethane, poly(ester urethane), poly(ether urethane), polyanhydrides, polyacetates, polycaprolactones, poly(orthoesters), polyamino acids, polyamides and blends and copolymers thereof. Additional useful biodegradable polymers include, without limitation stereopolymers of L-and D-lactic acid, copolymers of bis(para-carboxyphenoxy) propane and sebacic acid, sebacic acid copolymers, copolymers of caprolactone, poly(lactic acid)/poly(glycolic acid)/polyethyleneglycol copolymers, copolymers of polyurethane and poly(lactic acid), copolymers of alpha-amino acids, copolymers of alpha-amino acids and caproic acid, copolymers of alpha-benzyl glutamate and polyethylene glycol, copolymers of succinate and poly(glycols), polyphosphazene, poly(hydroxyalkanoates) and mixtures thereof. Binary and ternary systems also are contemplated.

In general, the material used to form a matrix is desirably configured so that it: (1) has mechanical properties that are suitable for the intended application; (2) remains sufficiently intact until tissue has in-grown and healed; (3) does not invoke an inflammatory or toxic response; (4) is metabolized in the body after fulfilling its purpose; (5) is easily processed into the desired final product to be formed; (6) demonstrates acceptable shelf-life; and (7) is easily sterilized.

In another embodiment, the population of cloaked or uncloaked cells can be administered by use of a scaffold. The composition, shape, and porosity of the scaffold may be any described above. Typically, these three-dimensional biomaterials contain the living cells attached to the scaffold, dispersed within the scaffold or incorporated in an extracellular matrix entrapped in the scaffold. Once implanted into the target region of the body, these implants become integrated with the host tissue, wherein the cells gradually become established.

Non-limiting examples of scaffolds that may be used include textile structures, such as weaves, knits, braids, meshes, non-wovens, and warped knits; porous foams, semi-porous foams, perforated films or sheets, microparticles, beads, and spheres and composite structures being a combination of the above structures. Nonwoven mats may, for example, be formed using fibers comprised of a synthetic absorbable copolymer of glycolic and lactic acids (PGA/PLA), sold under the tradename VICRYL sutures (Ethicon, Inc., Somerville, N.J.). Foams, composed of, for example, poly(epsilon-caprolactone)/poly(glycolic acid) (PCL/PGA) copolymer, formed by processes such as freeze-drying, or lyophilized, as discussed in U.S. Pat. No. 6,355,699, also may be utilized.

In another embodiment, the framework is a felt, which can be composed of a multifilament yarn made from a bioabsorbable material. The yarn can be made into a felt using standard textile processing techniques consisting of crimping, cutting, carding and needling. In another embodiment, cells are seeded onto foam scaffolds that may be used as composite structures.

The framework may be molded into a useful shape, such as to fill a tissue void. The framework can therefore be shaped to not only provide a channel for neural growth, but also provide a scaffold for the supporting and surrounding tissues, such as vascular tissue, muscle tissue, and the like. Furthermore, it will be appreciated that the population of cells may be cultured on pre-formed, non-degradable surgical or implantable devices.

Pharmaceutical compositions may include preparations made from cloaked or uncloaked cells that are formulated with a pharmaceutically acceptable carrier or medium. Suitable pharmaceutically acceptable carriers include any discussed within this disclosure, including but not limited to, water, salt solution (such as Ringer's solution), alcohols, oils, gelatins, polyvinyl pyrrolidine, carbohydrates such as lactose, amylose, or starch, fatty acid esters, and hydroxymethylcellulose. Such preparations can be sterilized, and if desired, mixed with auxiliary agents such as lubricants, preservatives, stabilizers, wetting agents, emulsifiers, salts for influencing osmotic pressure, buffers, and coloring agents. Pharmaceutical carriers suitable for use in the present invention are known in the art and are described, for example, in Pharmaceutical Sciences (17th Ed., Mack Pub. Co., Easton, Pa.) and WO 96/05309.

### Methods of Treatment

The cloaked and uncloaked cells and compositions described herein may be administered to a subject in need thereof (e.g., a subject waiting to receive a tissue or organ transplant, or a subject having or at risk of developing a disease or condition described herein) by a variety of routes, such as local administration to or near the site in need of a transplant, local administration to the site affected by the disease or condition (e.g., injection to or near the pancreas for the treatment of type 1 diabetes, direct administration to the central nervous system (CNS) (e.g., intracerebral, intraventricular, intrathecal, intracisternal, or stereotactic administration) for treating MS), intravenous, parenteral, intradermal, transdermal, intramuscular, intranasal, subcutaneous, percutaneous, intratracheal, intraperitoneal, intraarterial, intravascular, inhalation, perfusion, lavage, and oral administration. The most suitable route for administration in any given case will depend on the particular cells or composition administered, the patient, pharmaceutical formulation methods, administration methods (e.g., administration time and administration route), the patient's age, body weight, sex, severity of the disease being treated, the patient's diet, and the patient's excretion rate. Compositions may be administered once, or more than once (e.g., once annually, twice annually, three times annually, bi-monthly, or monthly). For local administration, the cloaked or uncloaked cells may be administered by any means that places the population of cells in a desired location, including catheter, syringe, shunt, stent, microcatheter, pump, implantation with a device, or implantation with a scaffold.

As described herein, before administration, the population of cells can be incubated in the presence of one or more factors, or under conditions, that stimulate stem cell differentiation into a desired cell type (e.g., a myelinating cell, an insulin producing cell, or other cell types described herein). Such factors are known in the art and the skilled artisan will appreciate that determination of suitable conditions for differentiation can be accomplished with routine experimentation. Such factors include growth or trophic factors, chemokines, cytokines, cellular products, demethylating agents, and other stimuli which are known to stimulate differentiation, for example, of stem cells along glial or islet cell pathways or lineages. Alternatively, the composition administered to the patient includes a population of cloaked cells with one or more factors that stimulate cell differentiation into a desired cell type, where the cell differentiation occurs in vivo at the tissue site.

Subjects that may be treated as described herein are subjects waiting to receive a transplant, subjects having an autoimmune disease or condition described herein (e.g., type 1 diabetes, MS, or a disease or condition listed in Table 2), or subject at risk of developing cancer or a bacterial, viral, fungal, or parasitic infection (e.g., a subject at risk due to family history, genetics, or environmental risk factors). The cells, compositions, and methods described herein can be used to treat a disease or condition in which the subject's immune system aberrantly mounts an immune response against the subject's own cells. The methods described herein may include a step of screening a subject for antigens that induce an immune response (e.g., immune cell activation, proliferation, polarization, differentiation, migration, degranulation, phagocytosis, or cytotoxicity) in immune cells isolated from the subject prior to treatment with or administration of the compositions described herein. The methods described herein may include a step of profiling a cell from an organ or tissue donor to identify donor alloantigens (e.g., donor alloantigens that can be expressed by a cloaked cell described herein to induce immune tolerance prior to a transplant). The methods described herein may also include a step of evaluating the symptoms of the disease or condition in a subject prior to treatment with or administration of the cloaked cells or compositions described herein. The subject can then be evaluated using the same diagnostic tests after administration of the cloaked cells or compositions to determine whether the subject's condition has improved. The compositions and methods described herein may be administered as a preventative treatment to subjects waiting to receive an organ or tissue transplant, to subjects at risk of developing an autoimmune disease or condition, or to subjects at risk of developing cancer or a bacterial, viral, fungal, or parasitic infection.

The cloaked cells, compositions, and methods described herein can be used to specifically modulate the immune response in a subject (e.g., to induce tolerance to a donor alloantigen in a subject prior to a tissue or organ transplant, to induce tolerance to a self-antigen in a subject suffering from an autoimmune disease or condition, or to induce tolerance to a cell before using an uncloaked version of the cell to stimulate an immune response to a non-self antigen in a subject who would benefit from immunization against said non-self antigen). The cloaked cells, compositions, and methods described herein can also be used to induce tolerance to an organ or tissue transplant in advance of a transplant, which may reduce the risk of rejection of the tissue or organ transplant, and reduce, minimize, or eliminate the need for immunosuppressive medications and HLA matching between the donor and recipient. Cloaked cells that express an antigen, such as a self-antigen that is aberrantly targeted by the immune system in a subject with an autoimmune disease or condition, compositions including such cells, or methods of administering such cells, may be used to induce immune tolerance to the antigen in a subject (e.g., an antigen that is associated with an autoimmune disease or condition, e.g., an antigen listed in Table 2). Cloaked cells can also be used to prepare a subject for a second step in a cell-based therapy, such as inducing tolerance to a cloaked cell before administering an uncloaked version of the same cell (e.g., inducing tolerance to a cloaked cell from a donor organ or tissue before a transplant, or inducing tolerance to a cloaked cell before administration of an uncloaked cell of the same type that has been modified to express a non-self antigen to stimulate a specific immune response to the non-self antigen).

Treatment may include administration of cloaked or uncloaked cells or a composition containing cloaked or uncloaked cells in various unit doses. Each unit dose will ordinarily contain a predetermined-quantity of the cloaked cells described herein. The quantity to be administered, and the particular route of administration and formulation, are within the skill of those in the clinical arts. A unit dose need not be administered as a single injection but may comprise continuous infusion over a set period of time. Dosing may be performed using a catheter, syringe, shunt, stent, microcatheter, pump, implantation with a device, or implantation with a scaffold. The number of cells administered may vary depending on whether the cells are administered to a tissue, organ, or body site associated with a disease or injury, or are administered subcutaneously to produce a cloaked or uncloaked subcutaneous tissue. For administration to a tissue, organ, or body site, the cloaked or uncloaked cells may be administered to the patient at a dose of, for example 1×10⁴ cells to 1×10¹⁰ cells (e.g., 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×106, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3 ×10⁹, 4×10⁹, 5×10⁹, 6×10⁹, 7×10⁹, 8×10⁹, 9×10⁹, 1×10¹⁰ cells). The number of cells administered will depend on the size of the recipient tissue, organ, or body site. For example, 2.5×10⁴ to 1×10⁵ cells (e.g., 2.5×10⁴, 3×10⁴, 4×10⁴, 5×10⁴, 6×10⁴, 7×10⁴, 8×10⁴, 9×10⁴, or 1×10⁵ cells) can be administered (e.g., injected) to the subretinal space of the eye or to a specific brain region; 1×10⁶ to 1×10⁸ cells (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸ cells) can be administered (e.g., injected) to a joint, with the quantity of cells depending on the size of the joint; and 5×10⁸ to 5×10⁹ cells (e.g., 5×10⁸, 6×10⁸, 7×10⁸, 8×10⁸, 9×10⁸, 1×10⁹, 2×10⁹, 3×10⁹, 4×10⁹, or 5×10⁹ cells) can be administered to the cardiac muscle. For creating cloaked or uncloaked subcutaneous tissue, 8×10⁸ cells to 3×10⁹ cells (e.g., 8×10⁸, 9×10⁸, 1×10⁹, 2×10⁹, 3×10⁹ cells) can be administered (e.g., injected) subcutaneously. Cloaked or uncloaked cells can be administered in two or more doses (e.g., two, three, four, five, or more different doses) or at the same dose two or more times (e.g., two, three, four, five, six, or more times over the course of an hour, day, week, month, or year). In some embodiments, the cloaked or uncloaked cells described herein are administered as a tissue (e.g., a tissue that has been grown and/or differentiated in vitro from cloaked or uncloaked cells). In some embodiments, the cloaked or uncloaked tissue is administered (e.g., implanted) with a gel, biocompatible matrix, or scaffold.

The compositions described herein are administered in an amount sufficient to prevent or reduce the risk of transplant rejection, to reduce, minimize, or eliminate the need for immunosuppressive drugs, to delay or prevent the onset of an autoimmune disease or condition described herein, to slow the progression of an autoimmune disease or condition described herein, to improve symptoms of an autoimmune disease or condition listed in Table 2 (e.g., to reduce symptoms of osteoarthritis or RA (e.g., reduce inflammation, joint pain, stiffness, or immobility); reduce symptoms of diabetes (e.g., improve insulin levels, reduce the need for regular insulin injections); reduce symptoms of multiple sclerosis (e.g., reduce numbness, weakness, or tingling, reduce tremor, reduce dizziness, improve vision, or improve gait)), or to induce the production of antibodies against a non-self, disease-associated antigen. Transplant rejection may be evaluated using standard methods known by those of skill in the art and may be reduced by 5% or more (e.g., 5%, 10%, 15%, 20%, 30%, 40%, 50%, 60%, 70%, 80%, 90%, 100% or more) compared to rates of transplant rejection typically observed without treatment. In some embodiments, administration of the cloaked cells or compositions described herein results in an equivalent outcome in transplant rejection as that observed in subjects administered immunosuppressive agent(s). Symptoms of diseases and conditions described herein can be evaluated using standard methods known to those of skill in the art and may be reduced (e.g., the subject's condition may be improved) by 5% or more (e.g., 5%, 10%, 15%, 20%, 30%, 40%, 50%, 60%, 70%, 80%, 90%, 100% or more) compared to symptoms prior to administration of the cloaked cells or compositions described herein or compared to outcomes observed in subjects treated with immunosuppressive agents. The production of antibodies directed against a non-self antigen can be measured using a blood sample collected from a subject and may be increased by 5% or more (e.g., 5%, 10%, 15%, 20%, 30%, 40%, 50%, 60%, 70%, 80%, 90%, 100% or more) compared to the amount of the antibody in a blood sample collected from the subject prior to treatment. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months or more following administration of the cloaked or uncloaked cell or composition depending on the dose and route of administration used for treatment. Depending on the outcome of the evaluation, the patient may receive additional treatments.

In some embodiments, the cloaked or uncloaked cells can be removed after they have elicited the desired therapeutic effect (e.g., after the cloaked cells have induced immune tolerance in the subject, or after the uncloaked cells have promoted the production of antibodies directed against a non-self antigen). The appropriate time for removing the cloaked or uncloaked cells can be determined by assessing an immune response of the subject (e.g., assessing the response of an immune cell isolated from the subject to an antigen to determine whether immune tolerance has been induced, or evaluating a blood sample collected from the subject to determine whether antibodies have been produced in response to a non-self antigen).

Cloaked or uncloaked cells can be removed surgically (e.g., if the cells were injected subcutaneously to produce a cloaked or uncloaked subcutaneous tissue, the cloaked or uncloaked subcutaneous tissue can be surgically removed). If the cloaked or uncloaked cells were modified to contain one or more systems for regulating cell division (e.g., modified to be FAILSAFE™ cells, e.g., by linking the expression of a CDL to an ALINK or EARC), the cells can be removed by administering an inducer of a negative selectable marker (e.g., the ALINK) or by ceasing to administer an activator of an inducible activator-based gene expression system (e.g., the EARC).

### Combination Therapy

In some embodiments, the cloaked or uncloaked cells described herein are administered in combination with one or more additional therapeutic agents. The additional therapeutic agent(s) can be administered after administration of the cloaked cells or concurrently with administration of the cloaked cells. The cloaked cells and additional therapeutic agents can also be administered simultaneously via co-formulation. The cloaked cells and therapeutic agent(s) can also be administered sequentially, such that the action of the cloaked cells and therapeutic agent(s) overlaps and their combined effect is such that the reduction in a symptom, or other parameter related to the disorder is greater than what would be observed with the cloaked cells or therapeutic agent delivered alone or in the absence of the other. The effect of the cloaked cells and therapeutic agent(s) can be partially additive, wholly additive, or greater than additive (e.g., synergistic). Sequential or substantially simultaneous administration of cloaked cells and therapeutic agent(s) can be effected by any appropriate route including, but not limited to oral routes, intravenous routes, intramuscular routes, local routes, or subcutaneous routes. The cloaked cells and therapeutic agent(s) can be administered by the same route or by different routes. For example, cloaked cells may be administered by subcutaneous injection while the additional therapeutic agent is administered orally. The cloaked cells may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before the additional therapeutic agent.

In one example, the additional therapeutic agent is an immunosuppressive agent(s) commonly given for organ or tissue transplant. The immunosuppressive agent(s) may be administered at a reduced dose relative to the standard dose. The immunosuppressive agent(s) may be an agent that is given immediately after transplantation to prevent acute rejection (e.g., methylprednisolone, atgam, thymoglobulin, OKT3, basiliximab, or daclizumab) or an immunosuppressive agent(s) used for maintenance (e.g., prednisone, a calcineurin inhibitor (e.g., cyclosporine, tacrolimus), Mycophenolate Mofetil, Azathioprine or Rapamycin). Other immunosuppressive agents given after organ transplantation include corticosteroids (e.g., methylprednisolone, dexamethasone, prednisolone), cytotoxic immunosuppressants (e.g., azathioprine, chlorambucil, cyclophosphamide, mercaptopurine, methotrexate), immunosuppressant antibodies (e.g., antithymocyte globulins, basiliximab, infliximab), sirolimus derivatives (e.g., everolimus, sirolimus), and anti-proliferative agents (e.g., mycophenolate mofetil, mycophenolate sodium, and azathioprine). In this case, the cloaked cell(s) is administered to or near the transplant site prior to organ or tissue transplantation to induce immune tolerance, and the immunosuppressive agent(s) is administered as an additional source of immunosuppression, if needed.

For use in treating inflammatory and autoimmune related diseases or conditions, the additional agent may be a disease-modifying anti-rheumatic drug (DMARD), a biologic response modifier (a type of DMARD), a corticosteroid, or a nonsteroidal anti-inflammatory medication (NSAID). In some embodiments, the additional agent is prednisone, prednisolone, methylprednisolone, methotrexate, hydroxychloroquine, sulfasalazine, leflunomide, cyclophosphamide, azathioprine, or a biologic such as tofacitinib, adalimumab, abatacept, anakinra, kineret, certolizumab, etanercept, golimumab, infliximab, rituximab or tocilizumab. In some embodiments, the additional agent is 6-mercaptopurine, 6-thioguanine, abatacept, adalimumab, alemtuzumab (Lemtrada), an aminosalicylate (5-aminoalicylic acid, sulfasalazine, mesalamine, balsalazide, olsalazine), an antibiotic, an anti-histamine, Anti-TNFa (infliximab, adalimumab, certolizumab pegol, natalizumab), azathioprine, belimumab, beta interferon, a calcineurin inhibitor, certolizumab, a corticosteroids, cromolyn, cyclosporin A, cyclosporine, dimethyl fumarate (tecfidera), etanercept, fingolimod (Gilenya), fumaric acid esters, glatiramer acetate (Copaxone), golimumab, hydroxyurea, IFNγ, IL-11, infliximab, leflunomide, leukotriene receptor antagonist, long-acting beta2 agonist, mitoxantrone, mycophenolate mofetil, natalizumab (tysabri), ocrelizumab, pimecrolimus, a probiotic (VSL #3), a retinoid, rituximab, salicylic acid, short-acting beta2 agonist, sulfasalazine, tacrolimus, teriflunomide (Aubagio), theophylline, tocilizumab, ustekinumab (anti-IL-12/IL-23), or vedolizumab (Anti alpha3 beta7 integrin). In this case, the cloaked cell(s) are administered to induce tolerance to an antigen (e.g., a self-antigen) aberrantly targeted by the immune system in connection with the autoimmune disease or condition, and the additional agent could be a compound or general anti-inflammatory agent (e.g., an NSAID or corticosteroid).

For example, if the disease is MS, the additional agent may be one or more of: interferon beta-1a, interferon beta-1b, gliatrimer acetate, daclizumab, teriflunomide, fingolimod, dimethyl fumarate, alemtuzumab, mitoxantrone, ocrelizumab, or natalizumab. The cloaked cell(s) administered could be stem cells or myelin producing cells (e.g., oligodendrocytes or Schwann cells). In some embodiments, the cloaked cell(s) can be modified to produce MOG and can be administered in combination with fingolimod.

For use in treating infectious disease, the additional agent may be an antiviral compound (e.g., vidarabine, acyclovir, gancyclovir, valgancyclovir, nucleoside-analog reverse transcriptase inhibitor (NRTI) (e.g., AZT (Zidovudine), ddl (Didanosine), ddC (Zalcitabine), d4T (Stavudine), or 3TC (Lamivudine)), non-nucleoside reverse transcriptase inhibitor (NNRTI) (e.g., (nevirapine or delavirdine), protease inhibitor (saquinavir, ritonavir, indinavir, or nelfinavir), ribavirin, or interferon); an antibacterial compound; an antifungal compound; an antiparasitic compound. The additional agent can be administered concurrently with or subsequently to uncloaked cell(s) to which the immune system has been tolerized that are modified to express a viral antigen.

For use in treating diabetes, the additional agent may be insulin, a sulfonylurea (e.g., chlorpropamide, glipizide, glyburide, glimepiride), a biguanide (e.g., metformin), a meglitinide (e.g., repaglinide, nateglinide), a thiazolidinedione (e.g., rosiglitazone, pioglitazone), a DPP-4 inhibitor (sitagliptin, saxagliptin, linagliptin, alogliptin), an SGLT2 inhibitor (e.g., canagliflozin, dapagliflozin), an alpha-glucosidase inhibitor (e.g., acarbose, miglitol), a bile acid sequestrant (e.g., colesevelam), aspirin, or a dietary regimen. The cloaked cell(s) administered could be stem cells or pancreatic beta cells that express a transgene encoding insulin and/or GAD65.

For use in treating cancer, the additional agent may be a checkpoint inhibitor, a chemotherapeutic drug, a biologic drug, a non-drug therapy (e.g., radiation therapy, cryotherapy, hyperthermia, or surgical excision or tumor tissue), or an anti-cancer vaccine. The additional agent can be administered concurrently with or subsequently to uncloaked cell(s) to which the immune system has been tolerized that are modified to express a cancer antigen

Checkpoint inhibitors can be broken down into at least 4 major categories: i) agents such as antibodies that block an inhibitory pathway directly on T cells or natural killer (NK) cells (e.g., PD-1 targeting antibodies such as nivolumab, pidilizumab/CT-011, and pembrolizumab, antibodies targeting TIM-3, and antibodies targeting LAG-3, 2B4, CD160, A2aR, BTLA, CGEN-15049, or KIR), ii) agents such as antibodies that activate stimulatory pathways directly on T cells or NK cells (e.g., antibodies targeting OX40, GITR, or 4-1BB), iii) agents such as antibodies that block a suppressive pathway on immune cells or rely on antibody-dependent cellular cytotoxicity to deplete suppressive populations of immune cells (e.g., CTLA-4 targeting antibodies such as ipilimumab or tremelimumab, antibodies targeting VISTA, and antibodies targeting PD-L2 (e.g., a PDL2/lg fusion protein such as AMP 224), Gr1, or Ly6G), and iv) agents such as antibodies or small molecules that block a suppressive pathway directly on cancer cells or that rely on antibody-dependent cellular cytotoxicity to enhance cytotoxicity to cancer cells (e.g., rituximab, antibodies or small molecules targeting PD-L1 (e.g., MPDL3280A/RG7446; MEDI4736; MSB0010718C; BMS 936559), and antibodies or small molecule inhibitors targeting B7-H3 (e.g., MGA271), B7-H4, Gal-9, or MUC1). In one embodiment, the inhibitor of checkpoint is an inhibitor (e.g., an inhibitory antibody or small molecule inhibitor) of HVEM, CD160, CHK 1, CHK2, B-7 family ligands, or a combination thereof. Such agents described herein can be designed and produced, e.g., by conventional methods known in the art (e.g., Templeton, Gene and Cell Therapy, 2015; Green and Sambrook, Molecular Cloning, 2012). In one embodiment, the inhibitor of checkpoint is an inhibitory antibody (e.g., a monospecific antibody such as a monoclonal antibody). The antibody may be, e.g., humanized or fully human. In other embodiments, the inhibitor of checkpoint is a fusion protein, e.g., an Fc-receptor fusion protein. In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein.

Chemotherapeutic agents include alkylating agents, antimetabolites, folic acid analogs, pyrimidine analogs, purine analogs and related inhibitors, vinca alkaloids, epipodopyyllotoxins, antibiotics, L-asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine;; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside(“Ara-C”); cyclophosphamide; thiotepa; taxoids, e.g.,, paclitaxel; chloranbucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the cloaked cells described herein. Suitable dosing regimens of combination chemotherapies are known in the art.

Anti-cancer biologics include cytokines (e.g., interferon or an interleukin (e.g., IL-2)) used in cancer treatment. In other embodiments the biologic is an anti-angiogenic agent, such as an anti-VEGF agent, e.g., bevacizumab. In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include Rituximab; Daclizumab; Basiliximab; Palivizumab; Infliximab; Trastuzumab; Gemtuzumab ozogamicin; Alemtuzumab; Ibritumomab tiuxetan; Adalimumab; Omalizumab; Tositumomab-I-131; Efalizumab; Cetuximab; Bevacizumab; Natalizumab; Tocilizumab; Panitumumab; Ranibizumab; Eculizumab; Certolizumab pegol; Golimumab; Canakinumab; Ustekinumab; Ofatumumab; Denosumab; Motavizumab; Raxibacumab; Belimumab; Ipilimumab; Brentuximab Vedotin; Pertuzumab; Ado-trastuzumab emtansine; and Obinutuzumab. Also included are antibody-drug conjugates.

### Kits

The invention also features a kit containing the cloaked cells described herein (e.g., cloaked cells expressing a set of the cloaking transgenes described herein (e.g., 1, 2, 3, 4, 5, 6, 7, or 8 of PD-L1, H2-M3, Cd47, Cd200, FasL, Ccl21b, Mfge8, and Spi6), optionally further expressing one or more of the following transgenes: TGF-β, Cd73, Cd39, Lag3, Il1r2, Ackr2, Tnfrsf22, Tnfrs23, Tnfrsf10, Dad1, and IFNγR1 d39). In one embodiment of the kit, the cloaked cells are further modified to express a polypeptide comprising an antigen (e.g., a donor alloantigen or a self-antigen associated with an autoimmune disease or condition). In another embodiment of the kit, the kit further includes uncloaked cells of the same type as the cloaked cells that are modified to express a polypeptide comprising a non-self, disease associated antigen (e.g., a cancer antigen, a bacterial antigen, a viral antigen, a fungal antigen, or a parasitic antigen). The cloaked or uncloaked cells may also contain one or more systems for regulating cell division (e.g., an ALINK or EARC system). The cloaked and/or uncloaked cells may be provided in a pharmaceutical composition. The kit may further include a syringe for administration of the cloaked and/or uncloaked cells or pharmaceutical composition and instructions for administering the cloaked cells or pharmaceutical composition for modulating an immune response described herein.

### Examples

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1: Administration of cloaked allogenic cells promotes immune tolerance of uncloaked allogenic cells

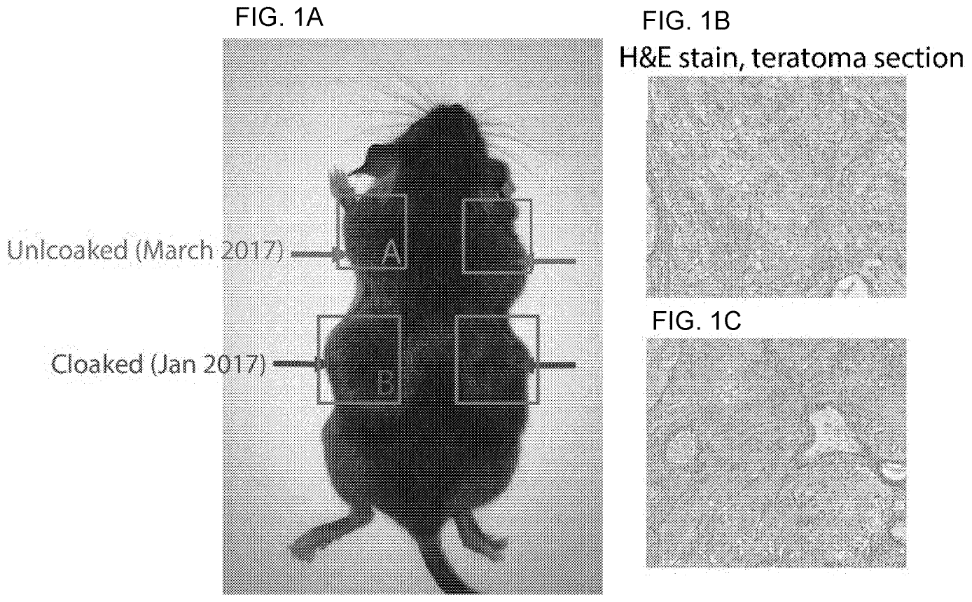

C3H mice received a subcutaneous injection of allogenic cloaked C57BL/6 embryonic stem cells (ESCs). The cloaked cells expressed all eight cloaking transgenes (Pd-L1, CD200, CD47, H2-M3, Fas-L, Mfge8, Spi6 and Ccl21) and were injected subcutaneously in each flank area. Approximately two months later, the same mice received a subcutaneous injection of uncloaked allogenic cells of the same type (e.g., allogenic cells of the same type from the same source that were not modified to express cloaking transgenes) in the shoulder area. As shown in FIG. 1A, the mouse injected with the cloaked and uncloaked allogenic cells retained both the cloaked cells (boxes labeled “A” near flanks) and the uncloaked cells (boxes labeled “B” near the shoulders). The teratomas formed by both the cloaked (FIG. 1C) and uncloaked (FIG. 1B) allogenic cells showed well differentiated tissues devoid of excessive host-derived lymphocyte infiltration or any sign or necrosis as visualized using H&E staining. These experiments were repeated with FVB/N animals as hosts for C57BL/6 derived ESCs with the same results. These data demonstrate that the initial injection of cloaked allogenic cells induced immune tolerance to the allogenic cells, preventing the immune system from rejecting the subsequently injected uncloaked allogenic cells. Therefore, these findings indicate that cell cloaking technology may be used to induce immune tolerance in a recipient in advance of an organ or tissue transplant.

### Example 2: Administration of Cloaked Cells Expressing an Alloantigen to Induce Immune Tolerance Prior to Organ Transplant

According to the methods disclosed herein, a physician of skill in the art can treat a subject, such as a human subject in need of an organ transplant (e.g., a kidney transplant), with cloaked cells expressing a polypeptide comprising an alloantigen from an organ donor (e.g., an HLA protein expressed by the kidney cells of the organ donor). To this end, a physician of skill in the art can administer to the human subject cloaked cells (e.g., stem cells or cloaked kidney cells) that express one or more (e.g., one, two, three, four, five, six, seven, or all eight) of PD-L1, HLA-G (H2-M3), Cd47, Cd200, FASLG (FasL), Ccl21 (Ccl21b), Mfge8, and Serpin B9 (Spi6) under the control of a constitutive promoter (e.g., CMV or CAG) and an HLA protein expressed by the organ donor under the control of a constitutive promoter (e.g., CMV or CAG). The cloaked cells may be administered to the subject, for example, by local administration near the kidney that will be replaced, or by subcutaneous injection (e.g., to create a cloaked subcutaneous tissue) to induce tolerance in the subject to the alloantigen from the donor providing the kidney. One million to one hundred billion cloaked cells (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, or 1×10¹¹ cloaked cells) can be administered near the kidney or injected subcutaneously.

Following administration of the cloaked cells to a patient, a practitioner of skill in the art can evaluate the subject's immune response to the donor alloantigen by a variety of methods. For example, a practitioner can monitor the response of an immune cell from the subject to the donor alloantigen in vitro. A finding that the subject's immune system does not mount an immune response against the donor alloantigen after administration of the cloaked cells indicates that the subject's immune system has been tolerized to the donor alloantigen. The physician may then proceed to transplant the kidney from the organ donor into the subject with the expectation that the subject is at low risk of rejecting the kidney and may not require immunosuppressive medication or may require a reduced dose of immunosuppressive medication relative to the standard dose. A finding of an equivalent outcome in transplant rejection as that observed in subjects administered immunosuppressive agent(s) indicates that the cloaked cell induced immune tolerance to the transplanted organ. Subsequent doses of cloaked cells can be determined and administered as needed. In some embodiments, the cloaked cells are further modified prior to administration to the subject to allow for control of their proliferation by linking the expression of a CDL to an ALINK or EARC (e.g., a negative selectable marker or an exogenous inducible transcriptional activator). For example, the cloaked cells can be modified to contain homozygous ALINKs (e.g., HSV-TK systems) in two CDL loci (e.g., Cdk1 and Top2A). After subcutaneous injection of the cloaked cells that express a polypeptide comprising a donor alloantigen and contain a mechanism for controlling proliferation, a practitioner of skill in the art can monitor the size of the cloaked subcutaneous tissue. If it appears that the cloaked subcutaneous tissue is becoming tumorigenic, the practitioner can administer ganciclovir to the subject to ablate the proliferating cloaked cells. The mechanism for controlling proliferation can also be used to ablate all of the cloaked cells after immune tolerance has been induced in the subject and the cells are no longer needed.

### Example 3: Administration of cloaked cells expressing MOG to a subject with Multiple Sclerosis

According to the methods disclosed herein, a physician of skill in the art can treat a subject, such as a human subject, with MS by inducing tolerance to MOG, an antigen implicated in the T cell damage to oligodendrocytes that occurs during the course of the disease. To this end, a physician of skill in the art can administer to the human subject cloaked cells (e.g., cloaked stem cells, cloaked oligodendrocytes, or cloaked stem cells that have been differentiated into oligodendrocytes) that express one or more (e.g., one, two, three, four, five, six, seven, or all eight) of PD-L1, HLA-G (H2-M3), Cd47, Cd200, FASLG (FasL), Ccl21 (Ccl21b), Mfge8, and Serpin B9 (Spi6) under the control of a constitutive promoter (e.g., CMV or CAG) and MOG under the control of a constitutive promoter (e.g., CMV or CAG). The cloaked cells may be administered to the subject, for example, by subcutaneous injection (e.g., to create a cloaked subcutaneous tissue), to treat MS. One million to three billion cloaked cells expressing MOG (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸, 2×10⁸, 3×10⁸, 4×10⁸, 5×10⁸, 6×10⁸, 7×10⁸, 8×10⁸, 9×10⁸, 1×10⁹, 2×109, or 3×10⁹ cloaked cells) can be administered subcutaneously.

Following administration of the cloaked cells to a subject, a practitioner of skill in the art can monitor the subject's improvement in response to the therapy by a variety of methods. For example, a physician can monitor symptoms of MS (e.g., tremor, coordination, gait, speech, vision, or feelings of numbness or tingling) in a subject with primary-progressive or relapsing-remitting MS, or a physician can monitor the occurrence of relapses in a subject with relapsing-remitting MS. A finding that the subject's symptoms of MS improve or stabilize (e.g., a finding that the symptoms do not worsen or progress), or a finding that the time between relapses increases or the occurrence of relapses decreases compared to measurements taken prior to administration of the cloaked cells indicates that the subject is responding favorably to the treatment. Subsequent doses can be determined and administered as needed.

In some embodiments, the cloaked cells are further modified prior to administration to the subject to allow for control of their proliferation by linking the expression of a CDL to an ALINK or EARC (e.g., a negative selectable marker or an exogenous inducible transcriptional activator). For example, the cloaked cells can be modified to contain homozygous ALINKs (e.g., HSV-TK systems) in two CDL loci (e.g., Cdk1 and Top2A). After subcutaneous injection of the cloaked cells that express MOG and contain a mechanism for controlling proliferation, a practitioner of skill in the art can monitor the size of the cloaked subcutaneous tissue. If it appears that the cloaked subcutaneous tissue is becoming tumorigenic, the practitioner can administer ganciclovir to the subject to ablate the proliferating cloaked cells. The mechanism for controlling proliferation can also be used to ablate all of the cloaked cells after immune tolerance has been induced in the subject and the cells are no longer needed.

### Example 4: Administration of Cloaked Cells Expressing Insulin or GAD65 to a Subject with Type 1 Diabetes

According to the methods disclosed herein, a physician of skill in the art can treat a subject, such as a human subject, with Type 1 diabetes by inducing immune tolerance to an antigen associated with damage to pancreatic beta cells by immune cells (e.g., insulin and/or GAD65). To this end, a physician of skill in the art can administer to the human subject cloaked cells (e.g., cloaked stem cells, cloaked pancreatic beta cells, or cloaked stem cells that have been differentiated into pancreatic beta cells) that express one or more (e.g., one, two, three, four, five, six, seven, or all eight) of PD-L1, HLA-G (H2-M3), Cd47, Cd200, FASLG (FasL), Ccl21 (Ccl21b), Mfge8, and Serpin B9 (Spi6) under the control of a constitutive promoter (e.g., CMV or CAG) and insulin and/or GAD65 under the control of a constitutive promoter (e.g., CMV or CAG). The cloaked cells may be administered to the subject, for example, by subcutaneous injection (e.g., to create a cloaked subcutaneous tissue), to treat Type 1 diabetes. One million to three billion cloaked cells expressing insulin and/or GAD65 (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸, 2×10⁸, 3×10⁸, 4×10⁸, 5×10⁸, 6×10⁸, 7×10⁸, 8×10⁸, 9×10⁸, 1×10⁹, 2×10⁹, or 3×10⁹ cloaked cells) can be administered subcutaneously or near or to the pancreas.

Following administration of the cloaked cells to a subject, a practitioner of skill in the art can monitor the subject's improvement in response to the therapy by a variety of methods. For example, a physician can monitor insulin levels or symptoms of Type 1 diabetes (e.g., unintended weight loss, fatigue, or blurred vision) using standard approaches. A finding that the subject's insulin levels are increased or the symptoms of Type 1 diabetes are reduced compared to measurements taken prior to administration of the cloaked cells indicates that the subject is responding favorably to the treatment. Subsequent doses can be determined and administered as needed.

In some embodiments, the cloaked cells are further modified prior to administration to the subject to allow for control of their proliferation by linking the expression of a CDL to an ALINK or EARC (e.g., a negative selectable marker or an exogenous inducible transcriptional activator). For example, the cloaked cells can be modified to contain homozygous ALINKs (e.g., HSV-TK systems) in two CDL loci (e.g., Cdk1 and Top2A). After subcutaneous injection of the cloaked cells that express insulin and/or GAD65 and contain a mechanism for controlling proliferation, a practitioner of skill in the art can monitor the size of the cloaked subcutaneous tissue. If it appears that the cloaked subcutaneous tissue is becoming tumorigenic, the practitioner can administer ganciclovir to the subject to ablate the proliferating cloaked cells. The mechanism for controlling proliferation can also be used to ablate all of the cloaked cells after immune tolerance has been induced in the subject and the cells are no longer needed.

### Example 5: Administration of Cloaked Cells Followed by Administration of Uncloaked Cells Expressing a Cancer Antigen to Induce Immunity to the Cancer Antigen

According to the methods disclosed herein, a physician of skill in the art can treat a subject, such as a human subject, at risk of developing cancer (e.g., breast cancer) by promoting the production of antibodies against a cancer antigen (e.g., a breast cancer antigen) by the immune system of the subject. To this end, a physician of skill in the art can administer to the human subject cloaked cells (e.g., cloaked allogeneic stem cells) that express one or more (e.g., one, two, three, four, five, six, seven, or all eight) of PD-L1, HLA-G (H2-M3), Cd47, Cd200, FASLG (FasL), Ccl21 (Ccl21b), Mfge8, and Serpin B9 (Spi6) under the control of a constitutive promoter (e.g., CMV or CAG). The cloaked cells may be administered to the subject, for example, by subcutaneous injection (e.g., to create a cloaked subcutaneous tissue), to induce immune tolerance to the cloaked cells. One million to three billion cloaked cells (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸, 2×10⁸, 3×10⁸, 4×10⁸, 5×10⁸, 6×10⁸, 7×10⁸, 8×10⁸, 9×10⁸, 1×10⁹, 2×10⁹, or 3×10⁹ cloaked cells) can be administered subcutaneously.

Following administration of the cloaked cells to a subject, a practitioner of skill in the art can measure an immune response of the subject to an uncloaked cell of the same type (e.g., measure a response of an immune cell isolated from the subject to an uncloaked cell of the same type in vitro). If the subject exhibits immune tolerance to the uncloaked cell (e.g., if the immune cell from the subject does not mount an immune response against the uncloaked cell), an uncloaked cell of the same type that is modified to express a polypeptide containing a cancer antigen (e.g., a breast cancer antigen) under the control of a constitutive promoter (e.g., CMV or CAG) can be administered to the subject. The uncloaked cells expressing a cancer antigen may be administered to the subject, for example, by subcutaneous injection (e.g., to create an uncloaked subcutaneous tissue), to induce an immune response to the cancer antigen (e.g., to promote the production of antibodies directed against the cancer antigen). One million to three billion uncloaked cells (e.g., 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, or 1×10⁸, 2×10⁸, 3×10⁸, 4×10⁸, 5×10⁸, 6×10⁸, 7×10⁸, 8×10⁸, 9×10⁸, 1×10⁹, 2×10⁹, or 3×10⁹ cloaked cells) can be administered subcutaneously or to or near the breast of the subject. A practitioner of skill in the art can determine whether an immune response has been stimulated in the subject by collecting a blood sample from the subject and assaying for the presence of antibodies directed against the cancer antigen. A finding that the blood sample from the subject contains antibodies directed against the cancer antigen indicates that the uncloaked cell has stimulated an immune response to the cancer antigen. Subsequent doses can be determined and administered as needed.

In some embodiments, the cloaked or uncloaked cells are further modified prior to administration to the subject to allow for control of their proliferation by linking the expression of a CDL to an ALINK or EARC (e.g., a negative selectable marker or an exogenous inducible transcriptional activator). For example, the cloaked or uncloaked cells can be modified to contain homozygous ALINKs (e.g., HSV-TK systems) in two CDL loci (e.g., Cdk1 and Top2A). After subcutaneous injection of the cloaked or uncloaked cells that contain a mechanism for controlling proliferation, a practitioner of skill in the art can monitor the size of the cloaked subcutaneous tissue. If it appears that the cloaked or uncloaked subcutaneous tissue is becoming tumorigenic, the practitioner can administer ganciclovir to the subject to ablate the proliferating cloaked or uncloaked cells. The mechanism for controlling proliferation can also be used to ablate all of the cloaked cells after immune tolerance has been induced in the subject and the cells are no longer needed, or to ablate the uncloaked cells after the subject's immune system has produced antibodies directed to the cancer antigen.

### OTHER EMBODIMENTS

Although the disclosure has been described with reference to certain specific embodiments, various modifications thereof will be apparent to those skilled in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all prior art recited herein are incorporated herein by reference in their entirety.

1. A cell genetically modified to comprise at least one mechanism for inducing immune tolerance to an antigen when administered to a subject, the genetically modified cell comprising a set of transgenes comprising two or more of the following genes: PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and a transgene encoding a polypeptide comprising an antigen.

2. The cell of claim 1, wherein the set of transgenes comprises three, four, five, six, seven, or all eight of the following genes: PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6.

3. The cell of claim 1, wherein the set of transgenes comprises PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, and Serpin B9 or Spi6.

4. The cell of any one of claims 1-3, further comprising one or more of the following transgenes: TGF-β, Cd73, Cd39, Lag3, Il1r2, Ackr2, Tnfrsf22, Tnfrs23, Tnfrsf10, Dad1, and IFNγR1 d39 or a gene encoding a biologic that acts as an agonist of TGF-β, Cd73, Cd39, Lag3, Il1r2, Ackr2, Tnfrsf22, Tnfrs23, Tnfrsf10, Dad1, or IFNγR1 d39.

5. The cell of claim 4, wherein the TGF-β or biologic is local acting in the graft environment.

6. The cell of any one of claims 1-5, wherein the cell is a stem cell, an autologous cell, a somatic cell, a germ cell, a syngeneic cell, or a cell amenable for genome editing.

7. The cell of claim 6, wherein the cell is an embryonic stem cell, a pluripotent stem cell, an induced pluripotent stem cell (iPSC), a hematopoietic stem cell, a mesenchymal stem cell, an endothelial stem cell, an epithelial stem cell, an adipose stem or progenitor cell, a germline stem cell, a lung stem or progenitor cells, a mammary stem cell, an olfactory adult stem cell, a hair follicle stem cell, an intestinal stem or progenitor cell, a multipotent stem cell, an amniotic stem cell, a cord blood stem cell, a neural stem or progenitor cell, an adult stem cell, a somatic stem cell, a tissue-specific stem cell, a totipotent stem cell, a fibroblast, a monocytic precursor, a B cell, an exocrine cell, a pancreatic progenitor, an endocrine progenitor, a hepatoblast, a myoblast, a preadipocyte, a hepatocyte, a chondrocyte, a smooth muscle cell, a K562 human erythroid leukemia cell line, a bone cell, a synovial cell, a tendon cell, a ligament cell, a meniscus cell, an adipose cell, a dendritic cell, a neutrophil, a basophil, a mast cell, a monocyte, an innate lymphoid cell, a natural killer cell, a skeletal muscle cell, a cardiac muscle cell, an erythroid-megakaryocytic cell, an eosinophil, a macrophage, a T cell, an islet beta-cell, a neuron, a cardiomyocyte, a blood cell, an exocrine progenitor, a ductal cell, an acinar cell, an alpha cell, a beta cell, a delta cell, a PP cell, a cholangiocyte, a white or brown adipocyte, a hormone-secreting cell, an epidermal keratinocyte, an epithelial cell, a kidney cell, a skeletal joint synovium cell, a periosteum cell, a perichondrium cell, a cartilage cell, an endothelial cell, a pericardium cell, a meningeal cell, a keratinocyte precursor cell, a keratinocyte stem cell, a pericyte, a glial cell, an ependymal cell, a cell isolated from an amniotic or placental membrane, a serosal cell, or a cell derived from skin, heart, brain or spinal cord, liver, lung, kidney, pancreas, bladder, bone marrow, spleen, intestine, or stomach.

8. The cell of any one of claims 1-7, wherein the antigen is a donor alloantigen.

9. The cell of claim 8, wherein the donor alloantigen is a blood group antigen or a histocompatibility antigen.

10. The cell of any one of claims 1-7, wherein the antigen is an antigen associated with an autoimmune disease or condition.

11. The cell of claim 10, wherein the antigen is a self-antigen to which the subject's immune system mounts an aberrant immune response.

12. The cell of claim 10, wherein the antigen is selected from an antigen listed in Table 2.

13. The cell of any one of claims 1-7, wherein the antigen is a food antigen or an allergen antigen.

14. The cell of any one of claims 1-13, wherein the antigen is not endogenous to the cell.

15. The cell of any one of claims 1-14, wherein the transgene encoding the polypeptide comprising the antigen is incorporated into the genome of the cell at a non-endogenous locus.

16. The cell of any one of claims 1-15, wherein the cell further comprises at least one mechanism for controlling cell proliferation, the genetically modified cell comprising:
a) a genetic modification of one or more cell division locus/loci (CDL), the CDL being one or more endogenous or exogenous loci whose transcription product(s) is expressed by dividing cells, the genetic modification being one or more of:
i) an ablation link (ALINK) system that comprises a DNA sequence encoding a negative selectable marker that is transcriptionally linked to a DNA sequence encoding the CDL; and
ii) an exogenous activator of regulation of a CDL (EARC) system that comprises an inducible activator-based gene expression system that is operably linked to the CDL.

17. The cell of claim 16, wherein the genetic modification of the CDL comprises performing targeted replacement of the CDL with one or more of:
a) a DNA vector comprising the ALINK system;
b) a DNA vector comprising the EARC system; and
c) a DNA vector comprising the ALINK system and the EARC system;
wherein the ALINK and/or EARC systems are each operably linked to the CDL.

18. The cell of claim 16 or 17, wherein the genetic modification of the CDL comprising the ALINK system is homozygous, heterozygous, hemizygous or compound heterozygous and/or wherein the genetic modification of the CDL comprising the EARC system results in activation of the CDL solely by an inducer of the inducible activator-based gene expression system.

19. The cell of any one of claims 16-18, wherein the CDL encodes a gene product that functions in one or more of: cell cycle, DNA replication, RNA transcription, protein translation, and metabolism.

20. The cell of claim 19, wherein the CDL is one or more of Cdk1/CDK1, Top2A/TOP2A, Cenpa/CENPA, Birc5/BIRC5, and Eef2/EEF2, preferably wherein the CDL is Cdk1 or CDK1.

21. The cell of claim 20, wherein the CDL is two or more of Cdk1/CDK1, Top2A/TOP2A, Cenpa/CENPA, Birc5/BIRC5, and Eef2/EEF2, preferably wherein the CDL is Cdk1/CDK1 and Top2A/TOP2A or Cdk1/CDK1 and Eef2/EEF2.

22. The cell of any one of claims 16-21, wherein the ALINK system comprises a herpes simplex virus-thymidine kinase/ganciclovir system, a cytosine deaminase/5-fluorocytosine system, a carboxyl esterase/irinotecan system or an iCasp9/AP1903 system, preferably wherein the ALINK system is a herpes simplex virus-thymidine kinase/ganciclovir system.

23. The cell of any one of claims 16 to 21, wherein the EARC system is a dox-bridge system, a cumate switch inducible system, an ecdysone inducible system, a radio wave inducible system, or a ligand-reversible dimerization system, preferably wherein the EARC system is a dox-bridge system.

24. The cell of any one of claims 1-23, wherein one or more of PD-L1, HLA-G or H2-M3, Cd47, Cd200, FASLG or FasL, Ccl21 or Ccl21b, Mfge8, and Serpin B9 or Spi6 is expressed at a level that is equal to or greater than the expression level of the corresponding endogenous gene in an activated leukocyte.

25. The cell of claim 24, wherein all eight of PD-L1, HLA-G or H2-M3, Cd47, Cd200, FASLG or FasL, Ccl21 or Ccl21b, Mfge8, and Serpin B9 or Spi6 are expressed at a level that is equal to or greater than the expression level of the corresponding endogenous gene in an activated leukocyte.

26. The cell of any one of claims 1-25, wherein one or more of PD-L1, HLA-G or H2-M3, Cd47, Cd200, FASLG or FasL, Ccl21 or Ccl21b, Mfge8, and Serpin B9 or Spi6 is expressed at a level that is in the top 5% of gene expression for all genes in the genome of the cell.

27. The cell of claim 26, wherein all eight of PD-L1, HLA-G or H2-M3, Cd47, Cd200, FASLG or FasL, Ccl21 or Ccl21b, Mfge8, and Serpin B9 or Spi6 are expressed at a level that is in the top 5% of gene expression for all genes in the genome of the cell.

28. The cell of any one of claims 1-27, wherein the PD-L1 transgene encodes a protein having at least 85% identity to the amino acid sequence of SEQ ID NO: 11 or SEQ ID NO: 12.

29. The cell of any one of claims 1-28, wherein the HLA-G or H2-M3 transgene encodes a protein having at least 85% identity to the amino acid sequence of SEQ ID NO: 16 or SEQ ID NO: 15.

30. The cell of any one of claims 1-29, wherein the Cd47 transgene encodes a protein having at least 85% identity to the amino acid sequence of SEQ ID NO: 3 or SEQ ID NO: 4.

31. The cell of any one of claims 1-30, wherein the CD200 transgene encodes a protein having at least 85% identity to the amino acid sequence of SEQ ID NO: 5 or SEQ ID NO: 6.

32. The cell of any one of claims 1-31, wherein the FASLG or FasL transgene encodes a protein having at least 85% identity to the amino acid sequence of SEQ ID NO: 10 or SEQ ID NO: 9.

33. The cell of any one of claims 1-32, wherein the Ccl21 or Ccl21b transgene encodes a protein having at least 85% identity to the amino acid sequence of SEQ ID NO: 2 or SEQ ID NO: 1.

34. The cell of any one of claims 1-33, wherein the Mfge8 transgene encodes a protein having at least 85% identity to the amino acid sequence of SEQ ID NO: 13 or SEQ ID NO: 14.

35. The cell of any one of claims 1-34, wherein the Serpin B9 or Spi6 transgene encodes a protein having at least 85% identity to the amino acid sequence of SEQ ID NO: 8 or SEQ ID NO: 7.

36. The cell of any one of claims 4-35, wherein the IFNγR1 d39 transgene encodes a protein having at least 85% identity to the amino acid sequence of SEQ ID NO: 17.

37. The cell of any one of claims 1-36, wherein the two or more transgenes are operably linked to a constitutive promoter.

38. The cell of any one of claims 1-37, wherein the transgene encoding the polypeptide comprising the antigen is operably linked to a constitutive promoter.

39. The cell of claim 37 or 38, wherein the constitutive promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

40. The cell of any one of claims 1-37, wherein the polypeptide comprising the antigen is expressed using an inducible expression system selected from the group consisting of a tetracycline response element, a light inducible system, a radiogenetic system, a cumate switch inducible system, an ecdysone inducible system, a destabilization domain system, or a ligand-reversible dimerization system.

41. The cell of claim 40, wherein the cell comprises an EARC system, and wherein expression of the EARC system and the polypeptide comprising the antigen is controlled by two different inducible expression systems.

42. The cell of any one of claims 1-41, wherein the polypeptide comprising the antigen is expressed at a level that corresponds to the expression level of the polypeptide in a target cell.

43. The cell of claim 42, wherein the antigen is a donor alloantigen and the target cell is a donor cell.

44. The cell of claim 42, wherein the antigen is a self-antigen to which the subject's immune system mounts an aberrant immune response and the target cell is a cell from the subject that endogenously expresses the polypeptide.

45. The cell of any one of claims 1-41, wherein the polypeptide comprising the antigen is expressed at a level that corresponds to the expression level of a polypeptide encoded by a housekeeping gene.

46. The cell of claim 45, wherein the polypeptide encoded by the housekeeping gene is β-actin, GAPDH, or Rosa26.

47. A population of genetically modified cells, wherein each of the cells is a cell of any one of claims 1-46.

48. A composition comprising the cell of any one of claims 1-46.

49. A composition comprising the population of cells of claim 47.

50. The composition of claim 48 or 49, further comprising a pharmaceutically acceptable excipient.

51. A kit comprising the cell of any one of claims 1-46 or the composition of any one of claims 48-50.

52. A method of inducing immune tolerance in a subject to a donor alloantigen, comprising:
(i) providing a donor cell expressing a polypeptide comprising the donor alloantigen; and
(ii) expressing in the cell a set of transgenes comprising two or more of the following genes: PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and
(iii) administering the cell to the subject to induce immune tolerance to the donor alloantigen.

53. A method of inducing immune tolerance in a subject to a donor alloantigen, comprising:
(i) providing a cell; and
(ii) expressing in the cell a set of transgenes comprising two or more of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6;
(iii) modifying the cell to express a transgene encoding a polypeptide comprising the donor alloantigen; and
(iv) administering the cell to the subject to induce immune tolerance to the donor alloantigen.

54. The method of claim 52 or 53, wherein the donor alloantigen is a blood group antigen or a histocompatibility antigen.

55. The method of claim 53 or 54, wherein the polypeptide comprising the donor alloantigen is expressed at a level corresponding to the expression level of the polypeptide comprising the donor alloantigen in a donor cell.

56. The method of any one of claims 52-55, wherein the cell is administered to or near a body site that will receive an organ or tissue transplant from the donor, or wherein the cell is administered to a subcutaneous site.

57. The method of any one of claims 52-56, wherein the method further comprises measuring a response of an immune cell from the subject to the donor alloantigen after administration of the cell.

58. The method of any one of claims 52-57, wherein the method prepares the subject to receive an organ or tissue transplant from the donor.

59. The method of any one of claims 52-58, wherein the method further comprises transplanting an organ or tissue from the donor into the subject after inducing immune tolerance to the donor alloantigen.

60. The method of claim 59, wherein the subject does not require treatment with an immunosuppressive medication.

61. The method of claim 59, wherein the method further comprises administering to the subject an immunosuppressive medication.

62. The method of claim 61, wherein the immunosuppressive medication is administered at a dose that is reduced relative to a standard dose of the immunosuppressive medication administered to a recipient of an organ or tissue transplant.

63. A method of inducing immune tolerance in a subject to an antigen, comprising:
(i) providing a cell; and
(ii) expressing in the cell a set of transgenes comprising two or more of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6;
(iii) expressing in the cell a transgene encoding a polypeptide comprising the antigen; and
(iv) administering the cell to the subject to induce immune tolerance to the antigen.

64. The method of claim 63, wherein the antigen is an antigen associated with an autoimmune disease or condition.

65. A method of treating a subject having or at risk of developing an autoimmune disease or condition, comprising administering to the subject a cell modified to express a transgene encoding a polypeptide comprising an antigen associated with the autoimmune disease or condition and a set of transgenes comprising two or more of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6.

66. The method of claim 64 or 65, wherein the antigen is a self-antigen to which the subject's immune system mounts an aberrant immune response.

67. The method of claim 66, wherein the polypeptide comprising the self-antigen is expressed at a level that corresponds to the endogenous expression level of the polypeptide in a cell of the subject.

68. The method of any one of claims 63-67, wherein the antigen is selected from an antigen listed in Table 2.

69. The method of any one of claims 63-65, wherein the antigen is a food antigen or an allergen antigen.

70. The method of any one of claims 63-69, wherein the cell is administered to or near a body site associated with an autoimmune disease or condition, or wherein the cell is administered to a subcutaneous site.

71. The method of any one of claims 63-70, wherein the method further comprises measuring a response of an immune cell from the subject to the antigen after administration of the cell.

72. The cell of claim 10 or the method of claim 64 or 65, wherein the autoimmune disease or condition is multiple sclerosis (MS) and the polypeptide comprising the antigen is myelin oligodendrocyte glycoprotein (MOG).

73. The cell of claim 10 or the method of claim 64 or 65, wherein the autoimmune disease or condition is type 1 diabetes and the polypeptide comprising the antigen is insulin or glutamic acid decarboxylase 65 (GAD65).

74. A method of inducing an immune response in a subject to an antigen, comprising:
(i) administering to the subject a first cell expressing a set of transgenes comprising two or more of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and
(ii) administering to the subject a second cell of the same type as the first cell that is modified to express a transgene encoding a polypeptide comprising the antigen and that does not express the set of transgenes.

75. The method of claim 74, wherein the method further comprises measuring a response of an immune cell from the subject to the first cell before administration of the second cell.

76. The method of claim 74 or 75, wherein the second cell is administered after inducing immune tolerance in the subject to the first cell.

77. The method of any one of claims 74-76, wherein the antigen is a non-self antigen.

78. The method of claim 77, wherein the non-self antigen is a cancer antigen, a viral antigen, a bacterial antigen, a fungal antigen, or a parasitic antigen.

79. The method of claim 78, wherein the cancer antigen is derived from a leukemia, lymphoma, sarcoma, carcinoma, blastoma, myeloma, melanoma, neuroma, glioma, liver cancer, bone cancer, lung cancer, brain cancer, bladder cancer, gastrointestinal cancer, urogenital cancer, breast cancer cardiac cancer, gynecological cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, skin cancer, thyroid cancer, bone and soft tissue sarcoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrial cancer, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, Hodgkin lymphoma, hypopharyngeal cancer, islet cell tumors, pancreatic neuroendocrine tumors, Wilms tumor and other childhood kidney tumors, langerhans cell histiocytosis, small cell lung cancer, cutaneous T cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-Hodgkin lymphoma (NHL), non-small cell lung cancer (NSCLC), epithelial ovarian cancer, germ cell ovarian cancer, low malignant potential ovarian cancer, pancreatic neuroendocrine tumors, papillomatosis, paraganglioma, paranasal sinus and nasal cavity cancer, parathyroid cancer, penile cancer, pharyngeal cancer, stomach cancer, renal cancer, pelvic cancer, bronchus cancer, oropharyngeal cancer, larynx cancer, biliary tract cancer, a cancer of the central nervous system, a cancer of the respiratory system, and a cancer of the urinary system, cancer of the peritoneum, hepatocellular cancer, hepatoma, endometrial or uterine carcinoma, salivary gland carcinoma, hepatic carcinoma, anal carcinoma, penile carcinoma, post-transplant lymphoproliferative disorder (PTLD), bladder carcinoma, a pharynx cancer, a tumor of the tongue, a synovial cell sarcoma, a neuroblastoma, pheochromocytoma, pituitary tumor, pleuropulmonary blastoma, primary peritoneal cancer, rectal cancer, retinoblastoma, rhabdomyosarcoma, salivary gland cancer, Kaposi sarcoma, rhabdomyosarcoma, Sezary syndrome, small intestine cancer, soft tissue sarcoma, throat cancer, thymoma and thymic carcinoma, thyroid cancer, transitional cell cancer of the renal pelvis and ureter, urethral cancer, endometrial uterine cancer, uterine sarcoma, vaginal cancer, vulvar cancer, Waldenström macroglobulinemia, meningioma, malignant mesothelioma, or a virally induced cancer.

80. The method of claim 78, wherein the viral antigen is derived from a virus of a viral family selected from the group consisting of Retroviridae, Flaviviridae, Arenaviridae, Bunyaviridae, Filoviridae, Togaviridae, Poxviridae, Herpesviridae, Orthomyxoviridae, Coronaviridae, Rhabdoviridae, Paramyxoviridae, Picornaviridae, Hepadnaviridae, Papillomaviridae, Parvoviridae, Astroviridae, Polyomaviridae, Caliciviridae, and Reoviridae.

81. The method of claim 78, wherein the viral antigen is derived from human immunodeficiency virus (HIV), human papillomavirus (HPV), hepatitis A virus (Hep A), hepatitis B virus (HBV), hepatitis C virus (HCV), Variola major, Variola minor, monkeypox virus, measles virus, rubella virus, mumps virus, varicella zoster virus (VZV), poliovirus, rabies virus, Japanese encephalitis virus, herpes simplex virus (HSV), cytomegalovirus (CMV), rotavirus, influenza, Ebola virus, yellow fever virus, Zika virus, or Marburg virus.

82. The method of claim 78, 80, or 81, wherein the polypeptide comprising the viral antigen is Gag, Pol, Nef, Tat, Rev, Vif, Vpr, Vpu, 9D antigen, or Env.

83. The method of claim 78, wherein the bacterial antigen is derived from Acinetobacter spp., Bacteroides distasonis, Bacteroides fragilis, Bacteroides ovatus, Bacteroides thetaiotaomicron, Bacteroides uniformis, Bacteroides vulgatus, B. cepacia, Citrobacter freundii, Citrobacter koseri, Clostridium clostridioforme, Clostridium perfringens, C. sordellii, Enterobacter aerogenes, Enterobacter cloacae, Enterococcus faecalis, Enterococcus spp. Escherichia coli, Eubacterium lentum, Fusobacterium spp., Haemophilus influenza, Haemophilus parainfluenzae, Klebsiella pneumoniae, Klebsiella oxytoca, Legionella pneumophilia, Moraxella catarrhalis, Morganella morganii, Mycoplasma spp., Peptostreptococcus spp., Porphyromonas saccharolytica, Prevotella bivia, Proteus mirabilis, Proteus vulgaris, Providencia rettgeri, Providencia stuartii, Pseudomonas aeruginosa, Serratia marcescens, Streptococcus anginosus, Staphylococcus aureus, Staphylococcus epidermidis, Stenotrophomonas maltophilia, Streptococcus agalactiae, Streptococcus constellatus, Streptococcus pneumoniae, Streptococcus pyogenes, Streptococcus pyogenes, Mycobacterium tuberculosis, Mycobacterium bovis, Mycobacterium africanum, Mycobacterium microti, Mycobacterium leprae, Salmonella typhimurium, Francisella tularensis, Brucella, Burkholderia mallei, Yersinia pestis, Corynebacterium diphtheria, Neisseria meningitidis, Bordetella pertussis, Clostridium tetani, or Bacillus anthracis.

84. The method of claim 78 or 83, wherein the polypeptide comprising the bacterial antigen is 10.4, 85A, 85B, 86C, CFP-10, Rv3871, ESAT-6, O antigen, H antigen, K antigen, or protective antigen (PA).

85. The method of claim 78, wherein the parasitic antigen is derived from Toxoplasma gondii, Plasmodium falciparum, Plasmodium vivax, Plasmodium ovale, Plasmodium malariae, Trypanosoma spp., or Legionella spp.

86. The method of claim 78 or 85, wherein the polypeptide comprising the parasitic antigen is circumsporozoite (CS) protein, gamete surface protein Pfs230 or Pfs48/45, or Liver Specific Antigen 1 or 3 (LSA-1 or LSA-3).

87. The method of claim 78, wherein the fungal antigen is derived from a mold pathogen from phylum Ascomycota, phylum Basidomycota, phylum Chytridiomycota, phylum, or phylum Oomycota in the Stramenopila kingdom.

88. The method of claim 87, wherein the fungal antigen is derived from Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

89. The method of claim 78, 87, or 88, wherein the polypeptide comprising the fungal antigen is a cell wall mannoprotein or a surface-expressed glycoprotein.

90. The method of any one of claims 74-89, wherein the polypeptide comprising the antigen is expressed at a level that corresponds to the expression level of a polypeptide encoded by a housekeeping gene.

91. The cell of claim 90, wherein the polypeptide encoded by the housekeeping gene is B-actin, GAPDH, or Rosa26.

92. The method of any one of claims 74-91, wherein the first cell and/or the second cell is administered to a subcutaneous site.

93. The method of any one of claims 74-92, wherein the method further comprises assessing a blood sample from the subject for antibodies directed against the antigen.

94. The method of any one of claims 74-93, wherein the method immunizes the subject to the antigen.

95. The method of any one of claims 74-94, wherein the method induces the production of antibodies directed against the antigen by the immune system of the subject.

96. The method of any one of claims 52-95, wherein the set of transgenes comprises three, four, five, six, seven, or all eight of the following genes: PD-L1, HLA-G or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6.

97. The method of any one of claims 52-95, wherein the set of transgenes comprises PD-L1, HLA-G or H2-M3, Cd47, Cd200, FASLG or FasL, Ccl21 or Ccl21b, Mfge8, and Serpin B9 or Spi6 or a gene encoding a gene encoding a biologic that acts as an agonist of PD-L1, HLA-G or H2-M3, Cd47, Cd200, FASLG or FasL, Ccl21 or Ccl21b, Mfge8, and Serpin B9 or Spi6.

98. The method of any one of claims 52-97, further comprising expressing one or more of the following transgenes: TGF-β, Cd73, Cd39, Lag3, Il1r2, Ackr2, Tnfrsf22, Tnfrs23, Tnfrsf10, Dad1, and IFNγR1 d39 or a gene encoding a biologic that acts as an agonist of TGF-β, Cd73, Cd39, Lag3, Il1r2, Ackr2, Tnfrsf22, Tnfrs23, Tnfrsf10, Dad1, or IFNγR1 d39.

99. The method of claim 98, wherein the TGF-β or biologic is local acting in the graft environment.

100. The method of any one of claims 52-99, wherein the cell is a stem cell, a germ cell, a somatic cell, a syngeneic cell, or a cell amenable for genome editing.

101. The method of claim 100, wherein the cell is an embryonic stem cell, an induced pluripotent stem cell, an induced pluripotent stem cell (iPSC), a hematopoietic stem cell, a mesenchymal stem cell, an endothelial stem cell, an epithelial stem cell, an adipose stem or progenitor cell, a germline stem cell, a lung stem or progenitor cells, a mammary stem cell, an olfactory adult stem cell, a hair follicle stem cell, an intestinal stem or progenitor cell, a multipotent stem cell, an amniotic stem cell, a cord blood stem cell, a neural stem or progenitor cell, an adult stem cell, a somatic stem cell, a tissue-specific stem cell, a totipotent stem cell, a fibroblast, a monocytic precursor, a B cell, an exocrine cell, a pancreatic progenitor, an endocrine progenitor, a hepatoblast, a myoblast, a preadipocyte, a hepatocyte, a chondrocyte, a smooth muscle cell, a K562 human erythroid leukemia cell line, a bone cell, a synovial cell, a tendon cell, a ligament cell, a meniscus cell, an adipose cell, a dendritic cell, a neutrophil, a basophil, a mast cell, a monocyte, an innate lymphoid cell, a natural killer cell, a skeletal muscle cell, a cardiac muscle cell, an erythroid-megakaryocytic cell, an eosinophil, a macrophage, a T cell, an islet beta-cell, a neuron, a cardiomyocyte, a blood cell, an exocrine progenitor, a ductal cell, an acinar cell, an alpha cell, a beta cell, a delta cell, a PP cell, a cholangiocyte, a white or brown adipocyte, a hormone-secreting cell, an epidermal keratinocyte, an epithelial cell, a kidney cell, a skeletal joint synovium cell, a periosteum cell, a perichondrium cell, a cartilage cell, an endothelial cell, a pericardium cell, a meningeal cell, a keratinocyte precursor cell, a keratinocyte stem cell, a pericyte, a glial cell, an ependymal cell, a cell isolated from an amniotic or placental membrane, a serosal cell, or a cell derived from skin, heart, brain or spinal cord, liver, lung, kidney, pancreas, bladder, bone marrow, spleen, intestine, or stomach.

102. The method of any one of claims 53-73, wherein the cell is an autologous cell.

103. The method of any one of claims 52-102, wherein the cell further comprises a genetic modification that allows for the control of cell proliferation, the genetic modification comprising:
a) a genetic modification of one or more cell division locus/loci (CDL), the CDL being one or more endogenous or exogenous loci whose transcription product(s) is expressed by dividing cells, the genetic modification being one or more of:
i) an ablation link (ALINK) system that comprises a DNA sequence encoding a negative selectable marker that is transcriptionally linked to a DNA sequence encoding the CDL; and
ii) an exogenous activator of regulation of a CDL (EARC) system that comprises an inducible activator-based gene expression system that is operably linked to the CDL.

104. The method of claim 103, wherein the genetic modification of the CDL comprises performing targeted replacement of the CDL with one or more of:
a) a DNA vector comprising the ALINK system;
b) a DNA vector comprising the EARC system; and
c) a DNA vector comprising the ALINK system and the EARC system;
wherein the ALINK and/or EARC systems are each operably linked to the CDL.

105. The method of claim 103 or 104, wherein the genetic modification of the CDL comprising the ALINK system is homozygous, heterozygous, hemizygous or compound heterozygous and/or wherein the genetic modification of the CDL comprising the EARC system results in activation of the CDL solely by an inducer of the inducible activator-based gene expression system.

106. The method of any one of claims 103-105, wherein the CDL encodes a gene product that functions in one or more of: cell cycle, DNA replication, RNA transcription, protein translation, and metabolism.

107. The method of claim 106, wherein the CDL is one or more of Cdk1/CDK1, Top2A/TOP2A, Cenpa/CENPA, Birc5/BIRC5, and Eef2/EEF2, preferably wherein the CDL is Cdk1 or CDK1.

108. The method of claim 107, wherein the CDL is two or more of Cdk1/CDK1, Top2A/TOP2A, Cenpa/CENPA, Birc5/BIRC5, and Eef2/EEF2, preferably wherein the CDL is Cdk1/CDK1 and Top2A/TOP2A or Cdk1/CDK1 and Eef2/EEF2.

109. The method of any one of claims 103-108, wherein the ALINK system comprises a herpes simplex virus-thymidine kinase/ganciclovir system, a cytosine deaminase/5-fluorocytosine system, a carboxyl esterase/irinotecan system or an iCasp9/AP1903 system, preferably wherein the ALINK system is a herpes simplex virus-thymidine kinase/ganciclovir system.

110. The method of any one of claims 103-108, wherein the EARC system is a dox-bridge system, a cumate switch inducible system, an ecdysone inducible system, a radio wave inducible system, or a ligand-reversible dimerization system, preferably wherein the EARC system is a dox-bridge system.

111. The method of any one of claims 103-110, wherein the method further comprises i) permitting proliferation of the cell comprising the ALINK system by maintaining the cell comprising the ALINK system in the absence of an inducer of the negative selectable marker or ablating and/or inhibiting proliferation of the cell comprising the ALINK system by exposing the cell comprising the ALINK system to the inducer of the negative selectable marker; and/or
ii) permitting proliferation of the cell comprising the EARC system by exposing the cell comprising the EARC system to an inducer of the inducible activator-based gene expression system or preventing or inhibiting proliferation of the cell comprising the EARC system by maintaining the cell comprising the EARC system in the absence of the inducer of the inducible activator-based gene expression system.

112. The method of any one of claims 52-111, wherein the method further comprises removing the cell expressing the polypeptide comprising the antigen after the immune system of the subject has responded to the antigen.

113. The method of claim 112, wherein the cell is removed surgically.

114. The method of claim 112, wherein the cell comprises an ALINK system, and wherein the cell is removed by ablating the cell comprising the ALINK system by exposing the cell comprising the ALINK system to the inducer of the negative selectable marker.

115. The method of any one of claims 52-114, wherein one or more of PD-L1, HLA-G or H2-M3, Cd47, Cd200, FASLG or FasL, Ccl21 or Ccl21b, Mfge8, and Serpin B9 or Spi6 is expressed at a level that is equal to or greater than the expression level of the corresponding endogenous gene in an activated leukocyte.

116. The method of claim 115, wherein all eight of PD-L1, HLA-G or H2-M3, Cd47, Cd200, FASLG or FasL, Ccl21 or Ccl21b, Mfge8, and Serpin B9 or Spi6 are expressed at a level that is equal to or greater than the expression level of the corresponding endogenous gene in an activated leukocyte.

117. The method of any one of claims 52-116, wherein one or more of PD-L1, HLA-G or H2-M3, Cd47, Cd200, FASLG or FasL, Ccl21 or Ccl21b, Mfge8, and Serpin B9 or Spi6 is expressed at a level that is in the top 5% of gene expression for all genes in the genome of the cell.

118. The method of claim 117, wherein all eight of PD-L1, HLA-G or H2-M3, Cd47, Cd200, FASLG or FasL, Ccl21 or Ccl21b, Mfge8, and Serpin B9 or Spi6 are expressed at a level that is in the top 5% of gene expression for all genes in the genome of the cell.

119. The method of any one of claims 52-118, wherein the PD-L1 transgene encodes a protein having at least 85% identity to the amino acid sequence of SEQ ID NO: 11 or SEQ ID NO: 12.

120. The method of any one of claims 52-119, wherein the HLA-G or H2-M3 transgene encodes a protein having at least 85% identity to the amino acid sequence of SEQ ID NO: 16 or SEQ ID NO: 15.

121. The method of any one of claims 52-120, wherein the Cd47 transgene encodes a protein having at least 85% identity to the amino acid sequence of SEQ ID NO: 3 or SEQ ID NO: 4.

122. The method of any one of claims 52-121, wherein the CD200 transgene encodes a protein having at least 85% identity to the amino acid sequence of SEQ ID NO: 5 or SEQ ID NO: 6.

123. The method of any one of claims 52-122, wherein the FASLG or FasL transgene encodes a protein having at least 85% identity to the amino acid sequence of SEQ ID NO: 10 or SEQ ID NO: 9.

124. The method of any one of claims 52-123, wherein the Ccl21 or Ccl21b transgene encodes a protein having at least 85% identity to the amino acid sequence of SEQ ID NO: 2 or SEQ ID NO: 1.

125. The method of any one of claims 52-124, wherein the Mfge8 transgene encodes a protein having at least 85% identity to the amino acid sequence of SEQ ID NO: 13 or SEQ ID NO: 14.

126. The method of any one of claims 52-125, wherein the Serpin B9 or Spi6 transgene encodes a protein having at least 85% identity to the amino acid sequence of SEQ ID NO: 8 or SEQ ID NO: 9.

127. The method of any one of claims 98-126, wherein the IFNγR1 d39 transgene encodes a protein having at least 85% identity to the amino acid sequence of SEQ ID NO: 17.

128. The method of any one of 52-127, wherein the two or more transgenes are operably linked to a constitutive promoter.

129. The method of claim 128, wherein the constitutive promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

130. The method of any one of claims 52-129, wherein the polypeptide comprising the antigen is expressed using an inducible expression system selected from the group consisting of a tetracycline response element, a light inducible system, a radiogenetic system, a cumate switch inducible system, an ecdysone inducible system, a destabilization domain system, or a ligand-reversible dimerization system.

131. The method of claim 130, wherein the cell comprises an EARC system, and wherein expression of the EARC system and the polypeptide comprising the antigen is controlled by two different inducible expression systems.

132. The method of any one of claims 52-129, wherein the polypeptide comprising the antigen is expressed using a constitutive promoter selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

133. The method of any one of claims 52-132, wherein the cells are administered intravenously, subcutaneously, intramuscularly, percutaneously, intradermally, parenterally, intraarterially, intravascularly, or by perfusion.

134. The method of any one of claims 52-132, wherein the cells are administered as a tissue.

135. The method of claim 134, wherein the tissue is administered with a gel, biocompatible matrix, or cellular scaffold.

136. The method of any one of claims 52-132, wherein the cells are administered in an amount of 25,000 to 5,000,000,000 cells.

137. The method of any one of claims 52-135, wherein the cells are administered in an amount of 800,000,000 to 3,000,000,000 cells.

138. The method of any one of claims 52-137, wherein the method further comprises administering an additional therapeutic agent.

139. The method of claim 138, wherein the additional therapeutic agent is administered after administration of the cells.

140. The method of claim 139, wherein the additional therapeutic agent is administered concurrently with administration of the cells.

141. The method of any one of claims 138-140, wherein the additional therapeutic agent is an immunosuppressive agent, a disease-modifying anti-rheumatic drug (DMARD), a biologic response modifier (a type of DMARD), a corticosteroid, or a nonsteroidal anti-inflammatory medication (NSAID), prednisone, prednisolone, methylprednisolone, methotrexate, hydroxychloroquine, sulfasalazine, leflunomide, cyclophosphamide, azathioprine, tofacitinib, adalimumab, abatacept, anakinra, kineret, certolizumab, etanercept, golimumab, infliximab, rituximab or tocilizumab, 6-mercaptopurine, 6-thioguanine, abatacept, adalimumab, alemtuzumab, an aminosalicylate, an antibiotic, an anti-histamine, Anti-TNFα, azathioprine, belimumab, beta interferon, a calcineurin inhibitor, certolizumab, a corticosteroid, cromolyn, cyclosporin A, cyclosporine, dimethyl fumarate, etanercept, fingolimod, fumaric acid esters, glatiramer acetate, golimumab, hydroxyurea, IFNγ, IL-11, leflunomide, leukotriene receptor antagonist, long-acting beta2 agonist, mitoxantrone, mycophenolate mofetil, natalizumab, ocrelizumab, pimecrolimus, a probiotic, a retinoid, salicylic acid, short-acting beta2 agonist, sulfasalazine, tacrolimus, teriflunomide, theophylline, tocilizumab, ustekinumab, vedolizumab, interferon beta-1b, gliatrimer acetate, daclizumab, teriflunomide, fingolimod, dimethyl fumarate, alemtuzumab, mitoxantrone, ocrelizumab, natalizumab, an antiviral compound, a nucleoside-analog reverse transcriptase inhibitor (NRTI), a non-nucleoside reverse transcriptase inhibitor (NNRTI), a protease inhibitor, an antibacterial compound, an antifungal compound, an antiparasitic compound, insulin, a sulfonylurea, a biguanide, a meglitinide, a thiazolidinedione, a DPP-4 inhibitor, an SGLT2 inhibitor, an alpha-glucosidase inhibitor, a bile acid sequestrant, aspirin, a dietary regimen, an immunosuppressive agent, a checkpoint inhibitor, a chemotherapeutic drug, a biologic drug, radiation therapy, cryotherapy, hyperthermia, surgical excision or tumor tissue, or an anti-cancer vaccine.

142. The method of any one of claims 1-141 wherein the subject is a mammal.

143. The method of claim 142, wherein the mammal is a mouse.

144. The method of claim 142, wherein the mammal is a human.

145. The cell of any one of claims 1-46 for use in a method of inducing immune tolerance in a subject to an antigen.

146. The cell of any one of claims 1-7, 10-12, and 14-46 for use in a method of treating a subject having or at risk of developing an autoimmune disease or condition.

147. Use of a combination of a first cell and a second cell for inducing an immune response in a subject to an antigen, the use comprising:
(i) administering to the subject a first cell expressing a set of transgenes comprising two or more of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and
(ii) administering to the subject a second cell of the same type as the first cell that is modified to express a transgene encoding a polypeptide comprising the antigen and that does not express the set of transgenes.

148. Use of the cell of any one of claims 1-46 in the manufacture of a medicament for inducing immune tolerance in a subject to an antigen.

149. Use of the cell of any one of claims 1-7, 10-12, and 14-46 in the manufacture of a medicament for treating a subject having or at risk of developing an autoimmune disease or condition.

150. Use of the cell of any one of claims 1-9 and 14-46 in the manufacture of a medicament for preparing a subject to receive an organ or tissue transplant.

151. Use of a first cell in the manufacture of a medicament for inducing an immune response in a subject to an antigen, characterized in that the medicament is for administration in a treatment regimen with a second cell, wherein the first cell expresses a set of transgenes comprising two or more of the following genes: PD-L1, HLAG or H2-M3, Cd47, Cd200, FASLG or FasL, Ccl21 or Ccl21b, Mfge8, and Serpin B9 or Spi6 or a gene encoding a biologic that acts as an agonist of PD-L1, HLA-G or H2-M3, Cd47, Cd200, FASLG or FasL, Ccl21 or Ccl21b, Mfge8, or Serpin B9 or Spi6; and wherein the second cell is a cell of the same type as the first cell that does not express the set of transgenes and that is modified to express a transgene encoding a polypeptide comprising the antigen.
